(19) United States
(12) Patent Application Publication
(10) Pub. No.: US 2011/0054246 A1
(43) Pub. Date: Mar. 3, 2011

(54) WHOLE GENOME SCAN TO DISCOVER QUANTITATIVE TRAI LOCI (QTL) AFFECTING GROWTH, BODY COMPOSITION, AND REPRODUCTION IN MATERNAL PIG LINES

(76) Inventors: Archie C. Clutter, St. Louis, MO (US); Fengxing Du, Chesterfield, MO (US); Natasha Vukasinovic, St. Louis, MO (US)

(21) Appl. No.: 12/550,269

(22) Filed: Aug. 28, 2009

(51) Int. Cl. A61D 19/02; C12Q 1/68; C07H 21/00; C40B 40/06; A61D 19/00; A61D 19/04

(52) U.S. Cl. 600/34; 435/6; 536/22.1; 506/16; 600/33; 600/35

(57) ABSTRACT

The present invention provides compositions and methods useful for determining an animal's genotype and to select animals having a desired genotype. Embodiments of the invention also provide isolated nucleic acid molecules and analytical kits that may be used to determine an animal's genotype. Furthermore, the present invention provides methods for improving desirable animal traits including improved growth, enhanced body composition, and improved reproductive traits. The invention also provides methods for allocating animals for predetermined uses, for picking potential parent animals for breeding, and for producing progeny animals. The instant invention also provides methods for identifying associations between single nucleotide polymorphisms (SNPs) and phenotypic traits. Also provided are methods for identifying genetic markers in allelic association with the SNP disclosed herein and for identifying the genotypes of a causative mutation underlying a quantitative trait locus (QTL).

### INCORPORATION OF SEQUENCE LISTING

A sequence listing is contained in the file named “Maternal Traits Jumbo.ST25.txt” which is 1,236,992 bytes (1.24 megabytes) (measured in MS-Windows XP) and was created on Nov. 17, 2006 and is located in computer readable form on a compact disc (in accordance with 37 C.F.R. §1. 52(e) and 37 C.F.R. §1. 1.821) is enclosed herewith and incorporated herein by reference.

### FIELD OF THE INVENTION

The invention relates to the enhancement of desirable characteristics in swine. More specifically, it relates to genetic markers and methods for improving swine genetics.

### BACKGROUND OF THE INVENTION

Improvement in genetic potential for the number of piglets born per litter has great potential to increase the economic efficiency of an integrated pork production system. Because the majority of cost in pork production is associated with animal feed, an additional primary economic driver is genetic improvement in the growth rate and overall feed efficiency of animals produced for market. Unfortunately, prolificacy of reproducing females and efficiency traits of market animals often have unfavorable genetic correlations. Furthermore, although these traits all have some degree of underlying genetic variation in commercial pig populations, the accuracy of selecting breeding animals with superior genetic merit for many of them is low due to low heritability or the inability to measure the trait directly on the candidate animal. In particular, litter size has a heritability of only ˜10% and can only be measured after females for breeding are typical selected to be kept in the herd. Similarly, feed intake is expensive and difficult to measure on individual animals and is typically selected for via correlated (growth and body composition) traits.

Genomics offers the potential for greater improvement in traits of pork production and quality through the discovery of genes, or genetic markers linked to genes, that account for genetic variation and can be used for more direct and accurate selection. Although markers with associations with pig litter size and production efficiency traits have been reported (see Rothschild and Plastow, 1999, AgBioTechNet 10:1-8 for a review), each of these traits is extremely complex; consequently, it is likely that a large number of genes and/or markers per trait must be elucidated to create significant value in selection. Only through comprehensive and powerful scans of the entire porcine genome can this goal be fully achieved.

The pigs harvested for pork around the world are typically produced by crossing closed breeding populations having distinct characteristics for maternal productivity (i.e., prolificacy), growth efficiency, and meat quality. These closed populations typically have inbreeding levels of less than 50% and genomes scans yielding markers that can be used across the population for selection require densely-spaced genetic markers; preferably including markers on most or all of the porcine chromosomes. The application of such a marker map to discover and finely-map genes contributing to variation in reproductive traits (including prolificacy), growth and body composition traits is described herein. The large number of resulting linked markers can be used in several methods of marker or marker-assisted selection, including whole-genome selection (Meuwissen et al., Genetics 2001), to improve the genetic merit of the population for these traits and the value created in the pork production chain.

### SUMMARY OF THE INVENTION

This section provides a non-exhaustive summary of embodiments of the present invention.

Various embodiments of the invention provide isolated nucleic acids having the sequence of alleles comprising single nucleotide polymorphisms (SNPs) that are provided by the present invention. Aspects of these embodiments of the invention provide for isolated nucleic acids identical to all or a portion of the genomic sequences surrounding the SNPs (described in Tables 1 and 2), or identical to all or portion of the complementary strands of the genomic sequences surrounding the SNPs (described in Tables 1 and 2). For example the sequence may be 100% identical or greater than 99%, 98%, 97%, 96%, 95%, or 90% with all or a portion of the sequences described by Tables 1 and 2 and the SEQUENCE LISTING. Alternatively, the isolated nucleic acid sequence may be 100%, greater than 99%, greater than 98%, greater than 97%, greater than 96%, greater than 95%, or greater than 90% identical with a contiguous segment of 17 or more nucleotides, 25 or more, 50 or more, or 100 or more nucleotides.

Other embodiments of the invention provide methods for evaluating an animal's genotype at one or more positions in the animal's genome. In various aspects of these embodiments the animal's genotype is evaluated at a position within a segment of DNA (an allele) that contains at least one SNP selected from the SNPs described in the Tables and Sequence Listing of the present application.

Other embodiments of the invention provide methods for allocating animals for use based on their genotype (e.g. allocating for use as a breeding animal or to be sold for finishing and/or slaughter). Various aspects of this embodiment of the invention provide methods that comprise: a) analyzing the animal's genomic sequence at one or more alleles (where the alleles analyzed each comprise at least one SNP) to determine the animal's genotype at each of those alleles; b) analyzing the genotype determined for each allele to determine which allele of the SNP is present; c) allocating the animal for use based on its genotype at one or more of the alleles analyzed. Various aspects of this embodiment of the invention provide methods for allocating animals for use based on a favorable association between the animal's genotype, at one or more SNP alleles disclosed in the present application, and a desired phenotype. Alternatively, the methods provide for not allocating an animal for a certain use because it has one or more SNP alleles that are either associated with undesirable phenotypes or are not associated with desirable phenotypes.

Other embodiments of the invention provide methods for selecting animals for use in breeding to produce progeny. Various aspects of these methods comprise: A) determining the genotype of at least one potential parent animal at one or more locus/loci, where at least one of the loci analyzed contains an allele of a SNP selected from the group of SNPs described in Tables 1 and 2. B) Analyzing the determined genotype at one or more positions for at least one animal to determine which of the SNP alleles is present. C) Correlating the analyzed allele(s) with one or more phenotypes. D) Allocating at least one animal for use to produce progeny.

Other embodiments of the invention provide methods for producing offspring animals (progeny animals). Aspects of this embodiment of the invention provide methods that comprise: breeding an animal that has been selected for breeding by methods described herein to produce offspring. The offspring may be produced by purely natural methods or through the use of any appropriate technical means, including but not limited to: artificial insemination; embryo transfer (ET), multiple ovulation embryo transfer (MOET), in vitro fertilization (IVF), or any combination thereof.

Other embodiments of the invention provide for databases or groups of databases, each database comprising lists of the nucleic acid sequences, which lists include a plurality of the SNPs described in Tables 1 and 2. Preferred aspects of this embodiment of the invention provide for databases comprising the sequences for 50 or more SNPs. Other aspects of these embodiments comprise methods for using a computer algorithm or algorithms that use one or more database(s), each database comprising a plurality of the SNPs described in Tables 1 and 2 to identify phenotypic traits associated with the inheritance of one or more alleles of the SNPs, and/or using such a database to aid in animal allocation.

Still other embodiments of the invention provide diagnostic kits and/or arrays for detecting one or more of the SNPs described in Tables 1 and 2.

Further embodiments of the invention provide methods for identifying associations between one or more of the SNPs described in Tables 1 and 2 and one or more phenotypic traits.

Additional embodiments of the invention provide methods for identifying other genetic markers that are in allelic association with one or more of the SNPs described in Tables 1 and 2.

### DEFINITIONS

The following definitions are provided to aid those skilled in the art to more readily understand and appreciate the full scope of the present invention. Nevertheless, as indicated in the definitions provided below, the definitions provided are not intended to be exclusive, unless so indicated. Rather, they are preferred definitions, provided to focus the skilled artisan on various illustrative embodiments of the invention.

As used herein the term “allelic association” preferably means: nonrandom deviation of f(AᵢBⱼ) from the product of f(Aᵢ) and f(Bⱼ), which is specifically defined by r²>0.2, where r² is measured from a reasonably large animal sample (e.g., ≧100) and defined as

$${r}^{2} = \frac{{[ f ( {A}_{1} {B}_{1} ) - f ( {A}_{1} ) f ( {B}_{1} ) ]}^{2}}{f ( {A}_{1} ) ( 1 - f ( {A}_{1} ) ) ( f ( {B}_{1} ) ( 1 - f ( {B}_{1} ) )} \qquad ( Equation 1 )$$

where A₁ represents an allele at one locus, B₁ represents an allele at another locus; f(A₁B₁) denotes frequency of having both A₁ and B₁, f(A₁) is the frequency of A₁, f(B₁) is the frequency of B₁ in a population.

As used herein the terms “allocating animals for use” and “allocation for use” preferably mean deciding how an animal will be used within a herd or that it will be removed from the herd to achieve desired herd management goals. For example, an animal might be allocated for use as a breeding animal or allocated for sale as a non-breeding animal (e.g. allocated to animals intended to be sold for meat). In certain aspects of the invention, animals may be allocated for use in sub-groups within the breeding programs that have very specific goals (e.g. to improve meat color or carcass composition). Accordingly, even within the group of animals allocated for breeding purposes, there may be more specific allocation for use to achieve more specific and/or specialized breeding goals.

As used herein the terms “animal” or “animals” preferably refer to pigs/swine.

As used herein the terms “composition” and “body composition” preferably refer to measurements of the physical characteristics of the animal/pig and/or its carcass or to the characteristics themselves.

As used herein the term “growth” refers to the measurement of various parameters associated with an increase in an animal's size/weight.

As used herein the term “linkage disequilibrium” preferably means allelic association wherein A₁ and B₁ (as used in the above definition of allelic association) are present on the same chromosome.

As used herein the term “meat color” refers to measurements of color of meat obtained from a slaughtered animal.

As used herein the term “meat quality” refers measurements of the palatability and/or eating qualities (e.g. marbling, texture, and tenderness) of meat from a slaughtered animal.

As used herein the term “natural breeding” preferably refers to mating animals without human intervention in the fertilization process. That is, without the use of mechanical or technical methods such as artificial insemination or embryo transfer. The term does not refer to selection of the parent animals.

As used herein the term “prolificacy traits” preferably refers to traits including, but not limited to: the total number of fully-formed piglets born; the number of piglets born alive; the number of fully-formed stillborn piglets born; and the number of underdeveloped (mummified) piglets born dead.

As used herein the term “quantitative trait” is used to denote a trait that is controlled by multiple (two or more, and often many) genes each of which contributes small to moderate effect on the trait. The observations on quantitative traits often follow a normal distribution.

As used herein the term “quantitative trait locus (QTL)” is used to describe a locus that contains polymorphism that has an effect on a quantitative trait.

As used herein the term “reproductive material” includes, but is not limited to semen, spermatozoa, ova, and zygote(s).

As used herein the term “reproductive traits” includes, but is not limited to: fertility (e.g., ovulation rate, inter-farrowing interval, and number of services per conception), prolificacy, and maternal ability of a female animal (e.g., piglet survival and weaning weight).

As used herein the term “single nucleotide polymorphism” or “SNP” refer to a location in an animal's genome that is polymorphic within the population. That is, within the population some individual animals have one type of base at that position, while others have a different base. For example, a SNP might refer to a location in the genome where some animals have a “G” in their DNA sequence, while others have a “T”.

As used herein the terms “hybridization under stringent conditions” and “stringent hybridization conditions” preferably mean conditions under which a “probe” will hybridize to its target sequence to a detectably greater degree than to other sequences (e.g., at least 5-fold over background). Stringent conditions are target-sequence-dependent and will differ depending on the structure of the polynucleotide. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing).

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringency may also be adjusted with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaC1/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. The duration of hybridization is generally less than about 24 hours, usually about 4 to about 12 hours.

Specificity is typically the function of post-hybridization washes, the critical factors being the ionic strength and temperature of the final wash solution. For DNA-DNA hybrids, the thermal melting point (Tₘ) can be approximated from the equation of Meinkoth and Wahl (1984) Anal. Biochem. 138:267-284: Tₘ=81.5° C.+16.6 (log M)+0.41 (% GC)−0.61 (% form)-500/L; where M is the molarity of monovalent cations, % GC is the percentage of guanine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ hybridization, and/or wash conditions can be adjusted to hybridize to sequences of the desired identity. For example, if sequences with 90% identity are sought, the Tₘ can be decreased 10° C. Generally, stringent conditions are selected to be about 5° C. lower than the Tₘ for the specific sequence and its complement at a defined ionic strength and pH. However, highly stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-interscience, New York). See also Sambrook et al. (1989) Molecular Cloning: A Laboratory Manual (2d ed., Cold Spring Harbor Laboratory Press, Plainview, N.Y.).

### ILLUSTRATIVE EMBODIMENTS OF THE INVENTION

Various embodiments of the present invention provide methods for evaluating an animal's (especially a pig's) genotype at one or more positions in the animal's genome. Aspects of these embodiments of the invention provide methods that comprise determining the animal's genomic sequence at one or more locations (loci) that contain single nucleotide polymorphisms (SNPs). Specifically, the invention provides methods for evaluating an animal's genotype by determining which of two or more alleles for the SNP are present for one or more SNPs selected from the group consisting of the SNPs described in Tables 1 and 2 of the instant application.

In preferred aspects of these embodiments the animal's genotype is evaluated to determine which allele is present for 10 or more SNPs selected from the group of SNPs described in Tables 1 and 2. More, preferably the animal's genotype is determined for positions corresponding with 25, 50, 100, 200, 500, or 1000, or more of the SNPs described in Tables 1 and 2.

In other aspects of this embodiment, the animal's genotype is analyzed with respect to at least 1, 10, 25, 50, 100, 200, 500, or more SNPs that have been shown to be associated with growth, composition, or reproduction, or any combination thereof (see Table 1 for a list of the SNPs associated with these traits). For example, embodiments of the invention provides a method for genotyping for a single SNP or for genotyping 10 or more, 25 or more, 50 or more, 100 or more, 200 or more, or 500 or more SNPs that have been determined to be significantly associated with growth.

In any embodiment of the invention the genomic sequence at the SNP locus may be determined by any means compatible with the present invention. Suitable means are well known to those skilled in the art and include, but are not limited to direct sequencing, sequencing by synthesis, primer extension, Matrix Assisted Laser Desorption/Ionization-Time Of Flight (MALDI-TOF) mass spectrometry, polymerase chain reaction-restriction fragment length polymorphism, microarray/multiplex array systems (e.g. those available from Affimetrix, Santa Clara, Calif.), and allele-specific hybridization.

Other embodiments of the invention provide methods for allocating animals for subsequent use (e.g. to be used as sire/dams or to be sold for immediate slaughter or to be sold for finishing or any other use). Various aspects of this embodiment of the invention comprise determining at least one animal's genotype for at least one SNP selected from the group of SNPs described in Tables 1 and 2 (methods for determining animals' genotypes for one or more SNPs are described supra). Thus, the animal's allocation for use may be determined based on its genotype at one or more, 10 or more, 25 or more, 50 or more, 100 or more, 300 or more, or 500 or more of the SNPs described in Tables 1 and 2. The instant invention provides embodiments where analysis of the genotypes of the SNPs described in Tables 1 and 2 is the only analysis done. Other embodiments provide methods where analysis of the SNPs disclosed herein is combined with any other desired type of genomic or phenotypic analysis (e.g. analysis of any genetic markers beyond those disclosed in the instant invention). Moreover, the SNPs analyzed may be selected from those SNPs only associated with growth, only composition, or only reproduction, or the analysis may be done for SNPs selected from any desired combination of these traits (either by themselves or with other traits).

According to various aspects of these embodiments of the invention, once the animal's genetic sequence for the selected SNP(s) have been determined, this information is evaluated to determine which allele of the SNP is present for at least one of the selected SNPs. Preferably the animal's allelic complement for all of the determined SNPs is evaluated. Finally, the animal is allocated for use based on its genotype for one or more of the SNP positions evaluated. Preferably, the allocation is made taking into account the animal's genotype at each of the SNPs evaluated, but its allocation may be based on any subset or subsets of the SNPs evaluated.

The allocation may be made based on any suitable criteria. For any SNP, a determination may be made as to whether one of the allele(s) is associated/correlated with desirable characteristics or associated with undesirable characteristics. This determination will often depend on the animal breed/line, and on the breeding or other herd management goals. Determination of which alleles are associated with desirable phenotypic characteristics can be made by any suitable means. Methods for determining these associations are well known in the art; moreover, aspects of the use of these methods are generally described in the EXAMPLES, below.

Phenotypic traits that may be associated with the SNPs of the current invention include, but are not limited to: growth traits, body and/or carcass composition, meat quality, meat color, health traits (resistance to disease and injury and stress tolerance), reproduction traits such as litter size, survival of young, breeding efficiency, and length of reproductive life of the female.

According to various aspects of this embodiment of the invention allocation for use of the animal may entail either positive selection for the animals having the desired genotype(s) (e.g. the animals with the desired genotypes are selected for breeding purposes), negative selection of animals having undesirable genotypes (e.g. animals with an undesirable genotypes are culled from the herd), or any combination of these methods. According to preferred aspects of this embodiment of the invention animals identified as having SNP alleles associated with desirable phenotypes are allocated for use consistent with that phenotype (e.g. allocated for breeding based on phenotypes positively associated with improved growth). Alternatively, animals that do not have SNP genotypes that are positively correlated with the desired phenotype (or possess SNP alleles that are negatively correlated with that phenotype) are not allocated for the same use as those with a positive correlation for the trait.

Other embodiments of the invention provide methods for selecting potential parent animals (i.e., allocation for breeding). Various aspects of this embodiment of the invention comprise determining at least one animal's genotype for at least one SNP selected from the group of SNPs described in Tables 1 and 2. Furthermore, determination of whether and how an animal will be used as a potential parent animal may be based on its genotype at one or more, 10 or more, 25 or more, 50 or more, 100 or more, 300 or more, or 500 or more of the SNPs described in Tables 1 and 2. Moreover, as with other types of allocation for use, various aspects of these embodiments of the invention provide methods where the only analysis done is to genotype the animal for one or more of the SNPs described in Tables 1 and 2. Other aspects of these embodiments provide methods where analysis of one or more SNPs disclosed herein is combined with any other desired genomic or phenotypic analysis (e.g. analysis of any genetic markers beyond those disclosed in the instant invention). Moreover, the SNP(s) analyzed may all be selected from those associated only with growth, or only associated with composition, or only associated with reproductive traits. Conversely, the analysis may be done for SNPs selected from any desired combination of these or a combination of these traits with other traits.

According to various aspects of these embodiments of the invention, once the animal's genetic sequence at the site of the selected SNP(s) have been determined, this information is evaluated to determine which allele of the SNP is present for at least one of the selected SNPs. Preferably the animal's allelic complement for all of the sequenced SNPs is evaluated. Additionally, the animal's allelic complement is analyzed and correlated with the probability that the animal's progeny will express one or more phenotypic traits. Finally, the animal is allocated for breeding use based on its genotype for one or more of the SNP positions evaluated and/or based on the probability that it will pass the desired genotype(s)/allele(s) to its progeny. Preferably, the breeding allocation is made taking into account the animal's genotype at each of the SNPs evaluated. However, the animal's breeding allocation may be based on any subset or subsets of the SNPs evaluated.

The breeding allocation may be made based on any suitable criteria. For example, breeding allocation may be made so as to increase the probability of enhancing a single certain desirable characteristic in a population (in preference to other characteristics); alternatively, the selection may be made so as to generally maximize overall production based on a combination of traits. The allocations chosen are dependent on the breeding goals.

Other embodiments of the instant invention provide methods for producing progeny animals. According to various aspects of this embodiment of the invention, the animals used to produce the progeny are those that have been allocated for breeding according to any of the embodiments of the current invention. Those using the animals to produce progeny may perform the necessary analysis or, alternatively, those producing the progeny may do so using animals where the analysis is done by another. The progeny may be produced by any appropriate means, including, but not limited to using: (i) natural breeding, (ii) artificial insemination, (iii) in vitro fertilization (IVF) or (iv) collecting semen/spermatozoa and/or at least one ovum from the animal and contacting it, respectively with ova/ovum or semen/spermatozoa from a second animal to produce a conceptus by any means.

According to preferred aspects of this embodiment of the invention the progeny are produced by a process comprising natural breeding. In other aspects of this embodiment the progeny are produced through a process comprising the use of standard artificial insemination (AI), in vitro fertilization, multiple ovulation embryo transfer (MOET), deep intrauterine insemination (DIUI), or any combination thereof.

Other embodiments of the invention provide for methods that comprise allocating an animal for breeding purposes and collecting/isolating genetic material from that animal: wherein genetic material includes but is not limited to: semen, spermatozoa, ovum, zygotes, blood, tissue, serum, DNA, and RNA.

It is understood that most efficient and effective use of the methods and information provided by the instant invention employ computer programs and/or electronically accessible databases that comprise all or a portion of the sequences disclosed in the instant application. Accordingly, the various embodiments of the instant invention provide for databases comprising all or a portion of the sequences corresponding to at least 10 SNPs described in Tables 1 and 2. In preferred aspect of these embodiments the databases comprise sequences for 25 or more, 50 or more, 100 or more, 200 or more, 500 or more, 1000 or more, or substantially all of the SNPs described in Tables 1 and 2.

It is further understood that efficient analysis and use of the methods and information provided by the instant invention will employ the use of automated genotyping; particularly when large numbers (e.g. 100s) of markers are evaluated. Any suitable method known in the art may be used to perform such genotyping, including, but not limited to the use of micro-arrays.

Other embodiments of the invention provide methods wherein one or more of the SNP sequence databases described herein are accessed by one or more computer-executable programs. Such methods include, but are not limited to, use of the databases by programs to analyze for an association between the SNP and a phenotypic trait, or other user-defined trait (e.g. traits measured using one or more metrics such as gene expression levels, protein expression levels, or chemical profiles), and programs used to allocate animals for breeding or market.

Other embodiments of the invention provide methods comprising collecting genetic material from an animal that has been allocated for breeding. Wherein the animal has been allocated for breeding by any of the methods disclosed as part of the instant invention.

Other embodiments of the invention are drawn to isolated nucleic acids comprising 17 or more contiguous nucleotides corresponding to any one of the SNP sequences described in Tables 1 and 2 (i.e. any of SEQ ID NO:1-2601 and the Tables). In preferred aspects of this embodiment of the invention the isolated nucleic acid comprises a nucleotide corresponding with the polymorphic site in the sequence (i.e. it contains the site that defines the SNP's polymorphism as described in Tables 1 and 2).

Other embodiments of the invention provide isolated nucleic acid molecules comprising 100 or more contiguous nucleotides which are at least 90 percent identical with any of SEQ ID NO:1-2601. In preferred aspects of this embodiment of the invention the isolated nucleic comprises the polymorphic site for one or more SNPs selected from the group of SNPs described in Tables 1 and 2.

Other embodiments of the invention provide for diagnostic kits or other diagnostic devices for determining which allele of a SNP is present in a sample; wherein the SNP(s) are selected from the group of SNPs described in Tables 1 and 2. In various aspects of this embodiment of the invention, the kit or device provides reagents/instruments to facilitate a determination as to whether nucleic acid corresponding to the SNP is present. Such kit/or device may further facilitate a determination as to which allele of the SNP is present. In certain aspects of this embodiment of the invention the kit or device comprises at least one nucleic acid oligonucleotide suitable for DNA amplification (e.g. through polymerase chain reaction). In other aspects of the invention the kit or device comprises a purified nucleic acid fragment capable of specifically hybridizing, under stringent conditions, with at least one allele of at least one SNP described in Tables 1 and 2.

In particularly preferred aspects of this embodiment of the invention the kit or device comprises at least one nucleic acid array (e.g. DNA micro-arrays) capable of determining which allele of one or more of the SNPs described in Tables 1 and 2 is present in a sample. Preferred aspects of this embodiment of the invention provide DNA micro-arrays capable of simultaneously determining which allele is present in a sample for 10 or more SNPs. Preferably, the DNA micro-array is capable of determining which SNP allele is present in a sample for 25 or more, 50 or more, 100 or more, 200 or more, 500 or more, or 1000 or more SNPs. Methods for making such arrays are known to those skilled in the art and such arrays are commercially available (e.g. from Affimetrix, Santa Clara, Calif.).

Other embodiments of the present invention provide methods for identifying, within a plurality of animals, an association between one or more of the SNPs described in Tables 1 and 2 and at least one phenotypic trait. In various aspects of this embodiment of the invention the method comprises: (a) measuring one or more phenotypic traits in a plurality of animals; (b) determining the genotype for at least one SNP for a plurality of animals, wherein at least one SNP genotype determined is selected from the group of SNPs described in Tables 1 and 2; and (c) statistically correlating the presence of at least one phenotypic trait with the presence of an allele of at least one SNP selected from the group of SNPs described in Tables 1 and 2; wherein the presence of different alleles for that SNP is associated with different phenotypic attributes or with distinguishable phenotypic variation. That is, where the presence of different alleles, for at least one SNP, is associated with a statistically distinct phenotypic difference in the animals possessing them. The distinct phenotype may be detectable either between individual animals or it may be detectable when pluralities of the animals having different genotypes are compared as groups.

Other embodiments of the instant invention provide methods of identifying genetic markers that are in allelic association with one or more of the SNPs described in Tables 1 and 2. According to various aspects of this embodiment of the invention the method comprises: (a) identifying a marker, B₁, that is suspected of being in allelic association with at least one marker, A₁, wherein A₁ is selected from the group of SNPs described in Tables 1 and 2; (b) determining whether A₁ and B₁ are in allelic association; wherein allelic association is determined to exist if r²>0.2 for Equation 1 for a population sample of at least 100 animals and wherein Equation 1 is:

$${r}^{2} = \frac{{[ f ( {A}_{1} {B}_{1} ) - f ( {A}_{1} ) f ( {B}_{1} ) ]}^{2}}{f ( {A}_{1} ) ( 1 - f ( {A}_{1} ) ) ( f ( {B}_{1} ) ( 1 - f ( {B}_{1} ) )} \qquad [ Equation 1 ]$$

and wherein for Equation 1 A₁ represents an allele at one locus (A SNP described by Tables 1 and 2); B₁ represents a genetic marker at another locus; f(A₁B₁) denotes frequency of having both A₁ and B₁; f(A₁) is the frequency of A₁ in the population, f(B₁) is the frequency of B₁ in the population. In preferred aspects of this embodiment of the invention B₁ is a SNP. Even more preferred aspects of this embodiment of the invention provide methods for identifying genetic markers in linkage disequilibrium with one or more SNPs selected from the group of SNPs described in Tables 1 and 2. In other aspects of these embodiments of the invention the value of r² is greater than 0.5, greater than 0.7, or greater than 0.9.

Additional embodiments of the invention provide for genetic markers that are in allelic association with one or more of the SNPs described in Tables 1 and 2. Markers provided as part of this embodiment of the invention may be identified by any suitable means known to those of ordinary skill in the art. A marker falls within this embodiment of the invention if it is determined to be in allelic association with one or more of the SNPs described in Tables 1 and 2 as defined by Equation 1, supra, where r² is greater than 0.2, greater than 0.5, greater than 0.7, or greater than 0.9. In preferred aspects of these embodiments of the invention the markers are in linkage disequilibrium with one or more of the SNPs described in Tables 1 and 2.

Genetic markers that are in allelic association with any of the SNPs described in the Tables may be identified by any suitable means known to those skilled in the art. For example, a genomic library may be screened using a probe specific for any of the sequences of the SNPs described in the Tables. In this way clones comprising at least a portion of that sequence can be identified and then up to 300 kilobases of 3′ and/or 5′ flanking chromosomal sequence can be determined. By this means, genetic markers in allelic association with the SNPs described in the Tables will be identified.

Other embodiments of the present invention provide methods for identifying genes that may be associated with phenotypic variation. According to various aspects of these embodiments, the chromosomal location of a SNP associated with a particular phenotypic variation can be determined, by means well known to those skilled in the art. Once the chromosomal location is determined genes suspected to be involved with determination of the phenotype can be analyzed. Such genes may be identified by sequencing adjacent portions of the chromosome or by comparison with analogous section of the human genetic map (or known genetic maps for other species). An early example of the existence of clusters of conserved genes is reviewed in Womack (1987), where genes mapping to the same chromosome in one species were observed to map to the same chromosome in other, closely related, species. As mapping resolution improved, reports of the conservation of gene structure and order within conserved chromosomal regions were published (for example, Grosz et al, 1992). More recently, large scale radiation hybrid mapping and BAC sequence have yielded chromosome-scale comparative mapping predictions between human and bovine genomes (Everts-van der Wind et al., 2005), between human and porcine genomes (Yasue et al., 2006) and among vertebrate genomes (Demars et al., 2006)

Other embodiments of the invention provide methods for identifying causal mutations that underlie one or more quantitative trait loci (QTL). Various aspects of this embodiment of the invention provide for the identification QTL that are in allelic association with one or more of the SNPs described in Tables 1 and 2. Once these SNPs are identified, it is within the ability of skilled artisans to identify mutations located proximal to such SNP(s). Further, one skilled in the art can identify genes located proximate to the identified SNP(s) and evaluate these genes to select those likely to contain the causal mutation. Once identified these genes and the surrounding sequence can be analyzed for the presence of mutations, in order to identify the causal mutation.

### EXAMPLES

The following examples are included to demonstrate general embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the invention.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied without departing from the concept and scope of the invention.

### Example 1

### Determining Associations Between Genetic Markers and Phenotypic Traits

Simultaneous discovery and fine-mapping on a genome-wide basis of genes underlying quantitative traits (Quantitative Trait Loci: QTL) requires genetic markers densely covering the entire genome. As described in this example, a whole-genome, dense-coverage marker map was constructed from microsatellite and single nucleotide polymorphism (SNP) markers with previous estimates of location in the pig genome, and from SNP markers with putative locations in the pig genome based on homology with human sequence and the human/pig comparative map. A new linkage-mapping software package was developed, as an extension of the CRIMAP software (Green et al., Washington University School of Medicine, St. Louis, 1990), to allow more efficient mapping of densely-spaced markers genome-wide in a pedigreed livestock population (Liu and Grosz Abstract C014; Grapes et al. Abstract W244; 2006 Proceedings of the XIV Plant and Animal Genome Conference, www.intl-pag.org). The new linkage mapping tools build on the basic mapping principles programmed in CRIMAP to improve efficiency through partitioning of large pedigrees, automation of chromosomal assignment and two-point linkage analysis, and merging of sub-maps into complete chromosomes. The resulting whole-genome discovery map (WGDM) included ˜5,926 markers and a map length of 2,373.6 cM for an average map density of 2.41 markers/cM. The average gap between markers was 0.44 cM and the largest gap was 11.1 cM. This map provided the basis for whole-genome discovery and fine-mapping of QTL contributing to variation in pig growth and carcass composition, and in female prolificacy traits.

### Discovery and Mapping Populations

Discovery and mapping populations can take many forms. The most effective for determining population-wide marker/QTL associations include a large and genetically diverse sample of individuals with phenotypic measurements of interest collected in a design that allows accounting for non-genetic effects and includes information regarding the pedigree of the individuals measured. In the present example, four populations were used to discovery and map QTL: two populations derived from different sources of the Landrace breed (England and Continental Europe), and two populations derived from different sources of the Large White breed (England and Continental Europe). Tissue samples for DNA extraction were collected from a total of approximately 3,000 animals from the aforementioned populations representing approximately equal number of samples per population; and representing multiple sire and grandsire families from each population. DNA samples were also collected from the sire of each progeny, and in some cases from additional members of the pedigree.

The female progeny sampled from these populations had been measured for several phenotypic traits including average daily gain in weight, ultrasonically-measured fat thickness and loin muscle area, number of piglets born alive, number of (fully-formed) stillborn piglets and number of mummified (underdeveloped) piglets born dead. DNA samples collected from the female progeny and members of the pedigree were genotyped for approximately 4,300 SNP markers that are distributed across the WGDM and that were previously determined to be informative for mapping purposes in these populations. For each animal, and each SNP marker, a genotype is generated reflecting the alleles present on the chromosome pair at that locus (e.g., AA, AB or BB) and is used in subsequent marker/trait association analyses.

### Phenotypic Analyses

The phenotypic measurements on individual animals for growth, body composition and litter traits were subjected to standard statistical procedures to remove erroneous and biologically impossible data, and to account for non-genetic sources of variation. For example in a first step, upper and lower biological limits can be determined for each trait by examining the upper and lower 2.5% and excluding data that are known to exceed the range of biological possibilities for the trait. In a second step, a general linear statistical model can be used for each trait that includes adjustment for known potential non-genetic effects. For example, all litter traits can be adjusted for the on-farm contemporary group to which the animal belonged (season-year-farm-building), the age at which the animal was first bred, the length of the pregnancy and the parity (i.e., first litter, second litter, etc.). Measurements of periodic growth rate can be adjusted for the beginning age of the animal and the number of days measured. Body composition measurements (e.g., lean percentage) can be adjusted for the weight of the animal at measurement. After adjustments for these non-genetic factors, the data can again be examined for outliers and values with a residual standard deviation of 4.0 or more excluded from analyses.

### QTL Discovery and Mapping Analyses

Mapping the location of QTL through associations between marker genotypes and phenotypic traits (pre-adjusted for non-genetic effects) can be approached in several ways, including simple regressions of each trait on the genotype for each SNP marker (e.g., trait regressed on number of “A” alleles: 0, 1, or 2) or analyses of families within the population to detect linkage between markers and QTL. The marker/trait associations most useful as animal selection tools are those of markers tightly linked to a QTL and based on marker/QTL allele configurations that are present in most individuals across the population.

These types of population-wide results can be obtained from linkage-disequilibrium (LD) mapping. When a mutation that can cause a trait difference first occurs, that mutation is in complete LD with all other alleles on that chromosome. Over time recombination breaks down the LD, but there is a relatively small frequency of recombination between the QTL and loci in very close proximity. Hence, individuals that share an allele of the QTL transmitted from some common ancestor (Identical By Decent: IBD) are likely to share alleles of closely linked markers. By using ordered marker genotypes along the chromosome (marker haplotypes with known parental origin) to determine the probabilities at each locus that individuals with phenotypic data are IBD, marker alleles in population-wide linkage with valuable QTL alleles can be identified.

In the present example, LD mapping was performed in the aforementioned discovery populations using statistical analyses that require knowledge of ordered genotypes. Ordered genotypes of an individual can be inferred partially or with certainty conditional on pedigree information and observed genotype data (Du and Hoeschele, 2000). With simple pedigrees such as a nuclear family and small numbers of linked markers, exact calculation of the probabilities of linkage phases is achievable, by evaluating all possible genotype configurations. The exact calculation quickly becomes infeasible as the size and complexity of the pedigree and number of linked markers increases. For example, there are totally 2^{k-}1 linkage phases when a parent has k linked heterozygous loci.

In part due to this computational difficulty, most methods have focused on identifying the most probable linkage phase. Windig and Meuwissen (2004) provide a computationally efficiency method for phase construction. They start with a likely phase derived with a simple rule based method, calculate the parental origin of the two alleles at a marker, and perform a switch if the probability of the switch is greater than 0.5. The process is repeated until the probability of a switch is less than 0.5 for all linked markers. In the present example, probabilities of parental linkage phases for the entire set of linked loci were determined by choosing small subsets of markers (e.g., 5 to 10) and considering all possible phases for these markers by calculating their probability conditional on the observed genotypes for all markers under evaluation. Only phases with likelihood ratio greater than some pre-specified threshold are retained, and the iteration of the method over the entire marker set produces a collection of likely phases and their probabilities conditional on the observed data.

To determine associations between haplotype probabilities and trait phenotypes, haplotypes of neighboring (and/or non-neighboring) markers across each chromosome were defined by setting the maximum length of a chromosomal interval and minimum and maximum number of markers to be included. Clearly, one needs to set similar parameters to form or define groups of marker loci for haplotype evaluation. The association between pre-adjusted trait phenotypes and haplotype was evaluated via a regression approach with the following model:

$${Y}_{k} = {\sum }_{i} {β}_{i} [ P ( {H}_{sik} ) + P ( {H}_{dik} ) ] + {e}_{k} \qquad [ 1 ]$$

where Yₖ is the preadjusted phenotype of one of the traits described above for individual k, and eₖ is the residual; P(Hₛᵢₖ) and P(H_{dik}) are the probability of paternal and maternal haplotype of individual k being haplotype i; P(Hₛᵢₖ, H_{dik}) is the probability of individual k has paternal haplotype i and maternal haplotype j; all β are corresponding regression coefficients.

Least-squares methods were used to estimate the effect of a haplotype on a phenotypic trait and the regular F-test used to test the significance of the effect. Permutation tests were performed based on phenotype permutation (10,000 to 50,000) within the whole sample for Type I error rate (p value) estimation.

### Example 2

### Use of Single Nucleotide Polymorphisms to Improve Offspring Traits

To improve the average genetic merit of a population for a chosen trait, one or more of the markers with significant association to that trait can be used in selection of breeding animals. In the case of each discovered locus, use of animals possessing a marker allele (or a haplotypes of multiple marker alleles) in population-wide LD with a favorable QTL allele will increase the breeding value of animals used in breeding, increase the frequency of that QTL allele in the population over time and thereby increase the average genetic merit of the population for that trait. This increased genetic merit can be disseminated to commercial populations for full realization of value.

For example, a closed genetic nucleus (GN) population of pigs may be maintained to produce boars and semen for use in a commercial maternal multiplier. The commercial maternal multiplier may use, for example, semen from Landrace boars to breed Large White females to produce Landrace x Large White females. These Landrace x Large White females can be bred with semen from a Terminal Boar Line to produce commercial market hogs. One-half the increased genetic merit in the Landrace GN from selection on genetic markers for litter traits will be expressed in the Landrace x Large White females, and ¼ of the increased genetic merit in the Landrace GN from selection on genetic markers for growth and leanness will be expressed in the commercial market hogs. A GN herd of 400 sows would be expected to produce approximately 8,000 offspring per year (4,000 boar offspring per year), typically according to a weekly breeding and farrowing schedule. If one assumes in this example that only semen from the highest ranking ½ of 4,000 GN boars produced is used to breed females in the multiplier herd, 2,000 Landrace GN boars per year are available to breed Large White females. At a service ratio of 200 sows serviced per boar, markers-assisted selection in the Landrace GN herd of 400 sows impacts the genetic merit for litter size traits of 400,000 Landrace x Large White females, and impacts genetic merit for growth and leanness of approximately 8,000,000 market hogs (assumes each of the 400,000 Landrace x Large White sows produces 20 market pigs per year).

The first step in using a SNP for estimation of breeding value and selection in the GN is collection of DNA from all offspring that will be candidates for selection as breeders in the GN or as breeders in other commercial populations (in the present example, the 8,000 offspring produced in the GN each year). One method is to capture shortly after farrowing a small bit of tail tissue from each piglet into a labeled (bar-coded) tube. The DNA extracted from this tissue can be used to assay an essentially unlimited number of SNP markers and the results can be included in selection decisions before the animal reaches breeding age.

One method for incorporating into selection decisions the markers (or marker haplotypes) determined to be in population-wide LD with valuable QTL alleles (see Example 1) is based on classical quantitative genetics and selection index theory (Falconer and Mackay, 1996; Dekkers and Chakraborty, 2001). To estimate the effect of the marker in the population targeted for selection, a random sample of animals with phenotypic measurements for the trait of interest can be analyzed with a mixed animal model with the marker fitted as a fixed effect or as a covariate (regression of phenotype on number of allele copies). Results from either method of fitting marker effects can be used to derive the allele substitution effects, and in turn the breeding value of the marker:

α₁=q[a+d(q−p)] [2]

α₂=−p[a+d(q−p)] [3]

α=a+d(q−p) [4]

g_{A1A1}=2(α₁) [5]

g_{A1A2}=(α₁)+(α₂) [6]

g_{A2A2}=2(α₂) [7]

where α₁ and α₂ are the average effects of alleles 1 and 2, respectively; U is the average effect of allele substitution; p and q are the frequencies in the population of alleles 1 and 2, respectively; a and d are additive and dominance effects, respectively; g_{A1A1}, g_{A1A2} and g_{A2A2} are the (marker) breeding values for animals with marker genotypes A1A1, A1 A2 and A2A2, respectively. The total trait breeding value for an animal is the sum of breeding values for each marker (or haplotype) considered and the residual polygenic breeding value:

EBVᵢⱼ=Σĝⱼ+Û [8]

where EBVᵢⱼ is the Estimated Trait Breeding Value for the i^{th} animal, Σĝⱼ is the marker breeding value summed from j=1 to n where n is the total number of markers (haplotypes) under consideration, and Ûᵢ is the polygenic breeding value for the i^{th} animal after fitting the marker genotype(s).

These methods can readily be extended to estimate breeding values for selection candidates for multiple traits, the breeding value for each trait including information from multiple markers (haplotypes), all within the context of selection index theory and specific breeding objectives that set the relative importance of each trait. Other methods also exist for optimizing marker information in estimation of breeding values for multiple traits, including random models that account for recombination between markers and QTL (e.g., Fernando and Grossman, 1989), and the potential inclusion of all discovered marker information in whole-genome selection (Meuwissen et al., Genetics 2001). Through any of these methods, the markers reported herein that have been determined to be in population-wide LD with valuable QTL alleles may be used to provide greater accuracy of selection, greater rate of genetic improvement, and greater value accumulation in the pork production chain.

### Example 3

### Identification of SNP

A nucleic acid sequence contains a SNP of the present invention if it comprises at least 20 consecutive nucleotides that include and/or are adjacent to a polymorphism described in Tables 1 and 2 and the Sequence Listing. Alternatively, a SNP of the present invention may be identified by a shorter stretch of consecutive nucleotides which include or are adjacent to a polymorphism which is described in Tables 1 and 2 and the Sequence Listing in instances where the shorter sequence of consecutive nucleotides is unique in the pig genome. A SNP site is usually characterized by the consensus sequence in which the polymorphic site is contained, the position of the polymorphic site, and the various alleles at the polymorphic site. “Consensus sequence” means DNA sequence constructed as the consensus at each nucleotide position of a cluster of aligned sequences. Such clusters are often used to identify SNP and Indel (insertion/deletion) polymorphisms in alleles at a locus. Consensus sequence can be based on either strand of DNA at the locus, and states the nucleotide base of either one of each SNP allele in the locus and the nucleotide bases of all Indels in the locus, or both SNP alleles using degenerate code (IUPAC code: M for A or C; R for A or G; W for A or T; S for C or G; Y for C or T; K for G or T; V for A or C or G; H for A or C or T; D for A or G or T; B for C or G or T; N for A or C or G or T; Additional code that we use include I for “-” or A; 0 for “-” or C; E for “-” or G; L for “-” or T; where “-” means a deletion). Thus, although a consensus sequence may not be a copy of an actual DNA sequence, a consensus sequence is useful for precisely designing primers and probes for actual polymorphisms in the locus.

Such SNP have a nucleic acid sequence having at least 90% sequence identity, more preferably at least 95% or even more preferably for some alleles at least 98% and in many cases at least 99% sequence identity, to the sequence of the same number of nucleotides in either strand of a segment of pig DNA which includes or is adjacent to the polymorphism. The nucleotide sequence of one strand of such a segment of pig DNA may be found in a sequence in the group consisting of SEQ ID NO:1 through SEQ ID NO:5211. It is understood by the very nature of polymorphisms that for at least some alleles there will be no identity at the polymorphic site itself. Thus, sequence identity can be determined for sequence that is exclusive of the polymorphism sequence. The polymorphisms in each locus are described in Tables 1 and 2.

Shown below are examples of public porcine SNPs that match each other: SNP ss 6337002 (SEQ ID NO:2602) was determined to be the same as ss16337593 (SEQ ID NO:2603) because 41 bases (with the polymorphic site at the middle) from each sequence match one another perfectly (match length=41, identity=100%).

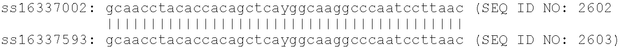

SNP ss16337555 (SEQ ID NO:2604) was determined to be the same as ss16338279 (SEQ ID NO:2605) because 41 bases (with the polymorphic site at the middle) from each sequence match one another at all bases except for one base (match length=41, identity=97%).

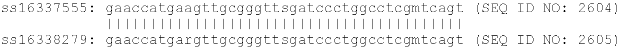

### Example 4

### Quantification of the Animal's Body and/or Carcass Composition

Quantifying an animal's body and/or carcass composition can be accomplished by measuring a number of phenotypes including, but not limited to: average backfat thickness (e.g. in inches or centimeters) measured on the carcass or on the live animal using ultrasound; loin muscle (eye) area (e.g. in²) measured on the carcass or on the live animal using ultrasound; fat depth, loin depth (millimeters) and carcass weight (pounds) measured on-line after slaughter using industry standard equipment (e.g., Fat-O-Meater); predicted lean percentage (%) calculated from backfat thickness, loin muscle depth or area, and body or carcass weight; carcass length (e.g. in inches); National Pork Producers Council (NPPC) marbling quality score (i.e., 1-5); Intramuscular fat content of the loin (%); primal cuts (loin, shoulder, belly, etc) as a percent of carcass weight (%); carcass yield as a percentage of live weight (%).

### Example 5

### Quantification of the Animal's Growth

Quantifying an animal's growth can be accomplished by measuring phenotypes including, but not limited to: average daily weight gain (e.g. lbs/day); average daily feed intake (lbs/day); feed conversion ratio (lbs feed per lb gain); growth from birth to any desired age (lb); and growth during a specified test period (e.g. from 21 to 165 days of age).

### Example 6

### Quantification of Meat Color

Quantifying meat color can be accomplished by measuring phenotypes including, but not limited to: Hunter colorimeter scores; change in Hunter colorimeter score over a 24 hour period; Japanese color score (1-5); and NPPC color quality score (1-5).

### Example 7

### Quantification of Meat Quality

Quantifying meat quality can be accomplished by measuring phenotypes including, but not limited to: Drip loss (%); purge loss (%); pH of loin or ham; moisture content in the loin (%); NPPC firmness quality score (1-5); Werner-Bratzler shear force (kg); and cooking loss (%).

### Example 8

### Quantification of the Animal's Prolificacy

Quantifying an animal's prolificacy can be accomplished by measuring phenotypes including, but not limited to (each trait measured within 24 hr of parturition): total number of fully-formed piglets born, number of piglets born alive, number of fully-formed but stillborn piglets, and number of (underdeveloped) mummified piglets born dead.

### REFERENCES

The references cited in this application, both above and below, are specifically incorporated herein by reference.

### Non-Patent Literature

Buys, N., G. Van den Abeele, A. Stinckens, J. Deley, M. Georges (2006): Effect of the IGF2-intron3-G3072A mutation on prolificacy in sows. Proc. 8^{th} World Cong. Genet. Appl. Livest. Prod., Belo Horizonte, Brazil.
Campbell, E. M. G., D. Normeman, G. A. Rohrer (2003): Fine mapping a quantitative trait locus affecting ovulation rate in swine on chromosome 8. J. Anim. Sci. 81:1706-1714.
Cassady, J. P., R. K. Johnson, D. Pomp, G. A. Rohrer, L. D. Van Vleck, E. K. Spiegel, K. M. Gilson (2001): Identification of quantitative trait loci affecting reproduction in pigs. J. Anim. Sci. 79:623-633.
Ciobanu, D C, Bastiaansen, J W M, Longergan, S M, Thomsen, H, Dekkers, J C M, Plastow, G S, and Rothschild, M F, (2004) J. Anim. Sci. 82:2829-39.
Davis, G P and DeNise, S K, (1998) J. Anim. Sci. 76:2331-39.
Dekkers, J C M, and Chakraborty, R, (2001) J. Anim. Sci. 79:2975-90.
Demars J, Riquet J, Feve K, Gautier M, Morisson M, Demeure O, Renard C, Chardon P, Milan D. (2006), BMC Genomics, 24:7-13
Du and Hoeschele, (2000) Genetics 156:2051-62.
Estany, J., D. Villalba, J. Tibau, J. Soller, D. Babot, J. L. Noguera (2002): Correlated response to selection for litter size in pigs: I. Growth, fat deposition, and feeding behavior traits. J. Anim. Sci. 80:2556-2565.
Everts-van der Wind A, Larkin D M, Green C A, Elliott J S, Olmstead C A, Chiu R, Schein J E, Marra M A, Womack J E, Lewin H A. (2005) Proc Natl Acad Sci USA, 20; 102(51):18526-31.
Falconer, D S, and Mackay, T F C, (1996) Introduction to Quantitative Genetics. Harlow, UK: Longman.
Fernando, R, and Grossman, M, (1989) Marker assisted selection using best linear unbiased prediction. Genetics Selection Evolution 21:467-77.
Franco, M M, Antunes, R C, Silva, H D, and Goulart, L R (2005) J. Appl. Genet. 46(2):195-200.
Grosz, M D, Womack, J E, and Skow, L C (1992) Genomics, 14(4):863-868.
Hayes, B, and Goddard, M E, (2001) Genet. Sel. Erol. 33:209-229.
Holl J, N. Vukasinovic, A. C. Clutter, S. D. Kachman, and R. K. Johnson (2006): Fine mapping to confirm andidentify reproductive QTL in research pig population. Proc. 8^{th} World Cong. Genet. Appl. Livest. Prod., Belo Horizonte, Brazil.
King, A. H., Z. Jiang, J. P. Gibson, C. S. Haley, A. L. Archibald (2003): Mapping quantitative trait loci affecting female reproductive traits on porcine chromosome 8. Biol. Reprod. 68:2172-2179.
Kwok P Y, Methods for genotyping single nucleotide polymorphisms, (2001), Anna. Rev. Genomics Hum. Genet., 2:235-258.
Linville, R. C., D. Pomp, R. K. Johnson, M. F. Rotschild (2001): Candidate gene analysis for loci affecting litter size and ovulation rate in swine. J. Anim. Sci. 79:60-67.
Meuwissen, T H E, Hayes, B J, and Goddard, M E, (2001) Genetics. 157:1819-29.
Rohrer, G. A., J. J. Ford, T. H. Wise, J. L. Vallet, R. K. Christenson (1999): Identification of quantitative trait loci affecting female reproductive traits in a multigenerational Meishan-White composite swine population. J. Anim. Sci. 77:1385-1391.
Rothschild, M. F. and G. S. Plastow (1999): Advances in pig genomics and industry applications. AgBioTechNet 10:1-8.
Rothschild, M. F., C. Jacoson, D. Vaske, C. Tuggle, L. Wang, T. Short, G. Eckardt, S. Sasaki, A. Vincent, D. McLaren, O. Southwood, H. van der Stein, A. Mileham, G. Plastow (1996): Proc. Nat. Acad. Sci. 93:201-205.
Rothschild and Plastow, (1999), AgBioTechNet 10:1-8
Short, T H, et al. (1997) J. Anim. Sci. 75:3138-3142.
Spelman, R J and Bovenhuis, H, (1998) Animal Genetics, 29:77-84.
Stearns, T M, Beever, J E, Southey, B R, Ellis, M, McKeith, F K and Rodriguez-Zas, S L, (2005) J. Anim. Sci. 83:1481-93.
Syvänen A C, Accessing genetic variation: genotyping single nucleotide polymorphisms, (2001) Nat. Rev. Genet. 2:930-942.
Verrier, E, (2001) Genet. Sel. Evol. 33:17-38.
Villanueva, B, Pong-Wong, R, Fernandez, J, and Toro, M A (2005) J. Anim. Sci. 83:1747-52.
Williams, J L, (2005), Rev. Sci. Tech. Off Int. Epiz. 24(1):379-391.
Windig, J J, and Meuwissen, T H E, (2004) J. Anim. Breed. Genet. 121:26-39.
Womack, J, (1987), Dev. Genet. 8(4):281-293.
Yasue H, Kiuchi S, Hiraiwa H, Ozawa A, Hayashi T, (2006), Cytogenet. Genome Res., 112(1-2):121-125.
Youngerman, S M, Saxton, A M, Oliver, S P, and Pighetti, G M, (2004) J. Dairy Sci. 87:2442-2448.

### Patent Literature

| Patent/Publication | | | Pub. |
|---|---|---|---|
| Number | Title | Inventors | Date |
| US2003129610A1 | New QTL's on chromosomes x, 2, 6 | de Koning, Dirk-Jan; Groenen, | Jul. 10, 2003 |
| | and 7 of pigs | Martinus Antonius, M.; van | |
| | | Arendonk, Johannus Antonius, M. | |
| US2003211512A1 | Novel ghrelin alleles and use of the | Rothschild, Max F.; Kim, | Nov. 13, 2003 |
| | same for genetically typing animals | Kwan-Suk; Anderson, Lloyd L. | |
| US2004029145A1 | Novel HMGA alleles and use of the | Rothschild, Max F.; Kim, | Feb. 12, 2004 |
| | same as genetic markers for growth, | Kwan-Suk; Nguyen, Nguyet | |
| | fatness, meat quality, and feed | Thu | |
| | efficiency traits | | |
| US2004101842A1 | Method to screen for improved meat | Haley, Christoper Simon; | May 27, 2004 |
| | characteristics in pigs | Matlin, Charlotte Anne | |
| US2004253594A1 | Genetic markers for improved | Tuggle, Christopher K; | Dec. 16, 2004 |
| | disease resistance in animals | Marklund, Lena; Stabel, Thomas J; | |
| | (nramp1) | Mellencamp, Martha A; | |
| | | Stumbaugh, Ambar | |
| US2004261138A1 | Genetic markers for improved meat | Rothschild, Max F.; Kim, Kwan | Dec. 23, 2004 |
| | characteristics in animals (mc4r) | Suk; Emnett, Rebecca S. | |
| US2005112648A1 | Genetic markers for pig backfat | Lin, En-Chung; Chen, Ming- | May 26, 2005 |
| | thickness | Yu; Huang, San-Yuan; Tsou, | |
| | | Hui-Liang; Lee, Wen-Chuan | |
| US2005142560A1 | Method for identifying and | Marquess, Foley Leigh-Shaw | Jun. 30, 2005 |
| | managing livestock by genotype | | |
| US7005153317A1 | Methods and systems for inferring | DeNise, Sue; Rosenfeld, | Jul. 14, 2005 |
| | traits to breed and manage non-beef | David; Kerr, Richard; Bates, | |
| | livestock | Stephen; Holm, Tom | |
| U.S. Pat. No. 5,550,024 | Genetic markers for pig litter size | Rothschild, Max F.; Tuggle, | Aug. 27, 1996 |
| | | Christopher K.; Jacobson, Carol D.; | |
| | | Vaske, David A.; | |
| | | Mileham, Alan J.; Plastow, | |
| | | Graham S. | |
| U.S. Pat. No. 5,935,784 | Prolactin receptor gene as a genetic | Rothschild, Max F.; Vincent, | Aug. 10, 1999 |
| | marker for increased litter size in | Amy L.; Tuggle, Christopher K. | |
| | pigs | | |
| U.S. Pat. No. 5,939,264 | Genes and genetic markers for | Rothschild, Max F.; Tuggle, | Aug. 17, 1999 |
| | improved reproductive traits in | Christopher K.; Messer, Lori A.; | |
| | animals | Yu, Tun-Ping | |
| U.S. Pat. No. 6,143,880 | Pig myogenin gene and method to | Soumillion, Ann Elisabeth; te | Nov. 7, 2000 |
| | identify polymorphisms related to | Pas, Marinus Frederik | |
| | muscle growth | | |
| U.S. Pat. No. 6,291,174 | DNA markers for pig litter size | Li, Ning; Wu, Changxin; | Sep. 18, 2001 |
| | | Zhao, Yaofeng | |
| U.S. Pat. No. 6,410,227 | DNA markers for litter size | Mileham, John Alan; Plastow, | Jun. 25, 2002 |
| | | Graham Stuart; Southwood, | |
| | | Olwen Irene | |
| U.S. Pat. No. 6,458,531 | Leptin receptor gene as a genetic | Rothschild, Max F.; Vincent, | Oct. 1, 2002 |
| | marker for leanness in pigs | Amy L.; Ernst, Catherine W. | |
| U.S. Pat. No. 6,803,190 | Melanocortin-4 receptor gene and | Rothschild, Max F.; Kim, | Oct. 12, 2004 |
| | use as a genetic marker for fat | Kwan Suk; Larson, Niels J. | |
| | content, weight gain, and/or feed | | |
| | consumption of animals | | |
| U.S. Pat. No. 6,846,632 | Method for rapidly identifying | Chang, Hsiu-Luan; Liaw, Ren- | Jan. 25, 2005 |
| | porcine estrogen receptor marker | Bao; Wu, Ming-Che | |
| U.S. Pat. No. 6,919,177 | Prkag3 alleles and use of the same | Rothschild, Max F.; Ciobanu, | Jul. 19, 2005 |
| | as genetic markers for reproductive | Daniel C.; Malek, Massoud; | |
| | and meat quality traits | Plastow, Graham | |
| WO0036143A2 | Selecting animals for parentally | ANDERSSON, Leif; | Jun. 22, 2000 |
| | imprinted traits | GEORGES, Michel; | |
| | | SPINCEMAILLE, Geert | |
| WO0042218A1 | Retinol binding protein 4 as a | ROTHSCHILD, Max, F.; | Jul. 20, 2000 |
| | genetic marker for increased litter | TUGGLE, Christopher, K.; | |
| | size | MESSER, Lori, A. | |
| WO0069882A1 | Genetic marker for meat quality, | GREGER, Douglas, L. | Nov. 23, 2000 |
| | growth, carcass and reproductive | | |
| | traits in livestock | | |
| WO04048606A2 | Porcine polymorphisms and | JØRGENSEN, Claus, Bottcher; | Jun. 10, 2004 |
| | methods for detecting them | CIRERA, Susanna; | |
| | | ARCHIBALD, Alan; | |
| | | ANDERSSON, Leif; | |
| | | FREDHOLM, Merete; | |
| | | EDFORS-LILJA, Inger | |
| WO05001032A2 | Fine mapping of chromosome 17 | ROTHSCHILD, Max F.,; | Jan. 6, 2005 |
| | quantitative trait loci and use of | RAMOS, Antonio; KIM, Kwan | |
| | same for marker assisted selection | Suk | |
| WO05078133A2 | Marker assisted best linear unbiased | WANG, Tianlin; LOHUIS, | Aug. 25, 2005 |
| | predicted (MA-BLUP): software | Michael, M.; KOJIMA, Cheryl, J.; | |
| | adaptions for practical applications | DU, Fengxing; BYATT, | |
| | for large breeding populations in | John, C. | |
| | farm animal species | | |
| WO06099055A2 | Sequence, polymorphisms, and | TUGGLE, Christopher, K.; | Sep. 21, 2006 |
| | marker test technology for disease | ZHAO, Shu-Hong; | |
| | resistance and growth (nfkb1) | MELLENCAMP, Martha, A.; | |
| | | BASTIAANSEN, John; | |
| | | GALINA-PANTOJA, Lucina | |
| WO06101623A2 | Cstfl and c20orf43 markers for | ROTHSCHILD, Max F.,; | Sep. 28, 2006 |
| | meat quality and growth rate in | RAMOS, Antonio Marcos Costa | |
| | animals | do Amaral | |
| WO9633288A1 | Gene marker associated with swine | DIDION, Bradley, A.; JOHNS, | Oct. 24, 1996 |
| | proliferacy | Mitrick, A. | |
| WO9914365A1 | The porcine adipocyte fatty acid- | GERBENS, Frans | Mar. 25, 1999 |
| | binding protein encoding gene and | | |
| | methods to localise, identify or | | |
| | mark genes or alleles or quantitative | | |
| | trait loci of farm animals | | |

### DESCRIPTION OF THE TABLES

Table 1: provides a list of phenotypic traits and the assigned identification numbers of SNPs found to be associated with each trait. The left column provides a counter to allow easier reading of the table. The “Trait” column lists the following traits: “COMPOSITION” (body composition), rows 1-1766; “GROWTH” rows 1768-2747; and “REPRODUCTION”, rows 2748-3718.

Table 2: provides the SEQ ID NO of the sequence associated with each of the SNPs listed in Table 1. The “SNP POSITION” column provides the position (nucleotide position number) of the SNP within the associated sequence (SEQ ID NO) in the Sequence Listing and the “SNP ALLELE 1” and “SNP ALLELE 2” columns provide the identity of the two nucleotides that occur most frequently at the SNP POSITION within the population analyzed.

TABLE 1

| ROW | | |
|---|---|---|
| NO. | TRAIT | SNP ID NO. |
| 1 | Composition | SsSNP2006B_365 |
| 2 | Composition | SsSNP2006B_465 |
| 3 | Composition | SsSNP2006B_1113 |
| 4 | Composition | SsSNP2006B_196 |
| 5 | Composition | SsSNP2006B_1841 |
| 6 | Composition | SsSNP2006B_471 |
| 7 | Composition | SsSNP2006B_1392 |
| 8 | Composition | SsSNP2006B_340 |
| 9 | Composition | SsSNP2006B_1377 |
| 10 | Composition | SsSNP2006B_890 |
| 11 | Composition | SsSNP2006B_1119 |
| 12 | Composition | SsSNP2006B_120 |
| 13 | Composition | SsSNP2006B_229 |
| 14 | Composition | SsSNP2006B_2332 |
| 15 | Composition | SsSNP2006B_2359 |
| 16 | Composition | SsSNP2006B_757 |
| 17 | Composition | SsSNP2006B_87 |
| 18 | Composition | SsSNP2006B_1905 |
| 19 | Composition | SsSNP2006B_96 |
| 20 | Composition | SsSNP2006B_2413 |
| 21 | Composition | SsSNP2006B_2539 |
| 22 | Composition | SsSNP2006B_1666 |
| 23 | Composition | SsSNP2006B_387 |
| 24 | Composition | SsSNP2006B_2550 |
| 25 | Composition | SsSNP2006B_1501 |
| 26 | Composition | SsSNP2006B_662 |
| 27 | Composition | SsSNP2006B_1263 |
| 28 | Composition | SsSNP2006B_1112 |
| 29 | Composition | SsSNP2006B_1491 |
| 30 | Composition | SsSNP2006B_23 |
| 31 | Composition | SsSNP2006B_256 |
| 32 | Composition | SsSNP2006B_950 |
| 33 | Composition | SsSNP2006B_969 |
| 34 | Composition | SsSNP2006B_1098 |
| 35 | Composition | SsSNP2006B_326 |
| 36 | Composition | SsSNP2006B_327 |
| 37 | Composition | SsSNP2006B_1290 |
| 38 | Composition | SsSNP2006B_1298 |
| 39 | Composition | SsSNP2006B_1840 |
| 40 | Composition | SsSNP2006B_1890 |
| 41 | Composition | SsSNP2006B_434 |
| 42 | Composition | SsSNP2006B_54 |
| 43 | Composition | SsSNP2006B_369 |
| 44 | Composition | SsSNP2006B_1872 |
| 45 | Composition | SsSNP2006B_76 |
| 46 | Composition | SsSNP2006B_1535 |
| 47 | Composition | SsSNP2006B_2396 |
| 48 | Composition | SsSNP2006B_13 |
| 49 | Composition | SsSNP2006B_1555 |
| 50 | Composition | SsSNP2006B_1132 |
| 51 | Composition | SsSNP2006B_1562 |
| 52 | Composition | SsSNP2006B_1464 |
| 53 | Composition | SsSNP2006B_1407 |
| 54 | Composition | SsSNP2006B_166 |
| 55 | Composition | SsSNP2006B_1394 |
| 56 | Composition | SsSNP2006B_1660 |
| 57 | Composition | SsSNP2006B_1092 |
| 58 | Composition | SsSNP2006B_2338 |
| 59 | Composition | SsSNP2006B_2417 |
| 60 | Composition | SsSNP2006B_2239 |
| 61 | Composition | SsSNP2006B_722 |
| 62 | Composition | SsSNP2006B_2145 |
| 63 | Composition | SsSNP2006B_1857 |
| 64 | Composition | SsSNP2006B_92 |
| 65 | Composition | SsSNP2006B_2344 |
| 66 | Composition | SsSNP2006B_2564 |
| 67 | Composition | SsSNP2006B_2130 |
| 68 | Composition | SsSNP2006B_2558 |
| 69 | Composition | SsSNP2006B_1574 |
| 70 | Composition | SsSNP2006B_1628 |
| 71 | Composition | SsSNP2006B_1275 |
| 72 | Composition | SsSNP2006B_1711 |
| 73 | Composition | SsSNP2006B_2507 |
| 74 | Composition | SsSNP2006B_1339 |
| 75 | Composition | SsSNP2006B_856 |
| 76 | Composition | SsSNP2006B_2248 |
| 77 | Composition | SsSNP2006B_17 |
| 78 | Composition | SsSNP2006B_1211 |
| 79 | Composition | SsSNP2006B_57 |
| 80 | Composition | SsSNP2006B_1083 |
| 81 | Composition | SsSNP2006B_1923 |
| 82 | Composition | SsSNP2006B_970 |
| 83 | Composition | SsSNP2006B_684 |
| 84 | Composition | SsSNP2006B_629 |
| 85 | Composition | SsSNP2006B_1195 |
| 86 | Composition | SsSNP2006B_304 |
| 87 | Composition | SsSNP2006B_1686 |
| 88 | Composition | SsSNP2006B_882 |
| 89 | Composition | SsSNP2006B_175 |
| 90 | Composition | SsSNP2006B_2330 |
| 91 | Composition | SsSNP2006B_583 |
| 92 | Composition | SsSNP2006B_1048 |
| 93 | Composition | SsSNP2006B_154 |
| 94 | Composition | SsSNP2006B_1717 |
| 95 | Composition | SsSNP2006B_2113 |
| 96 | Composition | SsSNP2006B_489 |
| 97 | Composition | SsSNP2006B_1603 |
| 98 | Composition | SsSNP2006B_868 |
| 99 | Composition | SsSNP2006B_2281 |
| 100 | Composition | SsSNP2006B_63 |
| 101 | Composition | SsSNP2006B_2058 |
| 102 | Composition | SsSNP2006B_158 |
| 103 | Composition | SsSNP2006B_2287 |
| 104 | Composition | SsSNP2006B_1969 |
| 105 | Composition | SsSNP2006B_1192 |
| 106 | Composition | SsSNP2006B_2566 |
| 107 | Composition | SsSNP2006B_2214 |
| 108 | Composition | SsSNP2006B_2402 |
| 109 | Composition | SsSNP2006B_1374 |
| 110 | Composition | SsSNP2006B_2474 |
| 111 | Composition | SsSNP2006B_1781 |
| 112 | Composition | SsSNP2006B_2012 |
| 113 | Composition | SsSNP2006B_2175 |
| 114 | Composition | SsSNP2006B_826 |
| 115 | Composition | SsSNP2006B_1619 |
| 116 | Composition | SsSNP2006B_855 |
| 117 | Composition | SsSNP2006B_66 |
| 118 | Composition | SsSNP2006B_1830 |
| 119 | Composition | SsSNP2006B_1499 |
| 120 | Composition | SsSNP2006B_500 |
| 121 | Composition | SsSNP2006B_862 |
| 122 | Composition | SsSNP2006B_2331 |
| 123 | Composition | SsSNP2006B_1993 |
| 124 | Composition | SsSNP2006B_1447 |
| 125 | Composition | SsSNP2006B_2278 |
| 126 | Composition | SsSNP2006B_2279 |
| 127 | Composition | SsSNP2006B_1986 |
| 128 | Composition | SsSNP2006B_358 |
| 129 | Composition | SsSNP2006B_1101 |
| 130 | Composition | SsSNP2006B_1436 |
| 131 | Composition | SsSNP2006B_2380 |
| 132 | Composition | SsSNP2006B_1508 |
| 133 | Composition | SsSNP2006B_1961 |
| 134 | Composition | SsSNP2006B_2348 |
| 135 | Composition | SsSNP2006B_2240 |
| 136 | Composition | SsSNP2006B_417 |
| 137 | Composition | SsSNP2006B_2505 |
| 138 | Composition | SsSNP2006B_1199 |
| 139 | Composition | SsSNP2006B_2048 |
| 140 | Composition | SsSNP2006B_1864 |
| 141 | Composition | SsSNP2006B_522 |
| 142 | Composition | SsSNP2006B_1060 |
| 143 | Composition | SsSNP2006B_1198 |
| 144 | Composition | SsSNP2006B_2457 |
| 145 | Composition | SsSNP2006B_2405 |
| 146 | Composition | SsSNP2006B_542 |
| 147 | Composition | SsSNP2006B_543 |
| 148 | Composition | SsSNP2006B_1521 |
| 149 | Composition | SsSNP2006B_1073 |
| 150 | Composition | SsSNP2006B_1891 |
| 151 | Composition | SsSNP2006B_1315 |
| 152 | Composition | SsSNP2006B_763 |
| 153 | Composition | SsSNP2006B_155 |
| 154 | Composition | SsSNP2006B_1797 |
| 155 | Composition | SsSNP2006B_615 |
| 156 | Composition | SsSNP2006B_671 |
| 157 | Composition | SsSNP2006B_784 |
| 158 | Composition | SsSNP2006B_2495 |
| 159 | Composition | SsSNP2006B_690 |
| 160 | Composition | SsSNP2006B_2050 |
| 161 | Composition | SsSNP2006B_589 |
| 162 | Composition | SsSNP2006B_1431 |
| 163 | Composition | SsSNP2006B_1109 |
| 164 | Composition | SsSNP2006B_2257 |
| 165 | Composition | SsSNP2006B_2285 |
| 166 | Composition | SsSNP2006B_176 |
| 167 | Composition | SsSNP2006B_189 |
| 168 | Composition | SsSNP2006B_289 |
| 169 | Composition | SsSNP2006B_1788 |
| 170 | Composition | SsSNP2006B_2406 |
| 171 | Composition | SsSNP2006B_1643 |
| 172 | Composition | SsSNP2006B_420 |
| 173 | Composition | SsSNP2006B_1220 |
| 174 | Composition | SsSNP2006B_928 |
| 175 | Composition | SsSNP2006B_1482 |
| 176 | Composition | SsSNP2006B_2534 |
| 177 | Composition | SsSNP2006B_676 |
| 178 | Composition | SsSNP2006B_314 |
| 179 | Composition | SsSNP2006B_2061 |
| 180 | Composition | SsSNP2006B_2366 |
| 181 | Composition | SsSNP2006B_549 |
| 182 | Composition | SsSNP2006B_1371 |
| 183 | Composition | SsSNP2006B_820 |
| 184 | Composition | SsSNP2006B_344 |
| 185 | Composition | SsSNP2006B_1773 |
| 186 | Composition | SsSNP2006B_296 |
| 187 | Composition | SsSNP2006B_15 |
| 188 | Composition | SsSNP2006B_688 |
| 189 | Composition | SsSNP2006B_2377 |
| 190 | Composition | SsSNP2006B_602 |
| 191 | Composition | SsSNP2006B_1897 |
| 192 | Composition | SsSNP2006B_770 |
| 193 | Composition | SsSNP2006B_2555 |
| 194 | Composition | SsSNP2006B_2249 |
| 195 | Composition | SsSNP2006B_331 |
| 196 | Composition | SsSNP2006B_1594 |
| 197 | Composition | SsSNP2006B_130 |
| 198 | Composition | SsSNP2006B_618 |
| 199 | Composition | SsSNP2006B_2138 |
| 200 | Composition | SsSNP2006B_1962 |
| 201 | Composition | SsSNP2006B_399 |
| 202 | Composition | SsSNP2006B_1372 |
| 203 | Composition | SsSNP2006B_608 |
| 204 | Composition | SsSNP2006B_2269 |
| 205 | Composition | SsSNP2006B_25 |
| 206 | Composition | SsSNP2006B_782 |
| 207 | Composition | SsSNP2006B_2270 |
| 208 | Composition | SsSNP2006B_2323 |
| 209 | Composition | SsSNP2006B_487 |
| 210 | Composition | SsSNP2006B_1892 |
| 211 | Composition | SsSNP2006B_86 |
| 212 | Composition | SsSNP2006B_978 |
| 213 | Composition | SsSNP2006B_472 |
| 214 | Composition | SsSNP2006B_2450 |
| 215 | Composition | SsSNP2006B_1809 |
| 216 | Composition | SsSNP2006B_567 |
| 217 | Composition | SsSNP2006B_1032 |
| 218 | Composition | SsSNP2006B_934 |
| 219 | Composition | SsSNP2006B_1880 |
| 220 | Composition | SsSNP2006B_1881 |
| 221 | Composition | SsSNP2006B_2052 |
| 222 | Composition | SsSNP2006B_208 |
| 223 | Composition | SsSNP2006B_1670 |
| 224 | Composition | SsSNP2006B_1949 |
| 225 | Composition | SsSNP2006B_2400 |
| 226 | Composition | SsSNP2006B_743 |
| 227 | Composition | SsSNP2006B_1176 |
| 228 | Composition | SsSNP2006B_730 |
| 229 | Composition | SsSNP2006B_1186 |
| 230 | Composition | SsSNP2006B_1755 |
| 231 | Composition | SsSNP2006B_3 |
| 232 | Composition | SsSNP2006B_936 |
| 233 | Composition | SsSNP2006B_517 |
| 234 | Composition | SsSNP2006B_1040 |
| 235 | Composition | SsSNP2006B_1716 |
| 236 | Composition | SsSNP2006B_566 |
| 237 | Composition | SsSNP2006B_1273 |
| 238 | Composition | SsSNP2006B_754 |
| 239 | Composition | SsSNP2006B_2401 |
| 240 | Composition | SsSNP2006B_1178 |
| 241 | Composition | SsSNP2006B_421 |
| 242 | Composition | SsSNP2006B_330 |
| 243 | Composition | SsSNP2006B_1077 |
| 244 | Composition | SsSNP2006B_2479 |
| 245 | Composition | SsSNP2006B_1492 |
| 246 | Composition | SsSNP2006B_2192 |
| 247 | Composition | SsSNP2006B_180 |
| 248 | Composition | SsSNP2006B_2044 |
| 249 | Composition | SsSNP2006B_768 |
| 250 | Composition | SsSNP2006B_2127 |
| 251 | Composition | SsSNP2006B_1338 |
| 252 | Composition | SsSNP2006B_2038 |
| 253 | Composition | SsSNP2006B_2378 |
| 254 | Composition | SsSNP2006B_2186 |
| 255 | Composition | SsSNP2006B_1657 |
| 256 | Composition | SsSNP2006B_1879 |
| 257 | Composition | SsSNP2006B_6 |
| 258 | Composition | SsSNP2006B_40 |
| 259 | Composition | SsSNP2006B_1103 |
| 260 | Composition | SsSNP2006B_550 |
| 261 | Composition | SsSNP2006B_1140 |
| 262 | Composition | SsSNP2006B_1966 |
| 263 | Composition | SsSNP2006B_1748 |
| 264 | Composition | SsSNP2006B_1801 |
| 265 | Composition | SsSNP2006B_1340 |
| 266 | Composition | SsSNP2006B_1855 |
| 267 | Composition | SsSNP2006B_1429 |
| 268 | Composition | SsSNP2006B_798 |
| 269 | Composition | SsSNP2006B_2571 |
| 270 | Composition | SsSNP2006B_179 |
| 271 | Composition | SsSNP2006B_1909 |
| 272 | Composition | SsSNP2006B_1503 |
| 273 | Composition | SsSNP2006B_2353 |
| 274 | Composition | SsSNP2006B_2029 |
| 275 | Composition | SsSNP2006B_1206 |
| 276 | Composition | SsSNP2006B_1715 |
| 277 | Composition | SsSNP2006B_693 |
| 278 | Composition | SsSNP2006B_984 |
| 279 | Composition | SsSNP2006B_656 |
| 280 | Composition | SsSNP2006B_560 |
| 281 | Composition | SsSNP2006B_159 |
| 282 | Composition | SsSNP2006B_247 |
| 283 | Composition | SsSNP2006B_2598 |
| 284 | Composition | SsSNP2006B_2073 |
| 285 | Composition | SsSNP2006B_81 |
| 286 | Composition | SsSNP2006B_1300 |
| 287 | Composition | SsSNP2006B_1869 |
| 288 | Composition | SsSNP2006B_1870 |
| 289 | Composition | SsSNP2006B_1556 |
| 290 | Composition | SsSNP2006B_2268 |
| 291 | Composition | SsSNP2006B_1995 |
| 292 | Composition | SsSNP2006B_1785 |
| 293 | Composition | SsSNP2006B_1587 |
| 294 | Composition | SsSNP2006B_609 |
| 295 | Composition | SsSNP2006B_626 |
| 296 | Composition | SsSNP2006B_2307 |
| 297 | Composition | SsSNP2006B_2069 |
| 298 | Composition | SsSNP2006B_1486 |
| 299 | Composition | SsSNP2006B_815 |
| 300 | Composition | SsSNP2006B_1114 |
| 301 | Composition | SsSNP2006B_745 |
| 302 | Composition | SsSNP2006B_360 |
| 303 | Composition | SsSNP2006B_691 |
| 304 | Composition | SsSNP2006B_1942 |
| 305 | Composition | SsSNP2006B_1931 |
| 306 | Composition | SsSNP2006B_643 |
| 307 | Composition | SsSNP2006B_481 |
| 308 | Composition | SsSNP2006B_389 |
| 309 | Composition | SsSNP2006B_1713 |
| 310 | Composition | SsSNP2006B_1177 |
| 311 | Composition | SsSNP2006B_1753 |
| 312 | Composition | SsSNP2006B_1107 |
| 313 | Composition | SsSNP2006B_731 |
| 314 | Composition | SsSNP2006B_1054 |
| 315 | Composition | SsSNP2006B_1172 |
| 316 | Composition | SsSNP2006B_2090 |
| 317 | Composition | SsSNP2006B_611 |
| 318 | Composition | SsSNP2006B_1327 |
| 319 | Composition | SsSNP2006B_974 |
| 320 | Composition | SsSNP2006B_1767 |
| 321 | Composition | SsSNP2006B_2296 |
| 322 | Composition | SsSNP2006B_1802 |
| 323 | Composition | SsSNP2006B_1764 |
| 324 | Composition | SsSNP2006B_303 |
| 325 | Composition | SsSNP2006B_895 |
| 326 | Composition | SsSNP2006B_2452 |
| 327 | Composition | SsSNP2006B_2141 |
| 328 | Composition | SsSNP2006B_2142 |
| 329 | Composition | SsSNP2006B_2234 |
| 330 | Composition | SsSNP2006B_552 |
| 331 | Composition | SsSNP2006B_2292 |
| 332 | Composition | SsSNP2006B_260 |
| 333 | Composition | SsSNP2006B_1637 |
| 334 | Composition | SsSNP2006B_2014 |
| 335 | Composition | SsSNP2006B_153 |
| 336 | Composition | SsSNP2006B_2535 |
| 337 | Composition | SsSNP2006B_1703 |
| 338 | Composition | SsSNP2006B_1219 |
| 339 | Composition | SsSNP2006B_819 |
| 340 | Composition | SsSNP2006B_630 |
| 341 | Composition | SsSNP2006B_1110 |
| 342 | Composition | SsSNP2006B_2164 |
| 343 | Composition | SsSNP2006B_2580 |
| 344 | Composition | SsSNP2006B_1082 |
| 345 | Composition | SsSNP2006B_1335 |
| 346 | Composition | SsSNP2006B_370 |
| 347 | Composition | SsSNP2006B_1121 |
| 348 | Composition | SsSNP2006B_1530 |
| 349 | Composition | SsSNP2006B_734 |
| 350 | Composition | SsSNP2006B_213 |
| 351 | Composition | SsSNP2006B_1252 |
| 352 | Composition | SsSNP2006B_2591 |
| 353 | Composition | SsSNP2006B_45 |
| 354 | Composition | SsSNP2006B_1829 |
| 355 | Composition | SsSNP2006B_1769 |
| 356 | Composition | SsSNP2006B_1956 |
| 357 | Composition | SsSNP2006B_2486 |
| 358 | Composition | SsSNP2006B_1691 |
| 359 | Composition | SsSNP2006B_2556 |
| 360 | Composition | SsSNP2006B_822 |
| 361 | Composition | SsSNP2006B_1738 |
| 362 | Composition | SsSNP2006B_1148 |
| 363 | Composition | SsSNP2006B_2272 |
| 364 | Composition | SsSNP2006B_1018 |
| 365 | Composition | SsSNP2006B_2097 |
| 366 | Composition | SsSNP2006B_1463 |
| 367 | Composition | SsSNP2006B_1616 |
| 368 | Composition | SsSNP2006B_112 |
| 369 | Composition | SsSNP2006B_712 |
| 370 | Composition | SsSNP2006B_911 |
| 371 | Composition | SsSNP2006B_1445 |
| 372 | Composition | SsSNP2006B_813 |
| 373 | Composition | SsSNP2006B_2242 |
| 374 | Composition | SsSNP2006B_860 |
| 375 | Composition | SsSNP2006B_2053 |
| 376 | Composition | SsSNP2006B_11 |
| 377 | Composition | SsSNP2006B_453 |
| 378 | Composition | SsSNP2006B_905 |
| 379 | Composition | SsSNP2006B_2221 |
| 380 | Composition | SsSNP2006B_1620 |
| 381 | Composition | SsSNP2006B_960 |
| 382 | Composition | SsSNP2006B_2557 |
| 383 | Composition | SsSNP2006B_470 |
| 384 | Composition | SsSNP2006B_491 |
| 385 | Composition | SsSNP2006B_1554 |
| 386 | Composition | SsSNP2006B_2513 |
| 387 | Composition | SsSNP2006B_749 |
| 388 | Composition | SsSNP2006B_2116 |
| 389 | Composition | SsSNP2006B_761 |
| 390 | Composition | SsSNP2006B_1689 |
| 391 | Composition | SsSNP2006B_753 |
| 392 | Composition | SsSNP2006B_1550 |
| 393 | Composition | SsSNP2006B_2388 |
| 394 | Composition | SsSNP2006B_2037 |
| 395 | Composition | SsSNP2006B_91 |
| 396 | Composition | SsSNP2006B_2487 |
| 397 | Composition | SsSNP2006B_777 |
| 398 | Composition | SsSNP2006B_962 |
| 399 | Composition | SsSNP2006B_788 |
| 400 | Composition | SsSNP2006B_317 |
| 401 | Composition | SsSNP2006B_2251 |
| 402 | Composition | SsSNP2006B_2379 |
| 403 | Composition | SsSNP2006B_2575 |
| 404 | Composition | SsSNP2006B_2157 |
| 405 | Composition | SsSNP2006B_569 |
| 406 | Composition | SsSNP2006B_2117 |
| 407 | Composition | SsSNP2006B_2447 |
| 408 | Composition | SsSNP2006B_293 |
| 409 | Composition | SsSNP2006B_46 |
| 410 | Composition | SsSNP2006B_2569 |
| 411 | Composition | SsSNP2006B_181 |
| 412 | Composition | SsSNP2006B_613 |
| 413 | Composition | SsSNP2006B_73 |
| 414 | Composition | SsSNP2006B_74 |
| 415 | Composition | SsSNP2006B_274 |
| 416 | Composition | SsSNP2006B_1640 |
| 417 | Composition | SsSNP2006B_140 |
| 418 | Composition | SsSNP2006B_9 |
| 419 | Composition | SsSNP2006B_1337 |
| 420 | Composition | SsSNP2006B_461 |
| 421 | Composition | SsSNP2006B_2091 |
| 422 | Composition | SsSNP2006B_535 |
| 423 | Composition | SsSNP2006B_341 |
| 424 | Composition | SsSNP2006B_2319 |
| 425 | Composition | SsSNP2006B_1557 |
| 426 | Composition | SsSNP2006B_322 |
| 427 | Composition | SsSNP2006B_1835 |
| 428 | Composition | SsSNP2006B_2590 |
| 429 | Composition | SsSNP2006B_2418 |
| 430 | Composition | SsSNP2006B_2467 |
| 431 | Composition | SsSNP2006B_720 |
| 432 | Composition | SsSNP2006B_719 |
| 433 | Composition | SsSNP2006B_1418 |
| 434 | Composition | SsSNP2006B_1259 |
| 435 | Composition | SsSNP2006B_2128 |
| 436 | Composition | SsSNP2006B_1062 |
| 437 | Composition | SsSNP2006B_2051 |
| 438 | Composition | SsSNP2006B_1185 |
| 439 | Composition | SsSNP2006B_2203 |
| 440 | Composition | SsSNP2006B_475 |
| 441 | Composition | SsSNP2006B_692 |
| 442 | Composition | SsSNP2006B_1050 |
| 443 | Composition | SsSNP2006B_273 |
| 444 | Composition | SsSNP2006B_908 |
| 445 | Composition | SsSNP2006B_2076 |
| 446 | Composition | SsSNP2006B_1059 |
| 447 | Composition | SsSNP2006B_2084 |
| 448 | Composition | SsSNP2006B_2092 |
| 449 | Composition | SsSNP2006B_34 |
| 450 | Composition | SsSNP2006B_1979 |
| 451 | Composition | SsSNP2006B_1980 |
| 452 | Composition | SsSNP2006B_1457 |
| 453 | Composition | SsSNP2006B_1663 |
| 454 | Composition | SsSNP2006B_198 |
| 455 | Composition | SsSNP2006B_695 |
| 456 | Composition | SsSNP2006B_1696 |
| 457 | Composition | SsSNP2006B_1047 |
| 458 | Composition | SsSNP2006B_1859 |
| 459 | Composition | SsSNP2006B_1551 |
| 460 | Composition | SsSNP2006B_125 |
| 461 | Composition | SsSNP2006B_126 |
| 462 | Composition | SsSNP2006B_426 |
| 463 | Composition | SsSNP2006B_2093 |
| 464 | Composition | SsSNP2006B_755 |
| 465 | Composition | SsSNP2006B_1967 |
| 466 | Composition | SsSNP2006B_2238 |
| 467 | Composition | SsSNP2006B_1861 |
| 468 | Composition | SsSNP2006B_1443 |
| 469 | Composition | SsSNP2006B_1588 |
| 470 | Composition | SsSNP2006B_1045 |
| 471 | Composition | SsSNP2006B_2000 |
| 472 | Composition | SsSNP2006B_1039 |
| 473 | Composition | SsSNP2006B_659 |
| 474 | Composition | SsSNP2006B_2563 |
| 475 | Composition | SsSNP2006B_206 |
| 476 | Composition | SsSNP2006B_1586 |
| 477 | Composition | SsSNP2006B_1770 |
| 478 | Composition | SsSNP2006B_2295 |
| 479 | Composition | SsSNP2006B_2100 |
| 480 | Composition | SsSNP2006B_1278 |
| 481 | Composition | SsSNP2006B_1268 |
| 482 | Composition | SsSNP2006B_68 |
| 483 | Composition | SsSNP2006B_1661 |
| 484 | Composition | SsSNP2006B_980 |
| 485 | Composition | SsSNP2006B_431 |
| 486 | Composition | SsSNP2006B_2599 |
| 487 | Composition | SsSNP2006B_1532 |
| 488 | Composition | SsSNP2006B_619 |
| 489 | Composition | SsSNP2006B_1481 |
| 490 | Composition | SsSNP2006B_1087 |
| 491 | Composition | SsSNP2006B_366 |
| 492 | Composition | SsSNP2006B_148 |
| 493 | Composition | SsSNP2006B_1156 |
| 494 | Composition | SsSNP2006B_2081 |
| 495 | Composition | SsSNP2006B_1150 |
| 496 | Composition | SsSNP2006B_1434 |
| 497 | Composition | SsSNP2006B_2551 |
| 498 | Composition | SsSNP2006B_2025 |
| 499 | Composition | SsSNP2006B_873 |
| 500 | Composition | SsSNP2006B_1784 |
| 501 | Composition | SsSNP2006B_2271 |
| 502 | Composition | SsSNP2006B_394 |
| 503 | Composition | SsSNP2006B_896 |
| 504 | Composition | SsSNP2006B_1028 |
| 505 | Composition | SsSNP2006B_65 |
| 506 | Composition | SsSNP2006B_779 |
| 507 | Composition | SsSNP2006B_328 |
| 508 | Composition | SsSNP2006B_1860 |
| 509 | Composition | SsSNP2006B_2408 |
| 510 | Composition | SsSNP2006B_1002 |
| 511 | Composition | SsSNP2006B_1902 |
| 512 | Composition | SsSNP2006B_1976 |
| 513 | Composition | SsSNP2006B_1634 |
| 514 | Composition | SsSNP2006B_2151 |
| 515 | Composition | SsSNP2006B_1330 |
| 516 | Composition | SsSNP2006B_26 |
| 517 | Composition | SsSNP2006B_1036 |
| 518 | Composition | SsSNP2006B_1813 |
| 519 | Composition | SsSNP2006B_1256 |
| 520 | Composition | SsSNP2006B_2529 |
| 521 | Composition | SsSNP2006B_169 |
| 522 | Composition | SsSNP2006B_1261 |
| 523 | Composition | SsSNP2006B_376 |
| 524 | Composition | SsSNP2006B_2364 |
| 525 | Composition | SsSNP2006B_795 |
| 526 | Composition | SsSNP2006B_414 |
| 527 | Composition | SsSNP2006B_1090 |
| 528 | Composition | SsSNP2006B_1687 |
| 529 | Composition | SsSNP2006B_174 |
| 530 | Composition | SsSNP2006B_893 |
| 531 | Composition | SsSNP2006B_894 |
| 532 | Composition | SsSNP2006B_2122 |
| 533 | Composition | SsSNP2006B_1202 |
| 534 | Composition | SsSNP2006B_523 |
| 535 | Composition | SsSNP2006B_377 |
| 536 | Composition | SsSNP2006B_2137 |
| 537 | Composition | SsSNP2006B_1732 |
| 538 | Composition | SsSNP2006B_2376 |
| 539 | Composition | SsSNP2006B_1987 |
| 540 | Composition | SsSNP2006B_2470 |
| 541 | Composition | SsSNP2006B_604 |
| 542 | Composition | SsSNP2006B_711 |
| 543 | Composition | SsSNP2006B_504 |
| 544 | Composition | SsSNP2006B_1601 |
| 545 | Composition | SsSNP2006B_505 |
| 546 | Composition | SsSNP2006B_1151 |
| 547 | Composition | SsSNP2006B_2309 |
| 548 | Composition | SsSNP2006B_1286 |
| 549 | Composition | SsSNP2006B_67 |
| 550 | Composition | SsSNP2006B_1913 |
| 551 | Composition | SsSNP2006B_1759 |
| 552 | Composition | SsSNP2006B_1439 |
| 553 | Composition | SsSNP2006B_685 |
| 554 | Composition | SsSNP2006B_1296 |
| 555 | Composition | SsSNP2006B_915 |
| 556 | Composition | SsSNP2006B_1080 |
| 557 | Composition | SsSNP2006B_2159 |
| 558 | Composition | SsSNP2006B_1242 |
| 559 | Composition | SsSNP2006B_2423 |
| 560 | Composition | SsSNP2006B_2167 |
| 561 | Composition | SsSNP2006B_1908 |
| 562 | Composition | SsSNP2006B_1975 |
| 563 | Composition | SsSNP2006B_2525 |
| 564 | Composition | SsSNP2006B_1741 |
| 565 | Composition | SsSNP2006B_1743 |
| 566 | Composition | SsSNP2006B_2149 |
| 567 | Composition | SsSNP2006B_1878 |
| 568 | Composition | SsSNP2006B_1188 |
| 569 | Composition | SsSNP2006B_1505 |
| 570 | Composition | SsSNP2006B_1948 |
| 571 | Composition | SsSNP2006B_1862 |
| 572 | Composition | SsSNP2006B_231 |
| 573 | Composition | SsSNP2006B_2245 |
| 574 | Composition | SsSNP2006B_2111 |
| 575 | Composition | SsSNP2006B_1939 |
| 576 | Composition | SsSNP2006B_1682 |
| 577 | Composition | SsSNP2006B_83 |
| 578 | Composition | SsSNP2006B_2109 |
| 579 | Composition | SsSNP2006B_1572 |
| 580 | Composition | SsSNP2006B_2011 |
| 581 | Composition | SsSNP2006B_607 |
| 582 | Composition | SsSNP2006B_1758 |
| 583 | Composition | SsSNP2006B_62 |
| 584 | Composition | SsSNP2006B_1836 |
| 585 | Composition | SsSNP2006B_1804 |
| 586 | Composition | SsSNP2006B_1791 |
| 587 | Composition | SsSNP2006B_1333 |
| 588 | Composition | SsSNP2006B_1569 |
| 589 | Composition | SsSNP2006B_1068 |
| 590 | Composition | SsSNP2006B_1357 |
| 591 | Composition | SsSNP2006B_1529 |
| 592 | Composition | SsSNP2006B_8 |
| 593 | Composition | SsSNP2006B_2517 |
| 594 | Composition | SsSNP2006B_445 |
| 595 | Composition | SsSNP2006B_1334 |
| 596 | Composition | SsSNP2006B_2259 |
| 597 | Composition | SsSNP2006B_1247 |
| 598 | Composition | SsSNP2006B_1779 |
| 599 | Composition | SsSNP2006B_2536 |
| 600 | Composition | SsSNP2006B_178 |
| 601 | Composition | SsSNP2006B_115 |
| 602 | Composition | SsSNP2006B_141 |
| 603 | Composition | SsSNP2006B_469 |
| 604 | Composition | SsSNP2006B_2162 |
| 605 | Composition | SsSNP2006B_228 |
| 606 | Composition | SsSNP2006B_1596 |
| 607 | Composition | SsSNP2006B_2462 |
| 608 | Composition | SsSNP2006B_1744 |
| 609 | Composition | SsSNP2006B_410 |
| 610 | Composition | SsSNP2006B_30 |
| 611 | Composition | SsSNP2006B_878 |
| 612 | Composition | SsSNP2006B_2057 |
| 613 | Composition | SsSNP2006B_476 |
| 614 | Composition | SsSNP2006B_699 |
| 615 | Composition | SsSNP2006B_848 |
| 616 | Composition | SsSNP2006B_428 |
| 617 | Composition | SsSNP2006B_2449 |
| 618 | Composition | SsSNP2006B_440 |
| 619 | Composition | SsSNP2006B_2163 |
| 620 | Composition | SsSNP2006B_49 |
| 621 | Composition | SsSNP2006B_448 |
| 622 | Composition | SsSNP2006B_1933 |
| 623 | Composition | SsSNP2006B_2211 |
| 624 | Composition | SsSNP2006B_2302 |
| 625 | Composition | SsSNP2006B_864 |
| 626 | Composition | SsSNP2006B_320 |
| 627 | Composition | SsSNP2006B_585 |
| 628 | Composition | SsSNP2006B_1216 |
| 629 | Composition | SsSNP2006B_1793 |
| 630 | Composition | SsSNP2006B_531 |
| 631 | Composition | SsSNP2006B_1825 |
| 632 | Composition | SsSNP2006B_177 |
| 633 | Composition | SsSNP2006B_1707 |
| 634 | Composition | SsSNP2006B_2346 |
| 635 | Composition | SsSNP2006B_2383 |
| 636 | Composition | SsSNP2006B_1776 |
| 637 | Composition | SsSNP2006B_1368 |
| 638 | Composition | SsSNP2006B_532 |
| 639 | Composition | SsSNP2006B_870 |
| 640 | Composition | SsSNP2006B_1985 |
| 641 | Composition | SsSNP2006B_541 |
| 642 | Composition | SsSNP2006B_641 |
| 643 | Composition | SsSNP2006B_402 |
| 644 | Composition | SsSNP2006B_598 |
| 645 | Composition | SsSNP2006B_1731 |
| 646 | Composition | SsSNP2006B_1871 |
| 647 | Composition | SsSNP2006B_875 |
| 648 | Composition | SsSNP2006B_1403 |
| 649 | Composition | SsSNP2006B_1426 |
| 650 | Composition | SsSNP2006B_441 |
| 651 | Composition | SsSNP2006B_2227 |
| 652 | Composition | SsSNP2006B_2015 |
| 653 | Composition | SsSNP2006B_904 |
| 654 | Composition | SsSNP2006B_1695 |
| 655 | Composition | SsSNP2006B_1342 |
| 656 | Composition | SsSNP2006B_2060 |
| 657 | Composition | SsSNP2006B_2010 |
| 658 | Composition | SsSNP2006B_1122 |
| 659 | Composition | SsSNP2006B_663 |
| 660 | Composition | SsSNP2006B_664 |
| 661 | Composition | SsSNP2006B_537 |
| 662 | Composition | SsSNP2006B_1935 |
| 663 | Composition | SsSNP2006B_1420 |
| 664 | Composition | SsSNP2006B_2002 |
| 665 | Composition | SsSNP2006B_1063 |
| 666 | Composition | SsSNP2006B_1623 |
| 667 | Composition | SsSNP2006B_1624 |
| 668 | Composition | SsSNP2006B_1904 |
| 669 | Composition | SsSNP2006B_2039 |
| 670 | Composition | SsSNP2006B_620 |
| 671 | Composition | SsSNP2006B_51 |
| 672 | Composition | SsSNP2006B_1549 |
| 673 | Composition | SsSNP2006B_1727 |
| 674 | Composition | SsSNP2006B_538 |
| 675 | Composition | SsSNP2006B_642 |
| 676 | Composition | SsSNP2006B_1356 |
| 677 | Composition | SsSNP2006B_1025 |
| 678 | Composition | SsSNP2006B_1561 |
| 679 | Composition | SsSNP2006B_2416 |
| 680 | Composition | SsSNP2006B_810 |
| 681 | Composition | SsSNP2006B_1160 |
| 682 | Composition | SsSNP2006B_1820 |
| 683 | Composition | SsSNP2006B_580 |
| 684 | Composition | SsSNP2006B_916 |
| 685 | Composition | SsSNP2006B_2432 |
| 686 | Composition | SsSNP2006B_116 |
| 687 | Composition | SsSNP2006B_222 |
| 688 | Composition | SsSNP2006B_2224 |
| 689 | Composition | SsSNP2006B_233 |
| 690 | Composition | SsSNP2006B_1568 |
| 691 | Composition | SsSNP2006B_214 |
| 692 | Composition | SsSNP2006B_201 |
| 693 | Composition | SsSNP2006B_1576 |
| 694 | Composition | SsSNP2006B_1281 |
| 695 | Composition | SsSNP2006B_559 |
| 696 | Composition | SsSNP2006B_418 |
| 697 | Composition | SsSNP2006B_450 |
| 698 | Composition | SsSNP2006B_1251 |
| 699 | Composition | SsSNP2006B_2586 |
| 700 | Composition | SsSNP2006B_1398 |
| 701 | Composition | SsSNP2006B_1461 |
| 702 | Composition | SsSNP2006B_301 |
| 703 | Composition | SsSNP2006B_2237 |
| 704 | Composition | SsSNP2006B_1100 |
| 705 | Composition | SsSNP2006B_106 |
| 706 | Composition | SsSNP2006B_2193 |
| 707 | Composition | SsSNP2006B_1514 |
| 708 | Composition | SsSNP2006B_2223 |
| 709 | Composition | SsSNP2006B_706 |
| 710 | Composition | SsSNP2006B_678 |
| 711 | Composition | SsSNP2006B_946 |
| 712 | Composition | SsSNP2006B_1435 |
| 713 | Composition | SsSNP2006B_1031 |
| 714 | Composition | SsSNP2006B_1706 |
| 715 | Composition | SsSNP2006B_2004 |
| 716 | Composition | SsSNP2006B_2231 |
| 717 | Composition | SsSNP2006B_1091 |
| 718 | Composition | SsSNP2006B_2204 |
| 719 | Composition | SsSNP2006B_335 |
| 720 | Composition | SsSNP2006B_1266 |
| 721 | Composition | SsSNP2006B_1239 |
| 722 | Composition | SsSNP2006B_562 |
| 723 | Composition | SsSNP2006B_716 |
| 724 | Composition | SsSNP2006B_2511 |
| 725 | Composition | SsSNP2006B_1452 |
| 726 | Composition | SsSNP2006B_1538 |
| 727 | Composition | SsSNP2006B_37 |
| 728 | Composition | SsSNP2006B_1506 |
| 729 | Composition | SsSNP2006B_443 |
| 730 | Composition | SsSNP2006B_2365 |
| 731 | Composition | SsSNP2006B_1817 |
| 732 | Composition | SsSNP2006B_2315 |
| 733 | Composition | SsSNP2006B_840 |
| 734 | Composition | SsSNP2006B_516 |
| 735 | Composition | SsSNP2006B_727 |
| 736 | Composition | SsSNP2006B_1837 |
| 737 | Composition | SsSNP2006B_1485 |
| 738 | Composition | SsSNP2006B_199 |
| 739 | Composition | SsSNP2006B_2065 |
| 740 | Composition | SsSNP2006B_1164 |
| 741 | Composition | SsSNP2006B_1925 |
| 742 | Composition | SsSNP2006B_1633 |
| 743 | Composition | SsSNP2006B_1806 |
| 744 | Composition | SsSNP2006B_1851 |
| 745 | Composition | SsSNP2006B_378 |
| 746 | Composition | SsSNP2006B_574 |
| 747 | Composition | SsSNP2006B_1427 |
| 748 | Composition | SsSNP2006B_547 |
| 749 | Composition | SsSNP2006B_351 |
| 750 | Composition | SsSNP2006B_594 |
| 751 | Composition | SsSNP2006B_1832 |
| 752 | Composition | SsSNP2006B_1652 |
| 753 | Composition | SsSNP2006B_1523 |
| 754 | Composition | SsSNP2006B_2009 |
| 755 | Composition | SsSNP2006B_1258 |
| 756 | Composition | SsSNP2006B_1875 |
| 757 | Composition | SsSNP2006B_1071 |
| 758 | Composition | SsSNP2006B_906 |
| 759 | Composition | SsSNP2006B_2064 |
| 760 | Composition | SsSNP2006B_1470 |
| 761 | Composition | SsSNP2006B_2342 |
| 762 | Composition | SsSNP2006B_2385 |
| 763 | Composition | SsSNP2006B_701 |
| 764 | Composition | SsSNP2006B_1945 |
| 765 | Composition | SsSNP2006B_1442 |
| 766 | Composition | SsSNP2006B_33 |
| 767 | Composition | SsSNP2006B_1417 |
| 768 | Composition | SsSNP2006B_2381 |
| 769 | Composition | SsSNP2006B_1712 |
| 770 | Composition | SsSNP2006B_2179 |
| 771 | Composition | SsSNP2006B_95 |
| 772 | Composition | SsSNP2006B_2355 |
| 773 | Composition | SsSNP2006B_578 |
| 774 | Composition | SsSNP2006B_1043 |
| 775 | Composition | SsSNP2006B_1167 |
| 776 | Composition | SsSNP2006B_1847 |
| 777 | Composition | SsSNP2006B_901 |
| 778 | Composition | SsSNP2006B_1927 |
| 779 | Composition | SsSNP2006B_1957 |
| 780 | Composition | SsSNP2006B_1912 |
| 781 | Composition | SsSNP2006B_914 |
| 782 | Composition | SsSNP2006B_2459 |
| 783 | Composition | SsSNP2006B_1313 |
| 784 | Composition | SsSNP2006B_849 |
| 785 | Composition | SsSNP2006B_1566 |
| 786 | Composition | SsSNP2006B_739 |
| 787 | Composition | SsSNP2006B_737 |
| 788 | Composition | SsSNP2006B_134 |
| 789 | Composition | SsSNP2006B_135 |
| 790 | Composition | SsSNP2006B_2398 |
| 791 | Composition | SsSNP2006B_2210 |
| 792 | Composition | SsSNP2006B_990 |
| 793 | Composition | SsSNP2006B_1684 |
| 794 | Composition | SsSNP2006B_2241 |
| 795 | Composition | SsSNP2006B_1577 |
| 796 | Composition | SsSNP2006B_379 |
| 797 | Composition | SsSNP2006B_1733 |
| 798 | Composition | SsSNP2006B_2308 |
| 799 | Composition | SsSNP2006B_1041 |
| 800 | Composition | SsSNP2006B_554 |
| 801 | Composition | SsSNP2006B_721 |
| 802 | Composition | SsSNP2006B_131 |
| 803 | Composition | SsSNP2006B_877 |
| 804 | Composition | SsSNP2006B_2195 |
| 805 | Composition | SsSNP2006B_1600 |
| 806 | Composition | SsSNP2006B_639 |
| 807 | Composition | SsSNP2006B_490 |
| 808 | Composition | SsSNP2006B_548 |
| 809 | Composition | SsSNP2006B_1542 |
| 810 | Composition | SsSNP2006B_1142 |
| 811 | Composition | SsSNP2006B_773 |
| 812 | Composition | SsSNP2006B_200 |
| 813 | Composition | SsSNP2006B_816 |
| 814 | Composition | SsSNP2006B_333 |
| 815 | Composition | SsSNP2006B_1782 |
| 816 | Composition | SsSNP2006B_2032 |
| 817 | Composition | SsSNP2006B_933 |
| 818 | Composition | SsSNP2006B_917 |
| 819 | Composition | SsSNP2006B_2018 |
| 820 | Composition | SsSNP2006B_1842 |
| 821 | Composition | SsSNP2006B_1472 |
| 822 | Composition | SsSNP2006B_2455 |
| 823 | Composition | SsSNP2006B_2341 |
| 824 | Composition | SsSNP2006B_2165 |
| 825 | Composition | SsSNP2006B_1636 |
| 826 | Composition | SsSNP2006B_295 |
| 827 | Composition | SsSNP2006B_2594 |
| 828 | Composition | SsSNP2006B_97 |
| 829 | Composition | SsSNP2006B_1749 |
| 830 | Composition | SsSNP2006B_2277 |
| 831 | Composition | SsSNP2006B_1714 |
| 832 | Composition | SsSNP2006B_1483 |
| 833 | Composition | SsSNP2006B_1380 |
| 834 | Composition | SsSNP2006B_1385 |
| 835 | Composition | SsSNP2006B_1648 |
| 836 | Composition | SsSNP2006B_1229 |
| 837 | Composition | SsSNP2006B_1618 |
| 838 | Composition | SsSNP2006B_1866 |
| 839 | Composition | SsSNP2006B_1051 |
| 840 | Composition | SsSNP2006B_1079 |
| 841 | Composition | SsSNP2006B_1000 |
| 842 | Composition | SsSNP2006B_114 |
| 843 | Composition | SsSNP2006B_1526 |
| 844 | Composition | SsSNP2006B_1567 |
| 845 | Composition | SsSNP2006B_1938 |
| 846 | Composition | SsSNP2006B_47 |
| 847 | Composition | SsSNP2006B_1974 |
| 848 | Composition | SsSNP2006B_983 |
| 849 | Composition | SsSNP2006B_1676 |
| 850 | Composition | SsSNP2006B_171 |
| 851 | Composition | SsSNP2006B_1721 |
| 852 | Composition | SsSNP2006B_744 |
| 853 | Composition | SsSNP2006B_824 |
| 854 | Composition | SsSNP2006B_927 |
| 855 | Composition | SsSNP2006B_2047 |
| 856 | Composition | SsSNP2006B_329 |
| 857 | Composition | SsSNP2006B_2232 |
| 858 | Composition | SsSNP2006B_1922 |
| 859 | Composition | SsSNP2006B_2572 |
| 860 | Composition | SsSNP2006B_1395 |
| 861 | Composition | SsSNP2006B_2003 |
| 862 | Composition | SsSNP2006B_1750 |
| 863 | Composition | SsSNP2006B_1422 |
| 864 | Composition | SsSNP2006B_1504 |
| 865 | Composition | SsSNP2006B_1893 |
| 866 | Composition | SsSNP2006B_146 |
| 867 | Composition | SsSNP2006B_496 |
| 868 | Composition | SsSNP2006B_1307 |
| 869 | Composition | SsSNP2006B_1301 |
| 870 | Composition | SsSNP2006B_2216 |
| 871 | Composition | SsSNP2006B_2456 |
| 872 | Composition | SsSNP2006B_132 |
| 873 | Composition | SsSNP2006B_648 |
| 874 | Composition | SsSNP2006B_332 |
| 875 | Composition | SsSNP2006B_1105 |
| 876 | Composition | SsSNP2006B_1664 |
| 877 | Composition | SsSNP2006B_2247 |
| 878 | Composition | SsSNP2006B_958 |
| 879 | Composition | SsSNP2006B_1305 |
| 880 | Composition | SsSNP2006B_2191 |
| 881 | Composition | SsSNP2006B_1314 |
| 882 | Composition | SsSNP2006B_1647 |
| 883 | Composition | SsSNP2006B_1867 |
| 884 | Composition | SsSNP2006B_1448 |
| 885 | Composition | SsSNP2006B_1646 |
| 886 | Composition | SsSNP2006B_1818 |
| 887 | Composition | SsSNP2006B_2172 |
| 888 | Composition | SsSNP2006B_2570 |
| 889 | Composition | SsSNP2006B_2129 |
| 890 | Composition | SsSNP2006B_1361 |
| 891 | Composition | SsSNP2006B_1952 |
| 892 | Composition | SsSNP2006B_843 |
| 893 | Composition | SsSNP2006B_2215 |
| 894 | Composition | SsSNP2006B_579 |
| 895 | Composition | SsSNP2006B_2103 |
| 896 | Composition | SsSNP2006B_424 |
| 897 | Composition | SsSNP2006B_651 |
| 898 | Composition | SsSNP2006B_1155 |
| 899 | Composition | SsSNP2006B_1921 |
| 900 | Composition | SsSNP2006B_75 |
| 901 | Composition | SsSNP2006B_1383 |
| 902 | Composition | SsSNP2006B_649 |
| 903 | Composition | SsSNP2006B_1174 |
| 904 | Composition | SsSNP2006B_1789 |
| 905 | Composition | SsSNP2006B_2352 |
| 906 | Composition | SsSNP2006B_350 |
| 907 | Composition | SsSNP2006B_545 |
| 908 | Composition | SsSNP2006B_1089 |
| 909 | Composition | SsSNP2006B_975 |
| 910 | Composition | SsSNP2006B_1170 |
| 911 | Composition | SsSNP2006B_582 |
| 912 | Composition | SsSNP2006B_2083 |
| 913 | Composition | SsSNP2006B_668 |
| 914 | Composition | SsSNP2006B_1883 |
| 915 | Composition | SsSNP2006B_2235 |
| 916 | Composition | SsSNP2006B_1500 |
| 917 | Composition | SsSNP2006B_1719 |
| 918 | Composition | SsSNP2006B_1915 |
| 919 | Composition | SsSNP2006B_1284 |
| 920 | Composition | SsSNP2006B_1364 |
| 921 | Composition | SsSNP2006B_1161 |
| 922 | Composition | SsSNP2006B_1546 |
| 923 | Composition | SsSNP2006B_1449 |
| 924 | Composition | SsSNP2006B_449 |
| 925 | Composition | SsSNP2006B_1895 |
| 926 | Composition | SsSNP2006B_71 |
| 927 | Composition | SsSNP2006B_363 |
| 928 | Composition | SsSNP2006B_93 |
| 929 | Composition | SsSNP2006B_948 |
| 930 | Composition | SsSNP2006B_349 |
| 931 | Composition | SsSNP2006B_1626 |
| 932 | Composition | SsSNP2006B_1865 |
| 933 | Composition | SsSNP2006B_1235 |
| 934 | Composition | SsSNP2006B_133 |
| 935 | Composition | SsSNP2006B_324 |
| 936 | Composition | SsSNP2006B_2121 |
| 937 | Composition | SsSNP2006B_1590 |
| 938 | Composition | SsSNP2006B_930 |
| 939 | Composition | SsSNP2006B_1812 |
| 940 | Composition | SsSNP2006B_1901 |
| 941 | Composition | SsSNP2006B_640 |
| 942 | Composition | SsSNP2006B_2340 |
| 943 | Composition | SsSNP2006B_963 |
| 944 | Composition | SsSNP2006B_1270 |
| 945 | Composition | SsSNP2006B_217 |
| 946 | Composition | SsSNP2006B_1898 |
| 947 | Composition | SsSNP2006B_2156 |
| 948 | Composition | SsSNP2006B_390 |
| 949 | Composition | SsSNP2006B_1667 |
| 950 | Composition | SsSNP2006B_573 |
| 951 | Composition | SsSNP2006B_861 |
| 952 | Composition | SsSNP2006B_460 |
| 953 | Composition | SsSNP2006B_2372 |
| 954 | Composition | SsSNP2006B_108 |
| 955 | Composition | SsSNP2006B_498 |
| 956 | Composition | SsSNP2006B_1496 |
| 957 | Composition | SsSNP2006B_572 |
| 958 | Composition | SsSNP2006B_311 |
| 959 | Composition | SsSNP2006B_2036 |
| 960 | Composition | SsSNP2006B_2078 |
| 961 | Composition | SsSNP2006B_2368 |
| 962 | Composition | SsSNP2006B_2369 |
| 963 | Composition | SsSNP2006B_466 |
| 964 | Composition | SsSNP2006B_2589 |
| 965 | Composition | SsSNP2006B_509 |
| 966 | Composition | SsSNP2006B_771 |
| 967 | Composition | SsSNP2006B_551 |
| 968 | Composition | SsSNP2006B_1846 |
| 969 | Composition | SsSNP2006B_1323 |
| 970 | Composition | SsSNP2006B_521 |
| 971 | Composition | SsSNP2006B_1742 |
| 972 | Composition | SsSNP2006B_2108 |
| 973 | Composition | SsSNP2006B_1929 |
| 974 | Composition | SsSNP2006B_1822 |
| 975 | Composition | SsSNP2006B_647 |
| 976 | Composition | SsSNP2006B_2071 |
| 977 | Composition | SsSNP2006B_338 |
| 978 | Composition | SsSNP2006B_513 |
| 979 | Composition | SsSNP2006B_1805 |
| 980 | Composition | SsSNP2006B_817 |
| 981 | Composition | SsSNP2006B_736 |
| 982 | Composition | SsSNP2006B_977 |
| 983 | Composition | SsSNP2006B_1988 |
| 984 | Composition | SsSNP2006B_1029 |
| 985 | Composition | SsSNP2006B_1798 |
| 986 | Composition | SsSNP2006B_2187 |
| 987 | Composition | SsSNP2006B_1728 |
| 988 | Composition | SsSNP2006B_48 |
| 989 | Composition | SsSNP2006B_943 |
| 990 | Composition | SsSNP2006B_1209 |
| 991 | Composition | SsSNP2006B_1477 |
| 992 | Composition | SsSNP2006B_1478 |
| 993 | Composition | SsSNP2006B_1414 |
| 994 | Composition | SsSNP2006B_1272 |
| 995 | Composition | SsSNP2006B_259 |
| 996 | Composition | SsSNP2006B_1180 |
| 997 | Composition | SsSNP2006B_1124 |
| 998 | Composition | SsSNP2006B_1163 |
| 999 | Composition | SsSNP2006B_577 |
| 1000 | Composition | SsSNP2006B_1355 |
| 1001 | Composition | SsSNP2006B_2013 |
| 1002 | Composition | SsSNP2006B_1848 |
| 1003 | Composition | SsSNP2006B_1360 |
| 1004 | Composition | SsSNP2006B_2431 |
| 1005 | Composition | SsSNP2006B_1004 |
| 1006 | Composition | SsSNP2006B_1223 |
| 1007 | Composition | SsSNP2006B_1224 |
| 1008 | Composition | SsSNP2006B_82 |
| 1009 | Composition | SsSNP2006B_2020 |
| 1010 | Composition | SsSNP2006B_1116 |
| 1011 | Composition | SsSNP2006B_1519 |
| 1012 | Composition | SsSNP2006B_1058 |
| 1013 | Composition | SsSNP2006B_361 |
| 1014 | Composition | SsSNP2006B_1886 |
| 1015 | Composition | SsSNP2006B_2514 |
| 1016 | Composition | SsSNP2006B_776 |
| 1017 | Composition | SsSNP2006B_2115 |
| 1018 | Composition | SsSNP2006B_2326 |
| 1019 | Composition | SsSNP2006B_869 |
| 1020 | Composition | SsSNP2006B_55 |
| 1021 | Composition | SsSNP2006B_1701 |
| 1022 | Composition | SsSNP2006B_250 |
| 1023 | Composition | SsSNP2006B_1130 |
| 1024 | Composition | SsSNP2006B_526 |
| 1025 | Composition | SsSNP2006B_184 |
| 1026 | Composition | SsSNP2006B_2243 |
| 1027 | Composition | SsSNP2006B_354 |
| 1028 | Composition | SsSNP2006B_2125 |
| 1029 | Composition | SsSNP2006B_1739 |
| 1030 | Composition | SsSNP2006B_2466 |
| 1031 | Composition | SsSNP2006B_2085 |
| 1032 | Composition | SsSNP2006B_1674 |
| 1033 | Composition | SsSNP2006B_593 |
| 1034 | Composition | SsSNP2006B_2171 |
| 1035 | Composition | SsSNP2006B_1516 |
| 1036 | Composition | SsSNP2006B_1049 |
| 1037 | Composition | SsSNP2006B_667 |
| 1038 | Composition | SsSNP2006B_2218 |
| 1039 | Composition | SsSNP2006B_486 |
| 1040 | Composition | SsSNP2006B_190 |
| 1041 | Composition | SsSNP2006B_1823 |
| 1042 | Composition | SsSNP2006B_679 |
| 1043 | Composition | SsSNP2006B_1391 |
| 1044 | Composition | SsSNP2006B_534 |
| 1045 | Composition | SsSNP2006B_995 |
| 1046 | Composition | SsSNP2006B_1128 |
| 1047 | Composition | SsSNP2006B_1679 |
| 1048 | Composition | SsSNP2006B_1455 |
| 1049 | Composition | SsSNP2006B_1022 |
| 1050 | Composition | SsSNP2006B_703 |
| 1051 | Composition | SsSNP2006B_601 |
| 1052 | Composition | SsSNP2006B_2538 |
| 1053 | Composition | SsSNP2006B_1215 |
| 1054 | Composition | SsSNP2006B_1033 |
| 1055 | Composition | SsSNP2006B_876 |
| 1056 | Composition | SsSNP2006B_1702 |
| 1057 | Composition | SsSNP2006B_99 |
| 1058 | Composition | SsSNP2006B_2446 |
| 1059 | Composition | SsSNP2006B_1970 |
| 1060 | Composition | SsSNP2006B_1807 |
| 1061 | Composition | SsSNP2006B_1553 |
| 1062 | Composition | SsSNP2006B_920 |
| 1063 | Composition | SsSNP2006B_1230 |
| 1064 | Composition | SsSNP2006B_1850 |
| 1065 | Composition | SsSNP2006B_2150 |
| 1066 | Composition | SsSNP2006B_845 |
| 1067 | Composition | SsSNP2006B_2436 |
| 1068 | Composition | SsSNP2006B_1078 |
| 1069 | Composition | SsSNP2006B_1139 |
| 1070 | Composition | SsSNP2006B_2079 |
| 1071 | Composition | SsSNP2006B_1539 |
| 1072 | Composition | SsSNP2006B_1540 |
| 1073 | Composition | SsSNP2006B_2343 |
| 1074 | Composition | SsSNP2006B_2445 |
| 1075 | Composition | SsSNP2006B_313 |
| 1076 | Composition | SsSNP2006B_1926 |
| 1077 | Composition | SsSNP2006B_1991 |
| 1078 | Composition | SsSNP2006B_796 |
| 1079 | Composition | SsSNP2006B_101 |
| 1080 | Composition | SsSNP2006B_838 |
| 1081 | Composition | SsSNP2006B_88 |
| 1082 | Composition | SsSNP2006B_1762 |
| 1083 | Composition | SsSNP2006B_2286 |
| 1084 | Composition | SsSNP2006B_165 |
| 1085 | Composition | SsSNP2006B_16 |
| 1086 | Composition | SsSNP2006B_645 |
| 1087 | Composition | SsSNP2006B_244 |
| 1088 | Composition | SsSNP2006B_1308 |
| 1089 | Composition | SsSNP2006B_380 |
| 1090 | Composition | SsSNP2006B_2336 |
| 1091 | Composition | SsSNP2006B_2420 |
| 1092 | Composition | SsSNP2006B_1808 |
| 1093 | Composition | SsSNP2006B_1409 |
| 1094 | Composition | SsSNP2006B_1469 |
| 1095 | Composition | SsSNP2006B_785 |
| 1096 | Composition | SsSNP2006B_1162 |
| 1097 | Composition | SsSNP2006B_2506 |
| 1098 | Composition | SsSNP2006B_1718 |
| 1099 | Composition | SsSNP2006B_2439 |
| 1100 | Composition | SsSNP2006B_652 |
| 1101 | Composition | SsSNP2006B_2043 |
| 1102 | Composition | SsSNP2006B_381 |
| 1103 | Composition | SsSNP2006B_612 |
| 1104 | Composition | SsSNP2006B_149 |
| 1105 | Composition | SsSNP2006B_266 |
| 1106 | Composition | SsSNP2006B_412 |
| 1107 | Composition | SsSNP2006B_157 |
| 1108 | Composition | SsSNP2006B_1425 |
| 1109 | Composition | SsSNP2006B_728 |
| 1110 | Composition | SsSNP2006B_1953 |
| 1111 | Composition | SsSNP2006B_1710 |
| 1112 | Composition | SsSNP2006B_765 |
| 1113 | Composition | SsSNP2006B_185 |
| 1114 | Composition | SsSNP2006B_2023 |
| 1115 | Composition | SsSNP2006B_968 |
| 1116 | Composition | SsSNP2006B_2155 |
| 1117 | Composition | SsSNP2006B_887 |
| 1118 | Composition | SsSNP2006B_1839 |
| 1119 | Composition | SsSNP2006B_2082 |
| 1120 | Composition | SsSNP2006B_622 |
| 1121 | Composition | SsSNP2006B_863 |
| 1122 | Composition | SsSNP2006B_1154 |
| 1123 | Composition | SsSNP2006B_1432 |
| 1124 | Composition | SsSNP2006B_1518 |
| 1125 | Composition | SsSNP2006B_2422 |
| 1126 | Composition | SsSNP2006B_1118 |
| 1127 | Composition | SsSNP2006B_1269 |
| 1128 | Composition | SsSNP2006B_1437 |
| 1129 | Composition | SsSNP2006B_224 |
| 1130 | Composition | SsSNP2006B_1012 |
| 1131 | Composition | SsSNP2006B_1578 |
| 1132 | Composition | SsSNP2006B_22 |
| 1133 | Composition | SsSNP2006B_119 |
| 1134 | Composition | SsSNP2006B_1709 |
| 1135 | Composition | SsSNP2006B_1932 |
| 1136 | Composition | SsSNP2006B_941 |
| 1137 | Composition | SsSNP2006B_1046 |
| 1138 | Composition | SsSNP2006B_880 |
| 1139 | Composition | SsSNP2006B_1565 |
| 1140 | Composition | SsSNP2006B_666 |
| 1141 | Composition | SsSNP2006B_2236 |
| 1142 | Composition | SsSNP2006B_778 |
| 1143 | Composition | SsSNP2006B_143 |
| 1144 | Composition | SsSNP2006B_1147 |
| 1145 | Composition | SsSNP2006B_479 |
| 1146 | Composition | SsSNP2006B_2490 |
| 1147 | Composition | SsSNP2006B_1075 |
| 1148 | Composition | SsSNP2006B_138 |
| 1149 | Composition | SsSNP2006B_530 |
| 1150 | Composition | SsSNP2006B_1786 |
| 1151 | Composition | SsSNP2006B_902 |
| 1152 | Composition | SsSNP2006B_170 |
| 1153 | Composition | SsSNP2006B_660 |
| 1154 | Composition | SsSNP2006B_272 |
| 1155 | Composition | SsSNP2006B_1700 |
| 1156 | Composition | SsSNP2006B_1914 |
| 1157 | Composition | SsSNP2006B_1379 |
| 1158 | Composition | SsSNP2006B_1632 |
| 1159 | Composition | SsSNP2006B_2519 |
| 1160 | Composition | SsSNP2006B_292 |
| 1161 | Composition | SsSNP2006B_252 |
| 1162 | Composition | SsSNP2006B_853 |
| 1163 | Composition | SsSNP2006B_2325 |
| 1164 | Composition | SsSNP2006B_103 |
| 1165 | Composition | SsSNP2006B_239 |
| 1166 | Composition | SsSNP2006B_240 |
| 1167 | Composition | SsSNP2006B_1393 |
| 1168 | Composition | SsSNP2006B_2601 |
| 1169 | Composition | SsSNP2006B_136 |
| 1170 | Composition | SsSNP2006B_137 |
| 1171 | Composition | SsSNP2006B_1423 |
| 1172 | Composition | SsSNP2006B_1763 |
| 1173 | Composition | SsSNP2006B_1589 |
| 1174 | Composition | SsSNP2006B_999 |
| 1175 | Composition | SsSNP2006B_1283 |
| 1176 | Composition | SsSNP2006B_829 |
| 1177 | Composition | SsSNP2006B_1225 |
| 1178 | Composition | SsSNP2006B_2438 |
| 1179 | Composition | SsSNP2006B_717 |
| 1180 | Composition | SsSNP2006B_1474 |
| 1181 | Composition | SsSNP2006B_2324 |
| 1182 | Composition | SsSNP2006B_294 |
| 1183 | Composition | SsSNP2006B_2131 |
| 1184 | Composition | SsSNP2006B_251 |
| 1185 | Composition | SsSNP2006B_1104 |
| 1186 | Composition | SsSNP2006B_2444 |
| 1187 | Composition | SsSNP2006B_644 |
| 1188 | Composition | SsSNP2006B_1428 |
| 1189 | Composition | SsSNP2006B_433 |
| 1190 | Composition | SsSNP2006B_1507 |
| 1191 | Composition | SsSNP2006B_1708 |
| 1192 | Composition | SsSNP2006B_2124 |
| 1193 | Composition | SsSNP2006B_1446 |
| 1194 | Composition | SsSNP2006B_2433 |
| 1195 | Composition | SsSNP2006B_2120 |
| 1196 | Composition | SsSNP2006B_10 |
| 1197 | Composition | SsSNP2006B_1111 |
| 1198 | Composition | SsSNP2006B_1471 |
| 1199 | Composition | SsSNP2006B_998 |
| 1200 | Composition | SsSNP2006B_707 |
| 1201 | Composition | SsSNP2006B_2040 |
| 1202 | Composition | SsSNP2006B_499 |
| 1203 | Composition | SsSNP2006B_2407 |
| 1204 | Composition | SsSNP2006B_929 |
| 1205 | Composition | SsSNP2006B_484 |
| 1206 | Composition | SsSNP2006B_781 |
| 1207 | Composition | SsSNP2006B_230 |
| 1208 | Composition | SsSNP2006B_2088 |
| 1209 | Composition | SsSNP2006B_2435 |
| 1210 | Composition | SsSNP2006B_724 |
| 1211 | Composition | SsSNP2006B_1877 |
| 1212 | Composition | SsSNP2006B_2189 |
| 1213 | Composition | SsSNP2006B_2573 |
| 1214 | Composition | SsSNP2006B_359 |
| 1215 | Composition | SsSNP2006B_2031 |
| 1216 | Composition | SsSNP2006B_1900 |
| 1217 | Composition | SsSNP2006B_1959 |
| 1218 | Composition | SsSNP2006B_1650 |
| 1219 | Composition | SsSNP2006B_2441 |
| 1220 | Composition | SsSNP2006B_1989 |
| 1221 | Composition | SsSNP2006B_2561 |
| 1222 | Composition | SsSNP2006B_987 |
| 1223 | Composition | SsSNP2006B_1168 |
| 1224 | Composition | SsSNP2006B_1456 |
| 1225 | Composition | SsSNP2006B_2523 |
| 1226 | Composition | SsSNP2006B_2104 |
| 1227 | Composition | SsSNP2006B_2524 |
| 1228 | Composition | SsSNP2006B_1250 |
| 1229 | Composition | SsSNP2006B_1487 |
| 1230 | Composition | SsSNP2006B_1658 |
| 1231 | Composition | SsSNP2006B_1153 |
| 1232 | Composition | SsSNP2006B_160 |
| 1233 | Composition | SsSNP2006B_2290 |
| 1234 | Composition | SsSNP2006B_1304 |
| 1235 | Composition | SsSNP2006B_2358 |
| 1236 | Composition | SsSNP2006B_2427 |
| 1237 | Composition | SsSNP2006B_447 |
| 1238 | Composition | SsSNP2006B_1917 |
| 1239 | Composition | SsSNP2006B_152 |
| 1240 | Composition | SsSNP2006B_1882 |
| 1241 | Composition | SsSNP2006B_2102 |
| 1242 | Composition | SsSNP2006B_2588 |
| 1243 | Composition | SsSNP2006B_596 |
| 1244 | Composition | SsSNP2006B_1289 |
| 1245 | Composition | SsSNP2006B_319 |
| 1246 | Composition | SsSNP2006B_1208 |
| 1247 | Composition | SsSNP2006B_191 |
| 1248 | Composition | SsSNP2006B_1735 |
| 1249 | Composition | SsSNP2006B_1736 |
| 1250 | Composition | SsSNP2006B_1790 |
| 1251 | Composition | SsSNP2006B_1668 |
| 1252 | Composition | SsSNP2006B_632 |
| 1253 | Composition | SsSNP2006B_1582 |
| 1254 | Composition | SsSNP2006B_1854 |
| 1255 | Composition | SsSNP2006B_468 |
| 1256 | Composition | SsSNP2006B_1688 |
| 1257 | Composition | SsSNP2006B_1125 |
| 1258 | Composition | SsSNP2006B_1918 |
| 1259 | Composition | SsSNP2006B_2475 |
| 1260 | Composition | SsSNP2006B_2304 |
| 1261 | Composition | SsSNP2006B_182 |
| 1262 | Composition | SsSNP2006B_1635 |
| 1263 | Composition | SsSNP2006B_321 |
| 1264 | Composition | SsSNP2006B_1184 |
| 1265 | Composition | SsSNP2006B_799 |
| 1266 | Composition | SsSNP2006B_2068 |
| 1267 | Composition | SsSNP2006B_318 |
| 1268 | Composition | SsSNP2006B_210 |
| 1269 | Composition | SsSNP2006B_2067 |
| 1270 | Composition | SsSNP2006B_309 |
| 1271 | Composition | SsSNP2006B_2531 |
| 1272 | Composition | SsSNP2006B_1493 |
| 1273 | Composition | SsSNP2006B_1910 |
| 1274 | Composition | SsSNP2006B_2316 |
| 1275 | Composition | SsSNP2006B_2443 |
| 1276 | Composition | SsSNP2006B_1194 |
| 1277 | Composition | SsSNP2006B_2212 |
| 1278 | Composition | SsSNP2006B_2207 |
| 1279 | Composition | SsSNP2006B_1868 |
| 1280 | Composition | SsSNP2006B_1401 |
| 1281 | Composition | SsSNP2006B_2136 |
| 1282 | Composition | SsSNP2006B_60 |
| 1283 | Composition | SsSNP2006B_792 |
| 1284 | Composition | SsSNP2006B_793 |
| 1285 | Composition | SsSNP2006B_2437 |
| 1286 | Composition | SsSNP2006B_1312 |
| 1287 | Composition | SsSNP2006B_1484 |
| 1288 | Composition | SsSNP2006B_2300 |
| 1289 | Composition | SsSNP2006B_1641 |
| 1290 | Composition | SsSNP2006B_713 |
| 1291 | Composition | SsSNP2006B_791 |
| 1292 | Composition | SsSNP2006B_299 |
| 1293 | Composition | SsSNP2006B_1021 |
| 1294 | Composition | SsSNP2006B_2034 |
| 1295 | Composition | SsSNP2006B_945 |
| 1296 | Composition | SsSNP2006B_2547 |
| 1297 | Composition | SsSNP2006B_851 |
| 1298 | Composition | SsSNP2006B_2403 |
| 1299 | Composition | SsSNP2006B_700 |
| 1300 | Composition | SsSNP2006B_939 |
| 1301 | Composition | SsSNP2006B_463 |
| 1302 | Composition | SsSNP2006B_865 |
| 1303 | Composition | SsSNP2006B_1123 |
| 1304 | Composition | SsSNP2006B_451 |
| 1305 | Composition | SsSNP2006B_985 |
| 1306 | Composition | SsSNP2006B_1903 |
| 1307 | Composition | SsSNP2006B_937 |
| 1308 | Composition | SsSNP2006B_1336 |
| 1309 | Composition | SsSNP2006B_493 |
| 1310 | Composition | SsSNP2006B_2562 |
| 1311 | Composition | SsSNP2006B_337 |
| 1312 | Composition | SsSNP2006B_2454 |
| 1313 | Composition | SsSNP2006B_1495 |
| 1314 | Composition | SsSNP2006B_922 |
| 1315 | Composition | SsSNP2006B_923 |
| 1316 | Composition | SsSNP2006B_2298 |
| 1317 | Composition | SsSNP2006B_207 |
| 1318 | Composition | SsSNP2006B_563 |
| 1319 | Composition | SsSNP2006B_1627 |
| 1320 | Composition | SsSNP2006B_1920 |
| 1321 | Composition | SsSNP2006B_2597 |
| 1322 | Composition | SsSNP2006B_1611 |
| 1323 | Composition | SsSNP2006B_1960 |
| 1324 | Composition | SsSNP2006B_1996 |
| 1325 | Composition | SsSNP2006B_623 |
| 1326 | Composition | SsSNP2006B_2255 |
| 1327 | Composition | SsSNP2006B_1697 |
| 1328 | Composition | SsSNP2006B_973 |
| 1329 | Composition | SsSNP2006B_2527 |
| 1330 | Composition | SsSNP2006B_1460 |
| 1331 | Composition | SsSNP2006B_831 |
| 1332 | Composition | SsSNP2006B_1381 |
| 1333 | Composition | SsSNP2006B_144 |
| 1334 | Composition | SsSNP2006B_616 |
| 1335 | Composition | SsSNP2006B_986 |
| 1336 | Composition | SsSNP2006B_1941 |
| 1337 | Composition | SsSNP2006B_2019 |
| 1338 | Composition | SsSNP2006B_2266 |
| 1339 | Composition | SsSNP2006B_306 |
| 1340 | Composition | SsSNP2006B_1293 |
| 1341 | Composition | SsSNP2006B_202 |
| 1342 | Composition | SsSNP2006B_591 |
| 1343 | Composition | SsSNP2006B_298 |
| 1344 | Composition | SsSNP2006B_1011 |
| 1345 | Composition | SsSNP2006B_834 |
| 1346 | Composition | SsSNP2006B_172 |
| 1347 | Composition | SsSNP2006B_1169 |
| 1348 | Composition | SsSNP2006B_2451 |
| 1349 | Composition | SsSNP2006B_258 |
| 1350 | Composition | SsSNP2006B_2440 |
| 1351 | Composition | SsSNP2006B_775 |
| 1352 | Composition | SsSNP2006B_1055 |
| 1353 | Composition | SsSNP2006B_1249 |
| 1354 | Composition | SsSNP2006B_1591 |
| 1355 | Composition | SsSNP2006B_1757 |
| 1356 | Composition | SsSNP2006B_2345 |
| 1357 | Composition | SsSNP2006B_1772 |
| 1358 | Composition | SsSNP2006B_587 |
| 1359 | Composition | SsSNP2006B_588 |
| 1360 | Composition | SsSNP2006B_803 |
| 1361 | Composition | SsSNP2006B_1346 |
| 1362 | Composition | SsSNP2006B_2382 |
| 1363 | Composition | SsSNP2006B_786 |
| 1364 | Composition | SsSNP2006B_1183 |
| 1365 | Composition | SsSNP2006B_889 |
| 1366 | Composition | SsSNP2006B_2184 |
| 1367 | Composition | SsSNP2006B_2153 |
| 1368 | Composition | SsSNP2006B_892 |
| 1369 | Composition | SsSNP2006B_2347 |
| 1370 | Composition | SsSNP2006B_2412 |
| 1371 | Composition | SsSNP2006B_1978 |
| 1372 | Composition | SsSNP2006B_2430 |
| 1373 | Composition | SsSNP2006B_1332 |
| 1374 | Composition | SsSNP2006B_367 |
| 1375 | Composition | SsSNP2006B_1324 |
| 1376 | Composition | SsSNP2006B_1548 |
| 1377 | Composition | SsSNP2006B_1171 |
| 1378 | Composition | SsSNP2006B_371 |
| 1379 | Composition | SsSNP2006B_1277 |
| 1380 | Composition | SsSNP2006B_104 |
| 1381 | Composition | SsSNP2006B_1605 |
| 1382 | Composition | SsSNP2006B_1072 |
| 1383 | Composition | SsSNP2006B_965 |
| 1384 | Composition | SsSNP2006B_1943 |
| 1385 | Composition | SsSNP2006B_110 |
| 1386 | Composition | SsSNP2006B_362 |
| 1387 | Composition | SsSNP2006B_1309 |
| 1388 | Composition | SsSNP2006B_1019 |
| 1389 | Composition | SsSNP2006B_1038 |
| 1390 | Composition | SsSNP2006B_866 |
| 1391 | Composition | SsSNP2006B_2066 |
| 1392 | Composition | SsSNP2006B_343 |
| 1393 | Composition | SsSNP2006B_1527 |
| 1394 | Composition | SsSNP2006B_297 |
| 1395 | Composition | SsSNP2006B_2528 |
| 1396 | Composition | SsSNP2006B_261 |
| 1397 | Composition | SsSNP2006B_2017 |
| 1398 | Composition | SsSNP2006B_215 |
| 1399 | Composition | SsSNP2006B_1274 |
| 1400 | Composition | SsSNP2006B_1751 |
| 1401 | Composition | SsSNP2006B_1316 |
| 1402 | Composition | SsSNP2006B_1800 |
| 1403 | Composition | SsSNP2006B_1008 |
| 1404 | Composition | SsSNP2006B_2035 |
| 1405 | Composition | SsSNP2006B_2169 |
| 1406 | Composition | SsSNP2006B_1573 |
| 1407 | Composition | SsSNP2006B_388 |
| 1408 | Composition | SsSNP2006B_1354 |
| 1409 | Composition | SsSNP2006B_241 |
| 1410 | Composition | SsSNP2006B_242 |
| 1411 | Composition | SsSNP2006B_386 |
| 1412 | Composition | SsSNP2006B_944 |
| 1413 | Composition | SsSNP2006B_2504 |
| 1414 | Composition | SsSNP2006B_2593 |
| 1415 | Composition | SsSNP2006B_1972 |
| 1416 | Composition | SsSNP2006B_1885 |
| 1417 | Composition | SsSNP2006B_811 |
| 1418 | Composition | SsSNP2006B_2317 |
| 1419 | Composition | SsSNP2006B_1950 |
| 1420 | Composition | SsSNP2006B_1597 |
| 1421 | Composition | SsSNP2006B_1117 |
| 1422 | Composition | SsSNP2006B_1940 |
| 1423 | Composition | SsSNP2006B_1053 |
| 1424 | Composition | SsSNP2006B_1061 |
| 1425 | Composition | SsSNP2006B_1321 |
| 1426 | Composition | SsSNP2006B_1746 |
| 1427 | Composition | SsSNP2006B_1816 |
| 1428 | Composition | SsSNP2006B_2246 |
| 1429 | Composition | SsSNP2006B_1629 |
| 1430 | Composition | SsSNP2006B_1102 |
| 1431 | Composition | SsSNP2006B_1291 |
| 1432 | Composition | SsSNP2006B_2110 |
| 1433 | Composition | SsSNP2006B_2399 |
| 1434 | Composition | SsSNP2006B_1402 |
| 1435 | Composition | SsSNP2006B_2114 |
| 1436 | Composition | SsSNP2006B_926 |
| 1437 | Composition | SsSNP2006B_2494 |
| 1438 | Composition | SsSNP2006B_1244 |
| 1439 | Composition | SsSNP2006B_2544 |
| 1440 | Composition | SsSNP2006B_467 |
| 1441 | Composition | SsSNP2006B_2585 |
| 1442 | Composition | SsSNP2006B_1433 |
| 1443 | Composition | SsSNP2006B_2488 |
| 1444 | Composition | SsSNP2006B_2230 |
| 1445 | Composition | SsSNP2006B_265 |
| 1446 | Composition | SsSNP2006B_107 |
| 1447 | Composition | SsSNP2006B_2263 |
| 1448 | Composition | SsSNP2006B_2329 |
| 1449 | Composition | SsSNP2006B_2360 |
| 1450 | Composition | SsSNP2006B_564 |
| 1451 | Composition | SsSNP2006B_1065 |
| 1452 | Composition | SsSNP2006B_1213 |
| 1453 | Composition | SsSNP2006B_1814 |
| 1454 | Composition | SsSNP2006B_483 |
| 1455 | Composition | SsSNP2006B_518 |
| 1456 | Composition | SsSNP2006B_78 |
| 1457 | Composition | SsSNP2006B_2209 |
| 1458 | Composition | SsSNP2006B_2086 |
| 1459 | Composition | SsSNP2006B_1593 |
| 1460 | Composition | SsSNP2006B_981 |
| 1461 | Composition | SsSNP2006B_988 |
| 1462 | Composition | SsSNP2006B_122 |
| 1463 | Composition | SsSNP2006B_2567 |
| 1464 | Composition | SsSNP2006B_1282 |
| 1465 | Composition | SsSNP2006B_2072 |
| 1466 | Composition | SsSNP2006B_2305 |
| 1467 | Composition | SsSNP2006B_310 |
| 1468 | Composition | SsSNP2006B_1074 |
| 1469 | Composition | SsSNP2006B_767 |
| 1470 | Composition | SsSNP2006B_1015 |
| 1471 | Composition | SsSNP2006B_1375 |
| 1472 | Composition | SsSNP2006B_794 |
| 1473 | Composition | SsSNP2006B_2256 |
| 1474 | Composition | SsSNP2006B_910 |
| 1475 | Composition | SsSNP2006B_1344 |
| 1476 | Composition | SsSNP2006B_1262 |
| 1477 | Composition | SsSNP2006B_575 |
| 1478 | Composition | SsSNP2006B_494 |
| 1479 | Composition | SsSNP2006B_2146 |
| 1480 | Composition | SsSNP2006B_565 |
| 1481 | Composition | SsSNP2006B_1656 |
| 1482 | Composition | SsSNP2006B_1173 |
| 1483 | Composition | SsSNP2006B_2197 |
| 1484 | Composition | SsSNP2006B_64 |
| 1485 | Composition | SsSNP2006B_942 |
| 1486 | Composition | SsSNP2006B_2168 |
| 1487 | Composition | SsSNP2006B_746 |
| 1488 | Composition | SsSNP2006B_1016 |
| 1489 | Composition | SsSNP2006B_1093 |
| 1490 | Composition | SsSNP2006B_1143 |
| 1491 | Composition | SsSNP2006B_72 |
| 1492 | Composition | SsSNP2006B_1345 |
| 1493 | Composition | SsSNP2006B_837 |
| 1494 | Composition | SsSNP2006B_80 |
| 1495 | Composition | SsSNP2006B_2390 |
| 1496 | Composition | SsSNP2006B_1558 |
| 1497 | Composition | SsSNP2006B_2464 |
| 1498 | Composition | SsSNP2006B_694 |
| 1499 | Composition | SsSNP2006B_952 |
| 1500 | Composition | SsSNP2006B_98 |
| 1501 | Composition | SsSNP2006B_527 |
| 1502 | Composition | SsSNP2006B_1570 |
| 1503 | Composition | SsSNP2006B_1720 |
| 1504 | Composition | SsSNP2006B_1003 |
| 1505 | Composition | SsSNP2006B_964 |
| 1506 | Composition | SsSNP2006B_2024 |
| 1507 | Composition | SsSNP2006B_2126 |
| 1508 | Composition | SsSNP2006B_903 |
| 1509 | Composition | SsSNP2006B_1756 |
| 1510 | Composition | SsSNP2006B_1838 |
| 1511 | Composition | SsSNP2006B_1136 |
| 1512 | Composition | SsSNP2006B_2339 |
| 1513 | Composition | SsSNP2006B_1685 |
| 1514 | Composition | SsSNP2006B_1649 |
| 1515 | Composition | SsSNP2006B_2410 |
| 1516 | Composition | SsSNP2006B_435 |
| 1517 | Composition | SsSNP2006B_2208 |
| 1518 | Composition | SsSNP2006B_127 |
| 1519 | Composition | SsSNP2006B_670 |
| 1520 | Composition | SsSNP2006B_145 |
| 1521 | Composition | SsSNP2006B_2574 |
| 1522 | Composition | SsSNP2006B_1152 |
| 1523 | Composition | SsSNP2006B_1236 |
| 1524 | Composition | SsSNP2006B_1287 |
| 1525 | Composition | SsSNP2006B_1876 |
| 1526 | Composition | SsSNP2006B_2059 |
| 1527 | Composition | SsSNP2006B_994 |
| 1528 | Composition | SsSNP2006B_2049 |
| 1529 | Composition | SsSNP2006B_2442 |
| 1530 | Composition | SsSNP2006B_1645 |
| 1531 | Composition | SsSNP2006B_2337 |
| 1532 | Composition | SsSNP2006B_255 |
| 1533 | Composition | SsSNP2006B_2303 |
| 1534 | Composition | SsSNP2006B_2318 |
| 1535 | Composition | SsSNP2006B_2518 |
| 1536 | Composition | SsSNP2006B_558 |
| 1537 | Composition | SsSNP2006B_283 |
| 1538 | Composition | SsSNP2006B_1845 |
| 1539 | Composition | SsSNP2006B_1774 |
| 1540 | Composition | SsSNP2006B_1200 |
| 1541 | Composition | SsSNP2006B_2480 |
| 1542 | Composition | SsSNP2006B_657 |
| 1543 | Composition | SsSNP2006B_2362 |
| 1544 | Composition | SsSNP2006B_503 |
| 1545 | Composition | SsSNP2006B_991 |
| 1546 | Composition | SsSNP2006B_1874 |
| 1547 | Composition | SsSNP2006B_812 |
| 1548 | Composition | SsSNP2006B_1608 |
| 1549 | Composition | SsSNP2006B_2046 |
| 1550 | Composition | SsSNP2006B_637 |
| 1551 | Composition | SsSNP2006B_714 |
| 1552 | Composition | SsSNP2006B_168 |
| 1553 | Composition | SsSNP2006B_167 |
| 1554 | Composition | SsSNP2006B_2333 |
| 1555 | Composition | SsSNP2006B_1159 |
| 1556 | Composition | SsSNP2006B_1166 |
| 1557 | Composition | SsSNP2006B_1675 |
| 1558 | Composition | SsSNP2006B_584 |
| 1559 | Composition | SsSNP2006B_938 |
| 1560 | Composition | SsSNP2006B_223 |
| 1561 | Composition | SsSNP2006B_2147 |
| 1562 | Composition | SsSNP2006B_1680 |
| 1563 | Composition | SsSNP2006B_1858 |
| 1564 | Composition | SsSNP2006B_698 |
| 1565 | Composition | SsSNP2006B_2022 |
| 1566 | Composition | SsSNP2006B_2173 |
| 1567 | Composition | SsSNP2006B_19 |
| 1568 | Composition | SsSNP2006B_61 |
| 1569 | Composition | SsSNP2006B_285 |
| 1570 | Composition | SsSNP2006B_1382 |
| 1571 | Composition | SsSNP2006B_1752 |
| 1572 | Composition | SsSNP2006B_1604 |
| 1573 | Composition | SsSNP2006B_2543 |
| 1574 | Composition | SsSNP2006B_31 |
| 1575 | Composition | SsSNP2006B_400 |
| 1576 | Composition | SsSNP2006B_401 |
| 1577 | Composition | SsSNP2006B_636 |
| 1578 | Composition | SsSNP2006B_628 |
| 1579 | Composition | SsSNP2006B_1844 |
| 1580 | Composition | SsSNP2006B_808 |
| 1581 | Composition | SsSNP2006B_809 |
| 1582 | Composition | SsSNP2006B_1241 |
| 1583 | Composition | SsSNP2006B_1267 |
| 1584 | Composition | SsSNP2006B_610 |
| 1585 | Composition | SsSNP2006B_769 |
| 1586 | Composition | SsSNP2006B_1302 |
| 1587 | Composition | SsSNP2006B_1190 |
| 1588 | Composition | SsSNP2006B_2313 |
| 1589 | Composition | SsSNP2006B_437 |
| 1590 | Composition | SsSNP2006B_1396 |
| 1591 | Composition | SsSNP2006B_1906 |
| 1592 | Composition | SsSNP2006B_718 |
| 1593 | Composition | SsSNP2006B_1 |
| 1594 | Composition | SsSNP2006B_2 |
| 1595 | Composition | SsSNP2006B_546 |
| 1596 | Composition | SsSNP2006B_2392 |
| 1597 | Composition | SsSNP2006B_1095 |
| 1598 | Composition | SsSNP2006B_2508 |
| 1599 | Composition | SsSNP2006B_1329 |
| 1600 | Composition | SsSNP2006B_2080 |
| 1601 | Composition | SsSNP2006B_1778 |
| 1602 | Composition | SsSNP2006B_621 |
| 1603 | Composition | SsSNP2006B_508 |
| 1604 | Composition | SsSNP2006B_118 |
| 1605 | Composition | SsSNP2006B_2583 |
| 1606 | Composition | SsSNP2006B_1411 |
| 1607 | Composition | SsSNP2006B_1412 |
| 1608 | Composition | SsSNP2006B_1621 |
| 1609 | Composition | SsSNP2006B_1705 |
| 1610 | Composition | SsSNP2006B_931 |
| 1611 | Composition | SsSNP2006B_2178 |
| 1612 | Composition | SsSNP2006B_1729 |
| 1613 | Composition | SsSNP2006B_1678 |
| 1614 | Composition | SsSNP2006B_1438 |
| 1615 | Composition | SsSNP2006B_316 |
| 1616 | Composition | SsSNP2006B_161 |
| 1617 | Composition | SsSNP2006B_806 |
| 1618 | Composition | SsSNP2006B_1509 |
| 1619 | Composition | SsSNP2006B_2582 |
| 1620 | Composition | SsSNP2006B_672 |
| 1621 | Composition | SsSNP2006B_2415 |
| 1622 | Composition | SsSNP2006B_1052 |
| 1623 | Composition | SsSNP2006B_1849 |
| 1624 | Composition | SsSNP2006B_2096 |
| 1625 | Composition | SsSNP2006B_5 |
| 1626 | Composition | SsSNP2006B_2314 |
| 1627 | Composition | SsSNP2006B_425 |
| 1628 | Composition | SsSNP2006B_345 |
| 1629 | Composition | SsSNP2006B_357 |
| 1630 | Composition | SsSNP2006B_1303 |
| 1631 | Composition | SsSNP2006B_599 |
| 1632 | Composition | SsSNP2006B_1056 |
| 1633 | Composition | SsSNP2006B_1388 |
| 1634 | Composition | SsSNP2006B_1541 |
| 1635 | Composition | SsSNP2006B_2371 |
| 1636 | Composition | SsSNP2006B_748 |
| 1637 | Composition | SsSNP2006B_1536 |
| 1638 | Composition | SsSNP2006B_1204 |
| 1639 | Composition | SsSNP2006B_1737 |
| 1640 | Composition | SsSNP2006B_352 |
| 1641 | Composition | SsSNP2006B_1218 |
| 1642 | Composition | SsSNP2006B_2577 |
| 1643 | Composition | SsSNP2006B_586 |
| 1644 | Composition | SsSNP2006B_1968 |
| 1645 | Composition | SsSNP2006B_2006 |
| 1646 | Composition | SsSNP2006B_1044 |
| 1647 | Composition | SsSNP2006B_1322 |
| 1648 | Composition | SsSNP2006B_789 |
| 1649 | Composition | SsSNP2006B_704 |
| 1650 | Composition | SsSNP2006B_1592 |
| 1651 | Composition | SsSNP2006B_1946 |
| 1652 | Composition | SsSNP2006B_1416 |
| 1653 | Composition | SsSNP2006B_2089 |
| 1654 | Composition | SsSNP2006B_1189 |
| 1655 | Composition | SsSNP2006B_918 |
| 1656 | Composition | SsSNP2006B_2472 |
| 1657 | Composition | SsSNP2006B_2473 |
| 1658 | Composition | SsSNP2006B_2194 |
| 1659 | Composition | SsSNP2006B_2546 |
| 1660 | Composition | SsSNP2006B_2182 |
| 1661 | Composition | SsSNP2006B_2098 |
| 1662 | Composition | SsSNP2006B_438 |
| 1663 | Composition | SsSNP2006B_1311 |
| 1664 | Composition | SsSNP2006B_405 |
| 1665 | Composition | SsSNP2006B_1288 |
| 1666 | Composition | SsSNP2006B_2596 |
| 1667 | Composition | SsSNP2006B_29 |
| 1668 | Composition | SsSNP2006B_764 |
| 1669 | Composition | SsSNP2006B_355 |
| 1670 | Composition | SsSNP2006B_300 |
| 1671 | Composition | SsSNP2006B_216 |
| 1672 | Composition | SsSNP2006B_814 |
| 1673 | Composition | SsSNP2006B_404 |
| 1674 | Composition | SsSNP2006B_2177 |
| 1675 | Composition | SsSNP2006B_2260 |
| 1676 | Composition | SsSNP2006B_85 |
| 1677 | Composition | SsSNP2006B_972 |
| 1678 | Composition | SsSNP2006B_624 |
| 1679 | Composition | SsSNP2006B_188 |
| 1680 | Composition | SsSNP2006B_2469 |
| 1681 | Composition | SsSNP2006B_279 |
| 1682 | Composition | SsSNP2006B_2252 |
| 1683 | Composition | SsSNP2006B_1405 |
| 1684 | Composition | SsSNP2006B_2484 |
| 1685 | Composition | SsSNP2006B_1353 |
| 1686 | Composition | SsSNP2006B_1413 |
| 1687 | Composition | SsSNP2006B_576 |
| 1688 | Composition | SsSNP2006B_1545 |
| 1689 | Composition | SsSNP2006B_1537 |
| 1690 | Composition | SsSNP2006B_544 |
| 1691 | Composition | SsSNP2006B_2311 |
| 1692 | Composition | SsSNP2006B_1210 |
| 1693 | Composition | SsSNP2006B_2041 |
| 1694 | Composition | SsSNP2006B_735 |
| 1695 | Composition | SsSNP2006B_2274 |
| 1696 | Composition | SsSNP2006B_1724 |
| 1697 | Composition | SsSNP2006B_1973 |
| 1698 | Composition | SsSNP2006B_1852 |
| 1699 | Composition | SsSNP2006B_520 |
| 1700 | Composition | SsSNP2006B_638 |
| 1701 | Composition | SsSNP2006B_900 |
| 1702 | Composition | SsSNP2006B_2070 |
| 1703 | Composition | SsSNP2006B_1639 |
| 1704 | Composition | SsSNP2006B_2463 |
| 1705 | Composition | SsSNP2006B_1363 |
| 1706 | Composition | SsSNP2006B_514 |
| 1707 | Composition | SsSNP2006B_2568 |
| 1708 | Composition | SsSNP2006B_12 |
| 1709 | Composition | SsSNP2006B_2042 |
| 1710 | Composition | SsSNP2006B_1254 |
| 1711 | Composition | SsSNP2006B_1799 |
| 1712 | Composition | SsSNP2006B_2350 |
| 1713 | Composition | SsSNP2006B_232 |
| 1714 | Composition | SsSNP2006B_407 |
| 1715 | Composition | SsSNP2006B_1534 |
| 1716 | Composition | SsSNP2006B_1343 |
| 1717 | Composition | SsSNP2006B_2421 |
| 1718 | Composition | SsSNP2006B_1515 |
| 1719 | Composition | SsSNP2006B_1810 |
| 1720 | Composition | SsSNP2006B_382 |
| 1721 | Composition | SsSNP2006B_570 |
| 1722 | Composition | SsSNP2006B_270 |
| 1723 | Composition | SsSNP2006B_1255 |
| 1724 | Composition | SsSNP2006B_872 |
| 1725 | Composition | SsSNP2006B_2026 |
| 1726 | Composition | SsSNP2006B_197 |
| 1727 | Composition | SsSNP2006B_529 |
| 1728 | Composition | SsSNP2006B_1598 |
| 1729 | Composition | SsSNP2006B_2219 |
| 1730 | Composition | SsSNP2006B_996 |
| 1731 | Composition | SsSNP2006B_1352 |
| 1732 | Composition | SsSNP2006B_246 |
| 1733 | Composition | SsSNP2006B_2275 |
| 1734 | Composition | SsSNP2006B_2276 |
| 1735 | Composition | SsSNP2006B_2139 |
| 1736 | Composition | SsSNP2006B_2027 |
| 1737 | Composition | SsSNP2006B_1158 |
| 1738 | Composition | SsSNP2006B_364 |
| 1739 | Composition | SsSNP2006B_1232 |
| 1740 | Composition | SsSNP2006B_1037 |
| 1741 | Composition | SsSNP2006B_2253 |
| 1742 | Composition | SsSNP2006B_2254 |
| 1743 | Composition | SsSNP2006B_1722 |
| 1744 | Composition | SsSNP2006B_2183 |
| 1745 | Composition | SsSNP2006B_187 |
| 1746 | Composition | SsSNP2006B_269 |
| 1747 | Composition | SsSNP2006B_1138 |
| 1748 | Composition | SsSNP2006B_173 |
| 1749 | Composition | SsSNP2006B_1299 |
| 1750 | Composition | SsSNP2006B_218 |
| 1751 | Composition | SsSNP2006B_1884 |
| 1752 | Composition | SsSNP2006B_2312 |
| 1753 | Composition | SsSNP2006B_1783 |
| 1754 | Composition | SsSNP2006B_308 |
| 1755 | Composition | SsSNP2006B_852 |
| 1756 | Composition | SsSNP2006B_1919 |
| 1757 | Composition | SsSNP2006B_1034 |
| 1758 | Composition | SsSNP2006B_1644 |
| 1759 | Composition | SsSNP2006B_2374 |
| 1760 | Composition | SsSNP2006B_1146 |
| 1761 | Composition | SsSNP2006B_84 |
| 1762 | Composition | SsSNP2006B_2250 |
| 1763 | Composition | SsSNP2006B_2133 |
| 1764 | Composition | SsSNP2006B_220 |
| 1765 | Composition | SsSNP2006B_409 |
| 1766 | Composition | SsSNP2006B_237 |
| 1767 | Growth | SsSNP2006B_365 |
| 1768 | Growth | SsSNP2006B_1841 |
| 1769 | Growth | SsSNP2006B_890 |
| 1770 | Growth | SsSNP2006B_120 |
| 1771 | Growth | SsSNP2006B_229 |
| 1772 | Growth | SsSNP2006B_87 |
| 1773 | Growth | SsSNP2006B_832 |
| 1774 | Growth | SsSNP2006B_1994 |
| 1775 | Growth | SsSNP2006B_96 |
| 1776 | Growth | SsSNP2006B_2414 |
| 1777 | Growth | SsSNP2006B_1257 |
| 1778 | Growth | SsSNP2006B_2476 |
| 1779 | Growth | SsSNP2006B_2413 |
| 1780 | Growth | SsSNP2006B_1419 |
| 1781 | Growth | SsSNP2006B_2491 |
| 1782 | Growth | SsSNP2006B_497 |
| 1783 | Growth | SsSNP2006B_1263 |
| 1784 | Growth | SsSNP2006B_2533 |
| 1785 | Growth | SsSNP2006B_194 |
| 1786 | Growth | SsSNP2006B_2226 |
| 1787 | Growth | SsSNP2006B_44 |
| 1788 | Growth | SsSNP2006B_1064 |
| 1789 | Growth | SsSNP2006B_1609 |
| 1790 | Growth | SsSNP2006B_1112 |
| 1791 | Growth | SsSNP2006B_1491 |
| 1792 | Growth | SsSNP2006B_1362 |
| 1793 | Growth | SsSNP2006B_885 |
| 1794 | Growth | SsSNP2006B_2542 |
| 1795 | Growth | SsSNP2006B_326 |
| 1796 | Growth | SsSNP2006B_327 |
| 1797 | Growth | SsSNP2006B_1872 |
| 1798 | Growth | SsSNP2006B_76 |
| 1799 | Growth | SsSNP2006B_1665 |
| 1800 | Growth | SsSNP2006B_2239 |
| 1801 | Growth | SsSNP2006B_722 |
| 1802 | Growth | SsSNP2006B_2145 |
| 1803 | Growth | SsSNP2006B_287 |
| 1804 | Growth | SsSNP2006B_406 |
| 1805 | Growth | SsSNP2006B_2045 |
| 1806 | Growth | SsSNP2006B_1794 |
| 1807 | Growth | SsSNP2006B_839 |
| 1808 | Growth | SsSNP2006B_2552 |
| 1809 | Growth | SsSNP2006B_36 |
| 1810 | Growth | SsSNP2006B_2344 |
| 1811 | Growth | SsSNP2006B_1466 |
| 1812 | Growth | SsSNP2006B_1237 |
| 1813 | Growth | SsSNP2006B_1238 |
| 1814 | Growth | SsSNP2006B_2522 |
| 1815 | Growth | SsSNP2006B_2248 |
| 1816 | Growth | SsSNP2006B_17 |
| 1817 | Growth | SsSNP2006B_1615 |
| 1818 | Growth | SsSNP2006B_828 |
| 1819 | Growth | SsSNP2006B_57 |
| 1820 | Growth | SsSNP2006B_970 |
| 1821 | Growth | SsSNP2006B_1228 |
| 1822 | Growth | SsSNP2006B_2387 |
| 1823 | Growth | SsSNP2006B_2361 |
| 1824 | Growth | SsSNP2006B_1924 |
| 1825 | Growth | SsSNP2006B_629 |
| 1826 | Growth | SsSNP2006B_1386 |
| 1827 | Growth | SsSNP2006B_1195 |
| 1828 | Growth | SsSNP2006B_882 |
| 1829 | Growth | SsSNP2006B_175 |
| 1830 | Growth | SsSNP2006B_2201 |
| 1831 | Growth | SsSNP2006B_2389 |
| 1832 | Growth | SsSNP2006B_708 |
| 1833 | Growth | SsSNP2006B_1603 |
| 1834 | Growth | SsSNP2006B_2281 |
| 1835 | Growth | SsSNP2006B_63 |
| 1836 | Growth | SsSNP2006B_2404 |
| 1837 | Growth | SsSNP2006B_2058 |
| 1838 | Growth | SsSNP2006B_158 |
| 1839 | Growth | SsSNP2006B_1348 |
| 1840 | Growth | SsSNP2006B_2287 |
| 1841 | Growth | SsSNP2006B_2566 |
| 1842 | Growth | SsSNP2006B_1826 |
| 1843 | Growth | SsSNP2006B_705 |
| 1844 | Growth | SsSNP2006B_827 |
| 1845 | Growth | SsSNP2006B_2214 |
| 1846 | Growth | SsSNP2006B_1374 |
| 1847 | Growth | SsSNP2006B_1187 |
| 1848 | Growth | SsSNP2006B_1410 |
| 1849 | Growth | SsSNP2006B_2012 |
| 1850 | Growth | SsSNP2006B_368 |
| 1851 | Growth | SsSNP2006B_2299 |
| 1852 | Growth | SsSNP2006B_485 |
| 1853 | Growth | SsSNP2006B_1599 |
| 1854 | Growth | SsSNP2006B_826 |
| 1855 | Growth | SsSNP2006B_855 |
| 1856 | Growth | SsSNP2006B_151 |
| 1857 | Growth | SsSNP2006B_500 |
| 1858 | Growth | SsSNP2006B_715 |
| 1859 | Growth | SsSNP2006B_862 |
| 1860 | Growth | SsSNP2006B_2331 |
| 1861 | Growth | SsSNP2006B_732 |
| 1862 | Growth | SsSNP2006B_1076 |
| 1863 | Growth | SsSNP2006B_2278 |
| 1864 | Growth | SsSNP2006B_2279 |
| 1865 | Growth | SsSNP2006B_1986 |
| 1866 | Growth | SsSNP2006B_2380 |
| 1867 | Growth | SsSNP2006B_1508 |
| 1868 | Growth | SsSNP2006B_1023 |
| 1869 | Growth | SsSNP2006B_1198 |
| 1870 | Growth | SsSNP2006B_2395 |
| 1871 | Growth | SsSNP2006B_1310 |
| 1872 | Growth | SsSNP2006B_2405 |
| 1873 | Growth | SsSNP2006B_542 |
| 1874 | Growth | SsSNP2006B_543 |
| 1875 | Growth | SsSNP2006B_155 |
| 1876 | Growth | SsSNP2006B_671 |
| 1877 | Growth | SsSNP2006B_690 |
| 1878 | Growth | SsSNP2006B_661 |
| 1879 | Growth | SsSNP2006B_1482 |
| 1880 | Growth | SsSNP2006B_2105 |
| 1881 | Growth | SsSNP2006B_2534 |
| 1882 | Growth | SsSNP2006B_314 |
| 1883 | Growth | SsSNP2006B_2061 |
| 1884 | Growth | SsSNP2006B_2366 |
| 1885 | Growth | SsSNP2006B_344 |
| 1886 | Growth | SsSNP2006B_302 |
| 1887 | Growth | SsSNP2006B_15 |
| 1888 | Growth | SsSNP2006B_688 |
| 1889 | Growth | SsSNP2006B_1796 |
| 1890 | Growth | SsSNP2006B_2377 |
| 1891 | Growth | SsSNP2006B_602 |
| 1892 | Growth | SsSNP2006B_2030 |
| 1893 | Growth | SsSNP2006B_1897 |
| 1894 | Growth | SsSNP2006B_770 |
| 1895 | Growth | SsSNP2006B_2249 |
| 1896 | Growth | SsSNP2006B_331 |
| 1897 | Growth | SsSNP2006B_2138 |
| 1898 | Growth | SsSNP2006B_1962 |
| 1899 | Growth | SsSNP2006B_399 |
| 1900 | Growth | SsSNP2006B_1349 |
| 1901 | Growth | SsSNP2006B_1319 |
| 1902 | Growth | SsSNP2006B_1320 |
| 1903 | Growth | SsSNP2006B_2270 |
| 1904 | Growth | SsSNP2006B_2323 |
| 1905 | Growth | SsSNP2006B_487 |
| 1906 | Growth | SsSNP2006B_1032 |
| 1907 | Growth | SsSNP2006B_1084 |
| 1908 | Growth | SsSNP2006B_1186 |
| 1909 | Growth | SsSNP2006B_1040 |
| 1910 | Growth | SsSNP2006B_1479 |
| 1911 | Growth | SsSNP2006B_38 |
| 1912 | Growth | SsSNP2006B_2044 |
| 1913 | Growth | SsSNP2006B_2186 |
| 1914 | Growth | SsSNP2006B_1657 |
| 1915 | Growth | SsSNP2006B_6 |
| 1916 | Growth | SsSNP2006B_40 |
| 1917 | Growth | SsSNP2006B_1103 |
| 1918 | Growth | SsSNP2006B_1560 |
| 1919 | Growth | SsSNP2006B_550 |
| 1920 | Growth | SsSNP2006B_687 |
| 1921 | Growth | SsSNP2006B_1066 |
| 1922 | Growth | SsSNP2006B_1429 |
| 1923 | Growth | SsSNP2006B_871 |
| 1924 | Growth | SsSNP2006B_179 |
| 1925 | Growth | SsSNP2006B_2161 |
| 1926 | Growth | SsSNP2006B_1909 |
| 1927 | Growth | SsSNP2006B_742 |
| 1928 | Growth | SsSNP2006B_1503 |
| 1929 | Growth | SsSNP2006B_1206 |
| 1930 | Growth | SsSNP2006B_1715 |
| 1931 | Growth | SsSNP2006B_1145 |
| 1932 | Growth | SsSNP2006B_560 |
| 1933 | Growth | SsSNP2006B_81 |
| 1934 | Growth | SsSNP2006B_1196 |
| 1935 | Growth | SsSNP2006B_2549 |
| 1936 | Growth | SsSNP2006B_1026 |
| 1937 | Growth | SsSNP2006B_2077 |
| 1938 | Growth | SsSNP2006B_1785 |
| 1939 | Growth | SsSNP2006B_774 |
| 1940 | Growth | SsSNP2006B_1587 |
| 1941 | Growth | SsSNP2006B_325 |
| 1942 | Growth | SsSNP2006B_1486 |
| 1943 | Growth | SsSNP2006B_815 |
| 1944 | Growth | SsSNP2006B_458 |
| 1945 | Growth | SsSNP2006B_360 |
| 1946 | Growth | SsSNP2006B_738 |
| 1947 | Growth | SsSNP2006B_1937 |
| 1948 | Growth | SsSNP2006B_2090 |
| 1949 | Growth | SsSNP2006B_974 |
| 1950 | Growth | SsSNP2006B_1767 |
| 1951 | Growth | SsSNP2006B_2296 |
| 1952 | Growth | SsSNP2006B_1802 |
| 1953 | Growth | SsSNP2006B_1764 |
| 1954 | Growth | SsSNP2006B_303 |
| 1955 | Growth | SsSNP2006B_895 |
| 1956 | Growth | SsSNP2006B_751 |
| 1957 | Growth | SsSNP2006B_45 |
| 1958 | Growth | SsSNP2006B_203 |
| 1959 | Growth | SsSNP2006B_2261 |
| 1960 | Growth | SsSNP2006B_696 |
| 1961 | Growth | SsSNP2006B_2486 |
| 1962 | Growth | SsSNP2006B_1625 |
| 1963 | Growth | SsSNP2006B_1692 |
| 1964 | Growth | SsSNP2006B_2556 |
| 1965 | Growth | SsSNP2006B_1018 |
| 1966 | Growth | SsSNP2006B_112 |
| 1967 | Growth | SsSNP2006B_712 |
| 1968 | Growth | SsSNP2006B_911 |
| 1969 | Growth | SsSNP2006B_2242 |
| 1970 | Growth | SsSNP2006B_860 |
| 1971 | Growth | SsSNP2006B_24 |
| 1972 | Growth | SsSNP2006B_35 |
| 1973 | Growth | SsSNP2006B_2557 |
| 1974 | Growth | SsSNP2006B_491 |
| 1975 | Growth | SsSNP2006B_2095 |
| 1976 | Growth | SsSNP2006B_2600 |
| 1977 | Growth | SsSNP2006B_1554 |
| 1978 | Growth | SsSNP2006B_1977 |
| 1979 | Growth | SsSNP2006B_2513 |
| 1980 | Growth | SsSNP2006B_2116 |
| 1981 | Growth | SsSNP2006B_2196 |
| 1982 | Growth | SsSNP2006B_935 |
| 1983 | Growth | SsSNP2006B_2388 |
| 1984 | Growth | SsSNP2006B_1014 |
| 1985 | Growth | SsSNP2006B_2037 |
| 1986 | Growth | SsSNP2006B_264 |
| 1987 | Growth | SsSNP2006B_962 |
| 1988 | Growth | SsSNP2006B_427 |
| 1989 | Growth | SsSNP2006B_2251 |
| 1990 | Growth | SsSNP2006B_2575 |
| 1991 | Growth | SsSNP2006B_43 |
| 1992 | Growth | SsSNP2006B_1337 |
| 1993 | Growth | SsSNP2006B_461 |
| 1994 | Growth | SsSNP2006B_356 |
| 1995 | Growth | SsSNP2006B_1990 |
| 1996 | Growth | SsSNP2006B_2267 |
| 1997 | Growth | SsSNP2006B_2418 |
| 1998 | Growth | SsSNP2006B_720 |
| 1999 | Growth | SsSNP2006B_1450 |
| 2000 | Growth | SsSNP2006B_719 |
| 2001 | Growth | SsSNP2006B_1418 |
| 2002 | Growth | SsSNP2006B_2144 |
| 2003 | Growth | SsSNP2006B_1185 |
| 2004 | Growth | SsSNP2006B_1564 |
| 2005 | Growth | SsSNP2006B_993 |
| 2006 | Growth | SsSNP2006B_1306 |
| 2007 | Growth | SsSNP2006B_2084 |
| 2008 | Growth | SsSNP2006B_249 |
| 2009 | Growth | SsSNP2006B_248 |
| 2010 | Growth | SsSNP2006B_2092 |
| 2011 | Growth | SsSNP2006B_2176 |
| 2012 | Growth | SsSNP2006B_1734 |
| 2013 | Growth | SsSNP2006B_695 |
| 2014 | Growth | SsSNP2006B_1696 |
| 2015 | Growth | SsSNP2006B_1047 |
| 2016 | Growth | SsSNP2006B_1859 |
| 2017 | Growth | SsSNP2006B_1551 |
| 2018 | Growth | SsSNP2006B_125 |
| 2019 | Growth | SsSNP2006B_126 |
| 2020 | Growth | SsSNP2006B_2356 |
| 2021 | Growth | SsSNP2006B_2099 |
| 2022 | Growth | SsSNP2006B_830 |
| 2023 | Growth | SsSNP2006B_1234 |
| 2024 | Growth | SsSNP2006B_2093 |
| 2025 | Growth | SsSNP2006B_755 |
| 2026 | Growth | SsSNP2006B_2238 |
| 2027 | Growth | SsSNP2006B_1443 |
| 2028 | Growth | SsSNP2006B_1588 |
| 2029 | Growth | SsSNP2006B_1045 |
| 2030 | Growth | SsSNP2006B_2000 |
| 2031 | Growth | SsSNP2006B_1165 |
| 2032 | Growth | SsSNP2006B_1586 |
| 2033 | Growth | SsSNP2006B_1010 |
| 2034 | Growth | SsSNP2006B_2100 |
| 2035 | Growth | SsSNP2006B_1278 |
| 2036 | Growth | SsSNP2006B_1441 |
| 2037 | Growth | SsSNP2006B_375 |
| 2038 | Growth | SsSNP2006B_473 |
| 2039 | Growth | SsSNP2006B_1268 |
| 2040 | Growth | SsSNP2006B_474 |
| 2041 | Growth | SsSNP2006B_1661 |
| 2042 | Growth | SsSNP2006B_1571 |
| 2043 | Growth | SsSNP2006B_1070 |
| 2044 | Growth | SsSNP2006B_50 |
| 2045 | Growth | SsSNP2006B_1532 |
| 2046 | Growth | SsSNP2006B_619 |
| 2047 | Growth | SsSNP2006B_976 |
| 2048 | Growth | SsSNP2006B_2025 |
| 2049 | Growth | SsSNP2006B_2537 |
| 2050 | Growth | SsSNP2006B_1784 |
| 2051 | Growth | SsSNP2006B_2271 |
| 2052 | Growth | SsSNP2006B_394 |
| 2053 | Growth | SsSNP2006B_1358 |
| 2054 | Growth | SsSNP2006B_2152 |
| 2055 | Growth | SsSNP2006B_226 |
| 2056 | Growth | SsSNP2006B_896 |
| 2057 | Growth | SsSNP2006B_1028 |
| 2058 | Growth | SsSNP2006B_65 |
| 2059 | Growth | SsSNP2006B_1834 |
| 2060 | Growth | SsSNP2006B_413 |
| 2061 | Growth | SsSNP2006B_779 |
| 2062 | Growth | SsSNP2006B_1634 |
| 2063 | Growth | SsSNP2006B_391 |
| 2064 | Growth | SsSNP2006B_2170 |
| 2065 | Growth | SsSNP2006B_1687 |
| 2066 | Growth | SsSNP2006B_893 |
| 2067 | Growth | SsSNP2006B_894 |
| 2068 | Growth | SsSNP2006B_2122 |
| 2069 | Growth | SsSNP2006B_478 |
| 2070 | Growth | SsSNP2006B_1983 |
| 2071 | Growth | SsSNP2006B_1439 |
| 2072 | Growth | SsSNP2006B_1296 |
| 2073 | Growth | SsSNP2006B_385 |
| 2074 | Growth | SsSNP2006B_2159 |
| 2075 | Growth | SsSNP2006B_2167 |
| 2076 | Growth | SsSNP2006B_1908 |
| 2077 | Growth | SsSNP2006B_1253 |
| 2078 | Growth | SsSNP2006B_2525 |
| 2079 | Growth | SsSNP2006B_741 |
| 2080 | Growth | SsSNP2006B_891 |
| 2081 | Growth | SsSNP2006B_1743 |
| 2082 | Growth | SsSNP2006B_1928 |
| 2083 | Growth | SsSNP2006B_2149 |
| 2084 | Growth | SsSNP2006B_7 |
| 2085 | Growth | SsSNP2006B_2357 |
| 2086 | Growth | SsSNP2006B_2320 |
| 2087 | Growth | SsSNP2006B_1878 |
| 2088 | Growth | SsSNP2006B_2492 |
| 2089 | Growth | SsSNP2006B_2140 |
| 2090 | Growth | SsSNP2006B_2245 |
| 2091 | Growth | SsSNP2006B_2111 |
| 2092 | Growth | SsSNP2006B_457 |
| 2093 | Growth | SsSNP2006B_1217 |
| 2094 | Growth | SsSNP2006B_1939 |
| 2095 | Growth | SsSNP2006B_436 |
| 2096 | Growth | SsSNP2006B_2205 |
| 2097 | Growth | SsSNP2006B_2206 |
| 2098 | Growth | SsSNP2006B_1954 |
| 2099 | Growth | SsSNP2006B_113 |
| 2100 | Growth | SsSNP2006B_1384 |
| 2101 | Growth | SsSNP2006B_947 |
| 2102 | Growth | SsSNP2006B_1276 |
| 2103 | Growth | SsSNP2006B_1572 |
| 2104 | Growth | SsSNP2006B_1333 |
| 2105 | Growth | SsSNP2006B_1569 |
| 2106 | Growth | SsSNP2006B_646 |
| 2107 | Growth | SsSNP2006B_1068 |
| 2108 | Growth | SsSNP2006B_1357 |
| 2109 | Growth | SsSNP2006B_1529 |
| 2110 | Growth | SsSNP2006B_8 |
| 2111 | Growth | SsSNP2006B_2517 |
| 2112 | Growth | SsSNP2006B_445 |
| 2113 | Growth | SsSNP2006B_1334 |
| 2114 | Growth | SsSNP2006B_2259 |
| 2115 | Growth | SsSNP2006B_1247 |
| 2116 | Growth | SsSNP2006B_1779 |
| 2117 | Growth | SsSNP2006B_2536 |
| 2118 | Growth | SsSNP2006B_141 |
| 2119 | Growth | SsSNP2006B_2462 |
| 2120 | Growth | SsSNP2006B_1744 |
| 2121 | Growth | SsSNP2006B_410 |
| 2122 | Growth | SsSNP2006B_878 |
| 2123 | Growth | SsSNP2006B_2057 |
| 2124 | Growth | SsSNP2006B_476 |
| 2125 | Growth | SsSNP2006B_699 |
| 2126 | Growth | SsSNP2006B_848 |
| 2127 | Growth | SsSNP2006B_428 |
| 2128 | Growth | SsSNP2006B_2449 |
| 2129 | Growth | SsSNP2006B_440 |
| 2130 | Growth | SsSNP2006B_2163 |
| 2131 | Growth | SsSNP2006B_49 |
| 2132 | Growth | SsSNP2006B_2211 |
| 2133 | Growth | SsSNP2006B_2595 |
| 2134 | Growth | SsSNP2006B_864 |
| 2135 | Growth | SsSNP2006B_1792 |
| 2136 | Growth | SsSNP2006B_1488 |
| 2137 | Growth | SsSNP2006B_1489 |
| 2138 | Growth | SsSNP2006B_1134 |
| 2139 | Growth | SsSNP2006B_1368 |
| 2140 | Growth | SsSNP2006B_870 |
| 2141 | Growth | SsSNP2006B_1985 |
| 2142 | Growth | SsSNP2006B_2397 |
| 2143 | Growth | SsSNP2006B_641 |
| 2144 | Growth | SsSNP2006B_1131 |
| 2145 | Growth | SsSNP2006B_234 |
| 2146 | Growth | SsSNP2006B_1730 |
| 2147 | Growth | SsSNP2006B_1871 |
| 2148 | Growth | SsSNP2006B_875 |
| 2149 | Growth | SsSNP2006B_677 |
| 2150 | Growth | SsSNP2006B_592 |
| 2151 | Growth | SsSNP2006B_1222 |
| 2152 | Growth | SsSNP2006B_1203 |
| 2153 | Growth | SsSNP2006B_446 |
| 2154 | Growth | SsSNP2006B_2143 |
| 2155 | Growth | SsSNP2006B_2010 |
| 2156 | Growth | SsSNP2006B_1024 |
| 2157 | Growth | SsSNP2006B_1659 |
| 2158 | Growth | SsSNP2006B_353 |
| 2159 | Growth | SsSNP2006B_606 |
| 2160 | Growth | SsSNP2006B_1606 |
| 2161 | Growth | SsSNP2006B_1122 |
| 2162 | Growth | SsSNP2006B_1221 |
| 2163 | Growth | SsSNP2006B_1584 |
| 2164 | Growth | SsSNP2006B_1406 |
| 2165 | Growth | SsSNP2006B_663 |
| 2166 | Growth | SsSNP2006B_664 |
| 2167 | Growth | SsSNP2006B_627 |
| 2168 | Growth | SsSNP2006B_686 |
| 2169 | Growth | SsSNP2006B_537 |
| 2170 | Growth | SsSNP2006B_1020 |
| 2171 | Growth | SsSNP2006B_2002 |
| 2172 | Growth | SsSNP2006B_1063 |
| 2173 | Growth | SsSNP2006B_464 |
| 2174 | Growth | SsSNP2006B_1704 |
| 2175 | Growth | SsSNP2006B_1513 |
| 2176 | Growth | SsSNP2006B_2483 |
| 2177 | Growth | SsSNP2006B_2008 |
| 2178 | Growth | SsSNP2006B_897 |
| 2179 | Growth | SsSNP2006B_810 |
| 2180 | Growth | SsSNP2006B_222 |
| 2181 | Growth | SsSNP2006B_561 |
| 2182 | Growth | SsSNP2006B_1576 |
| 2183 | Growth | SsSNP2006B_559 |
| 2184 | Growth | SsSNP2006B_418 |
| 2185 | Growth | SsSNP2006B_967 |
| 2186 | Growth | SsSNP2006B_1251 |
| 2187 | Growth | SsSNP2006B_2237 |
| 2188 | Growth | SsSNP2006B_1031 |
| 2189 | Growth | SsSNP2006B_1292 |
| 2190 | Growth | SsSNP2006B_2231 |
| 2191 | Growth | SsSNP2006B_335 |
| 2192 | Growth | SsSNP2006B_1239 |
| 2193 | Growth | SsSNP2006B_716 |
| 2194 | Growth | SsSNP2006B_1538 |
| 2195 | Growth | SsSNP2006B_37 |
| 2196 | Growth | SsSNP2006B_1817 |
| 2197 | Growth | SsSNP2006B_2465 |
| 2198 | Growth | SsSNP2006B_727 |
| 2199 | Growth | SsSNP2006B_1963 |
| 2200 | Growth | SsSNP2006B_41 |
| 2201 | Growth | SsSNP2006B_56 |
| 2202 | Growth | SsSNP2006B_2581 |
| 2203 | Growth | SsSNP2006B_1193 |
| 2204 | Growth | SsSNP2006B_1233 |
| 2205 | Growth | SsSNP2006B_1485 |
| 2206 | Growth | SsSNP2006B_2065 |
| 2207 | Growth | SsSNP2006B_1633 |
| 2208 | Growth | SsSNP2006B_1806 |
| 2209 | Growth | SsSNP2006B_378 |
| 2210 | Growth | SsSNP2006B_1205 |
| 2211 | Growth | SsSNP2006B_2576 |
| 2212 | Growth | SsSNP2006B_1144 |
| 2213 | Growth | SsSNP2006B_2258 |
| 2214 | Growth | SsSNP2006B_2064 |
| 2215 | Growth | SsSNP2006B_1226 |
| 2216 | Growth | SsSNP2006B_1945 |
| 2217 | Growth | SsSNP2006B_33 |
| 2218 | Growth | SsSNP2006B_1417 |
| 2219 | Growth | SsSNP2006B_1712 |
| 2220 | Growth | SsSNP2006B_2179 |
| 2221 | Growth | SsSNP2006B_282 |
| 2222 | Growth | SsSNP2006B_1043 |
| 2223 | Growth | SsSNP2006B_1847 |
| 2224 | Growth | SsSNP2006B_901 |
| 2225 | Growth | SsSNP2006B_914 |
| 2226 | Growth | SsSNP2006B_759 |
| 2227 | Growth | SsSNP2006B_1313 |
| 2228 | Growth | SsSNP2006B_849 |
| 2229 | Growth | SsSNP2006B_2283 |
| 2230 | Growth | SsSNP2006B_740 |
| 2231 | Growth | SsSNP2006B_2056 |
| 2232 | Growth | SsSNP2006B_1108 |
| 2233 | Growth | SsSNP2006B_134 |
| 2234 | Growth | SsSNP2006B_135 |
| 2235 | Growth | SsSNP2006B_2233 |
| 2236 | Growth | SsSNP2006B_590 |
| 2237 | Growth | SsSNP2006B_990 |
| 2238 | Growth | SsSNP2006B_1684 |
| 2239 | Growth | SsSNP2006B_128 |
| 2240 | Growth | SsSNP2006B_2241 |
| 2241 | Growth | SsSNP2006B_243 |
| 2242 | Growth | SsSNP2006B_379 |
| 2243 | Growth | SsSNP2006B_2195 |
| 2244 | Growth | SsSNP2006B_200 |
| 2245 | Growth | SsSNP2006B_816 |
| 2246 | Growth | SsSNP2006B_333 |
| 2247 | Growth | SsSNP2006B_2032 |
| 2248 | Growth | SsSNP2006B_933 |
| 2249 | Growth | SsSNP2006B_917 |
| 2250 | Growth | SsSNP2006B_124 |
| 2251 | Growth | SsSNP2006B_1069 |
| 2252 | Growth | SsSNP2006B_1472 |
| 2253 | Growth | SsSNP2006B_2341 |
| 2254 | Growth | SsSNP2006B_1636 |
| 2255 | Growth | SsSNP2006B_97 |
| 2256 | Growth | SsSNP2006B_2277 |
| 2257 | Growth | SsSNP2006B_1614 |
| 2258 | Growth | SsSNP2006B_114 |
| 2259 | Growth | SsSNP2006B_1526 |
| 2260 | Growth | SsSNP2006B_1567 |
| 2261 | Growth | SsSNP2006B_1938 |
| 2262 | Growth | SsSNP2006B_47 |
| 2263 | Growth | SsSNP2006B_1974 |
| 2264 | Growth | SsSNP2006B_983 |
| 2265 | Growth | SsSNP2006B_129 |
| 2266 | Growth | SsSNP2006B_1721 |
| 2267 | Growth | SsSNP2006B_2047 |
| 2268 | Growth | SsSNP2006B_1873 |
| 2269 | Growth | SsSNP2006B_2297 |
| 2270 | Growth | SsSNP2006B_1395 |
| 2271 | Growth | SsSNP2006B_2003 |
| 2272 | Growth | SsSNP2006B_1750 |
| 2273 | Growth | SsSNP2006B_1951 |
| 2274 | Growth | SsSNP2006B_1653 |
| 2275 | Growth | SsSNP2006B_821 |
| 2276 | Growth | SsSNP2006B_2545 |
| 2277 | Growth | SsSNP2006B_956 |
| 2278 | Growth | SsSNP2006B_1867 |
| 2279 | Growth | SsSNP2006B_1921 |
| 2280 | Growth | SsSNP2006B_1089 |
| 2281 | Growth | SsSNP2006B_1404 |
| 2282 | Growth | SsSNP2006B_772 |
| 2283 | Growth | SsSNP2006B_1915 |
| 2284 | Growth | SsSNP2006B_858 |
| 2285 | Growth | SsSNP2006B_2453 |
| 2286 | Growth | SsSNP2006B_1009 |
| 2287 | Growth | SsSNP2006B_1895 |
| 2288 | Growth | SsSNP2006B_363 |
| 2289 | Growth | SsSNP2006B_948 |
| 2290 | Growth | SsSNP2006B_2428 |
| 2291 | Growth | SsSNP2006B_1133 |
| 2292 | Growth | SsSNP2006B_133 |
| 2293 | Growth | SsSNP2006B_324 |
| 2294 | Growth | SsSNP2006B_1590 |
| 2295 | Growth | SsSNP2006B_1654 |
| 2296 | Growth | SsSNP2006B_1888 |
| 2297 | Growth | SsSNP2006B_1889 |
| 2298 | Growth | SsSNP2006B_2106 |
| 2299 | Growth | SsSNP2006B_1901 |
| 2300 | Growth | SsSNP2006B_640 |
| 2301 | Growth | SsSNP2006B_674 |
| 2302 | Growth | SsSNP2006B_2554 |
| 2303 | Growth | SsSNP2006B_760 |
| 2304 | Growth | SsSNP2006B_963 |
| 2305 | Growth | SsSNP2006B_1270 |
| 2306 | Growth | SsSNP2006B_217 |
| 2307 | Growth | SsSNP2006B_681 |
| 2308 | Growth | SsSNP2006B_2419 |
| 2309 | Growth | SsSNP2006B_390 |
| 2310 | Growth | SsSNP2006B_1667 |
| 2311 | Growth | SsSNP2006B_2512 |
| 2312 | Growth | SsSNP2006B_2372 |
| 2313 | Growth | SsSNP2006B_2036 |
| 2314 | Growth | SsSNP2006B_2503 |
| 2315 | Growth | SsSNP2006B_2078 |
| 2316 | Growth | SsSNP2006B_2368 |
| 2317 | Growth | SsSNP2006B_2369 |
| 2318 | Growth | SsSNP2006B_675 |
| 2319 | Growth | SsSNP2006B_689 |
| 2320 | Growth | SsSNP2006B_771 |
| 2321 | Growth | SsSNP2006B_551 |
| 2322 | Growth | SsSNP2006B_1822 |
| 2323 | Growth | SsSNP2006B_456 |
| 2324 | Growth | SsSNP2006B_2055 |
| 2325 | Growth | SsSNP2006B_2520 |
| 2326 | Growth | SsSNP2006B_338 |
| 2327 | Growth | SsSNP2006B_513 |
| 2328 | Growth | SsSNP2006B_150 |
| 2329 | Growth | SsSNP2006B_315 |
| 2330 | Growth | SsSNP2006B_1001 |
| 2331 | Growth | SsSNP2006B_1602 |
| 2332 | Growth | SsSNP2006B_1805 |
| 2333 | Growth | SsSNP2006B_951 |
| 2334 | Growth | SsSNP2006B_2112 |
| 2335 | Growth | SsSNP2006B_1768 |
| 2336 | Growth | SsSNP2006B_817 |
| 2337 | Growth | SsSNP2006B_736 |
| 2338 | Growth | SsSNP2006B_1642 |
| 2339 | Growth | SsSNP2006B_977 |
| 2340 | Growth | SsSNP2006B_1029 |
| 2341 | Growth | SsSNP2006B_1798 |
| 2342 | Growth | SsSNP2006B_1387 |
| 2343 | Growth | SsSNP2006B_1677 |
| 2344 | Growth | SsSNP2006B_1480 |
| 2345 | Growth | SsSNP2006B_1525 |
| 2346 | Growth | SsSNP2006B_943 |
| 2347 | Growth | SsSNP2006B_1207 |
| 2348 | Growth | SsSNP2006B_1477 |
| 2349 | Growth | SsSNP2006B_1478 |
| 2350 | Growth | SsSNP2006B_1414 |
| 2351 | Growth | SsSNP2006B_1124 |
| 2352 | Growth | SsSNP2006B_1581 |
| 2353 | Growth | SsSNP2006B_1454 |
| 2354 | Growth | SsSNP2006B_1297 |
| 2355 | Growth | SsSNP2006B_2020 |
| 2356 | Growth | SsSNP2006B_1116 |
| 2357 | Growth | SsSNP2006B_1519 |
| 2358 | Growth | SsSNP2006B_553 |
| 2359 | Growth | SsSNP2006B_776 |
| 2360 | Growth | SsSNP2006B_1512 |
| 2361 | Growth | SsSNP2006B_2326 |
| 2362 | Growth | SsSNP2006B_1701 |
| 2363 | Growth | SsSNP2006B_250 |
| 2364 | Growth | SsSNP2006B_2243 |
| 2365 | Growth | SsSNP2006B_1739 |
| 2366 | Growth | SsSNP2006B_2466 |
| 2367 | Growth | SsSNP2006B_2171 |
| 2368 | Growth | SsSNP2006B_429 |
| 2369 | Growth | SsSNP2006B_667 |
| 2370 | Growth | SsSNP2006B_2218 |
| 2371 | Growth | SsSNP2006B_225 |
| 2372 | Growth | SsSNP2006B_486 |
| 2373 | Growth | SsSNP2006B_190 |
| 2374 | Growth | SsSNP2006B_1823 |
| 2375 | Growth | SsSNP2006B_679 |
| 2376 | Growth | SsSNP2006B_1391 |
| 2377 | Growth | SsSNP2006B_534 |
| 2378 | Growth | SsSNP2006B_1128 |
| 2379 | Growth | SsSNP2006B_1679 |
| 2380 | Growth | SsSNP2006B_1455 |
| 2381 | Growth | SsSNP2006B_2301 |
| 2382 | Growth | SsSNP2006B_2306 |
| 2383 | Growth | SsSNP2006B_1022 |
| 2384 | Growth | SsSNP2006B_1494 |
| 2385 | Growth | SsSNP2006B_601 |
| 2386 | Growth | SsSNP2006B_1595 |
| 2387 | Growth | SsSNP2006B_99 |
| 2388 | Growth | SsSNP2006B_2446 |
| 2389 | Growth | SsSNP2006B_1970 |
| 2390 | Growth | SsSNP2006B_1807 |
| 2391 | Growth | SsSNP2006B_1553 |
| 2392 | Growth | SsSNP2006B_920 |
| 2393 | Growth | SsSNP2006B_2391 |
| 2394 | Growth | SsSNP2006B_1926 |
| 2395 | Growth | SsSNP2006B_1762 |
| 2396 | Growth | SsSNP2006B_165 |
| 2397 | Growth | SsSNP2006B_244 |
| 2398 | Growth | SsSNP2006B_2336 |
| 2399 | Growth | SsSNP2006B_697 |
| 2400 | Growth | SsSNP2006B_1718 |
| 2401 | Growth | SsSNP2006B_381 |
| 2402 | Growth | SsSNP2006B_149 |
| 2403 | Growth | SsSNP2006B_2560 |
| 2404 | Growth | SsSNP2006B_728 |
| 2405 | Growth | SsSNP2006B_1953 |
| 2406 | Growth | SsSNP2006B_961 |
| 2407 | Growth | SsSNP2006B_887 |
| 2408 | Growth | SsSNP2006B_111 |
| 2409 | Growth | SsSNP2006B_790 |
| 2410 | Growth | SsSNP2006B_1839 |
| 2411 | Growth | SsSNP2006B_622 |
| 2412 | Growth | SsSNP2006B_1947 |
| 2413 | Growth | SsSNP2006B_1154 |
| 2414 | Growth | SsSNP2006B_1432 |
| 2415 | Growth | SsSNP2006B_1118 |
| 2416 | Growth | SsSNP2006B_1490 |
| 2417 | Growth | SsSNP2006B_2198 |
| 2418 | Growth | SsSNP2006B_224 |
| 2419 | Growth | SsSNP2006B_1012 |
| 2420 | Growth | SsSNP2006B_859 |
| 2421 | Growth | SsSNP2006B_1453 |
| 2422 | Growth | SsSNP2006B_22 |
| 2423 | Growth | SsSNP2006B_1997 |
| 2424 | Growth | SsSNP2006B_536 |
| 2425 | Growth | SsSNP2006B_1655 |
| 2426 | Growth | SsSNP2006B_1046 |
| 2427 | Growth | SsSNP2006B_1565 |
| 2428 | Growth | SsSNP2006B_170 |
| 2429 | Growth | SsSNP2006B_1700 |
| 2430 | Growth | SsSNP2006B_1914 |
| 2431 | Growth | SsSNP2006B_1379 |
| 2432 | Growth | SsSNP2006B_1632 |
| 2433 | Growth | SsSNP2006B_2519 |
| 2434 | Growth | SsSNP2006B_292 |
| 2435 | Growth | SsSNP2006B_1559 |
| 2436 | Growth | SsSNP2006B_853 |
| 2437 | Growth | SsSNP2006B_1400 |
| 2438 | Growth | SsSNP2006B_680 |
| 2439 | Growth | SsSNP2006B_239 |
| 2440 | Growth | SsSNP2006B_240 |
| 2441 | Growth | SsSNP2006B_1423 |
| 2442 | Growth | SsSNP2006B_1522 |
| 2443 | Growth | SsSNP2006B_245 |
| 2444 | Growth | SsSNP2006B_823 |
| 2445 | Growth | SsSNP2006B_253 |
| 2446 | Growth | SsSNP2006B_1589 |
| 2447 | Growth | SsSNP2006B_999 |
| 2448 | Growth | SsSNP2006B_1544 |
| 2449 | Growth | SsSNP2006B_723 |
| 2450 | Growth | SsSNP2006B_2349 |
| 2451 | Growth | SsSNP2006B_800 |
| 2452 | Growth | SsSNP2006B_1225 |
| 2453 | Growth | SsSNP2006B_717 |
| 2454 | Growth | SsSNP2006B_430 |
| 2455 | Growth | SsSNP2006B_801 |
| 2456 | Growth | SsSNP2006B_802 |
| 2457 | Growth | SsSNP2006B_2324 |
| 2458 | Growth | SsSNP2006B_1651 |
| 2459 | Growth | SsSNP2006B_1331 |
| 2460 | Growth | SsSNP2006B_2120 |
| 2461 | Growth | SsSNP2006B_507 |
| 2462 | Growth | SsSNP2006B_10 |
| 2463 | Growth | SsSNP2006B_1471 |
| 2464 | Growth | SsSNP2006B_2040 |
| 2465 | Growth | SsSNP2006B_499 |
| 2466 | Growth | SsSNP2006B_2407 |
| 2467 | Growth | SsSNP2006B_484 |
| 2468 | Growth | SsSNP2006B_2160 |
| 2469 | Growth | SsSNP2006B_262 |
| 2470 | Growth | SsSNP2006B_1877 |
| 2471 | Growth | SsSNP2006B_2222 |
| 2472 | Growth | SsSNP2006B_2189 |
| 2473 | Growth | SsSNP2006B_1795 |
| 2474 | Growth | SsSNP2006B_1650 |
| 2475 | Growth | SsSNP2006B_634 |
| 2476 | Growth | SsSNP2006B_2441 |
| 2477 | Growth | SsSNP2006B_1579 |
| 2478 | Growth | SsSNP2006B_1989 |
| 2479 | Growth | SsSNP2006B_2523 |
| 2480 | Growth | SsSNP2006B_766 |
| 2481 | Growth | SsSNP2006B_1347 |
| 2482 | Growth | SsSNP2006B_992 |
| 2483 | Growth | SsSNP2006B_825 |
| 2484 | Growth | SsSNP2006B_2524 |
| 2485 | Growth | SsSNP2006B_1250 |
| 2486 | Growth | SsSNP2006B_1487 |
| 2487 | Growth | SsSNP2006B_1658 |
| 2488 | Growth | SsSNP2006B_1153 |
| 2489 | Growth | SsSNP2006B_160 |
| 2490 | Growth | SsSNP2006B_2290 |
| 2491 | Growth | SsSNP2006B_1304 |
| 2492 | Growth | SsSNP2006B_2358 |
| 2493 | Growth | SsSNP2006B_2427 |
| 2494 | Growth | SsSNP2006B_447 |
| 2495 | Growth | SsSNP2006B_1917 |
| 2496 | Growth | SsSNP2006B_152 |
| 2497 | Growth | SsSNP2006B_1882 |
| 2498 | Growth | SsSNP2006B_919 |
| 2499 | Growth | SsSNP2006B_1289 |
| 2500 | Growth | SsSNP2006B_319 |
| 2501 | Growth | SsSNP2006B_1208 |
| 2502 | Growth | SsSNP2006B_191 |
| 2503 | Growth | SsSNP2006B_454 |
| 2504 | Growth | SsSNP2006B_1197 |
| 2505 | Growth | SsSNP2006B_2213 |
| 2506 | Growth | SsSNP2006B_632 |
| 2507 | Growth | SsSNP2006B_959 |
| 2508 | Growth | SsSNP2006B_1688 |
| 2509 | Growth | SsSNP2006B_2304 |
| 2510 | Growth | SsSNP2006B_1635 |
| 2511 | Growth | SsSNP2006B_1184 |
| 2512 | Growth | SsSNP2006B_318 |
| 2513 | Growth | SsSNP2006B_2531 |
| 2514 | Growth | SsSNP2006B_2316 |
| 2515 | Growth | SsSNP2006B_2443 |
| 2516 | Growth | SsSNP2006B_1194 |
| 2517 | Growth | SsSNP2006B_1528 |
| 2518 | Growth | SsSNP2006B_2054 |
| 2519 | Growth | SsSNP2006B_2212 |
| 2520 | Growth | SsSNP2006B_2310 |
| 2521 | Growth | SsSNP2006B_1389 |
| 2522 | Growth | SsSNP2006B_2354 |
| 2523 | Growth | SsSNP2006B_515 |
| 2524 | Growth | SsSNP2006B_804 |
| 2525 | Growth | SsSNP2006B_1899 |
| 2526 | Growth | SsSNP2006B_2228 |
| 2527 | Growth | SsSNP2006B_415 |
| 2528 | Growth | SsSNP2006B_1631 |
| 2529 | Growth | SsSNP2006B_1911 |
| 2530 | Growth | SsSNP2006B_102 |
| 2531 | Growth | SsSNP2006B_1182 |
| 2532 | Growth | SsSNP2006B_2207 |
| 2533 | Growth | SsSNP2006B_1468 |
| 2534 | Growth | SsSNP2006B_1401 |
| 2535 | Growth | SsSNP2006B_792 |
| 2536 | Growth | SsSNP2006B_793 |
| 2537 | Growth | SsSNP2006B_2437 |
| 2538 | Growth | SsSNP2006B_1312 |
| 2539 | Growth | SsSNP2006B_2300 |
| 2540 | Growth | SsSNP2006B_1641 |
| 2541 | Growth | SsSNP2006B_713 |
| 2542 | Growth | SsSNP2006B_945 |
| 2543 | Growth | SsSNP2006B_525 |
| 2544 | Growth | SsSNP2006B_1336 |
| 2545 | Growth | SsSNP2006B_493 |
| 2546 | Growth | SsSNP2006B_2562 |
| 2547 | Growth | SsSNP2006B_105 |
| 2548 | Growth | SsSNP2006B_528 |
| 2549 | Growth | SsSNP2006B_563 |
| 2550 | Growth | SsSNP2006B_669 |
| 2551 | Growth | SsSNP2006B_1627 |
| 2552 | Growth | SsSNP2006B_1920 |
| 2553 | Growth | SsSNP2006B_1611 |
| 2554 | Growth | SsSNP2006B_1960 |
| 2555 | Growth | SsSNP2006B_1697 |
| 2556 | Growth | SsSNP2006B_1460 |
| 2557 | Growth | SsSNP2006B_482 |
| 2558 | Growth | SsSNP2006B_986 |
| 2559 | Growth | SsSNP2006B_306 |
| 2560 | Growth | SsSNP2006B_591 |
| 2561 | Growth | SsSNP2006B_1011 |
| 2562 | Growth | SsSNP2006B_205 |
| 2563 | Growth | SsSNP2006B_2451 |
| 2564 | Growth | SsSNP2006B_775 |
| 2565 | Growth | SsSNP2006B_1055 |
| 2566 | Growth | SsSNP2006B_1249 |
| 2567 | Growth | SsSNP2006B_1591 |
| 2568 | Growth | SsSNP2006B_1757 |
| 2569 | Growth | SsSNP2006B_1141 |
| 2570 | Growth | SsSNP2006B_2345 |
| 2571 | Growth | SsSNP2006B_587 |
| 2572 | Growth | SsSNP2006B_588 |
| 2573 | Growth | SsSNP2006B_1006 |
| 2574 | Growth | SsSNP2006B_1007 |
| 2575 | Growth | SsSNP2006B_1183 |
| 2576 | Growth | SsSNP2006B_367 |
| 2577 | Growth | SsSNP2006B_979 |
| 2578 | Growth | SsSNP2006B_346 |
| 2579 | Growth | SsSNP2006B_1943 |
| 2580 | Growth | SsSNP2006B_1563 |
| 2581 | Growth | SsSNP2006B_211 |
| 2582 | Growth | SsSNP2006B_2035 |
| 2583 | Growth | SsSNP2006B_2169 |
| 2584 | Growth | SsSNP2006B_147 |
| 2585 | Growth | SsSNP2006B_2593 |
| 2586 | Growth | SsSNP2006B_2360 |
| 2587 | Growth | SsSNP2006B_564 |
| 2588 | Growth | SsSNP2006B_1814 |
| 2589 | Growth | SsSNP2006B_518 |
| 2590 | Growth | SsSNP2006B_78 |
| 2591 | Growth | SsSNP2006B_2209 |
| 2592 | Growth | SsSNP2006B_2086 |
| 2593 | Growth | SsSNP2006B_2567 |
| 2594 | Growth | SsSNP2006B_156 |
| 2595 | Growth | SsSNP2006B_1015 |
| 2596 | Growth | SsSNP2006B_794 |
| 2597 | Growth | SsSNP2006B_276 |
| 2598 | Growth | SsSNP2006B_910 |
| 2599 | Growth | SsSNP2006B_275 |
| 2600 | Growth | SsSNP2006B_1344 |
| 2601 | Growth | SsSNP2006B_1262 |
| 2602 | Growth | SsSNP2006B_575 |
| 2603 | Growth | SsSNP2006B_323 |
| 2604 | Growth | SsSNP2006B_565 |
| 2605 | Growth | SsSNP2006B_2188 |
| 2606 | Growth | SsSNP2006B_2168 |
| 2607 | Growth | SsSNP2006B_1016 |
| 2608 | Growth | SsSNP2006B_72 |
| 2609 | Growth | SsSNP2006B_1345 |
| 2610 | Growth | SsSNP2006B_837 |
| 2611 | Growth | SsSNP2006B_397 |
| 2612 | Growth | SsSNP2006B_2390 |
| 2613 | Growth | SsSNP2006B_339 |
| 2614 | Growth | SsSNP2006B_2541 |
| 2615 | Growth | SsSNP2006B_1003 |
| 2616 | Growth | SsSNP2006B_1853 |
| 2617 | Growth | SsSNP2006B_655 |
| 2618 | Growth | SsSNP2006B_883 |
| 2619 | Growth | SsSNP2006B_1136 |
| 2620 | Growth | SsSNP2006B_2339 |
| 2621 | Growth | SsSNP2006B_912 |
| 2622 | Growth | SsSNP2006B_670 |
| 2623 | Growth | SsSNP2006B_2515 |
| 2624 | Growth | SsSNP2006B_227 |
| 2625 | Growth | SsSNP2006B_373 |
| 2626 | Growth | SsSNP2006B_2303 |
| 2627 | Growth | SsSNP2006B_2318 |
| 2628 | Growth | SsSNP2006B_558 |
| 2629 | Growth | SsSNP2006B_1845 |
| 2630 | Growth | SsSNP2006B_1200 |
| 2631 | Growth | SsSNP2006B_503 |
| 2632 | Growth | SsSNP2006B_991 |
| 2633 | Growth | SsSNP2006B_1613 |
| 2634 | Growth | SsSNP2006B_1874 |
| 2635 | Growth | SsSNP2006B_2046 |
| 2636 | Growth | SsSNP2006B_2501 |
| 2637 | Growth | SsSNP2006B_714 |
| 2638 | Growth | SsSNP2006B_1097 |
| 2639 | Growth | SsSNP2006B_1096 |
| 2640 | Growth | SsSNP2006B_2333 |
| 2641 | Growth | SsSNP2006B_2565 |
| 2642 | Growth | SsSNP2006B_384 |
| 2643 | Growth | SsSNP2006B_285 |
| 2644 | Growth | SsSNP2006B_850 |
| 2645 | Growth | SsSNP2006B_1424 |
| 2646 | Growth | SsSNP2006B_654 |
| 2647 | Growth | SsSNP2006B_628 |
| 2648 | Growth | SsSNP2006B_1421 |
| 2649 | Growth | SsSNP2006B_808 |
| 2650 | Growth | SsSNP2006B_809 |
| 2651 | Growth | SsSNP2006B_2497 |
| 2652 | Growth | SsSNP2006B_444 |
| 2653 | Growth | SsSNP2006B_1329 |
| 2654 | Growth | SsSNP2006B_2080 |
| 2655 | Growth | SsSNP2006B_268 |
| 2656 | Growth | SsSNP2006B_1617 |
| 2657 | Growth | SsSNP2006B_508 |
| 2658 | Growth | SsSNP2006B_1543 |
| 2659 | Growth | SsSNP2006B_931 |
| 2660 | Growth | SsSNP2006B_2178 |
| 2661 | Growth | SsSNP2006B_1678 |
| 2662 | Growth | SsSNP2006B_1833 |
| 2663 | Growth | SsSNP2006B_2582 |
| 2664 | Growth | SsSNP2006B_672 |
| 2665 | Growth | SsSNP2006B_1849 |
| 2666 | Growth | SsSNP2006B_90 |
| 2667 | Growth | SsSNP2006B_1303 |
| 2668 | Growth | SsSNP2006B_1137 |
| 2669 | Growth | SsSNP2006B_2429 |
| 2670 | Growth | SsSNP2006B_1458 |
| 2671 | Growth | SsSNP2006B_1475 |
| 2672 | Growth | SsSNP2006B_2001 |
| 2673 | Growth | SsSNP2006B_1204 |
| 2674 | Growth | SsSNP2006B_2006 |
| 2675 | Growth | SsSNP2006B_1044 |
| 2676 | Growth | SsSNP2006B_1322 |
| 2677 | Growth | SsSNP2006B_789 |
| 2678 | Growth | SsSNP2006B_704 |
| 2679 | Growth | SsSNP2006B_1946 |
| 2680 | Growth | SsSNP2006B_2546 |
| 2681 | Growth | SsSNP2006B_2098 |
| 2682 | Growth | SsSNP2006B_1510 |
| 2683 | Growth | SsSNP2006B_764 |
| 2684 | Growth | SsSNP2006B_355 |
| 2685 | Growth | SsSNP2006B_462 |
| 2686 | Growth | SsSNP2006B_1085 |
| 2687 | Growth | SsSNP2006B_432 |
| 2688 | Growth | SsSNP2006B_52 |
| 2689 | Growth | SsSNP2006B_216 |
| 2690 | Growth | SsSNP2006B_814 |
| 2691 | Growth | SsSNP2006B_404 |
| 2692 | Growth | SsSNP2006B_624 |
| 2693 | Growth | SsSNP2006B_2469 |
| 2694 | Growth | SsSNP2006B_279 |
| 2695 | Growth | SsSNP2006B_1405 |
| 2696 | Growth | SsSNP2006B_544 |
| 2697 | Growth | SsSNP2006B_2311 |
| 2698 | Growth | SsSNP2006B_1210 |
| 2699 | Growth | SsSNP2006B_2041 |
| 2700 | Growth | SsSNP2006B_1973 |
| 2701 | Growth | SsSNP2006B_2532 |
| 2702 | Growth | SsSNP2006B_520 |
| 2703 | Growth | SsSNP2006B_638 |
| 2704 | Growth | SsSNP2006B_2070 |
| 2705 | Growth | SsSNP2006B_658 |
| 2706 | Growth | SsSNP2006B_989 |
| 2707 | Growth | SsSNP2006B_1815 |
| 2708 | Growth | SsSNP2006B_2042 |
| 2709 | Growth | SsSNP2006B_1799 |
| 2710 | Growth | SsSNP2006B_2393 |
| 2711 | Growth | SsSNP2006B_1081 |
| 2712 | Growth | SsSNP2006B_1964 |
| 2713 | Growth | SsSNP2006B_625 |
| 2714 | Growth | SsSNP2006B_1515 |
| 2715 | Growth | SsSNP2006B_1255 |
| 2716 | Growth | SsSNP2006B_872 |
| 2717 | Growth | SsSNP2006B_1694 |
| 2718 | Growth | SsSNP2006B_2477 |
| 2719 | Growth | SsSNP2006B_459 |
| 2720 | Growth | SsSNP2006B_1671 |
| 2721 | Growth | SsSNP2006B_1672 |
| 2722 | Growth | SsSNP2006B_996 |
| 2723 | Growth | SsSNP2006B_408 |
| 2724 | Growth | SsSNP2006B_1856 |
| 2725 | Growth | SsSNP2006B_1971 |
| 2726 | Growth | SsSNP2006B_1005 |
| 2727 | Growth | SsSNP2006B_1440 |
| 2728 | Growth | SsSNP2006B_2335 |
| 2729 | Growth | SsSNP2006B_1158 |
| 2730 | Growth | SsSNP2006B_1232 |
| 2731 | Growth | SsSNP2006B_1037 |
| 2732 | Growth | SsSNP2006B_2253 |
| 2733 | Growth | SsSNP2006B_2254 |
| 2734 | Growth | SsSNP2006B_842 |
| 2735 | Growth | SsSNP2006B_997 |
| 2736 | Growth | SsSNP2006B_2280 |
| 2737 | Growth | SsSNP2006B_2183 |
| 2738 | Growth | SsSNP2006B_187 |
| 2739 | Growth | SsSNP2006B_269 |
| 2740 | Growth | SsSNP2006B_1299 |
| 2741 | Growth | SsSNP2006B_2132 |
| 2742 | Growth | SsSNP2006B_2180 |
| 2743 | Growth | SsSNP2006B_2135 |
| 2744 | Growth | SsSNP2006B_2294 |
| 2745 | Growth | SsSNP2006B_1644 |
| 2746 | Growth | SsSNP2006B_605 |
| 2747 | Growth | SsSNP2006B_1245 |
| 2748 | Reproduction | SsSNP2006B_392 |
| 2749 | Reproduction | SsSNP2006B_733 |
| 2750 | Reproduction | SsSNP2006B_2359 |
| 2751 | Reproduction | SsSNP2006B_164 |
| 2752 | Reproduction | SsSNP2006B_1257 |
| 2753 | Reproduction | SsSNP2006B_2476 |
| 2754 | Reproduction | SsSNP2006B_1127 |
| 2755 | Reproduction | SsSNP2006B_2328 |
| 2756 | Reproduction | SsSNP2006B_2413 |
| 2757 | Reproduction | SsSNP2006B_1419 |
| 2758 | Reproduction | SsSNP2006B_2491 |
| 2759 | Reproduction | SsSNP2006B_1944 |
| 2760 | Reproduction | SsSNP2006B_673 |
| 2761 | Reproduction | SsSNP2006B_1723 |
| 2762 | Reproduction | SsSNP2006B_662 |
| 2763 | Reproduction | SsSNP2006B_1777 |
| 2764 | Reproduction | SsSNP2006B_1863 |
| 2765 | Reproduction | SsSNP2006B_186 |
| 2766 | Reproduction | SsSNP2006B_1690 |
| 2767 | Reproduction | SsSNP2006B_1094 |
| 2768 | Reproduction | SsSNP2006B_1831 |
| 2769 | Reproduction | SsSNP2006B_495 |
| 2770 | Reproduction | SsSNP2006B_2174 |
| 2771 | Reproduction | SsSNP2006B_1610 |
| 2772 | Reproduction | SsSNP2006B_2553 |
| 2773 | Reproduction | SsSNP2006B_2200 |
| 2774 | Reproduction | SsSNP2006B_1984 |
| 2775 | Reproduction | SsSNP2006B_256 |
| 2776 | Reproduction | SsSNP2006B_1350 |
| 2777 | Reproduction | SsSNP2006B_1612 |
| 2778 | Reproduction | SsSNP2006B_885 |
| 2779 | Reproduction | SsSNP2006B_334 |
| 2780 | Reproduction | SsSNP2006B_1459 |
| 2781 | Reproduction | SsSNP2006B_2190 |
| 2782 | Reproduction | SsSNP2006B_1497 |
| 2783 | Reproduction | SsSNP2006B_2542 |
| 2784 | Reproduction | SsSNP2006B_383 |
| 2785 | Reproduction | SsSNP2006B_501 |
| 2786 | Reproduction | SsSNP2006B_1181 |
| 2787 | Reproduction | SsSNP2006B_2229 |
| 2788 | Reproduction | SsSNP2006B_726 |
| 2789 | Reproduction | SsSNP2006B_2101 |
| 2790 | Reproduction | SsSNP2006B_758 |
| 2791 | Reproduction | SsSNP2006B_77 |
| 2792 | Reproduction | SsSNP2006B_1265 |
| 2793 | Reproduction | SsSNP2006B_762 |
| 2794 | Reproduction | SsSNP2006B_1473 |
| 2795 | Reproduction | SsSNP2006B_1517 |
| 2796 | Reproduction | SsSNP2006B_1285 |
| 2797 | Reproduction | SsSNP2006B_18 |
| 2798 | Reproduction | SsSNP2006B_336 |
| 2799 | Reproduction | SsSNP2006B_2264 |
| 2800 | Reproduction | SsSNP2006B_653 |
| 2801 | Reproduction | SsSNP2006B_603 |
| 2802 | Reproduction | SsSNP2006B_1290 |
| 2803 | Reproduction | SsSNP2006B_1298 |
| 2804 | Reproduction | SsSNP2006B_1840 |
| 2805 | Reproduction | SsSNP2006B_434 |
| 2806 | Reproduction | SsSNP2006B_1129 |
| 2807 | Reproduction | SsSNP2006B_1665 |
| 2808 | Reproduction | SsSNP2006B_2396 |
| 2809 | Reproduction | SsSNP2006B_1013 |
| 2810 | Reproduction | SsSNP2006B_888 |
| 2811 | Reproduction | SsSNP2006B_163 |
| 2812 | Reproduction | SsSNP2006B_1547 |
| 2813 | Reproduction | SsSNP2006B_2265 |
| 2814 | Reproduction | SsSNP2006B_1408 |
| 2815 | Reproduction | SsSNP2006B_1317 |
| 2816 | Reproduction | SsSNP2006B_1394 |
| 2817 | Reproduction | SsSNP2006B_1092 |
| 2818 | Reproduction | SsSNP2006B_1896 |
| 2819 | Reproduction | SsSNP2006B_2239 |
| 2820 | Reproduction | SsSNP2006B_722 |
| 2821 | Reproduction | SsSNP2006B_406 |
| 2822 | Reproduction | SsSNP2006B_1351 |
| 2823 | Reproduction | SsSNP2006B_1857 |
| 2824 | Reproduction | SsSNP2006B_2552 |
| 2825 | Reproduction | SsSNP2006B_2154 |
| 2826 | Reproduction | SsSNP2006B_2507 |
| 2827 | Reproduction | SsSNP2006B_1339 |
| 2828 | Reproduction | SsSNP2006B_1237 |
| 2829 | Reproduction | SsSNP2006B_1238 |
| 2830 | Reproduction | SsSNP2006B_835 |
| 2831 | Reproduction | SsSNP2006B_836 |
| 2832 | Reproduction | SsSNP2006B_867 |
| 2833 | Reproduction | SsSNP2006B_1615 |
| 2834 | Reproduction | SsSNP2006B_1775 |
| 2835 | Reproduction | SsSNP2006B_2118 |
| 2836 | Reproduction | SsSNP2006B_347 |
| 2837 | Reproduction | SsSNP2006B_828 |
| 2838 | Reproduction | SsSNP2006B_1843 |
| 2839 | Reproduction | SsSNP2006B_1583 |
| 2840 | Reproduction | SsSNP2006B_58 |
| 2841 | Reproduction | SsSNP2006B_970 |
| 2842 | Reproduction | SsSNP2006B_1462 |
| 2843 | Reproduction | SsSNP2006B_1386 |
| 2844 | Reproduction | SsSNP2006B_1717 |
| 2845 | Reproduction | SsSNP2006B_708 |
| 2846 | Reproduction | SsSNP2006B_2281 |
| 2847 | Reproduction | SsSNP2006B_1348 |
| 2848 | Reproduction | SsSNP2006B_1366 |
| 2849 | Reproduction | SsSNP2006B_2175 |
| 2850 | Reproduction | SsSNP2006B_826 |
| 2851 | Reproduction | SsSNP2006B_2062 |
| 2852 | Reproduction | SsSNP2006B_2063 |
| 2853 | Reproduction | SsSNP2006B_2478 |
| 2854 | Reproduction | SsSNP2006B_2540 |
| 2855 | Reproduction | SsSNP2006B_1367 |
| 2856 | Reproduction | SsSNP2006B_2181 |
| 2857 | Reproduction | SsSNP2006B_581 |
| 2858 | Reproduction | SsSNP2006B_500 |
| 2859 | Reproduction | SsSNP2006B_841 |
| 2860 | Reproduction | SsSNP2006B_2331 |
| 2861 | Reproduction | SsSNP2006B_732 |
| 2862 | Reproduction | SsSNP2006B_219 |
| 2863 | Reproduction | SsSNP2006B_1993 |
| 2864 | Reproduction | SsSNP2006B_512 |
| 2865 | Reproduction | SsSNP2006B_1986 |
| 2866 | Reproduction | SsSNP2006B_1101 |
| 2867 | Reproduction | SsSNP2006B_1023 |
| 2868 | Reproduction | SsSNP2006B_1961 |
| 2869 | Reproduction | SsSNP2006B_1271 |
| 2870 | Reproduction | SsSNP2006B_2225 |
| 2871 | Reproduction | SsSNP2006B_2134 |
| 2872 | Reproduction | SsSNP2006B_2321 |
| 2873 | Reproduction | SsSNP2006B_2482 |
| 2874 | Reproduction | SsSNP2006B_2395 |
| 2875 | Reproduction | SsSNP2006B_874 |
| 2876 | Reproduction | SsSNP2006B_1310 |
| 2877 | Reproduction | SsSNP2006B_542 |
| 2878 | Reproduction | SsSNP2006B_543 |
| 2879 | Reproduction | SsSNP2006B_763 |
| 2880 | Reproduction | SsSNP2006B_2185 |
| 2881 | Reproduction | SsSNP2006B_690 |
| 2882 | Reproduction | SsSNP2006B_1431 |
| 2883 | Reproduction | SsSNP2006B_2257 |
| 2884 | Reproduction | SsSNP2006B_2285 |
| 2885 | Reproduction | SsSNP2006B_176 |
| 2886 | Reproduction | SsSNP2006B_420 |
| 2887 | Reproduction | SsSNP2006B_1220 |
| 2888 | Reproduction | SsSNP2006B_1754 |
| 2889 | Reproduction | SsSNP2006B_1482 |
| 2890 | Reproduction | SsSNP2006B_2105 |
| 2891 | Reproduction | SsSNP2006B_314 |
| 2892 | Reproduction | SsSNP2006B_395 |
| 2893 | Reproduction | SsSNP2006B_1773 |
| 2894 | Reproduction | SsSNP2006B_296 |
| 2895 | Reproduction | SsSNP2006B_1294 |
| 2896 | Reproduction | SsSNP2006B_2377 |
| 2897 | Reproduction | SsSNP2006B_2030 |
| 2898 | Reproduction | SsSNP2006B_100 |
| 2899 | Reproduction | SsSNP2006B_953 |
| 2900 | Reproduction | SsSNP2006B_1594 |
| 2901 | Reproduction | SsSNP2006B_130 |
| 2902 | Reproduction | SsSNP2006B_399 |
| 2903 | Reproduction | SsSNP2006B_1349 |
| 2904 | Reproduction | SsSNP2006B_782 |
| 2905 | Reproduction | SsSNP2006B_2270 |
| 2906 | Reproduction | SsSNP2006B_2323 |
| 2907 | Reproduction | SsSNP2006B_487 |
| 2908 | Reproduction | SsSNP2006B_1892 |
| 2909 | Reproduction | SsSNP2006B_86 |
| 2910 | Reproduction | SsSNP2006B_978 |
| 2911 | Reproduction | SsSNP2006B_472 |
| 2912 | Reproduction | SsSNP2006B_567 |
| 2913 | Reproduction | SsSNP2006B_1032 |
| 2914 | Reproduction | SsSNP2006B_1670 |
| 2915 | Reproduction | SsSNP2006B_1949 |
| 2916 | Reproduction | SsSNP2006B_2400 |
| 2917 | Reproduction | SsSNP2006B_730 |
| 2918 | Reproduction | SsSNP2006B_1186 |
| 2919 | Reproduction | SsSNP2006B_2192 |
| 2920 | Reproduction | SsSNP2006B_2044 |
| 2921 | Reproduction | SsSNP2006B_510 |
| 2922 | Reproduction | SsSNP2006B_971 |
| 2923 | Reproduction | SsSNP2006B_2378 |
| 2924 | Reproduction | SsSNP2006B_2007 |
| 2925 | Reproduction | SsSNP2006B_2016 |
| 2926 | Reproduction | SsSNP2006B_881 |
| 2927 | Reproduction | SsSNP2006B_2186 |
| 2928 | Reproduction | SsSNP2006B_1657 |
| 2929 | Reproduction | SsSNP2006B_1879 |
| 2930 | Reproduction | SsSNP2006B_6 |
| 2931 | Reproduction | SsSNP2006B_40 |
| 2932 | Reproduction | SsSNP2006B_2587 |
| 2933 | Reproduction | SsSNP2006B_1909 |
| 2934 | Reproduction | SsSNP2006B_742 |
| 2935 | Reproduction | SsSNP2006B_1503 |
| 2936 | Reproduction | SsSNP2006B_560 |
| 2937 | Reproduction | SsSNP2006B_247 |
| 2938 | Reproduction | SsSNP2006B_1771 |
| 2939 | Reproduction | SsSNP2006B_1114 |
| 2940 | Reproduction | SsSNP2006B_745 |
| 2941 | Reproduction | SsSNP2006B_360 |
| 2942 | Reproduction | SsSNP2006B_79 |
| 2943 | Reproduction | SsSNP2006B_1761 |
| 2944 | Reproduction | SsSNP2006B_2074 |
| 2945 | Reproduction | SsSNP2006B_2234 |
| 2946 | Reproduction | SsSNP2006B_271 |
| 2947 | Reproduction | SsSNP2006B_2292 |
| 2948 | Reproduction | SsSNP2006B_2014 |
| 2949 | Reproduction | SsSNP2006B_2509 |
| 2950 | Reproduction | SsSNP2006B_2510 |
| 2951 | Reproduction | SsSNP2006B_280 |
| 2952 | Reproduction | SsSNP2006B_1992 |
| 2953 | Reproduction | SsSNP2006B_2434 |
| 2954 | Reproduction | SsSNP2006B_2164 |
| 2955 | Reproduction | SsSNP2006B_2291 |
| 2956 | Reproduction | SsSNP2006B_1121 |
| 2957 | Reproduction | SsSNP2006B_1106 |
| 2958 | Reproduction | SsSNP2006B_1191 |
| 2959 | Reproduction | SsSNP2006B_2486 |
| 2960 | Reproduction | SsSNP2006B_1625 |
| 2961 | Reproduction | SsSNP2006B_822 |
| 2962 | Reproduction | SsSNP2006B_1738 |
| 2963 | Reproduction | SsSNP2006B_2272 |
| 2964 | Reproduction | SsSNP2006B_2097 |
| 2965 | Reproduction | SsSNP2006B_1616 |
| 2966 | Reproduction | SsSNP2006B_2242 |
| 2967 | Reproduction | SsSNP2006B_2053 |
| 2968 | Reproduction | SsSNP2006B_11 |
| 2969 | Reproduction | SsSNP2006B_2221 |
| 2970 | Reproduction | SsSNP2006B_2384 |
| 2971 | Reproduction | SsSNP2006B_209 |
| 2972 | Reproduction | SsSNP2006B_411 |
| 2973 | Reproduction | SsSNP2006B_797 |
| 2974 | Reproduction | SsSNP2006B_2496 |
| 2975 | Reproduction | SsSNP2006B_2468 |
| 2976 | Reproduction | SsSNP2006B_1550 |
| 2977 | Reproduction | SsSNP2006B_27 |
| 2978 | Reproduction | SsSNP2006B_91 |
| 2979 | Reproduction | SsSNP2006B_777 |
| 2980 | Reproduction | SsSNP2006B_1580 |
| 2981 | Reproduction | SsSNP2006B_788 |
| 2982 | Reproduction | SsSNP2006B_317 |
| 2983 | Reproduction | SsSNP2006B_14 |
| 2984 | Reproduction | SsSNP2006B_569 |
| 2985 | Reproduction | SsSNP2006B_2327 |
| 2986 | Reproduction | SsSNP2006B_1476 |
| 2987 | Reproduction | SsSNP2006B_181 |
| 2988 | Reproduction | SsSNP2006B_2220 |
| 2989 | Reproduction | SsSNP2006B_613 |
| 2990 | Reproduction | SsSNP2006B_73 |
| 2991 | Reproduction | SsSNP2006B_74 |
| 2992 | Reproduction | SsSNP2006B_286 |
| 2993 | Reproduction | SsSNP2006B_1630 |
| 2994 | Reproduction | SsSNP2006B_1030 |
| 2995 | Reproduction | SsSNP2006B_4 |
| 2996 | Reproduction | SsSNP2006B_1640 |
| 2997 | Reproduction | SsSNP2006B_140 |
| 2998 | Reproduction | SsSNP2006B_1638 |
| 2999 | Reproduction | SsSNP2006B_2592 |
| 3000 | Reproduction | SsSNP2006B_597 |
| 3001 | Reproduction | SsSNP2006B_720 |
| 3002 | Reproduction | SsSNP2006B_719 |
| 3003 | Reproduction | SsSNP2006B_1418 |
| 3004 | Reproduction | SsSNP2006B_1259 |
| 3005 | Reproduction | SsSNP2006B_1397 |
| 3006 | Reproduction | SsSNP2006B_1699 |
| 3007 | Reproduction | SsSNP2006B_398 |
| 3008 | Reproduction | SsSNP2006B_854 |
| 3009 | Reproduction | SsSNP2006B_783 |
| 3010 | Reproduction | SsSNP2006B_1185 |
| 3011 | Reproduction | SsSNP2006B_1280 |
| 3012 | Reproduction | SsSNP2006B_993 |
| 3013 | Reproduction | SsSNP2006B_1693 |
| 3014 | Reproduction | SsSNP2006B_1683 |
| 3015 | Reproduction | SsSNP2006B_2578 |
| 3016 | Reproduction | SsSNP2006B_949 |
| 3017 | Reproduction | SsSNP2006B_1306 |
| 3018 | Reproduction | SsSNP2006B_682 |
| 3019 | Reproduction | SsSNP2006B_477 |
| 3020 | Reproduction | SsSNP2006B_2084 |
| 3021 | Reproduction | SsSNP2006B_1979 |
| 3022 | Reproduction | SsSNP2006B_1980 |
| 3023 | Reproduction | SsSNP2006B_1861 |
| 3024 | Reproduction | SsSNP2006B_907 |
| 3025 | Reproduction | SsSNP2006B_1502 |
| 3026 | Reproduction | SsSNP2006B_1443 |
| 3027 | Reproduction | SsSNP2006B_1039 |
| 3028 | Reproduction | SsSNP2006B_659 |
| 3029 | Reproduction | SsSNP2006B_533 |
| 3030 | Reproduction | SsSNP2006B_1278 |
| 3031 | Reproduction | SsSNP2006B_1268 |
| 3032 | Reproduction | SsSNP2006B_474 |
| 3033 | Reproduction | SsSNP2006B_1532 |
| 3034 | Reproduction | SsSNP2006B_2521 |
| 3035 | Reproduction | SsSNP2006B_2551 |
| 3036 | Reproduction | SsSNP2006B_2025 |
| 3037 | Reproduction | SsSNP2006B_394 |
| 3038 | Reproduction | SsSNP2006B_1585 |
| 3039 | Reproduction | SsSNP2006B_1902 |
| 3040 | Reproduction | SsSNP2006B_1976 |
| 3041 | Reproduction | SsSNP2006B_1325 |
| 3042 | Reproduction | SsSNP2006B_1824 |
| 3043 | Reproduction | SsSNP2006B_924 |
| 3044 | Reproduction | SsSNP2006B_2394 |
| 3045 | Reproduction | SsSNP2006B_795 |
| 3046 | Reproduction | SsSNP2006B_1390 |
| 3047 | Reproduction | SsSNP2006B_1214 |
| 3048 | Reproduction | SsSNP2006B_539 |
| 3049 | Reproduction | SsSNP2006B_1090 |
| 3050 | Reproduction | SsSNP2006B_1135 |
| 3051 | Reproduction | SsSNP2006B_2170 |
| 3052 | Reproduction | SsSNP2006B_1687 |
| 3053 | Reproduction | SsSNP2006B_174 |
| 3054 | Reproduction | SsSNP2006B_709 |
| 3055 | Reproduction | SsSNP2006B_478 |
| 3056 | Reproduction | SsSNP2006B_2244 |
| 3057 | Reproduction | SsSNP2006B_2376 |
| 3058 | Reproduction | SsSNP2006B_53 |
| 3059 | Reproduction | SsSNP2006B_67 |
| 3060 | Reproduction | SsSNP2006B_1759 |
| 3061 | Reproduction | SsSNP2006B_1439 |
| 3062 | Reproduction | SsSNP2006B_685 |
| 3063 | Reproduction | SsSNP2006B_915 |
| 3064 | Reproduction | SsSNP2006B_1080 |
| 3065 | Reproduction | SsSNP2006B_857 |
| 3066 | Reproduction | SsSNP2006B_2159 |
| 3067 | Reproduction | SsSNP2006B_2423 |
| 3068 | Reproduction | SsSNP2006B_2167 |
| 3069 | Reproduction | SsSNP2006B_1908 |
| 3070 | Reproduction | SsSNP2006B_1981 |
| 3071 | Reproduction | SsSNP2006B_665 |
| 3072 | Reproduction | SsSNP2006B_1575 |
| 3073 | Reproduction | SsSNP2006B_1217 |
| 3074 | Reproduction | SsSNP2006B_1939 |
| 3075 | Reproduction | SsSNP2006B_2109 |
| 3076 | Reproduction | SsSNP2006B_2011 |
| 3077 | Reproduction | SsSNP2006B_607 |
| 3078 | Reproduction | SsSNP2006B_1758 |
| 3079 | Reproduction | SsSNP2006B_1836 |
| 3080 | Reproduction | SsSNP2006B_1804 |
| 3081 | Reproduction | SsSNP2006B_1791 |
| 3082 | Reproduction | SsSNP2006B_1569 |
| 3083 | Reproduction | SsSNP2006B_646 |
| 3084 | Reproduction | SsSNP2006B_1068 |
| 3085 | Reproduction | SsSNP2006B_1357 |
| 3086 | Reproduction | SsSNP2006B_284 |
| 3087 | Reproduction | SsSNP2006B_524 |
| 3088 | Reproduction | SsSNP2006B_1227 |
| 3089 | Reproduction | SsSNP2006B_1334 |
| 3090 | Reproduction | SsSNP2006B_1086 |
| 3091 | Reproduction | SsSNP2006B_178 |
| 3092 | Reproduction | SsSNP2006B_115 |
| 3093 | Reproduction | SsSNP2006B_469 |
| 3094 | Reproduction | SsSNP2006B_228 |
| 3095 | Reproduction | SsSNP2006B_1596 |
| 3096 | Reproduction | SsSNP2006B_2163 |
| 3097 | Reproduction | SsSNP2006B_235 |
| 3098 | Reproduction | SsSNP2006B_635 |
| 3099 | Reproduction | SsSNP2006B_864 |
| 3100 | Reproduction | SsSNP2006B_2584 |
| 3101 | Reproduction | SsSNP2006B_2087 |
| 3102 | Reproduction | SsSNP2006B_2217 |
| 3103 | Reproduction | SsSNP2006B_531 |
| 3104 | Reproduction | SsSNP2006B_1488 |
| 3105 | Reproduction | SsSNP2006B_1489 |
| 3106 | Reproduction | SsSNP2006B_1825 |
| 3107 | Reproduction | SsSNP2006B_2370 |
| 3108 | Reproduction | SsSNP2006B_177 |
| 3109 | Reproduction | SsSNP2006B_1934 |
| 3110 | Reproduction | SsSNP2006B_1511 |
| 3111 | Reproduction | SsSNP2006B_1134 |
| 3112 | Reproduction | SsSNP2006B_1707 |
| 3113 | Reproduction | SsSNP2006B_1465 |
| 3114 | Reproduction | SsSNP2006B_1669 |
| 3115 | Reproduction | SsSNP2006B_281 |
| 3116 | Reproduction | SsSNP2006B_750 |
| 3117 | Reproduction | SsSNP2006B_1958 |
| 3118 | Reproduction | SsSNP2006B_833 |
| 3119 | Reproduction | SsSNP2006B_955 |
| 3120 | Reproduction | SsSNP2006B_844 |
| 3121 | Reproduction | SsSNP2006B_2346 |
| 3122 | Reproduction | SsSNP2006B_2383 |
| 3123 | Reproduction | SsSNP2006B_1328 |
| 3124 | Reproduction | SsSNP2006B_1731 |
| 3125 | Reproduction | SsSNP2006B_1871 |
| 3126 | Reproduction | SsSNP2006B_875 |
| 3127 | Reproduction | SsSNP2006B_492 |
| 3128 | Reproduction | SsSNP2006B_710 |
| 3129 | Reproduction | SsSNP2006B_1318 |
| 3130 | Reproduction | SsSNP2006B_1403 |
| 3131 | Reproduction | SsSNP2006B_1426 |
| 3132 | Reproduction | SsSNP2006B_2123 |
| 3133 | Reproduction | SsSNP2006B_2227 |
| 3134 | Reproduction | SsSNP2006B_2010 |
| 3135 | Reproduction | SsSNP2006B_1584 |
| 3136 | Reproduction | SsSNP2006B_21 |
| 3137 | Reproduction | SsSNP2006B_663 |
| 3138 | Reproduction | SsSNP2006B_633 |
| 3139 | Reproduction | SsSNP2006B_2293 |
| 3140 | Reproduction | SsSNP2006B_1027 |
| 3141 | Reproduction | SsSNP2006B_686 |
| 3142 | Reproduction | SsSNP2006B_537 |
| 3143 | Reproduction | SsSNP2006B_1020 |
| 3144 | Reproduction | SsSNP2006B_1420 |
| 3145 | Reproduction | SsSNP2006B_28 |
| 3146 | Reproduction | SsSNP2006B_89 |
| 3147 | Reproduction | SsSNP2006B_2148 |
| 3148 | Reproduction | SsSNP2006B_631 |
| 3149 | Reproduction | SsSNP2006B_1025 |
| 3150 | Reproduction | SsSNP2006B_1561 |
| 3151 | Reproduction | SsSNP2006B_2416 |
| 3152 | Reproduction | SsSNP2006B_1160 |
| 3153 | Reproduction | SsSNP2006B_116 |
| 3154 | Reproduction | SsSNP2006B_222 |
| 3155 | Reproduction | SsSNP2006B_2224 |
| 3156 | Reproduction | SsSNP2006B_595 |
| 3157 | Reproduction | SsSNP2006B_2458 |
| 3158 | Reproduction | SsSNP2006B_1576 |
| 3159 | Reproduction | SsSNP2006B_238 |
| 3160 | Reproduction | SsSNP2006B_1451 |
| 3161 | Reproduction | SsSNP2006B_2237 |
| 3162 | Reproduction | SsSNP2006B_1100 |
| 3163 | Reproduction | SsSNP2006B_1740 |
| 3164 | Reproduction | SsSNP2006B_678 |
| 3165 | Reproduction | SsSNP2006B_1031 |
| 3166 | Reproduction | SsSNP2006B_1292 |
| 3167 | Reproduction | SsSNP2006B_195 |
| 3168 | Reproduction | SsSNP2006B_818 |
| 3169 | Reproduction | SsSNP2006B_954 |
| 3170 | Reproduction | SsSNP2006B_1091 |
| 3171 | Reproduction | SsSNP2006B_2526 |
| 3172 | Reproduction | SsSNP2006B_335 |
| 3173 | Reproduction | SsSNP2006B_2498 |
| 3174 | Reproduction | SsSNP2006B_568 |
| 3175 | Reproduction | SsSNP2006B_716 |
| 3176 | Reproduction | SsSNP2006B_2511 |
| 3177 | Reproduction | SsSNP2006B_1452 |
| 3178 | Reproduction | SsSNP2006B_1817 |
| 3179 | Reproduction | SsSNP2006B_2465 |
| 3180 | Reproduction | SsSNP2006B_2315 |
| 3181 | Reproduction | SsSNP2006B_2581 |
| 3182 | Reproduction | SsSNP2006B_502 |
| 3183 | Reproduction | SsSNP2006B_2065 |
| 3184 | Reproduction | SsSNP2006B_1851 |
| 3185 | Reproduction | SsSNP2006B_2258 |
| 3186 | Reproduction | SsSNP2006B_1442 |
| 3187 | Reproduction | SsSNP2006B_33 |
| 3188 | Reproduction | SsSNP2006B_1417 |
| 3189 | Reproduction | SsSNP2006B_2179 |
| 3190 | Reproduction | SsSNP2006B_282 |
| 3191 | Reproduction | SsSNP2006B_2355 |
| 3192 | Reproduction | SsSNP2006B_901 |
| 3193 | Reproduction | SsSNP2006B_2033 |
| 3194 | Reproduction | SsSNP2006B_2459 |
| 3195 | Reproduction | SsSNP2006B_600 |
| 3196 | Reproduction | SsSNP2006B_2283 |
| 3197 | Reproduction | SsSNP2006B_2500 |
| 3198 | Reproduction | SsSNP2006B_740 |
| 3199 | Reproduction | SsSNP2006B_134 |
| 3200 | Reproduction | SsSNP2006B_135 |
| 3201 | Reproduction | SsSNP2006B_590 |
| 3202 | Reproduction | SsSNP2006B_70 |
| 3203 | Reproduction | SsSNP2006B_128 |
| 3204 | Reproduction | SsSNP2006B_1930 |
| 3205 | Reproduction | SsSNP2006B_162 |
| 3206 | Reproduction | SsSNP2006B_554 |
| 3207 | Reproduction | SsSNP2006B_2195 |
| 3208 | Reproduction | SsSNP2006B_639 |
| 3209 | Reproduction | SsSNP2006B_1726 |
| 3210 | Reproduction | SsSNP2006B_490 |
| 3211 | Reproduction | SsSNP2006B_1673 |
| 3212 | Reproduction | SsSNP2006B_2489 |
| 3213 | Reproduction | SsSNP2006B_221 |
| 3214 | Reproduction | SsSNP2006B_200 |
| 3215 | Reproduction | SsSNP2006B_333 |
| 3216 | Reproduction | SsSNP2006B_2322 |
| 3217 | Reproduction | SsSNP2006B_2032 |
| 3218 | Reproduction | SsSNP2006B_933 |
| 3219 | Reproduction | SsSNP2006B_917 |
| 3220 | Reproduction | SsSNP2006B_1533 |
| 3221 | Reproduction | SsSNP2006B_2018 |
| 3222 | Reproduction | SsSNP2006B_2426 |
| 3223 | Reproduction | SsSNP2006B_966 |
| 3224 | Reproduction | SsSNP2006B_39 |
| 3225 | Reproduction | SsSNP2006B_2277 |
| 3226 | Reproduction | SsSNP2006B_1614 |
| 3227 | Reproduction | SsSNP2006B_1922 |
| 3228 | Reproduction | SsSNP2006B_254 |
| 3229 | Reproduction | SsSNP2006B_540 |
| 3230 | Reproduction | SsSNP2006B_109 |
| 3231 | Reproduction | SsSNP2006B_1951 |
| 3232 | Reproduction | SsSNP2006B_1126 |
| 3233 | Reproduction | SsSNP2006B_1819 |
| 3234 | Reproduction | SsSNP2006B_288 |
| 3235 | Reproduction | SsSNP2006B_139 |
| 3236 | Reproduction | SsSNP2006B_496 |
| 3237 | Reproduction | SsSNP2006B_2456 |
| 3238 | Reproduction | SsSNP2006B_932 |
| 3239 | Reproduction | SsSNP2006B_1017 |
| 3240 | Reproduction | SsSNP2006B_2545 |
| 3241 | Reproduction | SsSNP2006B_1105 |
| 3242 | Reproduction | SsSNP2006B_1664 |
| 3243 | Reproduction | SsSNP2006B_2247 |
| 3244 | Reproduction | SsSNP2006B_956 |
| 3245 | Reproduction | SsSNP2006B_75 |
| 3246 | Reproduction | SsSNP2006B_649 |
| 3247 | Reproduction | SsSNP2006B_1789 |
| 3248 | Reproduction | SsSNP2006B_350 |
| 3249 | Reproduction | SsSNP2006B_94 |
| 3250 | Reproduction | SsSNP2006B_1399 |
| 3251 | Reproduction | SsSNP2006B_1161 |
| 3252 | Reproduction | SsSNP2006B_1622 |
| 3253 | Reproduction | SsSNP2006B_1373 |
| 3254 | Reproduction | SsSNP2006B_1895 |
| 3255 | Reproduction | SsSNP2006B_93 |
| 3256 | Reproduction | SsSNP2006B_1279 |
| 3257 | Reproduction | SsSNP2006B_1865 |
| 3258 | Reproduction | SsSNP2006B_1725 |
| 3259 | Reproduction | SsSNP2006B_305 |
| 3260 | Reproduction | SsSNP2006B_725 |
| 3261 | Reproduction | SsSNP2006B_1812 |
| 3262 | Reproduction | SsSNP2006B_847 |
| 3263 | Reproduction | SsSNP2006B_2554 |
| 3264 | Reproduction | SsSNP2006B_1067 |
| 3265 | Reproduction | SsSNP2006B_760 |
| 3266 | Reproduction | SsSNP2006B_1270 |
| 3267 | Reproduction | SsSNP2006B_879 |
| 3268 | Reproduction | SsSNP2006B_1365 |
| 3269 | Reproduction | SsSNP2006B_683 |
| 3270 | Reproduction | SsSNP2006B_69 |
| 3271 | Reproduction | SsSNP2006B_861 |
| 3272 | Reproduction | SsSNP2006B_460 |
| 3273 | Reproduction | SsSNP2006B_108 |
| 3274 | Reproduction | SsSNP2006B_498 |
| 3275 | Reproduction | SsSNP2006B_2375 |
| 3276 | Reproduction | SsSNP2006B_572 |
| 3277 | Reproduction | SsSNP2006B_729 |
| 3278 | Reproduction | SsSNP2006B_267 |
| 3279 | Reproduction | SsSNP2006B_2493 |
| 3280 | Reproduction | SsSNP2006B_556 |
| 3281 | Reproduction | SsSNP2006B_150 |
| 3282 | Reproduction | SsSNP2006B_1805 |
| 3283 | Reproduction | SsSNP2006B_951 |
| 3284 | Reproduction | SsSNP2006B_1029 |
| 3285 | Reproduction | SsSNP2006B_1798 |
| 3286 | Reproduction | SsSNP2006B_1525 |
| 3287 | Reproduction | SsSNP2006B_1272 |
| 3288 | Reproduction | SsSNP2006B_422 |
| 3289 | Reproduction | SsSNP2006B_423 |
| 3290 | Reproduction | SsSNP2006B_2013 |
| 3291 | Reproduction | SsSNP2006B_957 |
| 3292 | Reproduction | SsSNP2006B_82 |
| 3293 | Reproduction | SsSNP2006B_2020 |
| 3294 | Reproduction | SsSNP2006B_982 |
| 3295 | Reproduction | SsSNP2006B_1662 |
| 3296 | Reproduction | SsSNP2006B_193 |
| 3297 | Reproduction | SsSNP2006B_1175 |
| 3298 | Reproduction | SsSNP2006B_846 |
| 3299 | Reproduction | SsSNP2006B_1260 |
| 3300 | Reproduction | SsSNP2006B_1157 |
| 3301 | Reproduction | SsSNP2006B_1739 |
| 3302 | Reproduction | SsSNP2006B_1498 |
| 3303 | Reproduction | SsSNP2006B_2202 |
| 3304 | Reproduction | SsSNP2006B_1430 |
| 3305 | Reproduction | SsSNP2006B_225 |
| 3306 | Reproduction | SsSNP2006B_679 |
| 3307 | Reproduction | SsSNP2006B_1679 |
| 3308 | Reproduction | SsSNP2006B_2005 |
| 3309 | Reproduction | SsSNP2006B_2166 |
| 3310 | Reproduction | SsSNP2006B_1494 |
| 3311 | Reproduction | SsSNP2006B_2538 |
| 3312 | Reproduction | SsSNP2006B_1595 |
| 3313 | Reproduction | SsSNP2006B_1369 |
| 3314 | Reproduction | SsSNP2006B_1370 |
| 3315 | Reproduction | SsSNP2006B_876 |
| 3316 | Reproduction | SsSNP2006B_2334 |
| 3317 | Reproduction | SsSNP2006B_1965 |
| 3318 | Reproduction | SsSNP2006B_2343 |
| 3319 | Reproduction | SsSNP2006B_1926 |
| 3320 | Reproduction | SsSNP2006B_165 |
| 3321 | Reproduction | SsSNP2006B_1745 |
| 3322 | Reproduction | SsSNP2006B_277 |
| 3323 | Reproduction | SsSNP2006B_2336 |
| 3324 | Reproduction | SsSNP2006B_2420 |
| 3325 | Reproduction | SsSNP2006B_805 |
| 3326 | Reproduction | SsSNP2006B_785 |
| 3327 | Reproduction | SsSNP2006B_2439 |
| 3328 | Reproduction | SsSNP2006B_381 |
| 3329 | Reproduction | SsSNP2006B_157 |
| 3330 | Reproduction | SsSNP2006B_1425 |
| 3331 | Reproduction | SsSNP2006B_185 |
| 3332 | Reproduction | SsSNP2006B_2023 |
| 3333 | Reproduction | SsSNP2006B_961 |
| 3334 | Reproduction | SsSNP2006B_291 |
| 3335 | Reproduction | SsSNP2006B_968 |
| 3336 | Reproduction | SsSNP2006B_1518 |
| 3337 | Reproduction | SsSNP2006B_2422 |
| 3338 | Reproduction | SsSNP2006B_2351 |
| 3339 | Reproduction | SsSNP2006B_940 |
| 3340 | Reproduction | SsSNP2006B_1490 |
| 3341 | Reproduction | SsSNP2006B_2028 |
| 3342 | Reproduction | SsSNP2006B_1269 |
| 3343 | Reproduction | SsSNP2006B_307 |
| 3344 | Reproduction | SsSNP2006B_1437 |
| 3345 | Reproduction | SsSNP2006B_2198 |
| 3346 | Reproduction | SsSNP2006B_1578 |
| 3347 | Reproduction | SsSNP2006B_119 |
| 3348 | Reproduction | SsSNP2006B_1655 |
| 3349 | Reproduction | SsSNP2006B_1932 |
| 3350 | Reproduction | SsSNP2006B_1444 |
| 3351 | Reproduction | SsSNP2006B_941 |
| 3352 | Reproduction | SsSNP2006B_1887 |
| 3353 | Reproduction | SsSNP2006B_1046 |
| 3354 | Reproduction | SsSNP2006B_1246 |
| 3355 | Reproduction | SsSNP2006B_1179 |
| 3356 | Reproduction | SsSNP2006B_880 |
| 3357 | Reproduction | SsSNP2006B_2409 |
| 3358 | Reproduction | SsSNP2006B_778 |
| 3359 | Reproduction | SsSNP2006B_278 |
| 3360 | Reproduction | SsSNP2006B_2499 |
| 3361 | Reproduction | SsSNP2006B_2490 |
| 3362 | Reproduction | SsSNP2006B_1359 |
| 3363 | Reproduction | SsSNP2006B_530 |
| 3364 | Reproduction | SsSNP2006B_272 |
| 3365 | Reproduction | SsSNP2006B_1632 |
| 3366 | Reproduction | SsSNP2006B_747 |
| 3367 | Reproduction | SsSNP2006B_2021 |
| 3368 | Reproduction | SsSNP2006B_2262 |
| 3369 | Reproduction | SsSNP2006B_253 |
| 3370 | Reproduction | SsSNP2006B_1378 |
| 3371 | Reproduction | SsSNP2006B_2363 |
| 3372 | Reproduction | SsSNP2006B_723 |
| 3373 | Reproduction | SsSNP2006B_348 |
| 3374 | Reproduction | SsSNP2006B_829 |
| 3375 | Reproduction | SsSNP2006B_2349 |
| 3376 | Reproduction | SsSNP2006B_800 |
| 3377 | Reproduction | SsSNP2006B_909 |
| 3378 | Reproduction | SsSNP2006B_1035 |
| 3379 | Reproduction | SsSNP2006B_442 |
| 3380 | Reproduction | SsSNP2006B_717 |
| 3381 | Reproduction | SsSNP2006B_2425 |
| 3382 | Reproduction | SsSNP2006B_1474 |
| 3383 | Reproduction | SsSNP2006B_1894 |
| 3384 | Reproduction | SsSNP2006B_117 |
| 3385 | Reproduction | SsSNP2006B_433 |
| 3386 | Reproduction | SsSNP2006B_519 |
| 3387 | Reproduction | SsSNP2006B_507 |
| 3388 | Reproduction | SsSNP2006B_2559 |
| 3389 | Reproduction | SsSNP2006B_2579 |
| 3390 | Reproduction | SsSNP2006B_707 |
| 3391 | Reproduction | SsSNP2006B_2367 |
| 3392 | Reproduction | SsSNP2006B_781 |
| 3393 | Reproduction | SsSNP2006B_1149 |
| 3394 | Reproduction | SsSNP2006B_262 |
| 3395 | Reproduction | SsSNP2006B_1877 |
| 3396 | Reproduction | SsSNP2006B_2573 |
| 3397 | Reproduction | SsSNP2006B_2031 |
| 3398 | Reproduction | SsSNP2006B_1795 |
| 3399 | Reproduction | SsSNP2006B_1900 |
| 3400 | Reproduction | SsSNP2006B_2460 |
| 3401 | Reproduction | SsSNP2006B_1650 |
| 3402 | Reproduction | SsSNP2006B_1531 |
| 3403 | Reproduction | SsSNP2006B_1989 |
| 3404 | Reproduction | SsSNP2006B_2107 |
| 3405 | Reproduction | SsSNP2006B_1456 |
| 3406 | Reproduction | SsSNP2006B_403 |
| 3407 | Reproduction | SsSNP2006B_121 |
| 3408 | Reproduction | SsSNP2006B_204 |
| 3409 | Reproduction | SsSNP2006B_312 |
| 3410 | Reproduction | SsSNP2006B_2523 |
| 3411 | Reproduction | SsSNP2006B_766 |
| 3412 | Reproduction | SsSNP2006B_1698 |
| 3413 | Reproduction | SsSNP2006B_1607 |
| 3414 | Reproduction | SsSNP2006B_2104 |
| 3415 | Reproduction | SsSNP2006B_2094 |
| 3416 | Reproduction | SsSNP2006B_756 |
| 3417 | Reproduction | SsSNP2006B_191 |
| 3418 | Reproduction | SsSNP2006B_1735 |
| 3419 | Reproduction | SsSNP2006B_1736 |
| 3420 | Reproduction | SsSNP2006B_393 |
| 3421 | Reproduction | SsSNP2006B_2213 |
| 3422 | Reproduction | SsSNP2006B_1955 |
| 3423 | Reproduction | SsSNP2006B_959 |
| 3424 | Reproduction | SsSNP2006B_1918 |
| 3425 | Reproduction | SsSNP2006B_2475 |
| 3426 | Reproduction | SsSNP2006B_2304 |
| 3427 | Reproduction | SsSNP2006B_182 |
| 3428 | Reproduction | SsSNP2006B_1635 |
| 3429 | Reproduction | SsSNP2006B_1681 |
| 3430 | Reproduction | SsSNP2006B_321 |
| 3431 | Reproduction | SsSNP2006B_1184 |
| 3432 | Reproduction | SsSNP2006B_2068 |
| 3433 | Reproduction | SsSNP2006B_2531 |
| 3434 | Reproduction | SsSNP2006B_1493 |
| 3435 | Reproduction | SsSNP2006B_2316 |
| 3436 | Reproduction | SsSNP2006B_571 |
| 3437 | Reproduction | SsSNP2006B_1631 |
| 3438 | Reproduction | SsSNP2006B_1911 |
| 3439 | Reproduction | SsSNP2006B_2437 |
| 3440 | Reproduction | SsSNP2006B_1916 |
| 3441 | Reproduction | SsSNP2006B_1484 |
| 3442 | Reproduction | SsSNP2006B_713 |
| 3443 | Reproduction | SsSNP2006B_1021 |
| 3444 | Reproduction | SsSNP2006B_2034 |
| 3445 | Reproduction | SsSNP2006B_257 |
| 3446 | Reproduction | SsSNP2006B_2403 |
| 3447 | Reproduction | SsSNP2006B_939 |
| 3448 | Reproduction | SsSNP2006B_787 |
| 3449 | Reproduction | SsSNP2006B_1903 |
| 3450 | Reproduction | SsSNP2006B_1821 |
| 3451 | Reproduction | SsSNP2006B_937 |
| 3452 | Reproduction | SsSNP2006B_1336 |
| 3453 | Reproduction | SsSNP2006B_1120 |
| 3454 | Reproduction | SsSNP2006B_105 |
| 3455 | Reproduction | SsSNP2006B_669 |
| 3456 | Reproduction | SsSNP2006B_2448 |
| 3457 | Reproduction | SsSNP2006B_614 |
| 3458 | Reproduction | SsSNP2006B_142 |
| 3459 | Reproduction | SsSNP2006B_1765 |
| 3460 | Reproduction | SsSNP2006B_1996 |
| 3461 | Reproduction | SsSNP2006B_623 |
| 3462 | Reproduction | SsSNP2006B_2527 |
| 3463 | Reproduction | SsSNP2006B_1460 |
| 3464 | Reproduction | SsSNP2006B_831 |
| 3465 | Reproduction | SsSNP2006B_616 |
| 3466 | Reproduction | SsSNP2006B_2266 |
| 3467 | Reproduction | SsSNP2006B_650 |
| 3468 | Reproduction | SsSNP2006B_1011 |
| 3469 | Reproduction | SsSNP2006B_1169 |
| 3470 | Reproduction | SsSNP2006B_2440 |
| 3471 | Reproduction | SsSNP2006B_452 |
| 3472 | Reproduction | SsSNP2006B_480 |
| 3473 | Reproduction | SsSNP2006B_1042 |
| 3474 | Reproduction | SsSNP2006B_2412 |
| 3475 | Reproduction | SsSNP2006B_1978 |
| 3476 | Reproduction | SsSNP2006B_367 |
| 3477 | Reproduction | SsSNP2006B_1548 |
| 3478 | Reproduction | SsSNP2006B_236 |
| 3479 | Reproduction | SsSNP2006B_1171 |
| 3480 | Reproduction | SsSNP2006B_2471 |
| 3481 | Reproduction | SsSNP2006B_617 |
| 3482 | Reproduction | SsSNP2006B_1605 |
| 3483 | Reproduction | SsSNP2006B_965 |
| 3484 | Reproduction | SsSNP2006B_1943 |
| 3485 | Reproduction | SsSNP2006B_110 |
| 3486 | Reproduction | SsSNP2006B_1019 |
| 3487 | Reproduction | SsSNP2006B_1038 |
| 3488 | Reproduction | SsSNP2006B_866 |
| 3489 | Reproduction | SsSNP2006B_2066 |
| 3490 | Reproduction | SsSNP2006B_555 |
| 3491 | Reproduction | SsSNP2006B_2424 |
| 3492 | Reproduction | SsSNP2006B_2158 |
| 3493 | Reproduction | SsSNP2006B_212 |
| 3494 | Reproduction | SsSNP2006B_1316 |
| 3495 | Reproduction | SsSNP2006B_1008 |
| 3496 | Reproduction | SsSNP2006B_2169 |
| 3497 | Reproduction | SsSNP2006B_921 |
| 3498 | Reproduction | SsSNP2006B_241 |
| 3499 | Reproduction | SsSNP2006B_242 |
| 3500 | Reproduction | SsSNP2006B_944 |
| 3501 | Reproduction | SsSNP2006B_2411 |
| 3502 | Reproduction | SsSNP2006B_32 |
| 3503 | Reproduction | SsSNP2006B_511 |
| 3504 | Reproduction | SsSNP2006B_1597 |
| 3505 | Reproduction | SsSNP2006B_1117 |
| 3506 | Reproduction | SsSNP2006B_1061 |
| 3507 | Reproduction | SsSNP2006B_1321 |
| 3508 | Reproduction | SsSNP2006B_1746 |
| 3509 | Reproduction | SsSNP2006B_1816 |
| 3510 | Reproduction | SsSNP2006B_2246 |
| 3511 | Reproduction | SsSNP2006B_1629 |
| 3512 | Reproduction | SsSNP2006B_1102 |
| 3513 | Reproduction | SsSNP2006B_123 |
| 3514 | Reproduction | SsSNP2006B_1907 |
| 3515 | Reproduction | SsSNP2006B_1291 |
| 3516 | Reproduction | SsSNP2006B_2110 |
| 3517 | Reproduction | SsSNP2006B_2399 |
| 3518 | Reproduction | SsSNP2006B_263 |
| 3519 | Reproduction | SsSNP2006B_1402 |
| 3520 | Reproduction | SsSNP2006B_2114 |
| 3521 | Reproduction | SsSNP2006B_926 |
| 3522 | Reproduction | SsSNP2006B_2494 |
| 3523 | Reproduction | SsSNP2006B_1244 |
| 3524 | Reproduction | SsSNP2006B_2544 |
| 3525 | Reproduction | SsSNP2006B_467 |
| 3526 | Reproduction | SsSNP2006B_2585 |
| 3527 | Reproduction | SsSNP2006B_1433 |
| 3528 | Reproduction | SsSNP2006B_2488 |
| 3529 | Reproduction | SsSNP2006B_265 |
| 3530 | Reproduction | SsSNP2006B_2263 |
| 3531 | Reproduction | SsSNP2006B_2329 |
| 3532 | Reproduction | SsSNP2006B_2360 |
| 3533 | Reproduction | SsSNP2006B_2209 |
| 3534 | Reproduction | SsSNP2006B_1593 |
| 3535 | Reproduction | SsSNP2006B_2530 |
| 3536 | Reproduction | SsSNP2006B_122 |
| 3537 | Reproduction | SsSNP2006B_884 |
| 3538 | Reproduction | SsSNP2006B_156 |
| 3539 | Reproduction | SsSNP2006B_1074 |
| 3540 | Reproduction | SsSNP2006B_767 |
| 3541 | Reproduction | SsSNP2006B_2502 |
| 3542 | Reproduction | SsSNP2006B_1015 |
| 3543 | Reproduction | SsSNP2006B_1375 |
| 3544 | Reproduction | SsSNP2006B_276 |
| 3545 | Reproduction | SsSNP2006B_910 |
| 3546 | Reproduction | SsSNP2006B_275 |
| 3547 | Reproduction | SsSNP2006B_1344 |
| 3548 | Reproduction | SsSNP2006B_1262 |
| 3549 | Reproduction | SsSNP2006B_1467 |
| 3550 | Reproduction | SsSNP2006B_2516 |
| 3551 | Reproduction | SsSNP2006B_964 |
| 3552 | Reproduction | SsSNP2006B_2024 |
| 3553 | Reproduction | SsSNP2006B_913 |
| 3554 | Reproduction | SsSNP2006B_655 |
| 3555 | Reproduction | SsSNP2006B_903 |
| 3556 | Reproduction | SsSNP2006B_1838 |
| 3557 | Reproduction | SsSNP2006B_1649 |
| 3558 | Reproduction | SsSNP2006B_912 |
| 3559 | Reproduction | SsSNP2006B_1376 |
| 3560 | Reproduction | SsSNP2006B_1240 |
| 3561 | Reproduction | SsSNP2006B_670 |
| 3562 | Reproduction | SsSNP2006B_886 |
| 3563 | Reproduction | SsSNP2006B_1520 |
| 3564 | Reproduction | SsSNP2006B_2119 |
| 3565 | Reproduction | SsSNP2006B_227 |
| 3566 | Reproduction | SsSNP2006B_2548 |
| 3567 | Reproduction | SsSNP2006B_342 |
| 3568 | Reproduction | SsSNP2006B_1236 |
| 3569 | Reproduction | SsSNP2006B_1760 |
| 3570 | Reproduction | SsSNP2006B_1287 |
| 3571 | Reproduction | SsSNP2006B_1876 |
| 3572 | Reproduction | SsSNP2006B_2059 |
| 3573 | Reproduction | SsSNP2006B_994 |
| 3574 | Reproduction | SsSNP2006B_2442 |
| 3575 | Reproduction | SsSNP2006B_752 |
| 3576 | Reproduction | SsSNP2006B_1099 |
| 3577 | Reproduction | SsSNP2006B_1415 |
| 3578 | Reproduction | SsSNP2006B_2518 |
| 3579 | Reproduction | SsSNP2006B_1845 |
| 3580 | Reproduction | SsSNP2006B_1243 |
| 3581 | Reproduction | SsSNP2006B_1295 |
| 3582 | Reproduction | SsSNP2006B_898 |
| 3583 | Reproduction | SsSNP2006B_503 |
| 3584 | Reproduction | SsSNP2006B_2386 |
| 3585 | Reproduction | SsSNP2006B_1613 |
| 3586 | Reproduction | SsSNP2006B_1874 |
| 3587 | Reproduction | SsSNP2006B_2046 |
| 3588 | Reproduction | SsSNP2006B_637 |
| 3589 | Reproduction | SsSNP2006B_714 |
| 3590 | Reproduction | SsSNP2006B_168 |
| 3591 | Reproduction | SsSNP2006B_1097 |
| 3592 | Reproduction | SsSNP2006B_167 |
| 3593 | Reproduction | SsSNP2006B_1096 |
| 3594 | Reproduction | SsSNP2006B_2333 |
| 3595 | Reproduction | SsSNP2006B_1166 |
| 3596 | Reproduction | SsSNP2006B_807 |
| 3597 | Reproduction | SsSNP2006B_1088 |
| 3598 | Reproduction | SsSNP2006B_2289 |
| 3599 | Reproduction | SsSNP2006B_1811 |
| 3600 | Reproduction | SsSNP2006B_1998 |
| 3601 | Reproduction | SsSNP2006B_1999 |
| 3602 | Reproduction | SsSNP2006B_2282 |
| 3603 | Reproduction | SsSNP2006B_1326 |
| 3604 | Reproduction | SsSNP2006B_1680 |
| 3605 | Reproduction | SsSNP2006B_384 |
| 3606 | Reproduction | SsSNP2006B_1524 |
| 3607 | Reproduction | SsSNP2006B_419 |
| 3608 | Reproduction | SsSNP2006B_19 |
| 3609 | Reproduction | SsSNP2006B_61 |
| 3610 | Reproduction | SsSNP2006B_285 |
| 3611 | Reproduction | SsSNP2006B_1382 |
| 3612 | Reproduction | SsSNP2006B_1604 |
| 3613 | Reproduction | SsSNP2006B_400 |
| 3614 | Reproduction | SsSNP2006B_401 |
| 3615 | Reproduction | SsSNP2006B_1982 |
| 3616 | Reproduction | SsSNP2006B_1057 |
| 3617 | Reproduction | SsSNP2006B_769 |
| 3618 | Reproduction | SsSNP2006B_2313 |
| 3619 | Reproduction | SsSNP2006B_925 |
| 3620 | Reproduction | SsSNP2006B_1212 |
| 3621 | Reproduction | SsSNP2006B_1778 |
| 3622 | Reproduction | SsSNP2006B_1543 |
| 3623 | Reproduction | SsSNP2006B_2284 |
| 3624 | Reproduction | SsSNP2006B_290 |
| 3625 | Reproduction | SsSNP2006B_59 |
| 3626 | Reproduction | SsSNP2006B_1412 |
| 3627 | Reproduction | SsSNP2006B_1341 |
| 3628 | Reproduction | SsSNP2006B_1621 |
| 3629 | Reproduction | SsSNP2006B_1438 |
| 3630 | Reproduction | SsSNP2006B_1264 |
| 3631 | Reproduction | SsSNP2006B_1509 |
| 3632 | Reproduction | SsSNP2006B_2314 |
| 3633 | Reproduction | SsSNP2006B_20 |
| 3634 | Reproduction | SsSNP2006B_455 |
| 3635 | Reproduction | SsSNP2006B_1137 |
| 3636 | Reproduction | SsSNP2006B_2371 |
| 3637 | Reproduction | SsSNP2006B_1204 |
| 3638 | Reproduction | SsSNP2006B_1737 |
| 3639 | Reproduction | SsSNP2006B_1827 |
| 3640 | Reproduction | SsSNP2006B_1828 |
| 3641 | Reproduction | SsSNP2006B_1766 |
| 3642 | Reproduction | SsSNP2006B_488 |
| 3643 | Reproduction | SsSNP2006B_2373 |
| 3644 | Reproduction | SsSNP2006B_586 |
| 3645 | Reproduction | SsSNP2006B_1189 |
| 3646 | Reproduction | SsSNP2006B_1201 |
| 3647 | Reproduction | SsSNP2006B_1231 |
| 3648 | Reproduction | SsSNP2006B_2472 |
| 3649 | Reproduction | SsSNP2006B_2473 |
| 3650 | Reproduction | SsSNP2006B_2288 |
| 3651 | Reproduction | SsSNP2006B_702 |
| 3652 | Reproduction | SsSNP2006B_1510 |
| 3653 | Reproduction | SsSNP2006B_396 |
| 3654 | Reproduction | SsSNP2006B_372 |
| 3655 | Reproduction | SsSNP2006B_192 |
| 3656 | Reproduction | SsSNP2006B_624 |
| 3657 | Reproduction | SsSNP2006B_1405 |
| 3658 | Reproduction | SsSNP2006B_1537 |
| 3659 | Reproduction | SsSNP2006B_374 |
| 3660 | Reproduction | SsSNP2006B_544 |
| 3661 | Reproduction | SsSNP2006B_183 |
| 3662 | Reproduction | SsSNP2006B_1724 |
| 3663 | Reproduction | SsSNP2006B_2199 |
| 3664 | Reproduction | SsSNP2006B_416 |
| 3665 | Reproduction | SsSNP2006B_899 |
| 3666 | Reproduction | SsSNP2006B_2273 |
| 3667 | Reproduction | SsSNP2006B_439 |
| 3668 | Reproduction | SsSNP2006B_2532 |
| 3669 | Reproduction | SsSNP2006B_42 |
| 3670 | Reproduction | SsSNP2006B_520 |
| 3671 | Reproduction | SsSNP2006B_638 |
| 3672 | Reproduction | SsSNP2006B_900 |
| 3673 | Reproduction | SsSNP2006B_1115 |
| 3674 | Reproduction | SsSNP2006B_2070 |
| 3675 | Reproduction | SsSNP2006B_1639 |
| 3676 | Reproduction | SsSNP2006B_658 |
| 3677 | Reproduction | SsSNP2006B_989 |
| 3678 | Reproduction | SsSNP2006B_2075 |
| 3679 | Reproduction | SsSNP2006B_1747 |
| 3680 | Reproduction | SsSNP2006B_1799 |
| 3681 | Reproduction | SsSNP2006B_506 |
| 3682 | Reproduction | SsSNP2006B_1081 |
| 3683 | Reproduction | SsSNP2006B_1552 |
| 3684 | Reproduction | SsSNP2006B_1780 |
| 3685 | Reproduction | SsSNP2006B_1810 |
| 3686 | Reproduction | SsSNP2006B_570 |
| 3687 | Reproduction | SsSNP2006B_1936 |
| 3688 | Reproduction | SsSNP2006B_197 |
| 3689 | Reproduction | SsSNP2006B_1598 |
| 3690 | Reproduction | SsSNP2006B_1671 |
| 3691 | Reproduction | SsSNP2006B_1672 |
| 3692 | Reproduction | SsSNP2006B_1787 |
| 3693 | Reproduction | SsSNP2006B_996 |
| 3694 | Reproduction | SsSNP2006B_1248 |
| 3695 | Reproduction | SsSNP2006B_246 |
| 3696 | Reproduction | SsSNP2006B_2276 |
| 3697 | Reproduction | SsSNP2006B_1158 |
| 3698 | Reproduction | SsSNP2006B_1232 |
| 3699 | Reproduction | SsSNP2006B_1722 |
| 3700 | Reproduction | SsSNP2006B_557 |
| 3701 | Reproduction | SsSNP2006B_187 |
| 3702 | Reproduction | SsSNP2006B_2132 |
| 3703 | Reproduction | SsSNP2006B_1884 |
| 3704 | Reproduction | SsSNP2006B_1783 |
| 3705 | Reproduction | SsSNP2006B_308 |
| 3706 | Reproduction | SsSNP2006B_1919 |
| 3707 | Reproduction | SsSNP2006B_1644 |
| 3708 | Reproduction | SsSNP2006B_2374 |
| 3709 | Reproduction | SsSNP2006B_780 |
| 3710 | Reproduction | SsSNP2006B_1803 |
| 3711 | Reproduction | SsSNP2006B_2481 |
| 3712 | Reproduction | SsSNP2006B_2461 |
| 3713 | Reproduction | SsSNP2006B_2485 |
| 3714 | Reproduction | SsSNP2006B_1245 |
| 3715 | Reproduction | SsSNP2006B_2250 |
| 3716 | Reproduction | SsSNP2006B_2133 |
| 3717 | Reproduction | SsSNP2006B_220 |
| 3718 | Reproduction | SsSNP2006B_409 |

TABLE 2

| SEQ ID NO | SNP ID NO. | SNP POSITION | SNP ALLELE 1 | SNP ALLELE 2 |
|---|---|---|---|---|
| SEQ ID NO: 1 | SsSNP2006B_1 | 121 | A | G |
| SEQ ID NO: 2 | SsSNP2006B_2 | 99 | A | C |
| SEQ ID NO: 3 | SsSNP2006B_3 | 121 | A | C |
| SEQ ID NO: 4 | SsSNP2006B_4 | 121 | A | G |
| SEQ ID NO: 5 | SsSNP2006B_5 | 121 | A | G |
| SEQ ID NO: 6 | SsSNP2006B_6 | 121 | A | G |
| SEQ ID NO: 7 | SsSNP2006B_7 | 121 | A | G |
| SEQ ID NO: 8 | SsSNP2006B_8 | 121 | A | T |
| SEQ ID NO: 9 | SsSNP2006B_9 | 121 | A | G |
| SEQ ID NO: 10 | SsSNP2006B_10 | 121 | A | G |
| SEQ ID NO: 11 | SsSNP2006B_11 | 121 | A | G |
| SEQ ID NO: 12 | SsSNP2006B_12 | 121 | A | G |
| SEQ ID NO: 13 | SsSNP2006B_13 | 121 | A | G |
| SEQ ID NO: 14 | SsSNP2006B_14 | 121 | A | G |
| SEQ ID NO: 15 | SsSNP2006B_15 | 121 | A | T |
| SEQ ID NO: 16 | SsSNP2006B_16 | 121 | A | C |
| SEQ ID NO: 17 | SsSNP2006B_17 | 121 | A | G |
| SEQ ID NO: 18 | SsSNP2006B_18 | 121 | A | C |
| SEQ ID NO: 19 | SsSNP2006B_19 | 121 | A | G |
| SEQ ID NO: 20 | SsSNP2006B_20 | 121 | A | G |
| SEQ ID NO: 21 | SsSNP2006B_21 | 121 | A | G |
| SEQ ID NO: 22 | SsSNP2006B_22 | 121 | A | T |
| SEQ ID NO: 23 | SsSNP2006B_23 | 121 | A | G |
| SEQ ID NO: 24 | SsSNP2006B_24 | 121 | A | G |
| SEQ ID NO: 25 | SsSNP2006B_25 | 121 | A | T |
| SEQ ID NO: 26 | SsSNP2006B_26 | 121 | A | G |
| SEQ ID NO: 27 | SsSNP2006B_27 | 121 | A | G |
| SEQ ID NO: 28 | SsSNP2006B_28 | 121 | A | G |
| SEQ ID NO: 29 | SsSNP2006B_29 | 121 | A | G |
| SEQ ID NO: 30 | SsSNP2006B_30 | 121 | A | G |
| SEQ ID NO: 31 | SsSNP2006B_31 | 121 | A | T |
| SEQ ID NO: 32 | SsSNP2006B_32 | 121 | A | G |
| SEQ ID NO: 33 | SsSNP2006B_33 | 121 | A | G |
| SEQ ID NO: 34 | SsSNP2006B_34 | 121 | A | G |
| SEQ ID NO: 35 | SsSNP2006B_35 | 121 | A | C |
| SEQ ID NO: 36 | SsSNP2006B_36 | 121 | A | T |
| SEQ ID NO: 37 | SsSNP2006B_37 | 121 | A | G |
| SEQ ID NO: 38 | SsSNP2006B_38 | 121 | A | G |
| SEQ ID NO: 39 | SsSNP2006B_39 | 121 | A | T |
| SEQ ID NO: 40 | SsSNP2006B_40 | 121 | A | G |
| SEQ ID NO: 41 | SsSNP2006B_41 | 121 | A | G |
| SEQ ID NO: 42 | SsSNP2006B_42 | 121 | A | G |
| SEQ ID NO: 43 | SsSNP2006B_43 | 121 | A | T |
| SEQ ID NO: 44 | SsSNP2006B_44 | 121 | A | C |
| SEQ ID NO: 45 | SsSNP2006B_45 | 121 | A | G |
| SEQ ID NO: 46 | SsSNP2006B_46 | 121 | A | G |
| SEQ ID NO: 47 | SsSNP2006B_47 | 121 | A | C |
| SEQ ID NO: 48 | SsSNP2006B_48 | 121 | A | G |
| SEQ ID NO: 49 | SsSNP2006B_49 | 121 | A | G |
| SEQ ID NO: 50 | SsSNP2006B_50 | 121 | A | G |
| SEQ ID NO: 51 | SsSNP2006B_51 | 121 | A | G |
| SEQ ID NO: 52 | SsSNP2006B_52 | 121 | A | G |
| SEQ ID NO: 53 | SsSNP2006B_53 | 121 | A | G |
| SEQ ID NO: 54 | SsSNP2006B_54 | 121 | A | C |
| SEQ ID NO: 55 | SsSNP2006B_55 | 121 | A | G |
| SEQ ID NO: 56 | SsSNP2006B_56 | 121 | A | G |
| SEQ ID NO: 57 | SsSNP2006B_57 | 121 | A | C |
| SEQ ID NO: 58 | SsSNP2006B_58 | 121 | A | T |
| SEQ ID NO: 59 | SsSNP2006B_59 | 121 | A | G |
| SEQ ID NO: 60 | SsSNP2006B_60 | 121 | A | G |
| SEQ ID NO: 61 | SsSNP2006B_61 | 121 | A | G |
| SEQ ID NO: 62 | SsSNP2006B_62 | 121 | A | C |
| SEQ ID NO: 63 | SsSNP2006B_63 | 121 | A | G |
| SEQ ID NO: 64 | SsSNP2006B_64 | 121 | A | G |
| SEQ ID NO: 65 | SsSNP2006B_65 | 121 | A | G |
| SEQ ID NO: 66 | SsSNP2006B_66 | 121 | A | G |
| SEQ ID NO: 67 | SsSNP2006B_67 | 121 | A | G |
| SEQ ID NO: 68 | SsSNP2006B_68 | 121 | A | G |
| SEQ ID NO: 69 | SsSNP2006B_69 | 121 | A | G |
| SEQ ID NO: 70 | SsSNP2006B_70 | 121 | A | G |
| SEQ ID NO: 71 | SsSNP2006B_71 | 121 | A | C |
| SEQ ID NO: 72 | SsSNP2006B_72 | 121 | A | G |
| SEQ ID NO: 73 | SsSNP2006B_73 | 121 | A | G |
| SEQ ID NO: 74 | SsSNP2006B_74 | 121 | A | G |
| SEQ ID NO: 75 | SsSNP2006B_75 | 121 | C | G |
| SEQ ID NO: 76 | SsSNP2006B_76 | 121 | A | G |
| SEQ ID NO: 77 | SsSNP2006B_77 | 121 | A | G |
| SEQ ID NO: 78 | SsSNP2006B_78 | 121 | A | G |
| SEQ ID NO: 79 | SsSNP2006B_79 | 121 | A | C |
| SEQ ID NO: 80 | SsSNP2006B_80 | 121 | A | G |
| SEQ ID NO: 81 | SsSNP2006B_81 | 121 | A | G |
| SEQ ID NO: 82 | SsSNP2006B_82 | 121 | A | G |
| SEQ ID NO: 83 | SsSNP2006B_83 | 121 | A | G |
| SEQ ID NO: 84 | SsSNP2006B_84 | 121 | C | G |
| SEQ ID NO: 85 | SsSNP2006B_85 | 121 | A | G |
| SEQ ID NO: 86 | SsSNP2006B_86 | 121 | A | G |
| SEQ ID NO: 87 | SsSNP2006B_87 | 121 | A | C |
| SEQ ID NO: 88 | SsSNP2006B_88 | 121 | A | G |
| SEQ ID NO: 89 | SsSNP2006B_89 | 121 | A | G |
| SEQ ID NO: 90 | SsSNP2006B_90 | 121 | A | G |
| SEQ ID NO: 91 | SsSNP2006B_91 | 121 | A | G |
| SEQ ID NO: 92 | SsSNP2006B_92 | 121 | C | G |
| SEQ ID NO: 93 | SsSNP2006B_93 | 121 | A | G |
| SEQ ID NO: 94 | SsSNP2006B_94 | 106 | A | G |
| SEQ ID NO: 95 | SsSNP2006B_95 | 121 | C | G |
| SEQ ID NO: 96 | SsSNP2006B_96 | 121 | A | G |
| SEQ ID NO: 97 | SsSNP2006B_97 | 121 | A | G |
| SEQ ID NO: 98 | SsSNP2006B_98 | 121 | A | G |
| SEQ ID NO: 99 | SsSNP2006B_99 | 121 | A | G |
| SEQ ID NO: 100 | SsSNP2006B_100 | 121 | A | G |
| SEQ ID NO: 101 | SsSNP2006B_101 | 121 | A | G |
| SEQ ID NO: 102 | SsSNP2006B_102 | 121 | A | G |
| SEQ ID NO: 103 | SsSNP2006B_103 | 121 | A | G |
| SEQ ID NO: 104 | SsSNP2006B_104 | 121 | A | G |
| SEQ ID NO: 105 | SsSNP2006B_105 | 121 | A | G |
| SEQ ID NO: 106 | SsSNP2006B_106 | 121 | A | G |
| SEQ ID NO: 107 | SsSNP2006B_107 | 121 | A | G |
| SEQ ID NO: 108 | SsSNP2006B_108 | 121 | A | G |
| SEQ ID NO: 109 | SsSNP2006B_109 | 121 | A | G |
| SEQ ID NO: 110 | SsSNP2006B_110 | 121 | A | G |
| SEQ ID NO: 111 | SsSNP2006B_111 | 42 | A | C |
| SEQ ID NO: 112 | SsSNP2006B_112 | 121 | A | G |
| SEQ ID NO: 113 | SsSNP2006B_113 | 121 | A | G |
| SEQ ID NO: 114 | SsSNP2006B_114 | 121 | A | G |
| SEQ ID NO: 115 | SsSNP2006B_115 | 121 | A | G |
| SEQ ID NO: 116 | SsSNP2006B_116 | 121 | A | G |
| SEQ ID NO: 117 | SsSNP2006B_117 | 121 | A | G |
| SEQ ID NO: 118 | SsSNP2006B_118 | 121 | A | G |
| SEQ ID NO: 119 | SsSNP2006B_119 | 121 | A | T |
| SEQ ID NO: 120 | SsSNP2006B_120 | 121 | A | G |
| SEQ ID NO: 121 | SsSNP2006B_121 | 121 | A | G |
| SEQ ID NO: 122 | SsSNP2006B_122 | 121 | A | G |
| SEQ ID NO: 123 | SsSNP2006B_123 | 121 | A | G |
| SEQ ID NO: 124 | SsSNP2006B_124 | 121 | A | C |
| SEQ ID NO: 125 | SsSNP2006B_125 | 121 | A | G |
| SEQ ID NO: 126 | SsSNP2006B_126 | 121 | A | T |
| SEQ ID NO: 127 | SsSNP2006B_127 | 121 | A | G |
| SEQ ID NO: 128 | SsSNP2006B_128 | 121 | A | G |
| SEQ ID NO: 129 | SsSNP2006B_129 | 121 | A | C |
| SEQ ID NO: 130 | SsSNP2006B_130 | 121 | A | G |
| SEQ ID NO: 131 | SsSNP2006B_131 | 121 | A | G |
| SEQ ID NO: 132 | SsSNP2006B_132 | 121 | A | G |
| SEQ ID NO: 133 | SsSNP2006B_133 | 121 | A | T |
| SEQ ID NO: 134 | SsSNP2006B_134 | 121 | C | G |
| SEQ ID NO: 135 | SsSNP2006B_135 | 121 | A | G |
| SEQ ID NO: 136 | SsSNP2006B_136 | 121 | A | G |
| SEQ ID NO: 137 | SsSNP2006B_137 | 121 | A | G |
| SEQ ID NO: 138 | SsSNP2006B_138 | 121 | A | G |
| SEQ ID NO: 139 | SsSNP2006B_139 | 121 | A | G |
| SEQ ID NO: 140 | SsSNP2006B_140 | 121 | C | G |
| SEQ ID NO: 141 | SsSNP2006B_141 | 121 | A | C |
| SEQ ID NO: 142 | SsSNP2006B_142 | 121 | A | G |
| SEQ ID NO: 143 | SsSNP2006B_143 | 121 | A | T |
| SEQ ID NO: 144 | SsSNP2006B_144 | 121 | A | G |
| SEQ ID NO: 145 | SsSNP2006B_145 | 121 | C | G |
| SEQ ID NO: 146 | SsSNP2006B_146 | 121 | A | G |
| SEQ ID NO: 147 | SsSNP2006B_147 | 121 | A | G |
| SEQ ID NO: 148 | SsSNP2006B_148 | 121 | A | G |
| SEQ ID NO: 149 | SsSNP2006B_149 | 121 | A | G |
| SEQ ID NO: 150 | SsSNP2006B_150 | 121 | A | G |
| SEQ ID NO: 151 | SsSNP2006B_151 | 121 | A | G |
| SEQ ID NO: 152 | SsSNP2006B_152 | 121 | A | G |
| SEQ ID NO: 153 | SsSNP2006B_153 | 121 | A | G |
| SEQ ID NO: 154 | SsSNP2006B_154 | 121 | A | T |
| SEQ ID NO: 155 | SsSNP2006B_155 | 121 | A | G |
| SEQ ID NO: 156 | SsSNP2006B_156 | 121 | A | C |
| SEQ ID NO: 157 | SsSNP2006B_157 | 121 | A | C |
| SEQ ID NO: 158 | SsSNP2006B_158 | 121 | A | G |
| SEQ ID NO: 159 | SsSNP2006B_159 | 121 | A | G |
| SEQ ID NO: 160 | SsSNP2006B_160 | 121 | A | G |
| SEQ ID NO: 161 | SsSNP2006B_161 | 121 | A | G |
| SEQ ID NO: 162 | SsSNP2006B_162 | 121 | A | G |
| SEQ ID NO: 163 | SsSNP2006B_163 | 121 | A | G |
| SEQ ID NO: 164 | SsSNP2006B_164 | 121 | A | C |
| SEQ ID NO: 165 | SsSNP2006B_165 | 121 | A | T |
| SEQ ID NO: 166 | SsSNP2006B_166 | 121 | A | C |
| SEQ ID NO: 167 | SsSNP2006B_167 | 121 | A | G |
| SEQ ID NO: 168 | SsSNP2006B_168 | 121 | A | C |
| SEQ ID NO: 169 | SsSNP2006B_169 | 121 | A | G |
| SEQ ID NO: 170 | SsSNP2006B_170 | 121 | A | G |
| SEQ ID NO: 171 | SsSNP2006B_171 | 121 | A | G |
| SEQ ID NO: 172 | SsSNP2006B_172 | 121 | A | G |
| SEQ ID NO: 173 | SsSNP2006B_173 | 121 | A | G |
| SEQ ID NO: 174 | SsSNP2006B_174 | 121 | A | G |
| SEQ ID NO: 175 | SsSNP2006B_175 | 121 | A | G |
| SEQ ID NO: 176 | SsSNP2006B_176 | 121 | A | G |
| SEQ ID NO: 177 | SsSNP2006B_177 | 121 | C | G |
| SEQ ID NO: 178 | SsSNP2006B_178 | 121 | A | G |
| SEQ ID NO: 179 | SsSNP2006B_179 | 121 | A | C |
| SEQ ID NO: 180 | SsSNP2006B_180 | 121 | A | G |
| SEQ ID NO: 181 | SsSNP2006B_181 | 121 | A | C |
| SEQ ID NO: 182 | SsSNP2006B_182 | 121 | A | G |
| SEQ ID NO: 183 | SsSNP2006B_183 | 121 | A | C |
| SEQ ID NO: 184 | SsSNP2006B_184 | 121 | A | G |
| SEQ ID NO: 185 | SsSNP2006B_185 | 121 | A | G |
| SEQ ID NO: 186 | SsSNP2006B_186 | 121 | A | G |
| SEQ ID NO: 187 | SsSNP2006B_187 | 121 | A | G |
| SEQ ID NO: 188 | SsSNP2006B_188 | 121 | A | G |
| SEQ ID NO: 189 | SsSNP2006B_189 | 121 | A | G |
| SEQ ID NO: 190 | SsSNP2006B_190 | 121 | A | G |
| SEQ ID NO: 191 | SsSNP2006B_191 | 121 | A | G |
| SEQ ID NO: 192 | SsSNP2006B_192 | 121 | A | C |
| SEQ ID NO: 193 | SsSNP2006B_193 | 121 | A | G |
| SEQ ID NO: 194 | SsSNP2006B_194 | 121 | A | G |
| SEQ ID NO: 195 | SsSNP2006B_195 | 121 | A | G |
| SEQ ID NO: 196 | SsSNP2006B_196 | 121 | A | G |
| SEQ ID NO: 197 | SsSNP2006B_197 | 121 | A | G |
| SEQ ID NO: 198 | SsSNP2006B_198 | 121 | A | G |
| SEQ ID NO: 199 | SsSNP2006B_199 | 121 | A | T |
| SEQ ID NO: 200 | SsSNP2006B_200 | 121 | A | G |
| SEQ ID NO: 201 | SsSNP2006B_201 | 121 | A | G |
| SEQ ID NO: 202 | SsSNP2006B_202 | 121 | A | T |
| SEQ ID NO: 203 | SsSNP2006B_203 | 121 | A | T |
| SEQ ID NO: 204 | SsSNP2006B_204 | 102 | A | G |
| SEQ ID NO: 205 | SsSNP2006B_205 | 121 | A | G |
| SEQ ID NO: 206 | SsSNP2006B_206 | 121 | A | G |
| SEQ ID NO: 207 | SsSNP2006B_207 | 121 | A | G |
| SEQ ID NO: 208 | SsSNP2006B_208 | 121 | A | G |
| SEQ ID NO: 209 | SsSNP2006B_209 | 121 | A | C |
| SEQ ID NO: 210 | SsSNP2006B_210 | 121 | A | G |
| SEQ ID NO: 211 | SsSNP2006B_211 | 121 | A | G |
| SEQ ID NO: 212 | SsSNP2006B_212 | 121 | A | G |
| SEQ ID NO: 213 | SsSNP2006B_213 | 121 | A | G |
| SEQ ID NO: 214 | SsSNP2006B_214 | 121 | A | G |
| SEQ ID NO: 215 | SsSNP2006B_215 | 121 | A | G |
| SEQ ID NO: 216 | SsSNP2006B_216 | 121 | A | G |
| SEQ ID NO: 217 | SsSNP2006B_217 | 121 | A | C |
| SEQ ID NO: 218 | SsSNP2006B_218 | 121 | C | G |
| SEQ ID NO: 219 | SsSNP2006B_219 | 121 | A | G |
| SEQ ID NO: 220 | SsSNP2006B_220 | 121 | A | G |
| SEQ ID NO: 221 | SsSNP2006B_221 | 121 | A | G |
| SEQ ID NO: 222 | SsSNP2006B_222 | 121 | A | C |
| SEQ ID NO: 223 | SsSNP2006B_223 | 121 | A | G |
| SEQ ID NO: 224 | SsSNP2006B_224 | 121 | A | G |
| SEQ ID NO: 225 | SsSNP2006B_225 | 121 | C | G |
| SEQ ID NO: 226 | SsSNP2006B_226 | 121 | A | G |
| SEQ ID NO: 227 | SsSNP2006B_227 | 121 | A | T |
| SEQ ID NO: 228 | SsSNP2006B_228 | 121 | A | G |
| SEQ ID NO: 229 | SsSNP2006B_229 | 121 | A | G |
| SEQ ID NO: 230 | SsSNP2006B_230 | 121 | A | C |
| SEQ ID NO: 231 | SsSNP2006B_231 | 121 | A | G |
| SEQ ID NO: 232 | SsSNP2006B_232 | 121 | A | G |
| SEQ ID NO: 233 | SsSNP2006B_233 | 121 | A | G |
| SEQ ID NO: 234 | SsSNP2006B_234 | 121 | A | T |
| SEQ ID NO: 235 | SsSNP2006B_235 | 121 | A | G |
| SEQ ID NO: 236 | SsSNP2006B_236 | 121 | A | G |
| SEQ ID NO: 237 | SsSNP2006B_237 | 121 | A | G |
| SEQ ID NO: 238 | SsSNP2006B_238 | 121 | A | G |
| SEQ ID NO: 239 | SsSNP2006B_239 | 102 | A | G |
| SEQ ID NO: 240 | SsSNP2006B_240 | 121 | C | G |
| SEQ ID NO: 241 | SsSNP2006B_241 | 121 | A | G |
| SEQ ID NO: 242 | SsSNP2006B_242 | 121 | A | G |
| SEQ ID NO: 243 | SsSNP2006B_243 | 121 | A | G |
| SEQ ID NO: 244 | SsSNP2006B_244 | 121 | A | G |
| SEQ ID NO: 245 | SsSNP2006B_245 | 121 | A | G |
| SEQ ID NO: 246 | SsSNP2006B_246 | 121 | A | G |
| SEQ ID NO: 247 | SsSNP2006B_247 | 121 | A | G |
| SEQ ID NO: 248 | SsSNP2006B_248 | 121 | A | G |
| SEQ ID NO: 249 | SsSNP2006B_249 | 121 | A | G |
| SEQ ID NO: 250 | SsSNP2006B_250 | 121 | A | G |
| SEQ ID NO: 251 | SsSNP2006B_251 | 121 | C | G |
| SEQ ID NO: 252 | SsSNP2006B_252 | 121 | A | G |
| SEQ ID NO: 253 | SsSNP2006B_253 | 121 | A | C |
| SEQ ID NO: 254 | SsSNP2006B_254 | 121 | A | C |
| SEQ ID NO: 255 | SsSNP2006B_255 | 121 | A | G |
| SEQ ID NO: 256 | SsSNP2006B_256 | 121 | A | G |
| SEQ ID NO: 257 | SsSNP2006B_257 | 121 | A | G |
| SEQ ID NO: 258 | SsSNP2006B_258 | 121 | C | G |
| SEQ ID NO: 259 | SsSNP2006B_259 | 121 | A | C |
| SEQ ID NO: 260 | SsSNP2006B_260 | 121 | A | G |
| SEQ ID NO: 261 | SsSNP2006B_261 | 121 | A | G |
| SEQ ID NO: 262 | SsSNP2006B_262 | 121 | A | G |
| SEQ ID NO: 263 | SsSNP2006B_263 | 121 | A | T |
| SEQ ID NO: 264 | SsSNP2006B_264 | 121 | A | G |
| SEQ ID NO: 265 | SsSNP2006B_265 | 121 | A | G |
| SEQ ID NO: 266 | SsSNP2006B_266 | 121 | A | G |
| SEQ ID NO: 267 | SsSNP2006B_267 | 121 | A | G |
| SEQ ID NO: 268 | SsSNP2006B_268 | 121 | A | G |
| SEQ ID NO: 269 | SsSNP2006B_269 | 121 | A | G |
| SEQ ID NO: 270 | SsSNP2006B_270 | 121 | A | C |
| SEQ ID NO: 271 | SsSNP2006B_271 | 121 | A | G |
| SEQ ID NO: 272 | SsSNP2006B_272 | 121 | A | G |
| SEQ ID NO: 273 | SsSNP2006B_273 | 121 | A | G |
| SEQ ID NO: 274 | SsSNP2006B_274 | 121 | A | G |
| SEQ ID NO: 275 | SsSNP2006B_275 | 121 | A | G |
| SEQ ID NO: 276 | SsSNP2006B_276 | 121 | A | G |
| SEQ ID NO: 277 | SsSNP2006B_277 | 121 | A | G |
| SEQ ID NO: 278 | SsSNP2006B_278 | 121 | A | G |
| SEQ ID NO: 279 | SsSNP2006B_279 | 121 | A | T |
| SEQ ID NO: 280 | SsSNP2006B_280 | 121 | A | G |
| SEQ ID NO: 281 | SsSNP2006B_281 | 121 | A | C |
| SEQ ID NO: 282 | SsSNP2006B_282 | 121 | A | G |
| SEQ ID NO: 283 | SsSNP2006B_283 | 121 | A | G |
| SEQ ID NO: 284 | SsSNP2006B_284 | 121 | A | C |
| SEQ ID NO: 285 | SsSNP2006B_285 | 121 | A | G |
| SEQ ID NO: 286 | SsSNP2006B_286 | 121 | A | G |
| SEQ ID NO: 287 | SsSNP2006B_287 | 121 | A | T |
| SEQ ID NO: 288 | SsSNP2006B_288 | 121 | A | G |
| SEQ ID NO: 289 | SsSNP2006B_289 | 121 | A | G |
| SEQ ID NO: 290 | SsSNP2006B_290 | 121 | A | G |
| SEQ ID NO: 291 | SsSNP2006B_291 | 121 | A | G |
| SEQ ID NO: 292 | SsSNP2006B_292 | 121 | A | G |
| SEQ ID NO: 293 | SsSNP2006B_293 | 121 | C | G |
| SEQ ID NO: 294 | SsSNP2006B_294 | 121 | A | T |
| SEQ ID NO: 295 | SsSNP2006B_295 | 121 | A | G |
| SEQ ID NO: 296 | SsSNP2006B_296 | 121 | A | C |
| SEQ ID NO: 297 | SsSNP2006B_297 | 121 | A | G |
| SEQ ID NO: 298 | SsSNP2006B_298 | 121 | A | G |
| SEQ ID NO: 299 | SsSNP2006B_299 | 121 | A | G |
| SEQ ID NO: 300 | SsSNP2006B_300 | 121 | A | G |
| SEQ ID NO: 301 | SsSNP2006B_301 | 121 | A | T |
| SEQ ID NO: 302 | SsSNP2006B_302 | 121 | A | G |
| SEQ ID NO: 303 | SsSNP2006B_303 | 121 | A | G |
| SEQ ID NO: 304 | SsSNP2006B_304 | 121 | A | G |
| SEQ ID NO: 305 | SsSNP2006B_305 | 121 | A | C |
| SEQ ID NO: 306 | SsSNP2006B_306 | 121 | A | T |
| SEQ ID NO: 307 | SsSNP2006B_307 | 121 | A | G |
| SEQ ID NO: 308 | SsSNP2006B_308 | 121 | A | G |
| SEQ ID NO: 309 | SsSNP2006B_309 | 121 | A | G |
| SEQ ID NO: 310 | SsSNP2006B_310 | 121 | A | G |
| SEQ ID NO: 311 | SsSNP2006B_311 | 121 | A | G |
| SEQ ID NO: 312 | SsSNP2006B_312 | 121 | A | C |
| SEQ ID NO: 313 | SsSNP2006B_313 | 121 | A | G |
| SEQ ID NO: 314 | SsSNP2006B_314 | 121 | A | G |
| SEQ ID NO: 315 | SsSNP2006B_315 | 121 | A | C |
| SEQ ID NO: 316 | SsSNP2006B_316 | 121 | C | G |
| SEQ ID NO: 317 | SsSNP2006B_317 | 121 | A | G |
| SEQ ID NO: 318 | SsSNP2006B_318 | 121 | A | C |
| SEQ ID NO: 319 | SsSNP2006B_319 | 121 | A | C |
| SEQ ID NO: 320 | SsSNP2006B_320 | 121 | A | G |
| SEQ ID NO: 321 | SsSNP2006B_321 | 121 | A | G |
| SEQ ID NO: 322 | SsSNP2006B_322 | 121 | A | C |
| SEQ ID NO: 323 | SsSNP2006B_323 | 121 | A | G |
| SEQ ID NO: 324 | SsSNP2006B_324 | 121 | A | G |
| SEQ ID NO: 325 | SsSNP2006B_325 | 121 | A | G |
| SEQ ID NO: 326 | SsSNP2006B_326 | 121 | A | G |
| SEQ ID NO: 327 | SsSNP2006B_327 | 121 | A | G |
| SEQ ID NO: 328 | SsSNP2006B_328 | 121 | A | C |
| SEQ ID NO: 329 | SsSNP2006B_329 | 121 | C | G |
| SEQ ID NO: 330 | SsSNP2006B_330 | 121 | A | G |
| SEQ ID NO: 331 | SsSNP2006B_331 | 121 | A | G |
| SEQ ID NO: 332 | SsSNP2006B_332 | 121 | A | G |
| SEQ ID NO: 333 | SsSNP2006B_333 | 121 | A | G |
| SEQ ID NO: 334 | SsSNP2006B_334 | 121 | A | G |
| SEQ ID NO: 335 | SsSNP2006B_335 | 121 | C | G |
| SEQ ID NO: 336 | SsSNP2006B_336 | 121 | A | G |
| SEQ ID NO: 337 | SsSNP2006B_337 | 121 | A | G |
| SEQ ID NO: 338 | SsSNP2006B_338 | 121 | A | C |
| SEQ ID NO: 339 | SsSNP2006B_339 | 121 | A | G |
| SEQ ID NO: 340 | SsSNP2006B_340 | 121 | A | C |
| SEQ ID NO: 341 | SsSNP2006B_341 | 121 | A | G |
| SEQ ID NO: 342 | SsSNP2006B_342 | 121 | A | G |
| SEQ ID NO: 343 | SsSNP2006B_343 | 121 | A | G |
| SEQ ID NO: 344 | SsSNP2006B_344 | 121 | A | G |
| SEQ ID NO: 345 | SsSNP2006B_345 | 121 | A | G |
| SEQ ID NO: 346 | SsSNP2006B_346 | 121 | A | G |
| SEQ ID NO: 347 | SsSNP2006B_347 | 121 | A | G |
| SEQ ID NO: 348 | SsSNP2006B_348 | 121 | A | C |
| SEQ ID NO: 349 | SsSNP2006B_349 | 121 | A | G |
| SEQ ID NO: 350 | SsSNP2006B_350 | 121 | A | G |
| SEQ ID NO: 351 | SsSNP2006B_351 | 121 | A | G |
| SEQ ID NO: 352 | SsSNP2006B_352 | 121 | A | G |
| SEQ ID NO: 353 | SsSNP2006B_353 | 121 | A | G |
| SEQ ID NO: 354 | SsSNP2006B_354 | 121 | A | G |
| SEQ ID NO: 355 | SsSNP2006B_355 | 121 | C | G |
| SEQ ID NO: 356 | SsSNP2006B_356 | 121 | A | G |
| SEQ ID NO: 357 | SsSNP2006B_357 | 121 | A | C |
| SEQ ID NO: 358 | SsSNP2006B_358 | 121 | A | C |
| SEQ ID NO: 359 | SsSNP2006B_359 | 121 | A | G |
| SEQ ID NO: 360 | SsSNP2006B_360 | 121 | A | G |
| SEQ ID NO: 361 | SsSNP2006B_361 | 121 | A | G |
| SEQ ID NO: 362 | SsSNP2006B_362 | 121 | A | G |
| SEQ ID NO: 363 | SsSNP2006B_363 | 121 | C | G |
| SEQ ID NO: 364 | SsSNP2006B_364 | 121 | A | C |
| SEQ ID NO: 365 | SsSNP2006B_365 | 121 | A | G |
| SEQ ID NO: 366 | SsSNP2006B_366 | 121 | A | G |
| SEQ ID NO: 367 | SsSNP2006B_367 | 121 | A | G |
| SEQ ID NO: 368 | SsSNP2006B_368 | 121 | A | G |
| SEQ ID NO: 369 | SsSNP2006B_369 | 121 | A | G |
| SEQ ID NO: 370 | SsSNP2006B_370 | 121 | C | G |
| SEQ ID NO: 371 | SsSNP2006B_371 | 121 | A | G |
| SEQ ID NO: 372 | SsSNP2006B_372 | 121 | A | G |
| SEQ ID NO: 373 | SsSNP2006B_373 | 121 | A | G |
| SEQ ID NO: 374 | SsSNP2006B_374 | 121 | A | G |
| SEQ ID NO: 375 | SsSNP2006B_375 | 121 | A | G |
| SEQ ID NO: 376 | SsSNP2006B_376 | 121 | A | G |
| SEQ ID NO: 377 | SsSNP2006B_377 | 121 | A | G |
| SEQ ID NO: 378 | SsSNP2006B_378 | 121 | A | G |
| SEQ ID NO: 379 | SsSNP2006B_379 | 121 | A | G |
| SEQ ID NO: 380 | SsSNP2006B_380 | 121 | C | G |
| SEQ ID NO: 381 | SsSNP2006B_381 | 121 | A | G |
| SEQ ID NO: 382 | SsSNP2006B_382 | 121 | A | G |
| SEQ ID NO: 383 | SsSNP2006B_383 | 110 | A | C |
| SEQ ID NO: 384 | SsSNP2006B_384 | 121 | A | G |
| SEQ ID NO: 385 | SsSNP2006B_385 | 121 | A | G |
| SEQ ID NO: 386 | SsSNP2006B_386 | 121 | A | C |
| SEQ ID NO: 387 | SsSNP2006B_387 | 121 | A | G |
| SEQ ID NO: 388 | SsSNP2006B_388 | 121 | A | G |
| SEQ ID NO: 389 | SsSNP2006B_389 | 121 | A | G |
| SEQ ID NO: 390 | SsSNP2006B_390 | 121 | A | G |
| SEQ ID NO: 391 | SsSNP2006B_391 | 121 | A | C |
| SEQ ID NO: 392 | SsSNP2006B_392 | 121 | A | G |
| SEQ ID NO: 393 | SsSNP2006B_393 | 121 | A | G |
| SEQ ID NO: 394 | SsSNP2006B_394 | 121 | A | G |
| SEQ ID NO: 395 | SsSNP2006B_395 | 121 | A | G |
| SEQ ID NO: 396 | SsSNP2006B_396 | 121 | A | C |
| SEQ ID NO: 397 | SsSNP2006B_397 | 121 | A | G |
| SEQ ID NO: 398 | SsSNP2006B_398 | 121 | C | G |
| SEQ ID NO: 399 | SsSNP2006B_399 | 121 | A | G |
| SEQ ID NO: 400 | SsSNP2006B_400 | 121 | A | C |
| SEQ ID NO: 401 | SsSNP2006B_401 | 121 | A | G |
| SEQ ID NO: 402 | SsSNP2006B_402 | 121 | A | T |
| SEQ ID NO: 403 | SsSNP2006B_403 | 121 | A | G |
| SEQ ID NO: 404 | SsSNP2006B_404 | 121 | A | G |
| SEQ ID NO: 405 | SsSNP2006B_405 | 121 | A | G |
| SEQ ID NO: 406 | SsSNP2006B_406 | 121 | A | G |
| SEQ ID NO: 407 | SsSNP2006B_407 | 121 | A | G |
| SEQ ID NO: 408 | SsSNP2006B_408 | 121 | A | C |
| SEQ ID NO: 409 | SsSNP2006B_409 | 121 | A | G |
| SEQ ID NO: 410 | SsSNP2006B_410 | 121 | A | G |
| SEQ ID NO: 411 | SsSNP2006B_411 | 121 | A | G |
| SEQ ID NO: 412 | SsSNP2006B_412 | 121 | A | G |
| SEQ ID NO: 413 | SsSNP2006B_413 | 121 | C | G |
| SEQ ID NO: 414 | SsSNP2006B_414 | 121 | A | G |
| SEQ ID NO: 415 | SsSNP2006B_415 | 121 | A | T |
| SEQ ID NO: 416 | SsSNP2006B_416 | 121 | A | C |
| SEQ ID NO: 417 | SsSNP2006B_417 | 121 | A | G |
| SEQ ID NO: 418 | SsSNP2006B_418 | 121 | A | G |
| SEQ ID NO: 419 | SsSNP2006B_419 | 121 | A | G |
| SEQ ID NO: 420 | SsSNP2006B_420 | 121 | A | G |
| SEQ ID NO: 421 | SsSNP2006B_421 | 121 | A | G |
| SEQ ID NO: 422 | SsSNP2006B_422 | 121 | A | G |
| SEQ ID NO: 423 | SsSNP2006B_423 | 121 | C | G |
| SEQ ID NO: 424 | SsSNP2006B_424 | 121 | A | G |
| SEQ ID NO: 425 | SsSNP2006B_425 | 121 | A | C |
| SEQ ID NO: 426 | SsSNP2006B_426 | 121 | A | G |
| SEQ ID NO: 427 | SsSNP2006B_427 | 121 | A | G |
| SEQ ID NO: 428 | SsSNP2006B_428 | 121 | A | G |
| SEQ ID NO: 429 | SsSNP2006B_429 | 121 | C | G |
| SEQ ID NO: 430 | SsSNP2006B_430 | 121 | A | G |
| SEQ ID NO: 431 | SsSNP2006B_431 | 105 | A | G |
| SEQ ID NO: 432 | SsSNP2006B_432 | 121 | A | G |
| SEQ ID NO: 433 | SsSNP2006B_433 | 121 | A | G |
| SEQ ID NO: 434 | SsSNP2006B_434 | 121 | A | G |
| SEQ ID NO: 435 | SsSNP2006B_435 | 121 | C | G |
| SEQ ID NO: 436 | SsSNP2006B_436 | 121 | A | C |
| SEQ ID NO: 437 | SsSNP2006B_437 | 121 | A | C |
| SEQ ID NO: 438 | SsSNP2006B_438 | 121 | A | G |
| SEQ ID NO: 439 | SsSNP2006B_439 | 121 | A | T |
| SEQ ID NO: 440 | SsSNP2006B_440 | 121 | A | G |
| SEQ ID NO: 441 | SsSNP2006B_441 | 121 | A | C |
| SEQ ID NO: 442 | SsSNP2006B_442 | 121 | A | G |
| SEQ ID NO: 443 | SsSNP2006B_443 | 121 | A | G |
| SEQ ID NO: 444 | SsSNP2006B_444 | 121 | A | C |
| SEQ ID NO: 445 | SsSNP2006B_445 | 121 | A | G |
| SEQ ID NO: 446 | SsSNP2006B_446 | 121 | A | G |
| SEQ ID NO: 447 | SsSNP2006B_447 | 121 | A | C |
| SEQ ID NO: 448 | SsSNP2006B_448 | 121 | A | G |
| SEQ ID NO: 449 | SsSNP2006B_449 | 121 | A | C |
| SEQ ID NO: 450 | SsSNP2006B_450 | 121 | A | G |
| SEQ ID NO: 451 | SsSNP2006B_451 | 121 | A | G |
| SEQ ID NO: 452 | SsSNP2006B_452 | 121 | A | G |
| SEQ ID NO: 453 | SsSNP2006B_453 | 121 | A | T |
| SEQ ID NO: 454 | SsSNP2006B_454 | 121 | A | T |
| SEQ ID NO: 455 | SsSNP2006B_455 | 121 | A | G |
| SEQ ID NO: 456 | SsSNP2006B_456 | 121 | C | G |
| SEQ ID NO: 457 | SsSNP2006B_457 | 121 | A | G |
| SEQ ID NO: 458 | SsSNP2006B_458 | 121 | A | C |
| SEQ ID NO: 459 | SsSNP2006B_459 | 121 | A | G |
| SEQ ID NO: 460 | SsSNP2006B_460 | 121 | A | G |
| SEQ ID NO: 461 | SsSNP2006B_461 | 121 | A | G |
| SEQ ID NO: 462 | SsSNP2006B_462 | 121 | A | G |
| SEQ ID NO: 463 | SsSNP2006B_463 | 121 | A | G |
| SEQ ID NO: 464 | SsSNP2006B_464 | 121 | A | C |
| SEQ ID NO: 465 | SsSNP2006B_465 | 121 | A | G |
| SEQ ID NO: 466 | SsSNP2006B_466 | 121 | A | T |
| SEQ ID NO: 467 | SsSNP2006B_467 | 121 | A | T |
| SEQ ID NO: 468 | SsSNP2006B_468 | 121 | A | G |
| SEQ ID NO: 469 | SsSNP2006B_469 | 121 | A | G |
| SEQ ID NO: 470 | SsSNP2006B_470 | 121 | A | G |
| SEQ ID NO: 471 | SsSNP2006B_471 | 121 | A | T |
| SEQ ID NO: 472 | SsSNP2006B_472 | 121 | A | G |
| SEQ ID NO: 473 | SsSNP2006B_473 | 121 | A | G |
| SEQ ID NO: 474 | SsSNP2006B_474 | 121 | A | G |
| SEQ ID NO: 475 | SsSNP2006B_475 | 121 | A | G |
| SEQ ID NO: 476 | SsSNP2006B_476 | 121 | A | T |
| SEQ ID NO: 477 | SsSNP2006B_477 | 121 | A | G |
| SEQ ID NO: 478 | SsSNP2006B_478 | 121 | A | G |
| SEQ ID NO: 479 | SsSNP2006B_479 | 121 | A | C |
| SEQ ID NO: 480 | SsSNP2006B_480 | 121 | A | C |
| SEQ ID NO: 481 | SsSNP2006B_481 | 121 | A | G |
| SEQ ID NO: 482 | SsSNP2006B_482 | 121 | A | G |
| SEQ ID NO: 483 | SsSNP2006B_483 | 121 | A | G |
| SEQ ID NO: 484 | SsSNP2006B_484 | 121 | A | G |
| SEQ ID NO: 485 | SsSNP2006B_485 | 121 | A | G |
| SEQ ID NO: 486 | SsSNP2006B_486 | 121 | A | G |
| SEQ ID NO: 487 | SsSNP2006B_487 | 121 | A | G |
| SEQ ID NO: 488 | SsSNP2006B_488 | 121 | A | C |
| SEQ ID NO: 489 | SsSNP2006B_489 | 121 | A | G |
| SEQ ID NO: 490 | SsSNP2006B_490 | 121 | A | G |
| SEQ ID NO: 491 | SsSNP2006B_491 | 121 | A | G |
| SEQ ID NO: 492 | SsSNP2006B_492 | 121 | A | G |
| SEQ ID NO: 493 | SsSNP2006B_493 | 121 | A | G |
| SEQ ID NO: 494 | SsSNP2006B_494 | 121 | A | G |
| SEQ ID NO: 495 | SsSNP2006B_495 | 121 | A | G |
| SEQ ID NO: 496 | SsSNP2006B_496 | 121 | C | G |
| SEQ ID NO: 497 | SsSNP2006B_497 | 121 | A | C |
| SEQ ID NO: 498 | SsSNP2006B_498 | 121 | A | G |
| SEQ ID NO: 499 | SsSNP2006B_499 | 121 | A | G |
| SEQ ID NO: 500 | SsSNP2006B_500 | 121 | A | G |
| SEQ ID NO: 501 | SsSNP2006B_501 | 121 | A | G |
| SEQ ID NO: 502 | SsSNP2006B_502 | 121 | A | C |
| SEQ ID NO: 503 | SsSNP2006B_503 | 121 | A | T |
| SEQ ID NO: 504 | SsSNP2006B_504 | 121 | A | G |
| SEQ ID NO: 505 | SsSNP2006B_505 | 121 | A | C |
| SEQ ID NO: 506 | SsSNP2006B_506 | 121 | A | G |
| SEQ ID NO: 507 | SsSNP2006B_507 | 121 | A | G |
| SEQ ID NO: 508 | SsSNP2006B_508 | 121 | A | G |
| SEQ ID NO: 509 | SsSNP2006B_509 | 121 | A | C |
| SEQ ID NO: 510 | SsSNP2006B_510 | 121 | C | G |
| SEQ ID NO: 511 | SsSNP2006B_511 | 121 | A | G |
| SEQ ID NO: 512 | SsSNP2006B_512 | 121 | A | G |
| SEQ ID NO: 513 | SsSNP2006B_513 | 121 | A | T |
| SEQ ID NO: 514 | SsSNP2006B_514 | 121 | A | G |
| SEQ ID NO: 515 | SsSNP2006B_515 | 121 | A | C |
| SEQ ID NO: 516 | SsSNP2006B_516 | 121 | C | G |
| SEQ ID NO: 517 | SsSNP2006B_517 | 121 | A | G |
| SEQ ID NO: 518 | SsSNP2006B_518 | 121 | A | C |
| SEQ ID NO: 519 | SsSNP2006B_519 | 121 | A | G |
| SEQ ID NO: 520 | SsSNP2006B_520 | 121 | A | G |
| SEQ ID NO: 521 | SsSNP2006B_521 | 121 | A | G |
| SEQ ID NO: 522 | SsSNP2006B_522 | 121 | A | G |
| SEQ ID NO: 523 | SsSNP2006B_523 | 121 | A | G |
| SEQ ID NO: 524 | SsSNP2006B_524 | 121 | A | G |
| SEQ ID NO: 525 | SsSNP2006B_525 | 121 | A | G |
| SEQ ID NO: 526 | SsSNP2006B_526 | 121 | A | G |
| SEQ ID NO: 527 | SsSNP2006B_527 | 121 | A | G |
| SEQ ID NO: 528 | SsSNP2006B_528 | 121 | A | T |
| SEQ ID NO: 529 | SsSNP2006B_529 | 121 | A | C |
| SEQ ID NO: 530 | SsSNP2006B_530 | 121 | A | G |
| SEQ ID NO: 531 | SsSNP2006B_531 | 121 | A | G |
| SEQ ID NO: 532 | SsSNP2006B_532 | 121 | A | G |
| SEQ ID NO: 533 | SsSNP2006B_533 | 121 | A | G |
| SEQ ID NO: 534 | SsSNP2006B_534 | 121 | A | C |
| SEQ ID NO: 535 | SsSNP2006B_535 | 121 | A | G |
| SEQ ID NO: 536 | SsSNP2006B_536 | 121 | A | G |
| SEQ ID NO: 537 | SsSNP2006B_537 | 121 | C | G |
| SEQ ID NO: 538 | SsSNP2006B_538 | 121 | A | G |
| SEQ ID NO: 539 | SsSNP2006B_539 | 121 | A | G |
| SEQ ID NO: 540 | SsSNP2006B_540 | 121 | A | G |
| SEQ ID NO: 541 | SsSNP2006B_541 | 121 | C | G |
| SEQ ID NO: 542 | SsSNP2006B_542 | 121 | C | G |
| SEQ ID NO: 543 | SsSNP2006B_543 | 121 | A | G |
| SEQ ID NO: 544 | SsSNP2006B_544 | 121 | A | G |
| SEQ ID NO: 545 | SsSNP2006B_545 | 121 | A | G |
| SEQ ID NO: 546 | SsSNP2006B_546 | 121 | A | C |
| SEQ ID NO: 547 | SsSNP2006B_547 | 121 | A | G |
| SEQ ID NO: 548 | SsSNP2006B_548 | 121 | A | G |
| SEQ ID NO: 549 | SsSNP2006B_549 | 121 | A | C |
| SEQ ID NO: 550 | SsSNP2006B_550 | 121 | A | G |
| SEQ ID NO: 551 | SsSNP2006B_551 | 121 | A | G |
| SEQ ID NO: 552 | SsSNP2006B_552 | 121 | A | G |
| SEQ ID NO: 553 | SsSNP2006B_553 | 121 | A | C |
| SEQ ID NO: 554 | SsSNP2006B_554 | 121 | C | G |
| SEQ ID NO: 555 | SsSNP2006B_555 | 121 | A | G |
| SEQ ID NO: 556 | SsSNP2006B_556 | 121 | A | C |
| SEQ ID NO: 557 | SsSNP2006B_557 | 121 | A | T |
| SEQ ID NO: 558 | SsSNP2006B_558 | 121 | A | G |
| SEQ ID NO: 559 | SsSNP2006B_559 | 121 | A | G |
| SEQ ID NO: 560 | SsSNP2006B_560 | 121 | A | G |
| SEQ ID NO: 561 | SsSNP2006B_561 | 121 | A | G |
| SEQ ID NO: 562 | SsSNP2006B_562 | 121 | A | G |
| SEQ ID NO: 563 | SsSNP2006B_563 | 121 | A | G |
| SEQ ID NO: 564 | SsSNP2006B_564 | 121 | A | C |
| SEQ ID NO: 565 | SsSNP2006B_565 | 121 | A | G |
| SEQ ID NO: 566 | SsSNP2006B_566 | 121 | A | T |
| SEQ ID NO: 567 | SsSNP2006B_567 | 121 | A | G |
| SEQ ID NO: 568 | SsSNP2006B_568 | 121 | A | T |
| SEQ ID NO: 569 | SsSNP2006B_569 | 121 | A | C |
| SEQ ID NO: 570 | SsSNP2006B_570 | 121 | A | G |
| SEQ ID NO: 571 | SsSNP2006B_571 | 121 | A | C |
| SEQ ID NO: 572 | SsSNP2006B_572 | 121 | A | G |
| SEQ ID NO: 573 | SsSNP2006B_573 | 121 | A | C |
| SEQ ID NO: 574 | SsSNP2006B_574 | 121 | A | G |
| SEQ ID NO: 575 | SsSNP2006B_575 | 121 | A | C |
| SEQ ID NO: 576 | SsSNP2006B_576 | 121 | A | G |
| SEQ ID NO: 577 | SsSNP2006B_577 | 121 | A | T |
| SEQ ID NO: 578 | SsSNP2006B_578 | 121 | A | G |
| SEQ ID NO: 579 | SsSNP2006B_579 | 121 | A | G |
| SEQ ID NO: 580 | SsSNP2006B_580 | 121 | A | T |
| SEQ ID NO: 581 | SsSNP2006B_581 | 121 | A | G |
| SEQ ID NO: 582 | SsSNP2006B_582 | 121 | A | G |
| SEQ ID NO: 583 | SsSNP2006B_583 | 121 | A | G |
| SEQ ID NO: 584 | SsSNP2006B_584 | 121 | A | G |
| SEQ ID NO: 585 | SsSNP2006B_585 | 121 | A | G |
| SEQ ID NO: 586 | SsSNP2006B_586 | 121 | A | G |
| SEQ ID NO: 587 | SsSNP2006B_587 | 121 | A | G |
| SEQ ID NO: 588 | SsSNP2006B_588 | 121 | A | G |
| SEQ ID NO: 589 | SsSNP2006B_589 | 121 | A | G |
| SEQ ID NO: 590 | SsSNP2006B_590 | 121 | A | C |
| SEQ ID NO: 591 | SsSNP2006B_591 | 121 | A | C |
| SEQ ID NO: 592 | SsSNP2006B_592 | 121 | A | C |
| SEQ ID NO: 593 | SsSNP2006B_593 | 121 | C | G |
| SEQ ID NO: 594 | SsSNP2006B_594 | 121 | A | G |
| SEQ ID NO: 595 | SsSNP2006B_595 | 121 | A | G |
| SEQ ID NO: 596 | SsSNP2006B_596 | 121 | A | G |
| SEQ ID NO: 597 | SsSNP2006B_597 | 121 | A | C |
| SEQ ID NO: 598 | SsSNP2006B_598 | 121 | A | G |
| SEQ ID NO: 599 | SsSNP2006B_599 | 121 | A | G |
| SEQ ID NO: 600 | SsSNP2006B_600 | 121 | A | G |
| SEQ ID NO: 601 | SsSNP2006B_601 | 121 | A | C |
| SEQ ID NO: 602 | SsSNP2006B_602 | 121 | C | G |
| SEQ ID NO: 603 | SsSNP2006B_603 | 121 | A | G |
| SEQ ID NO: 604 | SsSNP2006B_604 | 121 | A | C |
| SEQ ID NO: 605 | SsSNP2006B_605 | 121 | A | C |
| SEQ ID NO: 606 | SsSNP2006B_606 | 121 | A | G |
| SEQ ID NO: 607 | SsSNP2006B_607 | 121 | C | G |
| SEQ ID NO: 608 | SsSNP2006B_608 | 121 | A | G |
| SEQ ID NO: 609 | SsSNP2006B_609 | 121 | A | G |
| SEQ ID NO: 610 | SsSNP2006B_610 | 121 | A | G |
| SEQ ID NO: 611 | SsSNP2006B_611 | 121 | A | G |
| SEQ ID NO: 612 | SsSNP2006B_612 | 121 | A | G |
| SEQ ID NO: 613 | SsSNP2006B_613 | 121 | A | G |
| SEQ ID NO: 614 | SsSNP2006B_614 | 121 | A | G |
| SEQ ID NO: 615 | SsSNP2006B_615 | 121 | A | T |
| SEQ ID NO: 616 | SsSNP2006B_616 | 121 | A | G |
| SEQ ID NO: 617 | SsSNP2006B_617 | 121 | A | T |
| SEQ ID NO: 618 | SsSNP2006B_618 | 121 | A | G |
| SEQ ID NO: 619 | SsSNP2006B_619 | 121 | A | G |
| SEQ ID NO: 620 | SsSNP2006B_620 | 121 | A | G |
| SEQ ID NO: 621 | SsSNP2006B_621 | 121 | A | G |
| SEQ ID NO: 622 | SsSNP2006B_622 | 121 | A | G |
| SEQ ID NO: 623 | SsSNP2006B_623 | 121 | C | G |
| SEQ ID NO: 624 | SsSNP2006B_624 | 121 | C | G |
| SEQ ID NO: 625 | SsSNP2006B_625 | 121 | A | G |
| SEQ ID NO: 626 | SsSNP2006B_626 | 121 | A | G |
| SEQ ID NO: 627 | SsSNP2006B_627 | 121 | A | G |
| SEQ ID NO: 628 | SsSNP2006B_628 | 121 | A | G |
| SEQ ID NO: 629 | SsSNP2006B_629 | 121 | A | G |
| SEQ ID NO: 630 | SsSNP2006B_630 | 121 | C | G |
| SEQ ID NO: 631 | SsSNP2006B_631 | 121 | A | G |
| SEQ ID NO: 632 | SsSNP2006B_632 | 121 | A | G |
| SEQ ID NO: 633 | SsSNP2006B_633 | 121 | A | T |
| SEQ ID NO: 634 | SsSNP2006B_634 | 121 | C | G |
| SEQ ID NO: 635 | SsSNP2006B_635 | 121 | A | G |
| SEQ ID NO: 636 | SsSNP2006B_636 | 121 | A | C |
| SEQ ID NO: 637 | SsSNP2006B_637 | 121 | A | G |
| SEQ ID NO: 638 | SsSNP2006B_638 | 121 | A | G |
| SEQ ID NO: 639 | SsSNP2006B_639 | 121 | C | G |
| SEQ ID NO: 640 | SsSNP2006B_640 | 121 | A | G |
| SEQ ID NO: 641 | SsSNP2006B_641 | 121 | A | G |
| SEQ ID NO: 642 | SsSNP2006B_642 | 121 | A | G |
| SEQ ID NO: 643 | SsSNP2006B_643 | 121 | A | G |
| SEQ ID NO: 644 | SsSNP2006B_644 | 121 | A | G |
| SEQ ID NO: 645 | SsSNP2006B_645 | 121 | A | G |
| SEQ ID NO: 646 | SsSNP2006B_646 | 121 | A | C |
| SEQ ID NO: 647 | SsSNP2006B_647 | 121 | C | G |
| SEQ ID NO: 648 | SsSNP2006B_648 | 121 | A | G |
| SEQ ID NO: 649 | SsSNP2006B_649 | 121 | A | G |
| SEQ ID NO: 650 | SsSNP2006B_650 | 121 | A | G |
| SEQ ID NO: 651 | SsSNP2006B_651 | 121 | A | T |
| SEQ ID NO: 652 | SsSNP2006B_652 | 120 | A | G |
| SEQ ID NO: 653 | SsSNP2006B_653 | 121 | A | C |
| SEQ ID NO: 654 | SsSNP2006B_654 | 121 | A | G |
| SEQ ID NO: 655 | SsSNP2006B_655 | 121 | A | G |
| SEQ ID NO: 656 | SsSNP2006B_656 | 121 | A | G |
| SEQ ID NO: 657 | SsSNP2006B_657 | 121 | A | G |
| SEQ ID NO: 658 | SsSNP2006B_658 | 121 | A | G |
| SEQ ID NO: 659 | SsSNP2006B_659 | 121 | A | G |
| SEQ ID NO: 660 | SsSNP2006B_660 | 121 | A | C |
| SEQ ID NO: 661 | SsSNP2006B_661 | 121 | A | G |
| SEQ ID NO: 662 | SsSNP2006B_662 | 121 | A | G |
| SEQ ID NO: 663 | SsSNP2006B_663 | 121 | A | G |
| SEQ ID NO: 664 | SsSNP2006B_664 | 121 | A | T |
| SEQ ID NO: 665 | SsSNP2006B_665 | 121 | A | G |
| SEQ ID NO: 666 | SsSNP2006B_666 | 121 | A | C |
| SEQ ID NO: 667 | SsSNP2006B_667 | 121 | A | G |
| SEQ ID NO: 668 | SsSNP2006B_668 | 121 | A | G |
| SEQ ID NO: 669 | SsSNP2006B_669 | 121 | A | G |
| SEQ ID NO: 670 | SsSNP2006B_670 | 121 | A | G |
| SEQ ID NO: 671 | SsSNP2006B_671 | 121 | A | G |
| SEQ ID NO: 672 | SsSNP2006B_672 | 121 | A | C |
| SEQ ID NO: 673 | SsSNP2006B_673 | 121 | C | G |
| SEQ ID NO: 674 | SsSNP2006B_674 | 121 | C | G |
| SEQ ID NO: 675 | SsSNP2006B_675 | 121 | A | G |
| SEQ ID NO: 676 | SsSNP2006B_676 | 121 | A | G |
| SEQ ID NO: 677 | SsSNP2006B_677 | 121 | A | T |
| SEQ ID NO: 678 | SsSNP2006B_678 | 121 | C | G |
| SEQ ID NO: 679 | SsSNP2006B_679 | 121 | A | G |
| SEQ ID NO: 680 | SsSNP2006B_680 | 121 | A | G |
| SEQ ID NO: 681 | SsSNP2006B_681 | 121 | A | G |
| SEQ ID NO: 682 | SsSNP2006B_682 | 121 | A | G |
| SEQ ID NO: 683 | SsSNP2006B_683 | 121 | A | G |
| SEQ ID NO: 684 | SsSNP2006B_684 | 121 | C | G |
| SEQ ID NO: 685 | SsSNP2006B_685 | 121 | C | G |
| SEQ ID NO: 686 | SsSNP2006B_686 | 121 | A | C |
| SEQ ID NO: 687 | SsSNP2006B_687 | 121 | A | G |
| SEQ ID NO: 688 | SsSNP2006B_688 | 121 | A | G |
| SEQ ID NO: 689 | SsSNP2006B_689 | 121 | A | G |
| SEQ ID NO: 690 | SsSNP2006B_690 | 121 | A | G |
| SEQ ID NO: 691 | SsSNP2006B_691 | 121 | C | G |
| SEQ ID NO: 692 | SsSNP2006B_692 | 105 | C | G |
| SEQ ID NO: 693 | SsSNP2006B_693 | 121 | A | C |
| SEQ ID NO: 694 | SsSNP2006B_694 | 121 | A | G |
| SEQ ID NO: 695 | SsSNP2006B_695 | 121 | A | T |
| SEQ ID NO: 696 | SsSNP2006B_696 | 121 | A | G |
| SEQ ID NO: 697 | SsSNP2006B_697 | 121 | A | G |
| SEQ ID NO: 698 | SsSNP2006B_698 | 121 | A | T |
| SEQ ID NO: 699 | SsSNP2006B_699 | 121 | A | G |
| SEQ ID NO: 700 | SsSNP2006B_700 | 121 | A | G |
| SEQ ID NO: 701 | SsSNP2006B_701 | 121 | A | G |
| SEQ ID NO: 702 | SsSNP2006B_702 | 121 | A | G |
| SEQ ID NO: 703 | SsSNP2006B_703 | 121 | A | G |
| SEQ ID NO: 704 | SsSNP2006B_704 | 121 | A | G |
| SEQ ID NO: 705 | SsSNP2006B_705 | 121 | A | T |
| SEQ ID NO: 706 | SsSNP2006B_706 | 121 | A | G |
| SEQ ID NO: 707 | SsSNP2006B_707 | 121 | A | G |
| SEQ ID NO: 708 | SsSNP2006B_708 | 121 | A | G |
| SEQ ID NO: 709 | SsSNP2006B_709 | 121 | A | C |
| SEQ ID NO: 710 | SsSNP2006B_710 | 121 | C | G |
| SEQ ID NO: 711 | SsSNP2006B_711 | 121 | A | C |
| SEQ ID NO: 712 | SsSNP2006B_712 | 121 | A | C |
| SEQ ID NO: 713 | SsSNP2006B_713 | 121 | A | G |
| SEQ ID NO: 714 | SsSNP2006B_714 | 121 | A | C |
| SEQ ID NO: 715 | SsSNP2006B_715 | 121 | A | G |
| SEQ ID NO: 716 | SsSNP2006B_716 | 121 | A | G |
| SEQ ID NO: 717 | SsSNP2006B_717 | 121 | A | G |
| SEQ ID NO: 718 | SsSNP2006B_718 | 121 | A | C |
| SEQ ID NO: 719 | SsSNP2006B_719 | 121 | A | G |
| SEQ ID NO: 720 | SsSNP2006B_720 | 121 | A | G |
| SEQ ID NO: 721 | SsSNP2006B_721 | 121 | A | G |
| SEQ ID NO: 722 | SsSNP2006B_722 | 121 | C | G |
| SEQ ID NO: 723 | SsSNP2006B_723 | 121 | A | G |
| SEQ ID NO: 724 | SsSNP2006B_724 | 121 | A | G |
| SEQ ID NO: 725 | SsSNP2006B_725 | 121 | A | C |
| SEQ ID NO: 726 | SsSNP2006B_726 | 121 | A | T |
| SEQ ID NO: 727 | SsSNP2006B_727 | 121 | A | C |
| SEQ ID NO: 728 | SsSNP2006B_728 | 121 | A | G |
| SEQ ID NO: 729 | SsSNP2006B_729 | 121 | A | T |
| SEQ ID NO: 730 | SsSNP2006B_730 | 121 | A | G |
| SEQ ID NO: 731 | SsSNP2006B_731 | 121 | A | G |
| SEQ ID NO: 732 | SsSNP2006B_732 | 121 | A | G |
| SEQ ID NO: 733 | SsSNP2006B_733 | 121 | C | G |
| SEQ ID NO: 734 | SsSNP2006B_734 | 121 | C | G |
| SEQ ID NO: 735 | SsSNP2006B_735 | 121 | A | G |
| SEQ ID NO: 736 | SsSNP2006B_736 | 121 | A | G |
| SEQ ID NO: 737 | SsSNP2006B_737 | 121 | A | G |
| SEQ ID NO: 738 | SsSNP2006B_738 | 121 | A | G |
| SEQ ID NO: 739 | SsSNP2006B_739 | 121 | A | T |
| SEQ ID NO: 740 | SsSNP2006B_740 | 121 | A | C |
| SEQ ID NO: 741 | SsSNP2006B_741 | 121 | A | C |
| SEQ ID NO: 742 | SsSNP2006B_742 | 121 | C | G |
| SEQ ID NO: 743 | SsSNP2006B_743 | 121 | A | G |
| SEQ ID NO: 744 | SsSNP2006B_744 | 121 | A | G |
| SEQ ID NO: 745 | SsSNP2006B_745 | 121 | A | G |
| SEQ ID NO: 746 | SsSNP2006B_746 | 121 | A | G |
| SEQ ID NO: 747 | SsSNP2006B_747 | 121 | A | G |
| SEQ ID NO: 748 | SsSNP2006B_748 | 121 | A | G |
| SEQ ID NO: 749 | SsSNP2006B_749 | 121 | A | C |
| SEQ ID NO: 750 | SsSNP2006B_750 | 121 | A | G |
| SEQ ID NO: 751 | SsSNP2006B_751 | 121 | A | G |
| SEQ ID NO: 752 | SsSNP2006B_752 | 121 | A | G |
| SEQ ID NO: 753 | SsSNP2006B_753 | 121 | A | G |
| SEQ ID NO: 754 | SsSNP2006B_754 | 121 | A | G |
| SEQ ID NO: 755 | SsSNP2006B_755 | 121 | A | G |
| SEQ ID NO: 756 | SsSNP2006B_756 | 121 | A | G |
| SEQ ID NO: 757 | SsSNP2006B_757 | 121 | A | C |
| SEQ ID NO: 758 | SsSNP2006B_758 | 121 | C | G |
| SEQ ID NO: 759 | SsSNP2006B_759 | 121 | A | G |
| SEQ ID NO: 760 | SsSNP2006B_760 | 121 | A | G |
| SEQ ID NO: 761 | SsSNP2006B_761 | 121 | A | G |
| SEQ ID NO: 762 | SsSNP2006B_762 | 95 | A | G |
| SEQ ID NO: 763 | SsSNP2006B_763 | 121 | A | G |
| SEQ ID NO: 764 | SsSNP2006B_764 | 121 | A | G |
| SEQ ID NO: 765 | SsSNP2006B_765 | 121 | A | G |
| SEQ ID NO: 766 | SsSNP2006B_766 | 121 | A | G |
| SEQ ID NO: 767 | SsSNP2006B_767 | 121 | A | G |
| SEQ ID NO: 768 | SsSNP2006B_768 | 121 | A | T |
| SEQ ID NO: 769 | SsSNP2006B_769 | 121 | A | G |
| SEQ ID NO: 770 | SsSNP2006B_770 | 121 | A | G |
| SEQ ID NO: 771 | SsSNP2006B_771 | 121 | A | G |
| SEQ ID NO: 772 | SsSNP2006B_772 | 121 | A | G |
| SEQ ID NO: 773 | SsSNP2006B_773 | 121 | C | G |
| SEQ ID NO: 774 | SsSNP2006B_774 | 121 | A | C |
| SEQ ID NO: 775 | SsSNP2006B_775 | 121 | A | G |
| SEQ ID NO: 776 | SsSNP2006B_776 | 121 | C | G |
| SEQ ID NO: 777 | SsSNP2006B_777 | 121 | A | G |
| SEQ ID NO: 778 | SsSNP2006B_778 | 121 | A | G |
| SEQ ID NO: 779 | SsSNP2006B_779 | 121 | A | C |
| SEQ ID NO: 780 | SsSNP2006B_780 | 121 | A | G |
| SEQ ID NO: 781 | SsSNP2006B_781 | 121 | A | G |
| SEQ ID NO: 782 | SsSNP2006B_782 | 121 | A | G |
| SEQ ID NO: 783 | SsSNP2006B_783 | 121 | A | C |
| SEQ ID NO: 784 | SsSNP2006B_784 | 121 | A | C |
| SEQ ID NO: 785 | SsSNP2006B_785 | 121 | A | G |
| SEQ ID NO: 786 | SsSNP2006B_786 | 121 | A | G |
| SEQ ID NO: 787 | SsSNP2006B_787 | 121 | A | G |
| SEQ ID NO: 788 | SsSNP2006B_788 | 121 | A | G |
| SEQ ID NO: 789 | SsSNP2006B_789 | 121 | A | G |
| SEQ ID NO: 790 | SsSNP2006B_790 | 121 | A | G |
| SEQ ID NO: 791 | SsSNP2006B_791 | 121 | A | G |
| SEQ ID NO: 792 | SsSNP2006B_792 | 121 | A | G |
| SEQ ID NO: 793 | SsSNP2006B_793 | 121 | C | G |
| SEQ ID NO: 794 | SsSNP2006B_794 | 121 | C | G |
| SEQ ID NO: 795 | SsSNP2006B_795 | 121 | C | G |
| SEQ ID NO: 796 | SsSNP2006B_796 | 121 | A | G |
| SEQ ID NO: 797 | SsSNP2006B_797 | 121 | A | G |
| SEQ ID NO: 798 | SsSNP2006B_798 | 121 | A | C |
| SEQ ID NO: 799 | SsSNP2006B_799 | 121 | A | G |
| SEQ ID NO: 800 | SsSNP2006B_800 | 121 | A | G |
| SEQ ID NO: 801 | SsSNP2006B_801 | 121 | A | G |
| SEQ ID NO: 802 | SsSNP2006B_802 | 121 | A | G |
| SEQ ID NO: 803 | SsSNP2006B_803 | 121 | A | G |
| SEQ ID NO: 804 | SsSNP2006B_804 | 121 | A | G |
| SEQ ID NO: 805 | SsSNP2006B_805 | 121 | A | G |
| SEQ ID NO: 806 | SsSNP2006B_806 | 121 | A | G |
| SEQ ID NO: 807 | SsSNP2006B_807 | 121 | A | G |
| SEQ ID NO: 808 | SsSNP2006B_808 | 121 | C | G |
| SEQ ID NO: 809 | SsSNP2006B_809 | 121 | A | G |
| SEQ ID NO: 810 | SsSNP2006B_810 | 121 | A | G |
| SEQ ID NO: 811 | SsSNP2006B_811 | 121 | A | G |
| SEQ ID NO: 812 | SsSNP2006B_812 | 121 | A | G |
| SEQ ID NO: 813 | SsSNP2006B_813 | 121 | A | G |
| SEQ ID NO: 814 | SsSNP2006B_814 | 121 | A | G |
| SEQ ID NO: 815 | SsSNP2006B_815 | 121 | C | G |
| SEQ ID NO: 816 | SsSNP2006B_816 | 121 | A | C |
| SEQ ID NO: 817 | SsSNP2006B_817 | 121 | A | G |
| SEQ ID NO: 818 | SsSNP2006B_818 | 121 | A | G |
| SEQ ID NO: 819 | SsSNP2006B_819 | 121 | A | G |
| SEQ ID NO: 820 | SsSNP2006B_820 | 121 | A | C |
| SEQ ID NO: 821 | SsSNP2006B_821 | 121 | A | T |
| SEQ ID NO: 822 | SsSNP2006B_822 | 121 | A | G |
| SEQ ID NO: 823 | SsSNP2006B_823 | 121 | C | G |
| SEQ ID NO: 824 | SsSNP2006B_824 | 121 | A | G |
| SEQ ID NO: 825 | SsSNP2006B_825 | 121 | A | G |
| SEQ ID NO: 826 | SsSNP2006B_826 | 121 | A | G |
| SEQ ID NO: 827 | SsSNP2006B_827 | 121 | A | G |
| SEQ ID NO: 828 | SsSNP2006B_828 | 121 | A | G |
| SEQ ID NO: 829 | SsSNP2006B_829 | 121 | A | G |
| SEQ ID NO: 830 | SsSNP2006B_830 | 121 | A | G |
| SEQ ID NO: 831 | SsSNP2006B_831 | 121 | A | G |
| SEQ ID NO: 832 | SsSNP2006B_832 | 121 | A | G |
| SEQ ID NO: 833 | SsSNP2006B_833 | 121 | A | T |
| SEQ ID NO: 834 | SsSNP2006B_834 | 121 | A | G |
| SEQ ID NO: 835 | SsSNP2006B_835 | 121 | A | C |
| SEQ ID NO: 836 | SsSNP2006B_836 | 121 | A | G |
| SEQ ID NO: 837 | SsSNP2006B_837 | 121 | A | T |
| SEQ ID NO: 838 | SsSNP2006B_838 | 121 | A | C |
| SEQ ID NO: 839 | SsSNP2006B_839 | 121 | A | G |
| SEQ ID NO: 840 | SsSNP2006B_840 | 121 | A | G |
| SEQ ID NO: 841 | SsSNP2006B_841 | 121 | A | G |
| SEQ ID NO: 842 | SsSNP2006B_842 | 121 | A | G |
| SEQ ID NO: 843 | SsSNP2006B_843 | 121 | A | C |
| SEQ ID NO: 844 | SsSNP2006B_844 | 121 | A | G |
| SEQ ID NO: 845 | SsSNP2006B_845 | 121 | A | G |
| SEQ ID NO: 846 | SsSNP2006B_846 | 121 | A | G |
| SEQ ID NO: 847 | SsSNP2006B_847 | 121 | C | G |
| SEQ ID NO: 848 | SsSNP2006B_848 | 121 | A | G |
| SEQ ID NO: 849 | SsSNP2006B_849 | 121 | C | G |
| SEQ ID NO: 850 | SsSNP2006B_850 | 121 | A | G |
| SEQ ID NO: 851 | SsSNP2006B_851 | 121 | A | C |
| SEQ ID NO: 852 | SsSNP2006B_852 | 121 | A | T |
| SEQ ID NO: 853 | SsSNP2006B_853 | 121 | A | G |
| SEQ ID NO: 854 | SsSNP2006B_854 | 121 | A | G |
| SEQ ID NO: 855 | SsSNP2006B_855 | 121 | A | G |
| SEQ ID NO: 856 | SsSNP2006B_856 | 121 | A | G |
| SEQ ID NO: 857 | SsSNP2006B_857 | 121 | A | G |
| SEQ ID NO: 858 | SsSNP2006B_858 | 121 | A | G |
| SEQ ID NO: 859 | SsSNP2006B_859 | 121 | A | G |
| SEQ ID NO: 860 | SsSNP2006B_860 | 121 | A | G |
| SEQ ID NO: 861 | SsSNP2006B_861 | 121 | A | G |
| SEQ ID NO: 862 | SsSNP2006B_862 | 121 | A | C |
| SEQ ID NO: 863 | SsSNP2006B_863 | 121 | A | G |
| SEQ ID NO: 864 | SsSNP2006B_864 | 121 | A | G |
| SEQ ID NO: 865 | SsSNP2006B_865 | 121 | A | T |
| SEQ ID NO: 866 | SsSNP2006B_866 | 121 | A | G |
| SEQ ID NO: 867 | SsSNP2006B_867 | 121 | A | T |
| SEQ ID NO: 868 | SsSNP2006B_868 | 121 | A | G |
| SEQ ID NO: 869 | SsSNP2006B_869 | 121 | A | G |
| SEQ ID NO: 870 | SsSNP2006B_870 | 121 | A | G |
| SEQ ID NO: 871 | SsSNP2006B_871 | 121 | A | T |
| SEQ ID NO: 872 | SsSNP2006B_872 | 121 | A | C |
| SEQ ID NO: 873 | SsSNP2006B_873 | 121 | C | G |
| SEQ ID NO: 874 | SsSNP2006B_874 | 121 | A | G |
| SEQ ID NO: 875 | SsSNP2006B_875 | 121 | A | G |
| SEQ ID NO: 876 | SsSNP2006B_876 | 121 | A | G |
| SEQ ID NO: 877 | SsSNP2006B_877 | 121 | A | C |
| SEQ ID NO: 878 | SsSNP2006B_878 | 121 | A | G |
| SEQ ID NO: 879 | SsSNP2006B_879 | 121 | A | C |
| SEQ ID NO: 880 | SsSNP2006B_880 | 121 | A | G |
| SEQ ID NO: 881 | SsSNP2006B_881 | 121 | A | G |
| SEQ ID NO: 882 | SsSNP2006B_882 | 121 | A | G |
| SEQ ID NO: 883 | SsSNP2006B_883 | 121 | A | G |
| SEQ ID NO: 884 | SsSNP2006B_884 | 121 | A | G |
| SEQ ID NO: 885 | SsSNP2006B_885 | 112 | A | G |
| SEQ ID NO: 886 | SsSNP2006B_886 | 121 | A | C |
| SEQ ID NO: 887 | SsSNP2006B_887 | 121 | A | G |
| SEQ ID NO: 888 | SsSNP2006B_888 | 121 | A | T |
| SEQ ID NO: 889 | SsSNP2006B_889 | 121 | A | G |
| SEQ ID NO: 890 | SsSNP2006B_890 | 121 | A | G |
| SEQ ID NO: 891 | SsSNP2006B_891 | 121 | A | T |
| SEQ ID NO: 892 | SsSNP2006B_892 | 121 | A | G |
| SEQ ID NO: 893 | SsSNP2006B_893 | 121 | A | G |
| SEQ ID NO: 894 | SsSNP2006B_894 | 121 | A | G |
| SEQ ID NO: 895 | SsSNP2006B_895 | 121 | C | G |
| SEQ ID NO: 896 | SsSNP2006B_896 | 121 | A | G |
| SEQ ID NO: 897 | SsSNP2006B_897 | 121 | A | G |
| SEQ ID NO: 898 | SsSNP2006B_898 | 121 | A | C |
| SEQ ID NO: 899 | SsSNP2006B_899 | 121 | A | G |
| SEQ ID NO: 900 | SsSNP2006B_900 | 121 | A | G |
| SEQ ID NO: 901 | SsSNP2006B_901 | 121 | A | G |
| SEQ ID NO: 902 | SsSNP2006B_902 | 121 | A | G |
| SEQ ID NO: 903 | SsSNP2006B_903 | 121 | A | T |
| SEQ ID NO: 904 | SsSNP2006B_904 | 121 | A | G |
| SEQ ID NO: 905 | SsSNP2006B_905 | 121 | A | G |
| SEQ ID NO: 906 | SsSNP2006B_906 | 121 | A | G |
| SEQ ID NO: 907 | SsSNP2006B_907 | 121 | A | G |
| SEQ ID NO: 908 | SsSNP2006B_908 | 121 | A | G |
| SEQ ID NO: 909 | SsSNP2006B_909 | 121 | A | G |
| SEQ ID NO: 910 | SsSNP2006B_910 | 121 | A | G |
| SEQ ID NO: 911 | SsSNP2006B_911 | 121 | A | G |
| SEQ ID NO: 912 | SsSNP2006B_912 | 121 | A | G |
| SEQ ID NO: 913 | SsSNP2006B_913 | 121 | A | G |
| SEQ ID NO: 914 | SsSNP2006B_914 | 121 | A | T |
| SEQ ID NO: 915 | SsSNP2006B_915 | 121 | A | G |
| SEQ ID NO: 916 | SsSNP2006B_916 | 121 | A | G |
| SEQ ID NO: 917 | SsSNP2006B_917 | 121 | C | G |
| SEQ ID NO: 918 | SsSNP2006B_918 | 121 | A | G |
| SEQ ID NO: 919 | SsSNP2006B_919 | 121 | A | C |
| SEQ ID NO: 920 | SsSNP2006B_920 | 121 | A | G |
| SEQ ID NO: 921 | SsSNP2006B_921 | 121 | A | G |
| SEQ ID NO: 922 | SsSNP2006B_922 | 121 | A | T |
| SEQ ID NO: 923 | SsSNP2006B_923 | 121 | A | G |
| SEQ ID NO: 924 | SsSNP2006B_924 | 121 | A | C |
| SEQ ID NO: 925 | SsSNP2006B_925 | 121 | A | G |
| SEQ ID NO: 926 | SsSNP2006B_926 | 121 | A | G |
| SEQ ID NO: 927 | SsSNP2006B_927 | 121 | C | G |
| SEQ ID NO: 928 | SsSNP2006B_928 | 121 | A | C |
| SEQ ID NO: 929 | SsSNP2006B_929 | 121 | A | C |
| SEQ ID NO: 930 | SsSNP2006B_930 | 121 | A | C |
| SEQ ID NO: 931 | SsSNP2006B_931 | 121 | A | T |
| SEQ ID NO: 932 | SsSNP2006B_932 | 121 | A | G |
| SEQ ID NO: 933 | SsSNP2006B_933 | 121 | A | C |
| SEQ ID NO: 934 | SsSNP2006B_934 | 121 | A | G |
| SEQ ID NO: 935 | SsSNP2006B_935 | 121 | A | G |
| SEQ ID NO: 936 | SsSNP2006B_936 | 121 | A | G |
| SEQ ID NO: 937 | SsSNP2006B_937 | 121 | A | G |
| SEQ ID NO: 938 | SsSNP2006B_938 | 121 | A | G |
| SEQ ID NO: 939 | SsSNP2006B_939 | 121 | A | G |
| SEQ ID NO: 940 | SsSNP2006B_940 | 121 | A | G |
| SEQ ID NO: 941 | SsSNP2006B_941 | 121 | A | G |
| SEQ ID NO: 942 | SsSNP2006B_942 | 121 | A | G |
| SEQ ID NO: 943 | SsSNP2006B_943 | 121 | A | G |
| SEQ ID NO: 944 | SsSNP2006B_944 | 121 | A | G |
| SEQ ID NO: 945 | SsSNP2006B_945 | 121 | A | C |
| SEQ ID NO: 946 | SsSNP2006B_946 | 121 | A | G |
| SEQ ID NO: 947 | SsSNP2006B_947 | 121 | A | G |
| SEQ ID NO: 948 | SsSNP2006B_948 | 121 | A | G |
| SEQ ID NO: 949 | SsSNP2006B_949 | 121 | A | G |
| SEQ ID NO: 950 | SsSNP2006B_950 | 121 | A | G |
| SEQ ID NO: 951 | SsSNP2006B_951 | 121 | A | G |
| SEQ ID NO: 952 | SsSNP2006B_952 | 121 | C | G |
| SEQ ID NO: 953 | SsSNP2006B_953 | 121 | A | G |
| SEQ ID NO: 954 | SsSNP2006B_954 | 121 | A | G |
| SEQ ID NO: 955 | SsSNP2006B_955 | 121 | A | G |
| SEQ ID NO: 956 | SsSNP2006B_956 | 121 | A | C |
| SEQ ID NO: 957 | SsSNP2006B_957 | 121 | C | G |
| SEQ ID NO: 958 | SsSNP2006B_958 | 121 | A | G |
| SEQ ID NO: 959 | SsSNP2006B_959 | 121 | C | G |
| SEQ ID NO: 960 | SsSNP2006B_960 | 121 | A | G |
| SEQ ID NO: 961 | SsSNP2006B_961 | 121 | A | C |
| SEQ ID NO: 962 | SsSNP2006B_962 | 121 | A | G |
| SEQ ID NO: 963 | SsSNP2006B_963 | 121 | A | T |
| SEQ ID NO: 964 | SsSNP2006B_964 | 118 | C | G |
| SEQ ID NO: 965 | SsSNP2006B_965 | 121 | A | C |
| SEQ ID NO: 966 | SsSNP2006B_966 | 121 | A | G |
| SEQ ID NO: 967 | SsSNP2006B_967 | 121 | A | G |
| SEQ ID NO: 968 | SsSNP2006B_968 | 121 | A | C |
| SEQ ID NO: 969 | SsSNP2006B_969 | 121 | A | G |
| SEQ ID NO: 970 | SsSNP2006B_970 | 121 | A | C |
| SEQ ID NO: 971 | SsSNP2006B_971 | 121 | A | G |
| SEQ ID NO: 972 | SsSNP2006B_972 | 121 | A | T |
| SEQ ID NO: 973 | SsSNP2006B_973 | 121 | A | G |
| SEQ ID NO: 974 | SsSNP2006B_974 | 121 | C | G |
| SEQ ID NO: 975 | SsSNP2006B_975 | 121 | A | G |
| SEQ ID NO: 976 | SsSNP2006B_976 | 121 | A | C |
| SEQ ID NO: 977 | SsSNP2006B_977 | 121 | A | G |
| SEQ ID NO: 978 | SsSNP2006B_978 | 121 | A | C |
| SEQ ID NO: 979 | SsSNP2006B_979 | 121 | A | G |
| SEQ ID NO: 980 | SsSNP2006B_980 | 121 | C | G |
| SEQ ID NO: 981 | SsSNP2006B_981 | 121 | C | G |
| SEQ ID NO: 982 | SsSNP2006B_982 | 121 | A | G |
| SEQ ID NO: 983 | SsSNP2006B_983 | 121 | C | G |
| SEQ ID NO: 984 | SsSNP2006B_984 | 121 | A | G |
| SEQ ID NO: 985 | SsSNP2006B_985 | 121 | A | G |
| SEQ ID NO: 986 | SsSNP2006B_986 | 121 | A | G |
| SEQ ID NO: 987 | SsSNP2006B_987 | 121 | A | G |
| SEQ ID NO: 988 | SsSNP2006B_988 | 121 | A | C |
| SEQ ID NO: 989 | SsSNP2006B_989 | 121 | A | G |
| SEQ ID NO: 990 | SsSNP2006B_990 | 121 | A | C |
| SEQ ID NO: 991 | SsSNP2006B_991 | 121 | A | G |
| SEQ ID NO: 992 | SsSNP2006B_992 | 121 | A | G |
| SEQ ID NO: 993 | SsSNP2006B_993 | 121 | A | G |
| SEQ ID NO: 994 | SsSNP2006B_994 | 121 | A | G |
| SEQ ID NO: 995 | SsSNP2006B_995 | 121 | A | G |
| SEQ ID NO: 996 | SsSNP2006B_996 | 121 | A | G |
| SEQ ID NO: 997 | SsSNP2006B_997 | 121 | A | C |
| SEQ ID NO: 998 | SsSNP2006B_998 | 121 | A | G |
| SEQ ID NO: 999 | SsSNP2006B_999 | 121 | A | G |
| SEQ ID NO: 1000 | SsSNP2006B_1000 | 121 | A | G |
| SEQ ID NO: 1001 | SsSNP2006B_1001 | 121 | A | G |
| SEQ ID NO: 1002 | SsSNP2006B_1002 | 121 | A | G |
| SEQ ID NO: 1003 | SsSNP2006B_1003 | 121 | A | G |
| SEQ ID NO: 1004 | SsSNP2006B_1004 | 121 | A | C |
| SEQ ID NO: 1005 | SsSNP2006B_1005 | 121 | A | G |
| SEQ ID NO: 1006 | SsSNP2006B_1006 | 121 | A | G |
| SEQ ID NO: 1007 | SsSNP2006B_1007 | 121 | A | G |
| SEQ ID NO: 1008 | SsSNP2006B_1008 | 121 | A | G |
| SEQ ID NO: 1009 | SsSNP2006B_1009 | 121 | A | C |
| SEQ ID NO: 1010 | SsSNP2006B_1010 | 121 | A | C |
| SEQ ID NO: 1011 | SsSNP2006B_1011 | 121 | A | G |
| SEQ ID NO: 1012 | SsSNP2006B_1012 | 121 | A | G |
| SEQ ID NO: 1013 | SsSNP2006B_1013 | 121 | A | G |
| SEQ ID NO: 1014 | SsSNP2006B_1014 | 121 | A | G |
| SEQ ID NO: 1015 | SsSNP2006B_1015 | 121 | A | G |
| SEQ ID NO: 1016 | SsSNP2006B_1016 | 121 | A | C |
| SEQ ID NO: 1017 | SsSNP2006B_1017 | 121 | A | G |
| SEQ ID NO: 1018 | SsSNP2006B_1018 | 121 | A | G |
| SEQ ID NO: 1019 | SsSNP2006B_1019 | 121 | A | G |
| SEQ ID NO: 1020 | SsSNP2006B_1020 | 121 | A | G |
| SEQ ID NO: 1021 | SsSNP2006B_1021 | 121 | A | T |
| SEQ ID NO: 1022 | SsSNP2006B_1022 | 121 | A | C |
| SEQ ID NO: 1023 | SsSNP2006B_1023 | 121 | A | T |
| SEQ ID NO: 1024 | SsSNP2006B_1024 | 121 | A | G |
| SEQ ID NO: 1025 | SsSNP2006B_1025 | 121 | A | G |
| SEQ ID NO: 1026 | SsSNP2006B_1026 | 121 | A | G |
| SEQ ID NO: 1027 | SsSNP2006B_1027 | 121 | A | G |
| SEQ ID NO: 1028 | SsSNP2006B_1028 | 121 | A | G |
| SEQ ID NO: 1029 | SsSNP2006B_1029 | 121 | A | G |
| SEQ ID NO: 1030 | SsSNP2006B_1030 | 121 | C | G |
| SEQ ID NO: 1031 | SsSNP2006B_1031 | 121 | A | G |
| SEQ ID NO: 1032 | SsSNP2006B_1032 | 121 | A | G |
| SEQ ID NO: 1033 | SsSNP2006B_1033 | 121 | A | G |
| SEQ ID NO: 1034 | SsSNP2006B_1034 | 121 | A | C |
| SEQ ID NO: 1035 | SsSNP2006B_1035 | 121 | A | G |
| SEQ ID NO: 1036 | SsSNP2006B_1036 | 121 | A | G |
| SEQ ID NO: 1037 | SsSNP2006B_1037 | 121 | A | G |
| SEQ ID NO: 1038 | SsSNP2006B_1038 | 121 | A | G |
| SEQ ID NO: 1039 | SsSNP2006B_1039 | 121 | A | C |
| SEQ ID NO: 1040 | SsSNP2006B_1040 | 121 | A | T |
| SEQ ID NO: 1041 | SsSNP2006B_1041 | 121 | C | G |
| SEQ ID NO: 1042 | SsSNP2006B_1042 | 121 | A | G |
| SEQ ID NO: 1043 | SsSNP2006B_1043 | 121 | A | G |
| SEQ ID NO: 1044 | SsSNP2006B_1044 | 121 | A | G |
| SEQ ID NO: 1045 | SsSNP2006B_1045 | 117 | A | C |
| SEQ ID NO: 1046 | SsSNP2006B_1046 | 121 | A | G |
| SEQ ID NO: 1047 | SsSNP2006B_1047 | 121 | A | T |
| SEQ ID NO: 1048 | SsSNP2006B_1048 | 121 | A | G |
| SEQ ID NO: 1049 | SsSNP2006B_1049 | 121 | A | G |
| SEQ ID NO: 1050 | SsSNP2006B_1050 | 121 | A | G |
| SEQ ID NO: 1051 | SsSNP2006B_1051 | 121 | A | G |
| SEQ ID NO: 1052 | SsSNP2006B_1052 | 121 | A | G |
| SEQ ID NO: 1053 | SsSNP2006B_1053 | 121 | A | G |
| SEQ ID NO: 1054 | SsSNP2006B_1054 | 121 | A | G |
| SEQ ID NO: 1055 | SsSNP2006B_1055 | 121 | C | G |
| SEQ ID NO: 1056 | SsSNP2006B_1056 | 121 | A | G |
| SEQ ID NO: 1057 | SsSNP2006B_1057 | 121 | A | T |
| SEQ ID NO: 1058 | SsSNP2006B_1058 | 121 | A | G |
| SEQ ID NO: 1059 | SsSNP2006B_1059 | 121 | A | C |
| SEQ ID NO: 1060 | SsSNP2006B_1060 | 121 | A | C |
| SEQ ID NO: 1061 | SsSNP2006B_1061 | 121 | A | G |
| SEQ ID NO: 1062 | SsSNP2006B_1062 | 121 | A | G |
| SEQ ID NO: 1063 | SsSNP2006B_1063 | 121 | A | G |
| SEQ ID NO: 1064 | SsSNP2006B_1064 | 121 | A | G |
| SEQ ID NO: 1065 | SsSNP2006B_1065 | 121 | A | G |
| SEQ ID NO: 1066 | SsSNP2006B_1066 | 121 | A | G |
| SEQ ID NO: 1067 | SsSNP2006B_1067 | 121 | A | G |
| SEQ ID NO: 1068 | SsSNP2006B_1068 | 121 | C | G |
| SEQ ID NO: 1069 | SsSNP2006B_1069 | 121 | A | G |
| SEQ ID NO: 1070 | SsSNP2006B_1070 | 121 | A | G |
| SEQ ID NO: 1071 | SsSNP2006B_1071 | 121 | A | G |
| SEQ ID NO: 1072 | SsSNP2006B_1072 | 121 | A | G |
| SEQ ID NO: 1073 | SsSNP2006B_1073 | 121 | A | G |
| SEQ ID NO: 1074 | SsSNP2006B_1074 | 121 | A | G |
| SEQ ID NO: 1075 | SsSNP2006B_1075 | 121 | A | G |
| SEQ ID NO: 1076 | SsSNP2006B_1076 | 121 | A | G |
| SEQ ID NO: 1077 | SsSNP2006B_1077 | 121 | A | T |
| SEQ ID NO: 1078 | SsSNP2006B_1078 | 121 | A | G |
| SEQ ID NO: 1079 | SsSNP2006B_1079 | 121 | A | G |
| SEQ ID NO: 1080 | SsSNP2006B_1080 | 121 | A | G |
| SEQ ID NO: 1081 | SsSNP2006B_1081 | 121 | C | G |
| SEQ ID NO: 1082 | SsSNP2006B_1082 | 121 | A | G |
| SEQ ID NO: 1083 | SsSNP2006B_1083 | 121 | A | G |
| SEQ ID NO: 1084 | SsSNP2006B_1084 | 121 | A | G |
| SEQ ID NO: 1085 | SsSNP2006B_1085 | 121 | A | G |
| SEQ ID NO: 1086 | SsSNP2006B_1086 | 121 | A | G |
| SEQ ID NO: 1087 | SsSNP2006B_1087 | 121 | A | G |
| SEQ ID NO: 1088 | SsSNP2006B_1088 | 121 | A | G |
| SEQ ID NO: 1089 | SsSNP2006B_1089 | 121 | A | G |
| SEQ ID NO: 1090 | SsSNP2006B_1090 | 121 | A | G |
| SEQ ID NO: 1091 | SsSNP2006B_1091 | 121 | A | G |
| SEQ ID NO: 1092 | SsSNP2006B_1092 | 121 | A | G |
| SEQ ID NO: 1093 | SsSNP2006B_1093 | 121 | A | G |
| SEQ ID NO: 1094 | SsSNP2006B_1094 | 121 | A | G |
| SEQ ID NO: 1095 | SsSNP2006B_1095 | 121 | A | G |
| SEQ ID NO: 1096 | SsSNP2006B_1096 | 121 | A | G |
| SEQ ID NO: 1097 | SsSNP2006B_1097 | 121 | A | G |
| SEQ ID NO: 1098 | SsSNP2006B_1098 | 121 | A | C |
| SEQ ID NO: 1099 | SsSNP2006B_1099 | 121 | C | G |
| SEQ ID NO: 1100 | SsSNP2006B_1100 | 121 | A | C |
| SEQ ID NO: 1101 | SsSNP2006B_1101 | 121 | A | G |
| SEQ ID NO: 1102 | SsSNP2006B_1102 | 121 | A | G |
| SEQ ID NO: 1103 | SsSNP2006B_1103 | 121 | A | G |
| SEQ ID NO: 1104 | SsSNP2006B_1104 | 121 | A | G |
| SEQ ID NO: 1105 | SsSNP2006B_1105 | 121 | A | G |
| SEQ ID NO: 1106 | SsSNP2006B_1106 | 121 | A | G |
| SEQ ID NO: 1107 | SsSNP2006B_1107 | 121 | A | G |
| SEQ ID NO: 1108 | SsSNP2006B_1108 | 121 | A | G |
| SEQ ID NO: 1109 | SsSNP2006B_1109 | 121 | A | G |
| SEQ ID NO: 1110 | SsSNP2006B_1110 | 121 | A | G |
| SEQ ID NO: 1111 | SsSNP2006B_1111 | 121 | C | G |
| SEQ ID NO: 1112 | SsSNP2006B_1112 | 121 | A | G |
| SEQ ID NO: 1113 | SsSNP2006B_1113 | 121 | A | G |
| SEQ ID NO: 1114 | SsSNP2006B_1114 | 121 | A | C |
| SEQ ID NO: 1115 | SsSNP2006B_1115 | 121 | A | C |
| SEQ ID NO: 1116 | SsSNP2006B_1116 | 121 | A | G |
| SEQ ID NO: 1117 | SsSNP2006B_1117 | 121 | A | T |
| SEQ ID NO: 1118 | SsSNP2006B_1118 | 121 | A | T |
| SEQ ID NO: 1119 | SsSNP2006B_1119 | 121 | A | G |
| SEQ ID NO: 1120 | SsSNP2006B_1120 | 121 | A | G |
| SEQ ID NO: 1121 | SsSNP2006B_1121 | 121 | A | G |
| SEQ ID NO: 1122 | SsSNP2006B_1122 | 121 | A | C |
| SEQ ID NO: 1123 | SsSNP2006B_1123 | 121 | A | T |
| SEQ ID NO: 1124 | SsSNP2006B_1124 | 121 | A | C |
| SEQ ID NO: 1125 | SsSNP2006B_1125 | 121 | A | G |
| SEQ ID NO: 1126 | SsSNP2006B_1126 | 121 | C | G |
| SEQ ID NO: 1127 | SsSNP2006B_1127 | 121 | A | G |
| SEQ ID NO: 1128 | SsSNP2006B_1128 | 121 | A | G |
| SEQ ID NO: 1129 | SsSNP2006B_1129 | 121 | A | C |
| SEQ ID NO: 1130 | SsSNP2006B_1130 | 121 | A | G |
| SEQ ID NO: 1131 | SsSNP2006B_1131 | 121 | A | C |
| SEQ ID NO: 1132 | SsSNP2006B_1132 | 121 | A | G |
| SEQ ID NO: 1133 | SsSNP2006B_1133 | 121 | A | G |
| SEQ ID NO: 1134 | SsSNP2006B_1134 | 121 | A | G |
| SEQ ID NO: 1135 | SsSNP2006B_1135 | 121 | A | G |
| SEQ ID NO: 1136 | SsSNP2006B_1136 | 121 | A | C |
| SEQ ID NO: 1137 | SsSNP2006B_1137 | 121 | A | G |
| SEQ ID NO: 1138 | SsSNP2006B_1138 | 121 | A | G |
| SEQ ID NO: 1139 | SsSNP2006B_1139 | 121 | A | G |
| SEQ ID NO: 1140 | SsSNP2006B_1140 | 121 | A | C |
| SEQ ID NO: 1141 | SsSNP2006B_1141 | 121 | A | G |
| SEQ ID NO: 1142 | SsSNP2006B_1142 | 121 | A | G |
| SEQ ID NO: 1143 | SsSNP2006B_1143 | 121 | A | G |
| SEQ ID NO: 1144 | SsSNP2006B_1144 | 121 | A | G |
| SEQ ID NO: 1145 | SsSNP2006B_1145 | 121 | A | C |
| SEQ ID NO: 1146 | SsSNP2006B_1146 | 121 | A | G |
| SEQ ID NO: 1147 | SsSNP2006B_1147 | 121 | A | G |
| SEQ ID NO: 1148 | SsSNP2006B_1148 | 121 | A | G |
| SEQ ID NO: 1149 | SsSNP2006B_1149 | 121 | A | G |
| SEQ ID NO: 1150 | SsSNP2006B_1150 | 121 | A | G |
| SEQ ID NO: 1151 | SsSNP2006B_1151 | 121 | A | C |
| SEQ ID NO: 1152 | SsSNP2006B_1152 | 121 | A | G |
| SEQ ID NO: 1153 | SsSNP2006B_1153 | 121 | A | G |
| SEQ ID NO: 1154 | SsSNP2006B_1154 | 121 | A | G |
| SEQ ID NO: 1155 | SsSNP2006B_1155 | 121 | A | G |
| SEQ ID NO: 1156 | SsSNP2006B_1156 | 121 | A | G |
| SEQ ID NO: 1157 | SsSNP2006B_1157 | 121 | C | G |
| SEQ ID NO: 1158 | SsSNP2006B_1158 | 121 | A | G |
| SEQ ID NO: 1159 | SsSNP2006B_1159 | 121 | A | G |
| SEQ ID NO: 1160 | SsSNP2006B_1160 | 121 | A | G |
| SEQ ID NO: 1161 | SsSNP2006B_1161 | 121 | A | G |
| SEQ ID NO: 1162 | SsSNP2006B_1162 | 121 | A | G |
| SEQ ID NO: 1163 | SsSNP2006B_1163 | 121 | A | G |
| SEQ ID NO: 1164 | SsSNP2006B_1164 | 121 | A | G |
| SEQ ID NO: 1165 | SsSNP2006B_1165 | 121 | A | C |
| SEQ ID NO: 1166 | SsSNP2006B_1166 | 121 | A | G |
| SEQ ID NO: 1167 | SsSNP2006B_1167 | 121 | C | G |
| SEQ ID NO: 1168 | SsSNP2006B_1168 | 121 | A | G |
| SEQ ID NO: 1169 | SsSNP2006B_1169 | 121 | A | G |
| SEQ ID NO: 1170 | SsSNP2006B_1170 | 121 | A | G |
| SEQ ID NO: 1171 | SsSNP2006B_1171 | 121 | C | G |
| SEQ ID NO: 1172 | SsSNP2006B_1172 | 121 | C | G |
| SEQ ID NO: 1173 | SsSNP2006B_1173 | 121 | A | G |
| SEQ ID NO: 1174 | SsSNP2006B_1174 | 121 | A | G |
| SEQ ID NO: 1175 | SsSNP2006B_1175 | 121 | A | G |
| SEQ ID NO: 1176 | SsSNP2006B_1176 | 121 | A | G |
| SEQ ID NO: 1177 | SsSNP2006B_1177 | 121 | A | C |
| SEQ ID NO: 1178 | SsSNP2006B_1178 | 121 | A | G |
| SEQ ID NO: 1179 | SsSNP2006B_1179 | 121 | A | G |
| SEQ ID NO: 1180 | SsSNP2006B_1180 | 121 | A | G |
| SEQ ID NO: 1181 | SsSNP2006B_1181 | 121 | A | G |
| SEQ ID NO: 1182 | SsSNP2006B_1182 | 121 | A | G |
| SEQ ID NO: 1183 | SsSNP2006B_1183 | 121 | A | G |
| SEQ ID NO: 1184 | SsSNP2006B_1184 | 121 | A | G |
| SEQ ID NO: 1185 | SsSNP2006B_1185 | 121 | A | G |
| SEQ ID NO: 1186 | SsSNP2006B_1186 | 121 | A | G |
| SEQ ID NO: 1187 | SsSNP2006B_1187 | 121 | A | G |
| SEQ ID NO: 1188 | SsSNP2006B_1188 | 121 | A | G |
| SEQ ID NO: 1189 | SsSNP2006B_1189 | 121 | A | G |
| SEQ ID NO: 1190 | SsSNP2006B_1190 | 121 | A | C |
| SEQ ID NO: 1191 | SsSNP2006B_1191 | 121 | A | G |
| SEQ ID NO: 1192 | SsSNP2006B_1192 | 121 | A | G |
| SEQ ID NO: 1193 | SsSNP2006B_1193 | 121 | A | G |
| SEQ ID NO: 1194 | SsSNP2006B_1194 | 121 | A | G |
| SEQ ID NO: 1195 | SsSNP2006B_1195 | 121 | A | G |
| SEQ ID NO: 1196 | SsSNP2006B_1196 | 121 | A | G |
| SEQ ID NO: 1197 | SsSNP2006B_1197 | 121 | A | G |
| SEQ ID NO: 1198 | SsSNP2006B_1198 | 121 | A | G |
| SEQ ID NO: 1199 | SsSNP2006B_1199 | 121 | A | G |
| SEQ ID NO: 1200 | SsSNP2006B_1200 | 121 | A | G |
| SEQ ID NO: 1201 | SsSNP2006B_1201 | 121 | A | G |
| SEQ ID NO: 1202 | SsSNP2006B_1202 | 121 | A | G |
| SEQ ID NO: 1203 | SsSNP2006B_1203 | 121 | A | G |
| SEQ ID NO: 1204 | SsSNP2006B_1204 | 121 | A | G |
| SEQ ID NO: 1205 | SsSNP2006B_1205 | 121 | A | G |
| SEQ ID NO: 1206 | SsSNP2006B_1206 | 121 | A | G |
| SEQ ID NO: 1207 | SsSNP2006B_1207 | 121 | A | G |
| SEQ ID NO: 1208 | SsSNP2006B_1208 | 121 | A | G |
| SEQ ID NO: 1209 | SsSNP2006B_1209 | 121 | A | G |
| SEQ ID NO: 1210 | SsSNP2006B_1210 | 121 | A | T |
| SEQ ID NO: 1211 | SsSNP2006B_1211 | 121 | A | G |
| SEQ ID NO: 1212 | SsSNP2006B_1212 | 121 | A | C |
| SEQ ID NO: 1213 | SsSNP2006B_1213 | 121 | A | T |
| SEQ ID NO: 1214 | SsSNP2006B_1214 | 121 | A | G |
| SEQ ID NO: 1215 | SsSNP2006B_1215 | 121 | A | G |
| SEQ ID NO: 1216 | SsSNP2006B_1216 | 121 | A | C |
| SEQ ID NO: 1217 | SsSNP2006B_1217 | 121 | C | G |
| SEQ ID NO: 1218 | SsSNP2006B_1218 | 121 | C | G |
| SEQ ID NO: 1219 | SsSNP2006B_1219 | 121 | A | G |
| SEQ ID NO: 1220 | SsSNP2006B_1220 | 121 | A | T |
| SEQ ID NO: 1221 | SsSNP2006B_1221 | 121 | A | T |
| SEQ ID NO: 1222 | SsSNP2006B_1222 | 121 | A | G |
| SEQ ID NO: 1223 | SsSNP2006B_1223 | 121 | A | G |
| SEQ ID NO: 1224 | SsSNP2006B_1224 | 121 | A | G |
| SEQ ID NO: 1225 | SsSNP2006B_1225 | 121 | A | G |
| SEQ ID NO: 1226 | SsSNP2006B_1226 | 121 | A | G |
| SEQ ID NO: 1227 | SsSNP2006B_1227 | 121 | A | G |
| SEQ ID NO: 1228 | SsSNP2006B_1228 | 121 | A | G |
| SEQ ID NO: 1229 | SsSNP2006B_1229 | 121 | A | G |
| SEQ ID NO: 1230 | SsSNP2006B_1230 | 121 | A | G |
| SEQ ID NO: 1231 | SsSNP2006B_1231 | 121 | A | G |
| SEQ ID NO: 1232 | SsSNP2006B_1232 | 121 | A | G |
| SEQ ID NO: 1233 | SsSNP2006B_1233 | 121 | A | G |
| SEQ ID NO: 1234 | SsSNP2006B_1234 | 121 | A | G |
| SEQ ID NO: 1235 | SsSNP2006B_1235 | 121 | C | G |
| SEQ ID NO: 1236 | SsSNP2006B_1236 | 121 | A | G |
| SEQ ID NO: 1237 | SsSNP2006B_1237 | 121 | A | C |
| SEQ ID NO: 1238 | SsSNP2006B_1238 | 121 | A | C |
| SEQ ID NO: 1239 | SsSNP2006B_1239 | 121 | A | G |
| SEQ ID NO: 1240 | SsSNP2006B_1240 | 121 | A | G |
| SEQ ID NO: 1241 | SsSNP2006B_1241 | 121 | A | C |
| SEQ ID NO: 1242 | SsSNP2006B_1242 | 121 | A | C |
| SEQ ID NO: 1243 | SsSNP2006B_1243 | 121 | A | G |
| SEQ ID NO: 1244 | SsSNP2006B_1244 | 121 | A | G |
| SEQ ID NO: 1245 | SsSNP2006B_1245 | 121 | A | G |
| SEQ ID NO: 1246 | SsSNP2006B_1246 | 121 | A | G |
| SEQ ID NO: 1247 | SsSNP2006B_1247 | 121 | A | G |
| SEQ ID NO: 1248 | SsSNP2006B_1248 | 121 | A | G |
| SEQ ID NO: 1249 | SsSNP2006B_1249 | 121 | A | G |
| SEQ ID NO: 1250 | SsSNP2006B_1250 | 121 | A | T |
| SEQ ID NO: 1251 | SsSNP2006B_1251 | 121 | A | T |
| SEQ ID NO: 1252 | SsSNP2006B_1252 | 121 | A | C |
| SEQ ID NO: 1253 | SsSNP2006B_1253 | 121 | C | G |
| SEQ ID NO: 1254 | SsSNP2006B_1254 | 121 | A | C |
| SEQ ID NO: 1255 | SsSNP2006B_1255 | 121 | A | G |
| SEQ ID NO: 1256 | SsSNP2006B_1256 | 121 | A | G |
| SEQ ID NO: 1257 | SsSNP2006B_1257 | 121 | A | G |
| SEQ ID NO: 1258 | SsSNP2006B_1258 | 121 | A | C |
| SEQ ID NO: 1259 | SsSNP2006B_1259 | 121 | A | G |
| SEQ ID NO: 1260 | SsSNP2006B_1260 | 121 | A | G |
| SEQ ID NO: 1261 | SsSNP2006B_1261 | 121 | A | C |
| SEQ ID NO: 1262 | SsSNP2006B_1262 | 121 | A | C |
| SEQ ID NO: 1263 | SsSNP2006B_1263 | 121 | A | G |
| SEQ ID NO: 1264 | SsSNP2006B_1264 | 121 | A | C |
| SEQ ID NO: 1265 | SsSNP2006B_1265 | 121 | A | G |
| SEQ ID NO: 1266 | SsSNP2006B_1266 | 121 | A | G |
| SEQ ID NO: 1267 | SsSNP2006B_1267 | 121 | A | G |
| SEQ ID NO: 1268 | SsSNP2006B_1268 | 121 | A | G |
| SEQ ID NO: 1269 | SsSNP2006B_1269 | 121 | A | G |
| SEQ ID NO: 1270 | SsSNP2006B_1270 | 121 | A | G |
| SEQ ID NO: 1271 | SsSNP2006B_1271 | 121 | A | G |
| SEQ ID NO: 1272 | SsSNP2006B_1272 | 121 | A | G |
| SEQ ID NO: 1273 | SsSNP2006B_1273 | 121 | A | G |
| SEQ ID NO: 1274 | SsSNP2006B_1274 | 121 | A | G |
| SEQ ID NO: 1275 | SsSNP2006B_1275 | 121 | A | C |
| SEQ ID NO: 1276 | SsSNP2006B_1276 | 121 | A | G |
| SEQ ID NO: 1277 | SsSNP2006B_1277 | 121 | A | G |
| SEQ ID NO: 1278 | SsSNP2006B_1278 | 121 | A | G |
| SEQ ID NO: 1279 | SsSNP2006B_1279 | 121 | A | G |
| SEQ ID NO: 1280 | SsSNP2006B_1280 | 121 | A | G |
| SEQ ID NO: 1281 | SsSNP2006B_1281 | 121 | A | G |
| SEQ ID NO: 1282 | SsSNP2006B_1282 | 121 | A | G |
| SEQ ID NO: 1283 | SsSNP2006B_1283 | 121 | A | G |
| SEQ ID NO: 1284 | SsSNP2006B_1284 | 121 | C | G |
| SEQ ID NO: 1285 | SsSNP2006B_1285 | 121 | A | G |
| SEQ ID NO: 1286 | SsSNP2006B_1286 | 121 | A | G |
| SEQ ID NO: 1287 | SsSNP2006B_1287 | 121 | A | T |
| SEQ ID NO: 1288 | SsSNP2006B_1288 | 121 | A | G |
| SEQ ID NO: 1289 | SsSNP2006B_1289 | 121 | C | G |
| SEQ ID NO: 1290 | SsSNP2006B_1290 | 121 | A | G |
| SEQ ID NO: 1291 | SsSNP2006B_1291 | 121 | A | G |
| SEQ ID NO: 1292 | SsSNP2006B_1292 | 121 | A | G |
| SEQ ID NO: 1293 | SsSNP2006B_1293 | 121 | A | C |
| SEQ ID NO: 1294 | SsSNP2006B_1294 | 121 | A | C |
| SEQ ID NO: 1295 | SsSNP2006B_1295 | 121 | A | G |
| SEQ ID NO: 1296 | SsSNP2006B_1296 | 121 | C | G |
| SEQ ID NO: 1297 | SsSNP2006B_1297 | 121 | A | T |
| SEQ ID NO: 1298 | SsSNP2006B_1298 | 121 | A | G |
| SEQ ID NO: 1299 | SsSNP2006B_1299 | 121 | A | T |
| SEQ ID NO: 1300 | SsSNP2006B_1300 | 121 | C | G |
| SEQ ID NO: 1301 | SsSNP2006B_1301 | 121 | A | C |
| SEQ ID NO: 1302 | SsSNP2006B_1302 | 121 | A | G |
| SEQ ID NO: 1303 | SsSNP2006B_1303 | 121 | A | C |
| SEQ ID NO: 1304 | SsSNP2006B_1304 | 121 | A | T |
| SEQ ID NO: 1305 | SsSNP2006B_1305 | 121 | A | G |
| SEQ ID NO: 1306 | SsSNP2006B_1306 | 121 | A | C |
| SEQ ID NO: 1307 | SsSNP2006B_1307 | 121 | A | G |
| SEQ ID NO: 1308 | SsSNP2006B_1308 | 121 | A | G |
| SEQ ID NO: 1309 | SsSNP2006B_1309 | 121 | C | G |
| SEQ ID NO: 1310 | SsSNP2006B_1310 | 121 | A | G |
| SEQ ID NO: 1311 | SsSNP2006B_1311 | 121 | A | G |
| SEQ ID NO: 1312 | SsSNP2006B_1312 | 121 | A | G |
| SEQ ID NO: 1313 | SsSNP2006B_1313 | 121 | A | G |
| SEQ ID NO: 1314 | SsSNP2006B_1314 | 121 | A | C |
| SEQ ID NO: 1315 | SsSNP2006B_1315 | 121 | A | C |
| SEQ ID NO: 1316 | SsSNP2006B_1316 | 121 | A | G |
| SEQ ID NO: 1317 | SsSNP2006B_1317 | 121 | A | C |
| SEQ ID NO: 1318 | SsSNP2006B_1318 | 121 | A | C |
| SEQ ID NO: 1319 | SsSNP2006B_1319 | 121 | A | C |
| SEQ ID NO: 1320 | SsSNP2006B_1320 | 121 | A | G |
| SEQ ID NO: 1321 | SsSNP2006B_1321 | 121 | A | C |
| SEQ ID NO: 1322 | SsSNP2006B_1322 | 121 | A | G |
| SEQ ID NO: 1323 | SsSNP2006B_1323 | 121 | A | G |
| SEQ ID NO: 1324 | SsSNP2006B_1324 | 121 | A | G |
| SEQ ID NO: 1325 | SsSNP2006B_1325 | 121 | A | G |
| SEQ ID NO: 1326 | SsSNP2006B_1326 | 121 | A | G |
| SEQ ID NO: 1327 | SsSNP2006B_1327 | 121 | C | G |
| SEQ ID NO: 1328 | SsSNP2006B_1328 | 121 | A | G |
| SEQ ID NO: 1329 | SsSNP2006B_1329 | 121 | A | G |
| SEQ ID NO: 1330 | SsSNP2006B_1330 | 121 | A | G |
| SEQ ID NO: 1331 | SsSNP2006B_1331 | 121 | C | G |
| SEQ ID NO: 1332 | SsSNP2006B_1332 | 121 | A | G |
| SEQ ID NO: 1333 | SsSNP2006B_1333 | 121 | A | T |
| SEQ ID NO: 1334 | SsSNP2006B_1334 | 121 | A | T |
| SEQ ID NO: 1335 | SsSNP2006B_1335 | 121 | A | C |
| SEQ ID NO: 1336 | SsSNP2006B_1336 | 121 | A | G |
| SEQ ID NO: 1337 | SsSNP2006B_1337 | 121 | A | G |
| SEQ ID NO: 1338 | SsSNP2006B_1338 | 121 | A | C |
| SEQ ID NO: 1339 | SsSNP2006B_1339 | 121 | A | G |
| SEQ ID NO: 1340 | SsSNP2006B_1340 | 121 | A | G |
| SEQ ID NO: 1341 | SsSNP2006B_1341 | 121 | A | G |
| SEQ ID NO: 1342 | SsSNP2006B_1342 | 121 | A | G |
| SEQ ID NO: 1343 | SsSNP2006B_1343 | 121 | A | C |
| SEQ ID NO: 1344 | SsSNP2006B_1344 | 121 | A | C |
| SEQ ID NO: 1345 | SsSNP2006B_1345 | 121 | A | G |
| SEQ ID NO: 1346 | SsSNP2006B_1346 | 121 | A | T |
| SEQ ID NO: 1347 | SsSNP2006B_1347 | 121 | A | G |
| SEQ ID NO: 1348 | SsSNP2006B_1348 | 121 | A | C |
| SEQ ID NO: 1349 | SsSNP2006B_1349 | 121 | C | G |
| SEQ ID NO: 1350 | SsSNP2006B_1350 | 121 | A | G |
| SEQ ID NO: 1351 | SsSNP2006B_1351 | 121 | A | G |
| SEQ ID NO: 1352 | SsSNP2006B_1352 | 121 | A | G |
| SEQ ID NO: 1353 | SsSNP2006B_1353 | 121 | C | G |
| SEQ ID NO: 1354 | SsSNP2006B_1354 | 121 | A | G |
| SEQ ID NO: 1355 | SsSNP2006B_1355 | 121 | A | T |
| SEQ ID NO: 1356 | SsSNP2006B_1356 | 121 | A | G |
| SEQ ID NO: 1357 | SsSNP2006B_1357 | 121 | A | G |
| SEQ ID NO: 1358 | SsSNP2006B_1358 | 121 | A | C |
| SEQ ID NO: 1359 | SsSNP2006B_1359 | 121 | A | G |
| SEQ ID NO: 1360 | SsSNP2006B_1360 | 121 | A | T |
| SEQ ID NO: 1361 | SsSNP2006B_1361 | 121 | A | G |
| SEQ ID NO: 1362 | SsSNP2006B_1362 | 121 | A | G |
| SEQ ID NO: 1363 | SsSNP2006B_1363 | 121 | A | G |
| SEQ ID NO: 1364 | SsSNP2006B_1364 | 121 | A | G |
| SEQ ID NO: 1365 | SsSNP2006B_1365 | 121 | A | G |
| SEQ ID NO: 1366 | SsSNP2006B_1366 | 121 | A | G |
| SEQ ID NO: 1367 | SsSNP2006B_1367 | 121 | C | G |
| SEQ ID NO: 1368 | SsSNP2006B_1368 | 121 | A | T |
| SEQ ID NO: 1369 | SsSNP2006B_1369 | 121 | A | C |
| SEQ ID NO: 1370 | SsSNP2006B_1370 | 121 | A | G |
| SEQ ID NO: 1371 | SsSNP2006B_1371 | 121 | A | C |
| SEQ ID NO: 1372 | SsSNP2006B_1372 | 121 | A | G |
| SEQ ID NO: 1373 | SsSNP2006B_1373 | 121 | A | G |
| SEQ ID NO: 1374 | SsSNP2006B_1374 | 121 | A | G |
| SEQ ID NO: 1375 | SsSNP2006B_1375 | 121 | A | G |
| SEQ ID NO: 1376 | SsSNP2006B_1376 | 121 | A | G |
| SEQ ID NO: 1377 | SsSNP2006B_1377 | 121 | A | G |
| SEQ ID NO: 1378 | SsSNP2006B_1378 | 121 | A | T |
| SEQ ID NO: 1379 | SsSNP2006B_1379 | 121 | A | G |
| SEQ ID NO: 1380 | SsSNP2006B_1380 | 121 | A | G |
| SEQ ID NO: 1381 | SsSNP2006B_1381 | 121 | A | G |
| SEQ ID NO: 1382 | SsSNP2006B_1382 | 121 | A | G |
| SEQ ID NO: 1383 | SsSNP2006B_1383 | 121 | A | G |
| SEQ ID NO: 1384 | SsSNP2006B_1384 | 121 | A | G |
| SEQ ID NO: 1385 | SsSNP2006B_1385 | 121 | C | G |
| SEQ ID NO: 1386 | SsSNP2006B_1386 | 121 | A | G |
| SEQ ID NO: 1387 | SsSNP2006B_1387 | 121 | A | T |
| SEQ ID NO: 1388 | SsSNP2006B_1388 | 121 | A | G |
| SEQ ID NO: 1389 | SsSNP2006B_1389 | 121 | A | G |
| SEQ ID NO: 1390 | SsSNP2006B_1390 | 121 | A | G |
| SEQ ID NO: 1391 | SsSNP2006B_1391 | 121 | A | G |
| SEQ ID NO: 1392 | SsSNP2006B_1392 | 121 | A | G |
| SEQ ID NO: 1393 | SsSNP2006B_1393 | 121 | A | G |
| SEQ ID NO: 1394 | SsSNP2006B_1394 | 121 | A | G |
| SEQ ID NO: 1395 | SsSNP2006B_1395 | 121 | C | G |
| SEQ ID NO: 1396 | SsSNP2006B_1396 | 121 | A | G |
| SEQ ID NO: 1397 | SsSNP2006B_1397 | 121 | A | G |
| SEQ ID NO: 1398 | SsSNP2006B_1398 | 121 | A | G |
| SEQ ID NO: 1399 | SsSNP2006B_1399 | 121 | A | G |
| SEQ ID NO: 1400 | SsSNP2006B_1400 | 121 | A | G |
| SEQ ID NO: 1401 | SsSNP2006B_1401 | 121 | A | G |
| SEQ ID NO: 1402 | SsSNP2006B_1402 | 121 | A | G |
| SEQ ID NO: 1403 | SsSNP2006B_1403 | 121 | A | G |
| SEQ ID NO: 1404 | SsSNP2006B_1404 | 121 | A | G |
| SEQ ID NO: 1405 | SsSNP2006B_1405 | 121 | A | G |
| SEQ ID NO: 1406 | SsSNP2006B_1406 | 121 | A | G |
| SEQ ID NO: 1407 | SsSNP2006B_1407 | 121 | A | C |
| SEQ ID NO: 1408 | SsSNP2006B_1408 | 121 | A | G |
| SEQ ID NO: 1409 | SsSNP2006B_1409 | 121 | C | G |
| SEQ ID NO: 1410 | SsSNP2006B_1410 | 121 | A | C |
| SEQ ID NO: 1411 | SsSNP2006B_1411 | 121 | A | G |
| SEQ ID NO: 1412 | SsSNP2006B_1412 | 121 | A | G |
| SEQ ID NO: 1413 | SsSNP2006B_1413 | 121 | A | C |
| SEQ ID NO: 1414 | SsSNP2006B_1414 | 121 | A | C |
| SEQ ID NO: 1415 | SsSNP2006B_1415 | 121 | A | G |
| SEQ ID NO: 1416 | SsSNP2006B_1416 | 121 | A | G |
| SEQ ID NO: 1417 | SsSNP2006B_1417 | 121 | C | G |
| SEQ ID NO: 1418 | SsSNP2006B_1418 | 121 | A | G |
| SEQ ID NO: 1419 | SsSNP2006B_1419 | 121 | A | G |
| SEQ ID NO: 1420 | SsSNP2006B_1420 | 121 | A | G |
| SEQ ID NO: 1421 | SsSNP2006B_1421 | 121 | A | G |
| SEQ ID NO: 1422 | SsSNP2006B_1422 | 121 | A | G |
| SEQ ID NO: 1423 | SsSNP2006B_1423 | 121 | A | G |
| SEQ ID NO: 1424 | SsSNP2006B_1424 | 121 | A | G |
| SEQ ID NO: 1425 | SsSNP2006B_1425 | 121 | A | G |
| SEQ ID NO: 1426 | SsSNP2006B_1426 | 121 | A | G |
| SEQ ID NO: 1427 | SsSNP2006B_1427 | 79 | C | G |
| SEQ ID NO: 1428 | SsSNP2006B_1428 | 121 | A | C |
| SEQ ID NO: 1429 | SsSNP2006B_1429 | 121 | A | G |
| SEQ ID NO: 1430 | SsSNP2006B_1430 | 121 | A | G |
| SEQ ID NO: 1431 | SsSNP2006B_1431 | 121 | A | G |
| SEQ ID NO: 1432 | SsSNP2006B_1432 | 121 | A | G |
| SEQ ID NO: 1433 | SsSNP2006B_1433 | 121 | A | G |
| SEQ ID NO: 1434 | SsSNP2006B_1434 | 121 | A | T |
| SEQ ID NO: 1435 | SsSNP2006B_1435 | 121 | A | G |
| SEQ ID NO: 1436 | SsSNP2006B_1436 | 121 | A | G |
| SEQ ID NO: 1437 | SsSNP2006B_1437 | 121 | A | G |
| SEQ ID NO: 1438 | SsSNP2006B_1438 | 121 | A | G |
| SEQ ID NO: 1439 | SsSNP2006B_1439 | 121 | A | C |
| SEQ ID NO: 1440 | SsSNP2006B_1440 | 121 | A | G |
| SEQ ID NO: 1441 | SsSNP2006B_1441 | 121 | A | G |
| SEQ ID NO: 1442 | SsSNP2006B_1442 | 121 | C | G |
| SEQ ID NO: 1443 | SsSNP2006B_1443 | 121 | A | G |
| SEQ ID NO: 1444 | SsSNP2006B_1444 | 121 | A | G |
| SEQ ID NO: 1445 | SsSNP2006B_1445 | 121 | A | G |
| SEQ ID NO: 1446 | SsSNP2006B_1446 | 121 | A | G |
| SEQ ID NO: 1447 | SsSNP2006B_1447 | 121 | A | G |
| SEQ ID NO: 1448 | SsSNP2006B_1448 | 121 | A | G |
| SEQ ID NO: 1449 | SsSNP2006B_1449 | 121 | A | G |
| SEQ ID NO: 1450 | SsSNP2006B_1450 | 121 | A | G |
| SEQ ID NO: 1451 | SsSNP2006B_1451 | 121 | A | C |
| SEQ ID NO: 1452 | SsSNP2006B_1452 | 121 | A | G |
| SEQ ID NO: 1453 | SsSNP2006B_1453 | 121 | A | G |
| SEQ ID NO: 1454 | SsSNP2006B_1454 | 121 | C | G |
| SEQ ID NO: 1455 | SsSNP2006B_1455 | 102 | A | C |
| SEQ ID NO: 1456 | SsSNP2006B_1456 | 121 | A | C |
| SEQ ID NO: 1457 | SsSNP2006B_1457 | 121 | A | G |
| SEQ ID NO: 1458 | SsSNP2006B_1458 | 121 | C | G |
| SEQ ID NO: 1459 | SsSNP2006B_1459 | 121 | A | G |
| SEQ ID NO: 1460 | SsSNP2006B_1460 | 121 | A | G |
| SEQ ID NO: 1461 | SsSNP2006B_1461 | 121 | A | G |
| SEQ ID NO: 1462 | SsSNP2006B_1462 | 121 | C | G |
| SEQ ID NO: 1463 | SsSNP2006B_1463 | 121 | A | G |
| SEQ ID NO: 1464 | SsSNP2006B_1464 | 121 | A | C |
| SEQ ID NO: 1465 | SsSNP2006B_1465 | 121 | A | G |
| SEQ ID NO: 1466 | SsSNP2006B_1466 | 121 | A | G |
| SEQ ID NO: 1467 | SsSNP2006B_1467 | 121 | A | G |
| SEQ ID NO: 1468 | SsSNP2006B_1468 | 121 | A | G |
| SEQ ID NO: 1469 | SsSNP2006B_1469 | 121 | A | G |
| SEQ ID NO: 1470 | SsSNP2006B_1470 | 121 | A | G |
| SEQ ID NO: 1471 | SsSNP2006B_1471 | 121 | A | G |
| SEQ ID NO: 1472 | SsSNP2006B_1472 | 121 | A | G |
| SEQ ID NO: 1473 | SsSNP2006B_1473 | 121 | A | C |
| SEQ ID NO: 1474 | SsSNP2006B_1474 | 121 | A | G |
| SEQ ID NO: 1475 | SsSNP2006B_1475 | 121 | A | C |
| SEQ ID NO: 1476 | SsSNP2006B_1476 | 121 | A | G |
| SEQ ID NO: 1477 | SsSNP2006B_1477 | 121 | A | G |
| SEQ ID NO: 1478 | SsSNP2006B_1478 | 121 | A | G |
| SEQ ID NO: 1479 | SsSNP2006B_1479 | 121 | A | G |
| SEQ ID NO: 1480 | SsSNP2006B_1480 | 121 | A | C |
| SEQ ID NO: 1481 | SsSNP2006B_1481 | 121 | A | G |
| SEQ ID NO: 1482 | SsSNP2006B_1482 | 121 | A | G |
| SEQ ID NO: 1483 | SsSNP2006B_1483 | 121 | A | G |
| SEQ ID NO: 1484 | SsSNP2006B_1484 | 121 | A | G |
| SEQ ID NO: 1485 | SsSNP2006B_1485 | 121 | A | G |
| SEQ ID NO: 1486 | SsSNP2006B_1486 | 121 | A | C |
| SEQ ID NO: 1487 | SsSNP2006B_1487 | 121 | A | G |
| SEQ ID NO: 1488 | SsSNP2006B_1488 | 121 | A | G |
| SEQ ID NO: 1489 | SsSNP2006B_1489 | 121 | A | G |
| SEQ ID NO: 1490 | SsSNP2006B_1490 | 121 | A | C |
| SEQ ID NO: 1491 | SsSNP2006B_1491 | 121 | A | G |
| SEQ ID NO: 1492 | SsSNP2006B_1492 | 121 | A | G |
| SEQ ID NO: 1493 | SsSNP2006B_1493 | 121 | A | G |
| SEQ ID NO: 1494 | SsSNP2006B_1494 | 121 | A | T |
| SEQ ID NO: 1495 | SsSNP2006B_1495 | 121 | A | G |
| SEQ ID NO: 1496 | SsSNP2006B_1496 | 121 | A | G |
| SEQ ID NO: 1497 | SsSNP2006B_1497 | 121 | A | G |
| SEQ ID NO: 1498 | SsSNP2006B_1498 | 121 | A | G |
| SEQ ID NO: 1499 | SsSNP2006B_1499 | 121 | A | G |
| SEQ ID NO: 1500 | SsSNP2006B_1500 | 121 | A | C |
| SEQ ID NO: 1501 | SsSNP2006B_1501 | 121 | A | G |
| SEQ ID NO: 1502 | SsSNP2006B_1502 | 121 | A | G |
| SEQ ID NO: 1503 | SsSNP2006B_1503 | 121 | A | G |
| SEQ ID NO: 1504 | SsSNP2006B_1504 | 121 | A | C |
| SEQ ID NO: 1505 | SsSNP2006B_1505 | 121 | A | G |
| SEQ ID NO: 1506 | SsSNP2006B_1506 | 121 | A | C |
| SEQ ID NO: 1507 | SsSNP2006B_1507 | 121 | A | G |
| SEQ ID NO: 1508 | SsSNP2006B_1508 | 121 | A | C |
| SEQ ID NO: 1509 | SsSNP2006B_1509 | 121 | A | C |
| SEQ ID NO: 1510 | SsSNP2006B_1510 | 121 | A | C |
| SEQ ID NO: 1511 | SsSNP2006B_1511 | 121 | A | G |
| SEQ ID NO: 1512 | SsSNP2006B_1512 | 121 | A | C |
| SEQ ID NO: 1513 | SsSNP2006B_1513 | 121 | A | G |
| SEQ ID NO: 1514 | SsSNP2006B_1514 | 121 | A | G |
| SEQ ID NO: 1515 | SsSNP2006B_1515 | 121 | A | G |
| SEQ ID NO: 1516 | SsSNP2006B_1516 | 121 | A | G |
| SEQ ID NO: 1517 | SsSNP2006B_1517 | 121 | A | G |
| SEQ ID NO: 1518 | SsSNP2006B_1518 | 121 | A | G |
| SEQ ID NO: 1519 | SsSNP2006B_1519 | 121 | A | G |
| SEQ ID NO: 1520 | SsSNP2006B_1520 | 121 | A | C |
| SEQ ID NO: 1521 | SsSNP2006B_1521 | 121 | A | G |
| SEQ ID NO: 1522 | SsSNP2006B_1522 | 121 | A | G |
| SEQ ID NO: 1523 | SsSNP2006B_1523 | 121 | A | G |
| SEQ ID NO: 1524 | SsSNP2006B_1524 | 121 | A | G |
| SEQ ID NO: 1525 | SsSNP2006B_1525 | 121 | A | G |
| SEQ ID NO: 1526 | SsSNP2006B_1526 | 121 | A | T |
| SEQ ID NO: 1527 | SsSNP2006B_1527 | 121 | A | C |
| SEQ ID NO: 1528 | SsSNP2006B_1528 | 121 | A | G |
| SEQ ID NO: 1529 | SsSNP2006B_1529 | 121 | A | G |
| SEQ ID NO: 1530 | SsSNP2006B_1530 | 121 | A | G |
| SEQ ID NO: 1531 | SsSNP2006B_1531 | 121 | A | G |
| SEQ ID NO: 1532 | SsSNP2006B_1532 | 121 | A | T |
| SEQ ID NO: 1533 | SsSNP2006B_1533 | 30 | A | G |
| SEQ ID NO: 1534 | SsSNP2006B_1534 | 121 | A | G |
| SEQ ID NO: 1535 | SsSNP2006B_1535 | 121 | C | G |
| SEQ ID NO: 1536 | SsSNP2006B_1536 | 121 | A | G |
| SEQ ID NO: 1537 | SsSNP2006B_1537 | 121 | A | G |
| SEQ ID NO: 1538 | SsSNP2006B_1538 | 121 | A | C |
| SEQ ID NO: 1539 | SsSNP2006B_1539 | 121 | A | G |
| SEQ ID NO: 1540 | SsSNP2006B_1540 | 121 | A | G |
| SEQ ID NO: 1541 | SsSNP2006B_1541 | 121 | A | G |
| SEQ ID NO: 1542 | SsSNP2006B_1542 | 121 | A | G |
| SEQ ID NO: 1543 | SsSNP2006B_1543 | 121 | A | G |
| SEQ ID NO: 1544 | SsSNP2006B_1544 | 121 | A | G |
| SEQ ID NO: 1545 | SsSNP2006B_1545 | 121 | A | G |
| SEQ ID NO: 1546 | SsSNP2006B_1546 | 121 | A | T |
| SEQ ID NO: 1547 | SsSNP2006B_1547 | 106 | A | G |
| SEQ ID NO: 1548 | SsSNP2006B_1548 | 121 | A | G |
| SEQ ID NO: 1549 | SsSNP2006B_1549 | 121 | A | G |
| SEQ ID NO: 1550 | SsSNP2006B_1550 | 121 | A | G |
| SEQ ID NO: 1551 | SsSNP2006B_1551 | 121 | A | G |
| SEQ ID NO: 1552 | SsSNP2006B_1552 | 121 | A | G |
| SEQ ID NO: 1553 | SsSNP2006B_1553 | 121 | C | G |
| SEQ ID NO: 1554 | SsSNP2006B_1554 | 121 | A | G |
| SEQ ID NO: 1555 | SsSNP2006B_1555 | 121 | A | G |
| SEQ ID NO: 1556 | SsSNP2006B_1556 | 121 | A | G |
| SEQ ID NO: 1557 | SsSNP2006B_1557 | 121 | A | G |
| SEQ ID NO: 1558 | SsSNP2006B_1558 | 121 | A | C |
| SEQ ID NO: 1559 | SsSNP2006B_1559 | 121 | A | G |
| SEQ ID NO: 1560 | SsSNP2006B_1560 | 121 | A | G |
| SEQ ID NO: 1561 | SsSNP2006B_1561 | 121 | A | C |
| SEQ ID NO: 1562 | SsSNP2006B_1562 | 121 | A | G |
| SEQ ID NO: 1563 | SsSNP2006B_1563 | 121 | A | G |
| SEQ ID NO: 1564 | SsSNP2006B_1564 | 121 | A | G |
| SEQ ID NO: 1565 | SsSNP2006B_1565 | 121 | A | C |
| SEQ ID NO: 1566 | SsSNP2006B_1566 | 121 | A | G |
| SEQ ID NO: 1567 | SsSNP2006B_1567 | 121 | A | G |
| SEQ ID NO: 1568 | SsSNP2006B_1568 | 121 | A | C |
| SEQ ID NO: 1569 | SsSNP2006B_1569 | 121 | A | G |
| SEQ ID NO: 1570 | SsSNP2006B_1570 | 121 | A | G |
| SEQ ID NO: 1571 | SsSNP2006B_1571 | 121 | A | G |
| SEQ ID NO: 1572 | SsSNP2006B_1572 | 121 | A | G |
| SEQ ID NO: 1573 | SsSNP2006B_1573 | 121 | A | G |
| SEQ ID NO: 1574 | SsSNP2006B_1574 | 121 | A | G |
| SEQ ID NO: 1575 | SsSNP2006B_1575 | 121 | A | C |
| SEQ ID NO: 1576 | SsSNP2006B_1576 | 121 | A | G |
| SEQ ID NO: 1577 | SsSNP2006B_1577 | 121 | A | G |
| SEQ ID NO: 1578 | SsSNP2006B_1578 | 121 | A | G |
| SEQ ID NO: 1579 | SsSNP2006B_1579 | 121 | A | C |
| SEQ ID NO: 1580 | SsSNP2006B_1580 | 121 | A | G |
| SEQ ID NO: 1581 | SsSNP2006B_1581 | 121 | A | G |
| SEQ ID NO: 1582 | SsSNP2006B_1582 | 121 | A | G |
| SEQ ID NO: 1583 | SsSNP2006B_1583 | 121 | A | G |
| SEQ ID NO: 1584 | SsSNP2006B_1584 | 121 | A | G |
| SEQ ID NO: 1585 | SsSNP2006B_1585 | 121 | A | G |
| SEQ ID NO: 1586 | SsSNP2006B_1586 | 121 | A | G |
| SEQ ID NO: 1587 | SsSNP2006B_1587 | 121 | A | G |
| SEQ ID NO: 1588 | SsSNP2006B_1588 | 121 | A | G |
| SEQ ID NO: 1589 | SsSNP2006B_1589 | 121 | A | G |
| SEQ ID NO: 1590 | SsSNP2006B_1590 | 121 | A | G |
| SEQ ID NO: 1591 | SsSNP2006B_1591 | 121 | A | G |
| SEQ ID NO: 1592 | SsSNP2006B_1592 | 121 | A | G |
| SEQ ID NO: 1593 | SsSNP2006B_1593 | 121 | A | G |
| SEQ ID NO: 1594 | SsSNP2006B_1594 | 121 | A | G |
| SEQ ID NO: 1595 | SsSNP2006B_1595 | 121 | A | C |
| SEQ ID NO: 1596 | SsSNP2006B_1596 | 121 | A | G |
| SEQ ID NO: 1597 | SsSNP2006B_1597 | 121 | A | G |
| SEQ ID NO: 1598 | SsSNP2006B_1598 | 121 | C | G |
| SEQ ID NO: 1599 | SsSNP2006B_1599 | 121 | A | T |
| SEQ ID NO: 1600 | SsSNP2006B_1600 | 121 | A | G |
| SEQ ID NO: 1601 | SsSNP2006B_1601 | 121 | A | G |
| SEQ ID NO: 1602 | SsSNP2006B_1602 | 121 | A | G |
| SEQ ID NO: 1603 | SsSNP2006B_1603 | 121 | A | G |
| SEQ ID NO: 1604 | SsSNP2006B_1604 | 121 | A | G |
| SEQ ID NO: 1605 | SsSNP2006B_1605 | 121 | A | G |
| SEQ ID NO: 1606 | SsSNP2006B_1606 | 121 | A | G |
| SEQ ID NO: 1607 | SsSNP2006B_1607 | 121 | A | G |
| SEQ ID NO: 1608 | SsSNP2006B_1608 | 121 | A | G |
| SEQ ID NO: 1609 | SsSNP2006B_1609 | 121 | A | C |
| SEQ ID NO: 1610 | SsSNP2006B_1610 | 121 | A | G |
| SEQ ID NO: 1611 | SsSNP2006B_1611 | 121 | A | G |
| SEQ ID NO: 1612 | SsSNP2006B_1612 | 121 | A | G |
| SEQ ID NO: 1613 | SsSNP2006B_1613 | 121 | A | C |
| SEQ ID NO: 1614 | SsSNP2006B_1614 | 121 | A | G |
| SEQ ID NO: 1615 | SsSNP2006B_1615 | 121 | A | G |
| SEQ ID NO: 1616 | SsSNP2006B_1616 | 121 | A | G |
| SEQ ID NO: 1617 | SsSNP2006B_1617 | 121 | A | C |
| SEQ ID NO: 1618 | SsSNP2006B_1618 | 121 | A | G |
| SEQ ID NO: 1619 | SsSNP2006B_1619 | 121 | A | G |
| SEQ ID NO: 1620 | SsSNP2006B_1620 | 121 | A | G |
| SEQ ID NO: 1621 | SsSNP2006B_1621 | 63 | A | C |
| SEQ ID NO: 1622 | SsSNP2006B_1622 | 121 | A | G |
| SEQ ID NO: 1623 | SsSNP2006B_1623 | 121 | A | G |
| SEQ ID NO: 1624 | SsSNP2006B_1624 | 121 | A | G |
| SEQ ID NO: 1625 | SsSNP2006B_1625 | 121 | A | G |
| SEQ ID NO: 1626 | SsSNP2006B_1626 | 121 | A | G |
| SEQ ID NO: 1627 | SsSNP2006B_1627 | 121 | A | G |
| SEQ ID NO: 1628 | SsSNP2006B_1628 | 121 | A | G |
| SEQ ID NO: 1629 | SsSNP2006B_1629 | 121 | A | G |
| SEQ ID NO: 1630 | SsSNP2006B_1630 | 121 | A | C |
| SEQ ID NO: 1631 | SsSNP2006B_1631 | 121 | A | T |
| SEQ ID NO: 1632 | SsSNP2006B_1632 | 121 | A | G |
| SEQ ID NO: 1633 | SsSNP2006B_1633 | 121 | A | G |
| SEQ ID NO: 1634 | SsSNP2006B_1634 | 121 | A | G |
| SEQ ID NO: 1635 | SsSNP2006B_1635 | 121 | A | G |
| SEQ ID NO: 1636 | SsSNP2006B_1636 | 121 | A | G |
| SEQ ID NO: 1637 | SsSNP2006B_1637 | 121 | A | G |
| SEQ ID NO: 1638 | SsSNP2006B_1638 | 121 | A | G |
| SEQ ID NO: 1639 | SsSNP2006B_1639 | 121 | A | T |
| SEQ ID NO: 1640 | SsSNP2006B_1640 | 121 | A | G |
| SEQ ID NO: 1641 | SsSNP2006B_1641 | 121 | A | G |
| SEQ ID NO: 1642 | SsSNP2006B_1642 | 121 | A | G |
| SEQ ID NO: 1643 | SsSNP2006B_1643 | 121 | A | G |
| SEQ ID NO: 1644 | SsSNP2006B_1644 | 121 | A | G |
| SEQ ID NO: 1645 | SsSNP2006B_1645 | 121 | A | G |
| SEQ ID NO: 1646 | SsSNP2006B_1646 | 121 | A | G |
| SEQ ID NO: 1647 | SsSNP2006B_1647 | 121 | A | G |
| SEQ ID NO: 1648 | SsSNP2006B_1648 | 121 | A | G |
| SEQ ID NO: 1649 | SsSNP2006B_1649 | 121 | A | G |
| SEQ ID NO: 1650 | SsSNP2006B_1650 | 121 | A | G |
| SEQ ID NO: 1651 | SsSNP2006B_1651 | 121 | A | G |
| SEQ ID NO: 1652 | SsSNP2006B_1652 | 121 | A | G |
| SEQ ID NO: 1653 | SsSNP2006B_1653 | 121 | A | C |
| SEQ ID NO: 1654 | SsSNP2006B_1654 | 121 | A | T |
| SEQ ID NO: 1655 | SsSNP2006B_1655 | 121 | C | G |
| SEQ ID NO: 1656 | SsSNP2006B_1656 | 121 | A | G |
| SEQ ID NO: 1657 | SsSNP2006B_1657 | 121 | A | G |
| SEQ ID NO: 1658 | SsSNP2006B_1658 | 44 | A | G |
| SEQ ID NO: 1659 | SsSNP2006B_1659 | 121 | A | C |
| SEQ ID NO: 1660 | SsSNP2006B_1660 | 121 | A | G |
| SEQ ID NO: 1661 | SsSNP2006B_1661 | 121 | A | C |
| SEQ ID NO: 1662 | SsSNP2006B_1662 | 121 | A | G |
| SEQ ID NO: 1663 | SsSNP2006B_1663 | 121 | A | G |
| SEQ ID NO: 1664 | SsSNP2006B_1664 | 121 | A | C |
| SEQ ID NO: 1665 | SsSNP2006B_1665 | 121 | A | C |
| SEQ ID NO: 1666 | SsSNP2006B_1666 | 121 | A | G |
| SEQ ID NO: 1667 | SsSNP2006B_1667 | 121 | A | C |
| SEQ ID NO: 1668 | SsSNP2006B_1668 | 121 | A | G |
| SEQ ID NO: 1669 | SsSNP2006B_1669 | 121 | A | C |
| SEQ ID NO: 1670 | SsSNP2006B_1670 | 121 | A | G |
| SEQ ID NO: 1671 | SsSNP2006B_1671 | 121 | A | G |
| SEQ ID NO: 1672 | SsSNP2006B_1672 | 121 | A | G |
| SEQ ID NO: 1673 | SsSNP2006B_1673 | 121 | A | G |
| SEQ ID NO: 1674 | SsSNP2006B_1674 | 121 | A | C |
| SEQ ID NO: 1675 | SsSNP2006B_1675 | 121 | C | G |
| SEQ ID NO: 1676 | SsSNP2006B_1676 | 121 | A | G |
| SEQ ID NO: 1677 | SsSNP2006B_1677 | 121 | A | G |
| SEQ ID NO: 1678 | SsSNP2006B_1678 | 121 | A | C |
| SEQ ID NO: 1679 | SsSNP2006B_1679 | 121 | A | G |
| SEQ ID NO: 1680 | SsSNP2006B_1680 | 121 | A | G |
| SEQ ID NO: 1681 | SsSNP2006B_1681 | 121 | A | C |
| SEQ ID NO: 1682 | SsSNP2006B_1682 | 121 | A | G |
| SEQ ID NO: 1683 | SsSNP2006B_1683 | 121 | A | G |
| SEQ ID NO: 1684 | SsSNP2006B_1684 | 121 | A | G |
| SEQ ID NO: 1685 | SsSNP2006B_1685 | 121 | A | G |
| SEQ ID NO: 1686 | SsSNP2006B_1686 | 121 | A | G |
| SEQ ID NO: 1687 | SsSNP2006B_1687 | 121 | A | G |
| SEQ ID NO: 1688 | SsSNP2006B_1688 | 121 | A | G |
| SEQ ID NO: 1689 | SsSNP2006B_1689 | 121 | A | G |
| SEQ ID NO: 1690 | SsSNP2006B_1690 | 121 | A | G |
| SEQ ID NO: 1691 | SsSNP2006B_1691 | 121 | A | T |
| SEQ ID NO: 1692 | SsSNP2006B_1692 | 121 | A | G |
| SEQ ID NO: 1693 | SsSNP2006B_1693 | 121 | A | G |
| SEQ ID NO: 1694 | SsSNP2006B_1694 | 121 | A | G |
| SEQ ID NO: 1695 | SsSNP2006B_1695 | 121 | A | G |
| SEQ ID NO: 1696 | SsSNP2006B_1696 | 121 | A | T |
| SEQ ID NO: 1697 | SsSNP2006B_1697 | 121 | A | G |
| SEQ ID NO: 1698 | SsSNP2006B_1698 | 121 | A | G |
| SEQ ID NO: 1699 | SsSNP2006B_1699 | 121 | A | G |
| SEQ ID NO: 1700 | SsSNP2006B_1700 | 121 | A | G |
| SEQ ID NO: 1701 | SsSNP2006B_1701 | 121 | A | G |
| SEQ ID NO: 1702 | SsSNP2006B_1702 | 121 | A | G |
| SEQ ID NO: 1703 | SsSNP2006B_1703 | 121 | A | G |
| SEQ ID NO: 1704 | SsSNP2006B_1704 | 121 | A | C |
| SEQ ID NO: 1705 | SsSNP2006B_1705 | 121 | C | G |
| SEQ ID NO: 1706 | SsSNP2006B_1706 | 121 | A | G |
| SEQ ID NO: 1707 | SsSNP2006B_1707 | 121 | A | G |
| SEQ ID NO: 1708 | SsSNP2006B_1708 | 121 | A | G |
| SEQ ID NO: 1709 | SsSNP2006B_1709 | 121 | A | G |
| SEQ ID NO: 1710 | SsSNP2006B_1710 | 121 | A | G |
| SEQ ID NO: 1711 | SsSNP2006B_1711 | 121 | A | G |
| SEQ ID NO: 1712 | SsSNP2006B_1712 | 121 | A | C |
| SEQ ID NO: 1713 | SsSNP2006B_1713 | 121 | A | C |
| SEQ ID NO: 1714 | SsSNP2006B_1714 | 121 | A | G |
| SEQ ID NO: 1715 | SsSNP2006B_1715 | 121 | A | C |
| SEQ ID NO: 1716 | SsSNP2006B_1716 | 121 | A | G |
| SEQ ID NO: 1717 | SsSNP2006B_1717 | 121 | A | G |
| SEQ ID NO: 1718 | SsSNP2006B_1718 | 121 | A | T |
| SEQ ID NO: 1719 | SsSNP2006B_1719 | 121 | A | G |
| SEQ ID NO: 1720 | SsSNP2006B_1720 | 121 | A | T |
| SEQ ID NO: 1721 | SsSNP2006B_1721 | 121 | A | C |
| SEQ ID NO: 1722 | SsSNP2006B_1722 | 121 | A | G |
| SEQ ID NO: 1723 | SsSNP2006B_1723 | 121 | A | G |
| SEQ ID NO: 1724 | SsSNP2006B_1724 | 121 | C | G |
| SEQ ID NO: 1725 | SsSNP2006B_1725 | 121 | A | G |
| SEQ ID NO: 1726 | SsSNP2006B_1726 | 121 | A | C |
| SEQ ID NO: 1727 | SsSNP2006B_1727 | 121 | A | T |
| SEQ ID NO: 1728 | SsSNP2006B_1728 | 121 | A | G |
| SEQ ID NO: 1729 | SsSNP2006B_1729 | 121 | A | C |
| SEQ ID NO: 1730 | SsSNP2006B_1730 | 121 | A | G |
| SEQ ID NO: 1731 | SsSNP2006B_1731 | 121 | A | G |
| SEQ ID NO: 1732 | SsSNP2006B_1732 | 121 | A | G |
| SEQ ID NO: 1733 | SsSNP2006B_1733 | 121 | A | C |
| SEQ ID NO: 1734 | SsSNP2006B_1734 | 121 | C | G |
| SEQ ID NO: 1735 | SsSNP2006B_1735 | 121 | A | G |
| SEQ ID NO: 1736 | SsSNP2006B_1736 | 121 | A | C |
| SEQ ID NO: 1737 | SsSNP2006B_1737 | 121 | A | G |
| SEQ ID NO: 1738 | SsSNP2006B_1738 | 121 | A | G |
| SEQ ID NO: 1739 | SsSNP2006B_1739 | 121 | A | G |
| SEQ ID NO: 1740 | SsSNP2006B_1740 | 121 | A | C |
| SEQ ID NO: 1741 | SsSNP2006B_1741 | 121 | A | G |
| SEQ ID NO: 1742 | SsSNP2006B_1742 | 121 | A | G |
| SEQ ID NO: 1743 | SsSNP2006B_1743 | 121 | C | G |
| SEQ ID NO: 1744 | SsSNP2006B_1744 | 121 | A | G |
| SEQ ID NO: 1745 | SsSNP2006B_1745 | 121 | A | G |
| SEQ ID NO: 1746 | SsSNP2006B_1746 | 121 | A | G |
| SEQ ID NO: 1747 | SsSNP2006B_1747 | 121 | A | T |
| SEQ ID NO: 1748 | SsSNP2006B_1748 | 121 | A | G |
| SEQ ID NO: 1749 | SsSNP2006B_1749 | 121 | A | G |
| SEQ ID NO: 1750 | SsSNP2006B_1750 | 121 | A | C |
| SEQ ID NO: 1751 | SsSNP2006B_1751 | 121 | A | T |
| SEQ ID NO: 1752 | SsSNP2006B_1752 | 121 | A | G |
| SEQ ID NO: 1753 | SsSNP2006B_1753 | 121 | A | G |
| SEQ ID NO: 1754 | SsSNP2006B_1754 | 121 | A | T |
| SEQ ID NO: 1755 | SsSNP2006B_1755 | 121 | A | G |
| SEQ ID NO: 1756 | SsSNP2006B_1756 | 121 | A | G |
| SEQ ID NO: 1757 | SsSNP2006B_1757 | 121 | A | G |
| SEQ ID NO: 1758 | SsSNP2006B_1758 | 121 | A | G |
| SEQ ID NO: 1759 | SsSNP2006B_1759 | 121 | A | G |
| SEQ ID NO: 1760 | SsSNP2006B_1760 | 121 | A | C |
| SEQ ID NO: 1761 | SsSNP2006B_1761 | 121 | A | G |
| SEQ ID NO: 1762 | SsSNP2006B_1762 | 121 | A | G |
| SEQ ID NO: 1763 | SsSNP2006B_1763 | 121 | A | G |
| SEQ ID NO: 1764 | SsSNP2006B_1764 | 121 | C | G |
| SEQ ID NO: 1765 | SsSNP2006B_1765 | 121 | A | G |
| SEQ ID NO: 1766 | SsSNP2006B_1766 | 121 | A | C |
| SEQ ID NO: 1767 | SsSNP2006B_1767 | 121 | A | G |
| SEQ ID NO: 1768 | SsSNP2006B_1768 | 121 | A | G |
| SEQ ID NO: 1769 | SsSNP2006B_1769 | 121 | A | G |
| SEQ ID NO: 1770 | SsSNP2006B_1770 | 121 | A | G |
| SEQ ID NO: 1771 | SsSNP2006B_1771 | 121 | A | G |
| SEQ ID NO: 1772 | SsSNP2006B_1772 | 121 | A | G |
| SEQ ID NO: 1773 | SsSNP2006B_1773 | 121 | A | G |
| SEQ ID NO: 1774 | SsSNP2006B_1774 | 121 | A | G |
| SEQ ID NO: 1775 | SsSNP2006B_1775 | 121 | A | G |
| SEQ ID NO: 1776 | SsSNP2006B_1776 | 121 | A | C |
| SEQ ID NO: 1777 | SsSNP2006B_1777 | 121 | A | G |
| SEQ ID NO: 1778 | SsSNP2006B_1778 | 121 | A | G |
| SEQ ID NO: 1779 | SsSNP2006B_1779 | 121 | A | G |
| SEQ ID NO: 1780 | SsSNP2006B_1780 | 121 | A | C |
| SEQ ID NO: 1781 | SsSNP2006B_1781 | 121 | A | G |
| SEQ ID NO: 1782 | SsSNP2006B_1782 | 121 | A | G |
| SEQ ID NO: 1783 | SsSNP2006B_1783 | 121 | A | G |
| SEQ ID NO: 1784 | SsSNP2006B_1784 | 121 | A | G |
| SEQ ID NO: 1785 | SsSNP2006B_1785 | 121 | A | G |
| SEQ ID NO: 1786 | SsSNP2006B_1786 | 121 | A | G |
| SEQ ID NO: 1787 | SsSNP2006B_1787 | 121 | A | G |
| SEQ ID NO: 1788 | SsSNP2006B_1788 | 121 | A | C |
| SEQ ID NO: 1789 | SsSNP2006B_1789 | 121 | A | T |
| SEQ ID NO: 1790 | SsSNP2006B_1790 | 121 | C | G |
| SEQ ID NO: 1791 | SsSNP2006B_1791 | 121 | A | G |
| SEQ ID NO: 1792 | SsSNP2006B_1792 | 121 | A | G |
| SEQ ID NO: 1793 | SsSNP2006B_1793 | 121 | A | G |
| SEQ ID NO: 1794 | SsSNP2006B_1794 | 121 | A | G |
| SEQ ID NO: 1795 | SsSNP2006B_1795 | 121 | A | G |
| SEQ ID NO: 1796 | SsSNP2006B_1796 | 121 | A | G |
| SEQ ID NO: 1797 | SsSNP2006B_1797 | 121 | A | G |
| SEQ ID NO: 1798 | SsSNP2006B_1798 | 121 | A | G |
| SEQ ID NO: 1799 | SsSNP2006B_1799 | 121 | A | G |
| SEQ ID NO: 1800 | SsSNP2006B_1800 | 121 | A | G |
| SEQ ID NO: 1801 | SsSNP2006B_1801 | 121 | A | G |
| SEQ ID NO: 1802 | SsSNP2006B_1802 | 97 | A | G |
| SEQ ID NO: 1803 | SsSNP2006B_1803 | 121 | A | G |
| SEQ ID NO: 1804 | SsSNP2006B_1804 | 121 | A | C |
| SEQ ID NO: 1805 | SsSNP2006B_1805 | 121 | A | C |
| SEQ ID NO: 1806 | SsSNP2006B_1806 | 121 | A | G |
| SEQ ID NO: 1807 | SsSNP2006B_1807 | 121 | A | G |
| SEQ ID NO: 1808 | SsSNP2006B_1808 | 121 | A | G |
| SEQ ID NO: 1809 | SsSNP2006B_1809 | 121 | A | C |
| SEQ ID NO: 1810 | SsSNP2006B_1810 | 121 | C | G |
| SEQ ID NO: 1811 | SsSNP2006B_1811 | 121 | A | G |
| SEQ ID NO: 1812 | SsSNP2006B_1812 | 121 | A | G |
| SEQ ID NO: 1813 | SsSNP2006B_1813 | 121 | A | G |
| SEQ ID NO: 1814 | SsSNP2006B_1814 | 121 | A | G |
| SEQ ID NO: 1815 | SsSNP2006B_1815 | 121 | A | G |
| SEQ ID NO: 1816 | SsSNP2006B_1816 | 121 | A | G |
| SEQ ID NO: 1817 | SsSNP2006B_1817 | 121 | A | G |
| SEQ ID NO: 1818 | SsSNP2006B_1818 | 121 | A | G |
| SEQ ID NO: 1819 | SsSNP2006B_1819 | 121 | A | G |
| SEQ ID NO: 1820 | SsSNP2006B_1820 | 121 | A | G |
| SEQ ID NO: 1821 | SsSNP2006B_1821 | 121 | C | G |
| SEQ ID NO: 1822 | SsSNP2006B_1822 | 121 | A | C |
| SEQ ID NO: 1823 | SsSNP2006B_1823 | 121 | A | G |
| SEQ ID NO: 1824 | SsSNP2006B_1824 | 121 | A | G |
| SEQ ID NO: 1825 | SsSNP2006B_1825 | 121 | A | C |
| SEQ ID NO: 1826 | SsSNP2006B_1826 | 121 | A | G |
| SEQ ID NO: 1827 | SsSNP2006B_1827 | 121 | A | G |
| SEQ ID NO: 1828 | SsSNP2006B_1828 | 121 | A | G |
| SEQ ID NO: 1829 | SsSNP2006B_1829 | 121 | C | G |
| SEQ ID NO: 1830 | SsSNP2006B_1830 | 121 | A | G |
| SEQ ID NO: 1831 | SsSNP2006B_1831 | 121 | A | G |
| SEQ ID NO: 1832 | SsSNP2006B_1832 | 121 | A | G |
| SEQ ID NO: 1833 | SsSNP2006B_1833 | 121 | A | G |
| SEQ ID NO: 1834 | SsSNP2006B_1834 | 121 | A | C |
| SEQ ID NO: 1835 | SsSNP2006B_1835 | 121 | C | G |
| SEQ ID NO: 1836 | SsSNP2006B_1836 | 121 | A | G |
| SEQ ID NO: 1837 | SsSNP2006B_1837 | 121 | A | G |
| SEQ ID NO: 1838 | SsSNP2006B_1838 | 121 | A | T |
| SEQ ID NO: 1839 | SsSNP2006B_1839 | 121 | A | C |
| SEQ ID NO: 1840 | SsSNP2006B_1840 | 121 | A | C |
| SEQ ID NO: 1841 | SsSNP2006B_1841 | 121 | A | G |
| SEQ ID NO: 1842 | SsSNP2006B_1842 | 121 | A | G |
| SEQ ID NO: 1843 | SsSNP2006B_1843 | 121 | A | T |
| SEQ ID NO: 1844 | SsSNP2006B_1844 | 121 | A | C |
| SEQ ID NO: 1845 | SsSNP2006B_1845 | 121 | A | G |
| SEQ ID NO: 1846 | SsSNP2006B_1846 | 121 | A | G |
| SEQ ID NO: 1847 | SsSNP2006B_1847 | 121 | A | T |
| SEQ ID NO: 1848 | SsSNP2006B_1848 | 121 | A | G |
| SEQ ID NO: 1849 | SsSNP2006B_1849 | 121 | A | G |
| SEQ ID NO: 1850 | SsSNP2006B_1850 | 121 | A | G |
| SEQ ID NO: 1851 | SsSNP2006B_1851 | 121 | A | G |
| SEQ ID NO: 1852 | SsSNP2006B_1852 | 121 | A | T |
| SEQ ID NO: 1853 | SsSNP2006B_1853 | 121 | A | G |
| SEQ ID NO: 1854 | SsSNP2006B_1854 | 121 | A | G |
| SEQ ID NO: 1855 | SsSNP2006B_1855 | 121 | A | G |
| SEQ ID NO: 1856 | SsSNP2006B_1856 | 121 | A | G |
| SEQ ID NO: 1857 | SsSNP2006B_1857 | 121 | A | G |
| SEQ ID NO: 1858 | SsSNP2006B_1858 | 121 | A | G |
| SEQ ID NO: 1859 | SsSNP2006B_1859 | 121 | A | G |
| SEQ ID NO: 1860 | SsSNP2006B_1860 | 121 | A | C |
| SEQ ID NO: 1861 | SsSNP2006B_1861 | 121 | A | G |
| SEQ ID NO: 1862 | SsSNP2006B_1862 | 121 | A | G |
| SEQ ID NO: 1863 | SsSNP2006B_1863 | 121 | A | G |
| SEQ ID NO: 1864 | SsSNP2006B_1864 | 94 | C | G |
| SEQ ID NO: 1865 | SsSNP2006B_1865 | 121 | A | C |
| SEQ ID NO: 1866 | SsSNP2006B_1866 | 121 | A | G |
| SEQ ID NO: 1867 | SsSNP2006B_1867 | 121 | A | G |
| SEQ ID NO: 1868 | SsSNP2006B_1868 | 120 | C | G |
| SEQ ID NO: 1869 | SsSNP2006B_1869 | 121 | A | G |
| SEQ ID NO: 1870 | SsSNP2006B_1870 | 121 | A | G |
| SEQ ID NO: 1871 | SsSNP2006B_1871 | 121 | A | G |
| SEQ ID NO: 1872 | SsSNP2006B_1872 | 121 | A | G |
| SEQ ID NO: 1873 | SsSNP2006B_1873 | 121 | A | G |
| SEQ ID NO: 1874 | SsSNP2006B_1874 | 121 | A | G |
| SEQ ID NO: 1875 | SsSNP2006B_1875 | 121 | A | G |
| SEQ ID NO: 1876 | SsSNP2006B_1876 | 121 | A | G |
| SEQ ID NO: 1877 | SsSNP2006B_1877 | 121 | A | G |
| SEQ ID NO: 1878 | SsSNP2006B_1878 | 121 | A | G |
| SEQ ID NO: 1879 | SsSNP2006B_1879 | 121 | A | G |
| SEQ ID NO: 1880 | SsSNP2006B_1880 | 121 | A | G |
| SEQ ID NO: 1881 | SsSNP2006B_1881 | 121 | A | G |
| SEQ ID NO: 1882 | SsSNP2006B_1882 | 80 | C | G |
| SEQ ID NO: 1883 | SsSNP2006B_1883 | 121 | A | C |
| SEQ ID NO: 1884 | SsSNP2006B_1884 | 121 | A | G |
| SEQ ID NO: 1885 | SsSNP2006B_1885 | 121 | A | G |
| SEQ ID NO: 1886 | SsSNP2006B_1886 | 121 | A | G |
| SEQ ID NO: 1887 | SsSNP2006B_1887 | 121 | A | T |
| SEQ ID NO: 1888 | SsSNP2006B_1888 | 121 | A | G |
| SEQ ID NO: 1889 | SsSNP2006B_1889 | 121 | A | G |
| SEQ ID NO: 1890 | SsSNP2006B_1890 | 121 | A | G |
| SEQ ID NO: 1891 | SsSNP2006B_1891 | 121 | A | G |
| SEQ ID NO: 1892 | SsSNP2006B_1892 | 121 | A | G |
| SEQ ID NO: 1893 | SsSNP2006B_1893 | 121 | A | G |
| SEQ ID NO: 1894 | SsSNP2006B_1894 | 121 | A | G |
| SEQ ID NO: 1895 | SsSNP2006B_1895 | 121 | A | C |
| SEQ ID NO: 1896 | SsSNP2006B_1896 | 121 | A | G |
| SEQ ID NO: 1897 | SsSNP2006B_1897 | 121 | A | G |
| SEQ ID NO: 1898 | SsSNP2006B_1898 | 121 | A | G |
| SEQ ID NO: 1899 | SsSNP2006B_1899 | 121 | C | G |
| SEQ ID NO: 1900 | SsSNP2006B_1900 | 121 | A | C |
| SEQ ID NO: 1901 | SsSNP2006B_1901 | 121 | A | G |
| SEQ ID NO: 1902 | SsSNP2006B_1902 | 121 | A | G |
| SEQ ID NO: 1903 | SsSNP2006B_1903 | 121 | A | G |
| SEQ ID NO: 1904 | SsSNP2006B_1904 | 121 | A | G |
| SEQ ID NO: 1905 | SsSNP2006B_1905 | 121 | A | T |
| SEQ ID NO: 1906 | SsSNP2006B_1906 | 121 | A | G |
| SEQ ID NO: 1907 | SsSNP2006B_1907 | 121 | A | G |
| SEQ ID NO: 1908 | SsSNP2006B_1908 | 121 | A | G |
| SEQ ID NO: 1909 | SsSNP2006B_1909 | 121 | A | G |
| SEQ ID NO: 1910 | SsSNP2006B_1910 | 121 | A | C |
| SEQ ID NO: 1911 | SsSNP2006B_1911 | 121 | A | G |
| SEQ ID NO: 1912 | SsSNP2006B_1912 | 121 | A | G |
| SEQ ID NO: 1913 | SsSNP2006B_1913 | 121 | A | G |
| SEQ ID NO: 1914 | SsSNP2006B_1914 | 121 | A | C |
| SEQ ID NO: 1915 | SsSNP2006B_1915 | 121 | C | G |
| SEQ ID NO: 1916 | SsSNP2006B_1916 | 121 | A | C |
| SEQ ID NO: 1917 | SsSNP2006B_1917 | 121 | A | T |
| SEQ ID NO: 1918 | SsSNP2006B_1918 | 121 | A | G |
| SEQ ID NO: 1919 | SsSNP2006B_1919 | 121 | A | C |
| SEQ ID NO: 1920 | SsSNP2006B_1920 | 121 | A | G |
| SEQ ID NO: 1921 | SsSNP2006B_1921 | 121 | A | C |
| SEQ ID NO: 1922 | SsSNP2006B_1922 | 121 | A | G |
| SEQ ID NO: 1923 | SsSNP2006B_1923 | 121 | A | C |
| SEQ ID NO: 1924 | SsSNP2006B_1924 | 121 | A | G |
| SEQ ID NO: 1925 | SsSNP2006B_1925 | 121 | A | G |
| SEQ ID NO: 1926 | SsSNP2006B_1926 | 121 | C | G |
| SEQ ID NO: 1927 | SsSNP2006B_1927 | 121 | A | C |
| SEQ ID NO: 1928 | SsSNP2006B_1928 | 121 | A | G |
| SEQ ID NO: 1929 | SsSNP2006B_1929 | 121 | A | G |
| SEQ ID NO: 1930 | SsSNP2006B_1930 | 121 | A | C |
| SEQ ID NO: 1931 | SsSNP2006B_1931 | 121 | A | G |
| SEQ ID NO: 1932 | SsSNP2006B_1932 | 121 | A | T |
| SEQ ID NO: 1933 | SsSNP2006B_1933 | 121 | A | G |
| SEQ ID NO: 1934 | SsSNP2006B_1934 | 121 | A | C |
| SEQ ID NO: 1935 | SsSNP2006B_1935 | 121 | A | G |
| SEQ ID NO: 1936 | SsSNP2006B_1936 | 121 | A | G |
| SEQ ID NO: 1937 | SsSNP2006B_1937 | 121 | A | C |
| SEQ ID NO: 1938 | SsSNP2006B_1938 | 121 | A | G |
| SEQ ID NO: 1939 | SsSNP2006B_1939 | 121 | A | C |
| SEQ ID NO: 1940 | SsSNP2006B_1940 | 121 | A | G |
| SEQ ID NO: 1941 | SsSNP2006B_1941 | 121 | A | G |
| SEQ ID NO: 1942 | SsSNP2006B_1942 | 121 | A | C |
| SEQ ID NO: 1943 | SsSNP2006B_1943 | 121 | A | G |
| SEQ ID NO: 1944 | SsSNP2006B_1944 | 121 | A | C |
| SEQ ID NO: 1945 | SsSNP2006B_1945 | 121 | A | C |
| SEQ ID NO: 1946 | SsSNP2006B_1946 | 121 | A | G |
| SEQ ID NO: 1947 | SsSNP2006B_1947 | 121 | A | G |
| SEQ ID NO: 1948 | SsSNP2006B_1948 | 121 | A | G |
| SEQ ID NO: 1949 | SsSNP2006B_1949 | 121 | A | T |
| SEQ ID NO: 1950 | SsSNP2006B_1950 | 121 | A | G |
| SEQ ID NO: 1951 | SsSNP2006B_1951 | 121 | A | G |
| SEQ ID NO: 1952 | SsSNP2006B_1952 | 121 | A | G |
| SEQ ID NO: 1953 | SsSNP2006B_1953 | 121 | A | G |
| SEQ ID NO: 1954 | SsSNP2006B_1954 | 121 | A | G |
| SEQ ID NO: 1955 | SsSNP2006B_1955 | 121 | A | G |
| SEQ ID NO: 1956 | SsSNP2006B_1956 | 121 | A | G |
| SEQ ID NO: 1957 | SsSNP2006B_1957 | 121 | A | G |
| SEQ ID NO: 1958 | SsSNP2006B_1958 | 121 | A | G |
| SEQ ID NO: 1959 | SsSNP2006B_1959 | 121 | A | G |
| SEQ ID NO: 1960 | SsSNP2006B_1960 | 121 | A | T |
| SEQ ID NO: 1961 | SsSNP2006B_1961 | 121 | A | G |
| SEQ ID NO: 1962 | SsSNP2006B_1962 | 121 | A | G |
| SEQ ID NO: 1963 | SsSNP2006B_1963 | 121 | A | G |
| SEQ ID NO: 1964 | SsSNP2006B_1964 | 121 | A | G |
| SEQ ID NO: 1965 | SsSNP2006B_1965 | 121 | A | G |
| SEQ ID NO: 1966 | SsSNP2006B_1966 | 121 | A | G |
| SEQ ID NO: 1967 | SsSNP2006B_1967 | 121 | A | G |
| SEQ ID NO: 1968 | SsSNP2006B_1968 | 121 | A | G |
| SEQ ID NO: 1969 | SsSNP2006B_1969 | 121 | A | G |
| SEQ ID NO: 1970 | SsSNP2006B_1970 | 121 | A | T |
| SEQ ID NO: 1971 | SsSNP2006B_1971 | 121 | C | G |
| SEQ ID NO: 1972 | SsSNP2006B_1972 | 121 | A | G |
| SEQ ID NO: 1973 | SsSNP2006B_1973 | 121 | A | T |
| SEQ ID NO: 1974 | SsSNP2006B_1974 | 121 | C | G |
| SEQ ID NO: 1975 | SsSNP2006B_1975 | 121 | A | G |
| SEQ ID NO: 1976 | SsSNP2006B_1976 | 121 | A | C |
| SEQ ID NO: 1977 | SsSNP2006B_1977 | 121 | C | G |
| SEQ ID NO: 1978 | SsSNP2006B_1978 | 121 | A | G |
| SEQ ID NO: 1979 | SsSNP2006B_1979 | 121 | A | T |
| SEQ ID NO: 1980 | SsSNP2006B_1980 | 121 | A | G |
| SEQ ID NO: 1981 | SsSNP2006B_1981 | 121 | A | G |
| SEQ ID NO: 1982 | SsSNP2006B_1982 | 121 | A | G |
| SEQ ID NO: 1983 | SsSNP2006B_1983 | 121 | A | G |
| SEQ ID NO: 1984 | SsSNP2006B_1984 | 121 | C | G |
| SEQ ID NO: 1985 | SsSNP2006B_1985 | 121 | A | G |
| SEQ ID NO: 1986 | SsSNP2006B_1986 | 121 | A | T |
| SEQ ID NO: 1987 | SsSNP2006B_1987 | 121 | A | G |
| SEQ ID NO: 1988 | SsSNP2006B_1988 | 121 | A | G |
| SEQ ID NO: 1989 | SsSNP2006B_1989 | 121 | A | G |
| SEQ ID NO: 1990 | SsSNP2006B_1990 | 121 | A | G |
| SEQ ID NO: 1991 | SsSNP2006B_1991 | 121 | A | G |
| SEQ ID NO: 1992 | SsSNP2006B_1992 | 121 | A | G |
| SEQ ID NO: 1993 | SsSNP2006B_1993 | 121 | C | G |
| SEQ ID NO: 1994 | SsSNP2006B_1994 | 121 | A | G |
| SEQ ID NO: 1995 | SsSNP2006B_1995 | 121 | A | G |
| SEQ ID NO: 1996 | SsSNP2006B_1996 | 121 | A | G |
| SEQ ID NO: 1997 | SsSNP2006B_1997 | 121 | A | G |
| SEQ ID NO: 1998 | SsSNP2006B_1998 | 121 | A | G |
| SEQ ID NO: 1999 | SsSNP2006B_1999 | 121 | A | G |
| SEQ ID NO: 2000 | SsSNP2006B_2000 | 121 | C | G |
| SEQ ID NO: 2001 | SsSNP2006B_2001 | 121 | C | G |
| SEQ ID NO: 2002 | SsSNP2006B_2002 | 121 | A | G |
| SEQ ID NO: 2003 | SsSNP2006B_2003 | 121 | A | G |
| SEQ ID NO: 2004 | SsSNP2006B_2004 | 121 | A | G |
| SEQ ID NO: 2005 | SsSNP2006B_2005 | 121 | A | C |
| SEQ ID NO: 2006 | SsSNP2006B_2006 | 121 | A | G |
| SEQ ID NO: 2007 | SsSNP2006B_2007 | 121 | A | T |
| SEQ ID NO: 2008 | SsSNP2006B_2008 | 121 | A | G |
| SEQ ID NO: 2009 | SsSNP2006B_2009 | 121 | A | G |
| SEQ ID NO: 2010 | SsSNP2006B_2010 | 121 | A | G |
| SEQ ID NO: 2011 | SsSNP2006B_2011 | 121 | A | G |
| SEQ ID NO: 2012 | SsSNP2006B_2012 | 121 | A | T |
| SEQ ID NO: 2013 | SsSNP2006B_2013 | 121 | A | G |
| SEQ ID NO: 2014 | SsSNP2006B_2014 | 121 | C | G |
| SEQ ID NO: 2015 | SsSNP2006B_2015 | 121 | A | G |
| SEQ ID NO: 2016 | SsSNP2006B_2016 | 121 | A | G |
| SEQ ID NO: 2017 | SsSNP2006B_2017 | 121 | A | C |
| SEQ ID NO: 2018 | SsSNP2006B_2018 | 121 | A | G |
| SEQ ID NO: 2019 | SsSNP2006B_2019 | 121 | A | G |
| SEQ ID NO: 2020 | SsSNP2006B_2020 | 121 | A | G |
| SEQ ID NO: 2021 | SsSNP2006B_2021 | 121 | A | T |
| SEQ ID NO: 2022 | SsSNP2006B_2022 | 121 | A | G |
| SEQ ID NO: 2023 | SsSNP2006B_2023 | 121 | A | G |
| SEQ ID NO: 2024 | SsSNP2006B_2024 | 121 | C | G |
| SEQ ID NO: 2025 | SsSNP2006B_2025 | 121 | A | C |
| SEQ ID NO: 2026 | SsSNP2006B_2026 | 121 | A | G |
| SEQ ID NO: 2027 | SsSNP2006B_2027 | 121 | A | T |
| SEQ ID NO: 2028 | SsSNP2006B_2028 | 121 | A | G |
| SEQ ID NO: 2029 | SsSNP2006B_2029 | 121 | A | C |
| SEQ ID NO: 2030 | SsSNP2006B_2030 | 121 | A | C |
| SEQ ID NO: 2031 | SsSNP2006B_2031 | 121 | C | G |
| SEQ ID NO: 2032 | SsSNP2006B_2032 | 121 | A | G |
| SEQ ID NO: 2033 | SsSNP2006B_2033 | 121 | A | G |
| SEQ ID NO: 2034 | SsSNP2006B_2034 | 121 | A | G |
| SEQ ID NO: 2035 | SsSNP2006B_2035 | 121 | A | G |
| SEQ ID NO: 2036 | SsSNP2006B_2036 | 121 | A | G |
| SEQ ID NO: 2037 | SsSNP2006B_2037 | 121 | A | G |
| SEQ ID NO: 2038 | SsSNP2006B_2038 | 121 | A | G |
| SEQ ID NO: 2039 | SsSNP2006B_2039 | 121 | A | G |
| SEQ ID NO: 2040 | SsSNP2006B_2040 | 121 | A | G |
| SEQ ID NO: 2041 | SsSNP2006B_2041 | 121 | A | G |
| SEQ ID NO: 2042 | SsSNP2006B_2042 | 121 | C | G |
| SEQ ID NO: 2043 | SsSNP2006B_2043 | 121 | A | G |
| SEQ ID NO: 2044 | SsSNP2006B_2044 | 121 | A | G |
| SEQ ID NO: 2045 | SsSNP2006B_2045 | 121 | A | G |
| SEQ ID NO: 2046 | SsSNP2006B_2046 | 121 | A | G |
| SEQ ID NO: 2047 | SsSNP2006B_2047 | 121 | A | G |
| SEQ ID NO: 2048 | SsSNP2006B_2048 | 121 | A | G |
| SEQ ID NO: 2049 | SsSNP2006B_2049 | 121 | A | G |
| SEQ ID NO: 2050 | SsSNP2006B_2050 | 121 | A | G |
| SEQ ID NO: 2051 | SsSNP2006B_2051 | 121 | A | G |
| SEQ ID NO: 2052 | SsSNP2006B_2052 | 121 | A | C |
| SEQ ID NO: 2053 | SsSNP2006B_2053 | 121 | A | G |
| SEQ ID NO: 2054 | SsSNP2006B_2054 | 121 | A | G |
| SEQ ID NO: 2055 | SsSNP2006B_2055 | 121 | A | G |
| SEQ ID NO: 2056 | SsSNP2006B_2056 | 121 | A | G |
| SEQ ID NO: 2057 | SsSNP2006B_2057 | 121 | C | G |
| SEQ ID NO: 2058 | SsSNP2006B_2058 | 121 | A | G |
| SEQ ID NO: 2059 | SsSNP2006B_2059 | 121 | A | G |
| SEQ ID NO: 2060 | SsSNP2006B_2060 | 121 | A | C |
| SEQ ID NO: 2061 | SsSNP2006B_2061 | 121 | A | G |
| SEQ ID NO: 2062 | SsSNP2006B_2062 | 121 | A | G |
| SEQ ID NO: 2063 | SsSNP2006B_2063 | 121 | A | G |
| SEQ ID NO: 2064 | SsSNP2006B_2064 | 121 | C | G |
| SEQ ID NO: 2065 | SsSNP2006B_2065 | 121 | A | G |
| SEQ ID NO: 2066 | SsSNP2006B_2066 | 121 | A | C |
| SEQ ID NO: 2067 | SsSNP2006B_2067 | 121 | A | G |
| SEQ ID NO: 2068 | SsSNP2006B_2068 | 121 | A | G |
| SEQ ID NO: 2069 | SsSNP2006B_2069 | 121 | A | C |
| SEQ ID NO: 2070 | SsSNP2006B_2070 | 121 | A | C |
| SEQ ID NO: 2071 | SsSNP2006B_2071 | 121 | A | G |
| SEQ ID NO: 2072 | SsSNP2006B_2072 | 121 | A | G |
| SEQ ID NO: 2073 | SsSNP2006B_2073 | 121 | A | G |
| SEQ ID NO: 2074 | SsSNP2006B_2074 | 121 | A | G |
| SEQ ID NO: 2075 | SsSNP2006B_2075 | 121 | A | G |
| SEQ ID NO: 2076 | SsSNP2006B_2076 | 121 | A | G |
| SEQ ID NO: 2077 | SsSNP2006B_2077 | 121 | A | C |
| SEQ ID NO: 2078 | SsSNP2006B_2078 | 121 | A | G |
| SEQ ID NO: 2079 | SsSNP2006B_2079 | 121 | A | G |
| SEQ ID NO: 2080 | SsSNP2006B_2080 | 121 | A | G |
| SEQ ID NO: 2081 | SsSNP2006B_2081 | 121 | A | G |
| SEQ ID NO: 2082 | SsSNP2006B_2082 | 121 | A | G |
| SEQ ID NO: 2083 | SsSNP2006B_2083 | 121 | A | G |
| SEQ ID NO: 2084 | SsSNP2006B_2084 | 121 | A | C |
| SEQ ID NO: 2085 | SsSNP2006B_2085 | 121 | C | G |
| SEQ ID NO: 2086 | SsSNP2006B_2086 | 121 | A | C |
| SEQ ID NO: 2087 | SsSNP2006B_2087 | 121 | A | G |
| SEQ ID NO: 2088 | SsSNP2006B_2088 | 121 | A | G |
| SEQ ID NO: 2089 | SsSNP2006B_2089 | 121 | A | G |
| SEQ ID NO: 2090 | SsSNP2006B_2090 | 121 | A | G |
| SEQ ID NO: 2091 | SsSNP2006B_2091 | 121 | A | G |
| SEQ ID NO: 2092 | SsSNP2006B_2092 | 121 | A | G |
| SEQ ID NO: 2093 | SsSNP2006B_2093 | 121 | A | G |
| SEQ ID NO: 2094 | SsSNP2006B_2094 | 121 | A | G |
| SEQ ID NO: 2095 | SsSNP2006B_2095 | 121 | A | G |
| SEQ ID NO: 2096 | SsSNP2006B_2096 | 121 | A | G |
| SEQ ID NO: 2097 | SsSNP2006B_2097 | 121 | A | C |
| SEQ ID NO: 2098 | SsSNP2006B_2098 | 121 | A | G |
| SEQ ID NO: 2099 | SsSNP2006B_2099 | 121 | A | G |
| SEQ ID NO: 2100 | SsSNP2006B_2100 | 121 | A | G |
| SEQ ID NO: 2101 | SsSNP2006B_2101 | 121 | A | G |
| SEQ ID NO: 2102 | SsSNP2006B_2102 | 121 | A | T |
| SEQ ID NO: 2103 | SsSNP2006B_2103 | 85 | A | G |
| SEQ ID NO: 2104 | SsSNP2006B_2104 | 121 | A | C |
| SEQ ID NO: 2105 | SsSNP2006B_2105 | 121 | A | G |
| SEQ ID NO: 2106 | SsSNP2006B_2106 | 121 | A | G |
| SEQ ID NO: 2107 | SsSNP2006B_2107 | 121 | A | G |
| SEQ ID NO: 2108 | SsSNP2006B_2108 | 121 | A | G |
| SEQ ID NO: 2109 | SsSNP2006B_2109 | 121 | A | C |
| SEQ ID NO: 2110 | SsSNP2006B_2110 | 121 | A | G |
| SEQ ID NO: 2111 | SsSNP2006B_2111 | 121 | A | G |
| SEQ ID NO: 2112 | SsSNP2006B_2112 | 121 | A | G |
| SEQ ID NO: 2113 | SsSNP2006B_2113 | 121 | A | C |
| SEQ ID NO: 2114 | SsSNP2006B_2114 | 121 | A | G |
| SEQ ID NO: 2115 | SsSNP2006B_2115 | 121 | A | G |
| SEQ ID NO: 2116 | SsSNP2006B_2116 | 121 | A | T |
| SEQ ID NO: 2117 | SsSNP2006B_2117 | 121 | A | C |
| SEQ ID NO: 2118 | SsSNP2006B_2118 | 121 | A | G |
| SEQ ID NO: 2119 | SsSNP2006B_2119 | 121 | A | T |
| SEQ ID NO: 2120 | SsSNP2006B_2120 | 121 | A | G |
| SEQ ID NO: 2121 | SsSNP2006B_2121 | 121 | A | G |
| SEQ ID NO: 2122 | SsSNP2006B_2122 | 121 | A | G |
| SEQ ID NO: 2123 | SsSNP2006B_2123 | 121 | A | G |
| SEQ ID NO: 2124 | SsSNP2006B_2124 | 121 | A | G |
| SEQ ID NO: 2125 | SsSNP2006B_2125 | 121 | C | G |
| SEQ ID NO: 2126 | SsSNP2006B_2126 | 121 | A | G |
| SEQ ID NO: 2127 | SsSNP2006B_2127 | 121 | C | G |
| SEQ ID NO: 2128 | SsSNP2006B_2128 | 121 | C | G |
| SEQ ID NO: 2129 | SsSNP2006B_2129 | 121 | A | G |
| SEQ ID NO: 2130 | SsSNP2006B_2130 | 121 | A | G |
| SEQ ID NO: 2131 | SsSNP2006B_2131 | 121 | A | G |
| SEQ ID NO: 2132 | SsSNP2006B_2132 | 121 | A | G |
| SEQ ID NO: 2133 | SsSNP2006B_2133 | 121 | A | G |
| SEQ ID NO: 2134 | SsSNP2006B_2134 | 121 | A | G |
| SEQ ID NO: 2135 | SsSNP2006B_2135 | 121 | A | C |
| SEQ ID NO: 2136 | SsSNP2006B_2136 | 121 | A | G |
| SEQ ID NO: 2137 | SsSNP2006B_2137 | 121 | A | G |
| SEQ ID NO: 2138 | SsSNP2006B_2138 | 121 | A | G |
| SEQ ID NO: 2139 | SsSNP2006B_2139 | 121 | A | T |
| SEQ ID NO: 2140 | SsSNP2006B_2140 | 121 | A | G |
| SEQ ID NO: 2141 | SsSNP2006B_2141 | 121 | C | G |
| SEQ ID NO: 2142 | SsSNP2006B_2142 | 121 | A | G |
| SEQ ID NO: 2143 | SsSNP2006B_2143 | 121 | A | G |
| SEQ ID NO: 2144 | SsSNP2006B_2144 | 121 | A | G |
| SEQ ID NO: 2145 | SsSNP2006B_2145 | 121 | A | C |
| SEQ ID NO: 2146 | SsSNP2006B_2146 | 121 | A | G |
| SEQ ID NO: 2147 | SsSNP2006B_2147 | 121 | A | C |
| SEQ ID NO: 2148 | SsSNP2006B_2148 | 121 | A | C |
| SEQ ID NO: 2149 | SsSNP2006B_2149 | 121 | A | G |
| SEQ ID NO: 2150 | SsSNP2006B_2150 | 121 | A | G |
| SEQ ID NO: 2151 | SsSNP2006B_2151 | 121 | A | G |
| SEQ ID NO: 2152 | SsSNP2006B_2152 | 121 | A | G |
| SEQ ID NO: 2153 | SsSNP2006B_2153 | 121 | A | G |
| SEQ ID NO: 2154 | SsSNP2006B_2154 | 121 | A | G |
| SEQ ID NO: 2155 | SsSNP2006B_2155 | 121 | A | G |
| SEQ ID NO: 2156 | SsSNP2006B_2156 | 121 | A | G |
| SEQ ID NO: 2157 | SsSNP2006B_2157 | 121 | C | G |
| SEQ ID NO: 2158 | SsSNP2006B_2158 | 121 | A | C |
| SEQ ID NO: 2159 | SsSNP2006B_2159 | 121 | A | G |
| SEQ ID NO: 2160 | SsSNP2006B_2160 | 121 | A | G |
| SEQ ID NO: 2161 | SsSNP2006B_2161 | 122 | A | G |
| SEQ ID NO: 2162 | SsSNP2006B_2162 | 121 | A | C |
| SEQ ID NO: 2163 | SsSNP2006B_2163 | 121 | A | G |
| SEQ ID NO: 2164 | SsSNP2006B_2164 | 121 | A | G |
| SEQ ID NO: 2165 | SsSNP2006B_2165 | 121 | A | C |
| SEQ ID NO: 2166 | SsSNP2006B_2166 | 121 | A | T |
| SEQ ID NO: 2167 | SsSNP2006B_2167 | 121 | A | G |
| SEQ ID NO: 2168 | SsSNP2006B_2168 | 121 | C | G |
| SEQ ID NO: 2169 | SsSNP2006B_2169 | 121 | A | G |
| SEQ ID NO: 2170 | SsSNP2006B_2170 | 121 | A | G |
| SEQ ID NO: 2171 | SsSNP2006B_2171 | 121 | A | G |
| SEQ ID NO: 2172 | SsSNP2006B_2172 | 121 | A | G |
| SEQ ID NO: 2173 | SsSNP2006B_2173 | 77 | A | C |
| SEQ ID NO: 2174 | SsSNP2006B_2174 | 121 | C | G |
| SEQ ID NO: 2175 | SsSNP2006B_2175 | 121 | C | G |
| SEQ ID NO: 2176 | SsSNP2006B_2176 | 121 | A | C |
| SEQ ID NO: 2177 | SsSNP2006B_2177 | 121 | A | C |
| SEQ ID NO: 2178 | SsSNP2006B_2178 | 121 | A | C |
| SEQ ID NO: 2179 | SsSNP2006B_2179 | 121 | A | C |
| SEQ ID NO: 2180 | SsSNP2006B_2180 | 121 | A | G |
| SEQ ID NO: 2181 | SsSNP2006B_2181 | 121 | A | G |
| SEQ ID NO: 2182 | SsSNP2006B_2182 | 121 | A | G |
| SEQ ID NO: 2183 | SsSNP2006B_2183 | 121 | C | G |
| SEQ ID NO: 2184 | SsSNP2006B_2184 | 121 | A | G |
| SEQ ID NO: 2185 | SsSNP2006B_2185 | 121 | A | G |
| SEQ ID NO: 2186 | SsSNP2006B_2186 | 121 | A | G |
| SEQ ID NO: 2187 | SsSNP2006B_2187 | 121 | A | G |
| SEQ ID NO: 2188 | SsSNP2006B_2188 | 121 | A | G |
| SEQ ID NO: 2189 | SsSNP2006B_2189 | 121 | A | G |
| SEQ ID NO: 2190 | SsSNP2006B_2190 | 121 | A | C |
| SEQ ID NO: 2191 | SsSNP2006B_2191 | 121 | A | G |
| SEQ ID NO: 2192 | SsSNP2006B_2192 | 121 | A | G |
| SEQ ID NO: 2193 | SsSNP2006B_2193 | 121 | A | C |
| SEQ ID NO: 2194 | SsSNP2006B_2194 | 121 | A | C |
| SEQ ID NO: 2195 | SsSNP2006B_2195 | 121 | A | C |
| SEQ ID NO: 2196 | SsSNP2006B_2196 | 121 | A | G |
| SEQ ID NO: 2197 | SsSNP2006B_2197 | 121 | A | G |
| SEQ ID NO: 2198 | SsSNP2006B_2198 | 121 | A | G |
| SEQ ID NO: 2199 | SsSNP2006B_2199 | 121 | A | G |
| SEQ ID NO: 2200 | SsSNP2006B_2200 | 121 | A | T |
| SEQ ID NO: 2201 | SsSNP2006B_2201 | 121 | A | G |
| SEQ ID NO: 2202 | SsSNP2006B_2202 | 121 | A | G |
| SEQ ID NO: 2203 | SsSNP2006B_2203 | 121 | A | G |
| SEQ ID NO: 2204 | SsSNP2006B_2204 | 114 | C | G |
| SEQ ID NO: 2205 | SsSNP2006B_2205 | 121 | A | C |
| SEQ ID NO: 2206 | SsSNP2006B_2206 | 121 | A | G |
| SEQ ID NO: 2207 | SsSNP2006B_2207 | 121 | A | G |
| SEQ ID NO: 2208 | SsSNP2006B_2208 | 121 | A | G |
| SEQ ID NO: 2209 | SsSNP2006B_2209 | 121 | A | G |
| SEQ ID NO: 2210 | SsSNP2006B_2210 | 121 | A | G |
| SEQ ID NO: 2211 | SsSNP2006B_2211 | 121 | A | T |
| SEQ ID NO: 2212 | SsSNP2006B_2212 | 121 | A | G |
| SEQ ID NO: 2213 | SsSNP2006B_2213 | 121 | A | G |
| SEQ ID NO: 2214 | SsSNP2006B_2214 | 121 | A | G |
| SEQ ID NO: 2215 | SsSNP2006B_2215 | 121 | C | G |
| SEQ ID NO: 2216 | SsSNP2006B_2216 | 121 | A | G |
| SEQ ID NO: 2217 | SsSNP2006B_2217 | 121 | A | G |
| SEQ ID NO: 2218 | SsSNP2006B_2218 | 121 | A | G |
| SEQ ID NO: 2219 | SsSNP2006B_2219 | 121 | A | C |
| SEQ ID NO: 2220 | SsSNP2006B_2220 | 121 | A | G |
| SEQ ID NO: 2221 | SsSNP2006B_2221 | 121 | A | G |
| SEQ ID NO: 2222 | SsSNP2006B_2222 | 121 | A | G |
| SEQ ID NO: 2223 | SsSNP2006B_2223 | 121 | A | C |
| SEQ ID NO: 2224 | SsSNP2006B_2224 | 121 | A | G |
| SEQ ID NO: 2225 | SsSNP2006B_2225 | 121 | A | G |
| SEQ ID NO: 2226 | SsSNP2006B_2226 | 121 | C | G |
| SEQ ID NO: 2227 | SsSNP2006B_2227 | 121 | A | G |
| SEQ ID NO: 2228 | SsSNP2006B_2228 | 121 | A | G |
| SEQ ID NO: 2229 | SsSNP2006B_2229 | 121 | C | G |
| SEQ ID NO: 2230 | SsSNP2006B_2230 | 121 | A | G |
| SEQ ID NO: 2231 | SsSNP2006B_2231 | 121 | A | G |
| SEQ ID NO: 2232 | SsSNP2006B_2232 | 121 | A | G |
| SEQ ID NO: 2233 | SsSNP2006B_2233 | 121 | A | G |
| SEQ ID NO: 2234 | SsSNP2006B_2234 | 121 | A | G |
| SEQ ID NO: 2235 | SsSNP2006B_2235 | 121 | C | G |
| SEQ ID NO: 2236 | SsSNP2006B_2236 | 121 | A | G |
| SEQ ID NO: 2237 | SsSNP2006B_2237 | 121 | A | G |
| SEQ ID NO: 2238 | SsSNP2006B_2238 | 121 | A | G |
| SEQ ID NO: 2239 | SsSNP2006B_2239 | 121 | A | G |
| SEQ ID NO: 2240 | SsSNP2006B_2240 | 121 | A | C |
| SEQ ID NO: 2241 | SsSNP2006B_2241 | 121 | A | G |
| SEQ ID NO: 2242 | SsSNP2006B_2242 | 121 | A | G |
| SEQ ID NO: 2243 | SsSNP2006B_2243 | 121 | A | C |
| SEQ ID NO: 2244 | SsSNP2006B_2244 | 121 | A | G |
| SEQ ID NO: 2245 | SsSNP2006B_2245 | 121 | A | G |
| SEQ ID NO: 2246 | SsSNP2006B_2246 | 121 | A | C |
| SEQ ID NO: 2247 | SsSNP2006B_2247 | 121 | A | G |
| SEQ ID NO: 2248 | SsSNP2006B_2248 | 121 | A | C |
| SEQ ID NO: 2249 | SsSNP2006B_2249 | 121 | A | G |
| SEQ ID NO: 2250 | SsSNP2006B_2250 | 121 | A | G |
| SEQ ID NO: 2251 | SsSNP2006B_2251 | 121 | A | C |
| SEQ ID NO: 2252 | SsSNP2006B_2252 | 121 | A | G |
| SEQ ID NO: 2253 | SsSNP2006B_2253 | 121 | C | G |
| SEQ ID NO: 2254 | SsSNP2006B_2254 | 121 | A | C |
| SEQ ID NO: 2255 | SsSNP2006B_2255 | 121 | A | G |
| SEQ ID NO: 2256 | SsSNP2006B_2256 | 121 | A | G |
| SEQ ID NO: 2257 | SsSNP2006B_2257 | 121 | A | G |
| SEQ ID NO: 2258 | SsSNP2006B_2258 | 121 | A | G |
| SEQ ID NO: 2259 | SsSNP2006B_2259 | 121 | A | G |
| SEQ ID NO: 2260 | SsSNP2006B_2260 | 121 | A | C |
| SEQ ID NO: 2261 | SsSNP2006B_2261 | 121 | A | G |
| SEQ ID NO: 2262 | SsSNP2006B_2262 | 121 | A | C |
| SEQ ID NO: 2263 | SsSNP2006B_2263 | 121 | A | G |
| SEQ ID NO: 2264 | SsSNP2006B_2264 | 121 | A | G |
| SEQ ID NO: 2265 | SsSNP2006B_2265 | 121 | A | G |
| SEQ ID NO: 2266 | SsSNP2006B_2266 | 121 | A | G |
| SEQ ID NO: 2267 | SsSNP2006B_2267 | 121 | A | G |
| SEQ ID NO: 2268 | SsSNP2006B_2268 | 121 | A | C |
| SEQ ID NO: 2269 | SsSNP2006B_2269 | 121 | A | C |
| SEQ ID NO: 2270 | SsSNP2006B_2270 | 121 | A | G |
| SEQ ID NO: 2271 | SsSNP2006B_2271 | 121 | A | G |
| SEQ ID NO: 2272 | SsSNP2006B_2272 | 121 | A | G |
| SEQ ID NO: 2273 | SsSNP2006B_2273 | 121 | A | T |
| SEQ ID NO: 2274 | SsSNP2006B_2274 | 121 | A | G |
| SEQ ID NO: 2275 | SsSNP2006B_2275 | 121 | A | G |
| SEQ ID NO: 2276 | SsSNP2006B_2276 | 121 | A | G |
| SEQ ID NO: 2277 | SsSNP2006B_2277 | 121 | A | G |
| SEQ ID NO: 2278 | SsSNP2006B_2278 | 121 | C | G |
| SEQ ID NO: 2279 | SsSNP2006B_2279 | 121 | A | G |
| SEQ ID NO: 2280 | SsSNP2006B_2280 | 121 | A | C |
| SEQ ID NO: 2281 | SsSNP2006B_2281 | 121 | A | G |
| SEQ ID NO: 2282 | SsSNP2006B_2282 | 121 | A | G |
| SEQ ID NO: 2283 | SsSNP2006B_2283 | 121 | A | G |
| SEQ ID NO: 2284 | SsSNP2006B_2284 | 121 | A | C |
| SEQ ID NO: 2285 | SsSNP2006B_2285 | 121 | A | G |
| SEQ ID NO: 2286 | SsSNP2006B_2286 | 121 | A | C |
| SEQ ID NO: 2287 | SsSNP2006B_2287 | 121 | A | G |
| SEQ ID NO: 2288 | SsSNP2006B_2288 | 121 | A | C |
| SEQ ID NO: 2289 | SsSNP2006B_2289 | 121 | A | G |
| SEQ ID NO: 2290 | SsSNP2006B_2290 | 121 | C | G |
| SEQ ID NO: 2291 | SsSNP2006B_2291 | 121 | A | G |
| SEQ ID NO: 2292 | SsSNP2006B_2292 | 121 | A | G |
| SEQ ID NO: 2293 | SsSNP2006B_2293 | 121 | A | G |
| SEQ ID NO: 2294 | SsSNP2006B_2294 | 121 | A | G |
| SEQ ID NO: 2295 | SsSNP2006B_2295 | 121 | A | G |
| SEQ ID NO: 2296 | SsSNP2006B_2296 | 121 | A | G |
| SEQ ID NO: 2297 | SsSNP2006B_2297 | 121 | A | G |
| SEQ ID NO: 2298 | SsSNP2006B_2298 | 121 | A | G |
| SEQ ID NO: 2299 | SsSNP2006B_2299 | 121 | A | G |
| SEQ ID NO: 2300 | SsSNP2006B_2300 | 121 | C | G |
| SEQ ID NO: 2301 | SsSNP2006B_2301 | 121 | A | G |
| SEQ ID NO: 2302 | SsSNP2006B_2302 | 121 | A | G |
| SEQ ID NO: 2303 | SsSNP2006B_2303 | 41 | A | G |
| SEQ ID NO: 2304 | SsSNP2006B_2304 | 121 | A | G |
| SEQ ID NO: 2305 | SsSNP2006B_2305 | 121 | A | G |
| SEQ ID NO: 2306 | SsSNP2006B_2306 | 121 | A | C |
| SEQ ID NO: 2307 | SsSNP2006B_2307 | 121 | A | G |
| SEQ ID NO: 2308 | SsSNP2006B_2308 | 121 | A | C |
| SEQ ID NO: 2309 | SsSNP2006B_2309 | 121 | A | G |
| SEQ ID NO: 2310 | SsSNP2006B_2310 | 121 | A | C |
| SEQ ID NO: 2311 | SsSNP2006B_2311 | 121 | A | G |
| SEQ ID NO: 2312 | SsSNP2006B_2312 | 121 | A | G |
| SEQ ID NO: 2313 | SsSNP2006B_2313 | 121 | C | G |
| SEQ ID NO: 2314 | SsSNP2006B_2314 | 121 | A | G |
| SEQ ID NO: 2315 | SsSNP2006B_2315 | 121 | A | G |
| SEQ ID NO: 2316 | SsSNP2006B_2316 | 121 | A | C |
| SEQ ID NO: 2317 | SsSNP2006B_2317 | 121 | A | G |
| SEQ ID NO: 2318 | SsSNP2006B_2318 | 121 | A | G |
| SEQ ID NO: 2319 | SsSNP2006B_2319 | 121 | A | G |
| SEQ ID NO: 2320 | SsSNP2006B_2320 | 121 | A | G |
| SEQ ID NO: 2321 | SsSNP2006B_2321 | 121 | A | G |
| SEQ ID NO: 2322 | SsSNP2006B_2322 | 121 | C | G |
| SEQ ID NO: 2323 | SsSNP2006B_2323 | 121 | A | G |
| SEQ ID NO: 2324 | SsSNP2006B_2324 | 121 | A | G |
| SEQ ID NO: 2325 | SsSNP2006B_2325 | 121 | A | G |
| SEQ ID NO: 2326 | SsSNP2006B_2326 | 121 | A | T |
| SEQ ID NO: 2327 | SsSNP2006B_2327 | 121 | A | G |
| SEQ ID NO: 2328 | SsSNP2006B_2328 | 121 | A | G |
| SEQ ID NO: 2329 | SsSNP2006B_2329 | 121 | A | G |
| SEQ ID NO: 2330 | SsSNP2006B_2330 | 121 | A | G |
| SEQ ID NO: 2331 | SsSNP2006B_2331 | 121 | A | G |
| SEQ ID NO: 2332 | SsSNP2006B_2332 | 121 | A | G |
| SEQ ID NO: 2333 | SsSNP2006B_2333 | 121 | A | C |
| SEQ ID NO: 2334 | SsSNP2006B_2334 | 121 | A | G |
| SEQ ID NO: 2335 | SsSNP2006B_2335 | 121 | A | G |
| SEQ ID NO: 2336 | SsSNP2006B_2336 | 121 | A | G |
| SEQ ID NO: 2337 | SsSNP2006B_2337 | 121 | A | G |
| SEQ ID NO: 2338 | SsSNP2006B_2338 | 121 | A | T |
| SEQ ID NO: 2339 | SsSNP2006B_2339 | 121 | A | G |
| SEQ ID NO: 2340 | SsSNP2006B_2340 | 121 | A | G |
| SEQ ID NO: 2341 | SsSNP2006B_2341 | 121 | A | G |
| SEQ ID NO: 2342 | SsSNP2006B_2342 | 121 | A | G |
| SEQ ID NO: 2343 | SsSNP2006B_2343 | 121 | A | G |
| SEQ ID NO: 2344 | SsSNP2006B_2344 | 121 | C | G |
| SEQ ID NO: 2345 | SsSNP2006B_2345 | 121 | C | G |
| SEQ ID NO: 2346 | SsSNP2006B_2346 | 121 | A | G |
| SEQ ID NO: 2347 | SsSNP2006B_2347 | 121 | A | G |
| SEQ ID NO: 2348 | SsSNP2006B_2348 | 121 | A | G |
| SEQ ID NO: 2349 | SsSNP2006B_2349 | 121 | A | G |
| SEQ ID NO: 2350 | SsSNP2006B_2350 | 121 | A | T |
| SEQ ID NO: 2351 | SsSNP2006B_2351 | 121 | A | G |
| SEQ ID NO: 2352 | SsSNP2006B_2352 | 121 | A | G |
| SEQ ID NO: 2353 | SsSNP2006B_2353 | 121 | A | C |
| SEQ ID NO: 2354 | SsSNP2006B_2354 | 121 | A | G |
| SEQ ID NO: 2355 | SsSNP2006B_2355 | 121 | A | G |
| SEQ ID NO: 2356 | SsSNP2006B_2356 | 121 | C | G |
| SEQ ID NO: 2357 | SsSNP2006B_2357 | 121 | A | C |
| SEQ ID NO: 2358 | SsSNP2006B_2358 | 121 | A | C |
| SEQ ID NO: 2359 | SsSNP2006B_2359 | 121 | A | G |
| SEQ ID NO: 2360 | SsSNP2006B_2360 | 121 | A | C |
| SEQ ID NO: 2361 | SsSNP2006B_2361 | 121 | A | T |
| SEQ ID NO: 2362 | SsSNP2006B_2362 | 121 | A | C |
| SEQ ID NO: 2363 | SsSNP2006B_2363 | 121 | A | C |
| SEQ ID NO: 2364 | SsSNP2006B_2364 | 121 | A | G |
| SEQ ID NO: 2365 | SsSNP2006B_2365 | 121 | A | G |
| SEQ ID NO: 2366 | SsSNP2006B_2366 | 121 | A | G |
| SEQ ID NO: 2367 | SsSNP2006B_2367 | 121 | A | G |
| SEQ ID NO: 2368 | SsSNP2006B_2368 | 121 | A | G |
| SEQ ID NO: 2369 | SsSNP2006B_2369 | 121 | A | G |
| SEQ ID NO: 2370 | SsSNP2006B_2370 | 121 | A | G |
| SEQ ID NO: 2371 | SsSNP2006B_2371 | 121 | C | G |
| SEQ ID NO: 2372 | SsSNP2006B_2372 | 121 | A | G |
| SEQ ID NO: 2373 | SsSNP2006B_2373 | 121 | A | G |
| SEQ ID NO: 2374 | SsSNP2006B_2374 | 121 | A | T |
| SEQ ID NO: 2375 | SsSNP2006B_2375 | 121 | A | G |
| SEQ ID NO: 2376 | SsSNP2006B_2376 | 121 | A | T |
| SEQ ID NO: 2377 | SsSNP2006B_2377 | 121 | A | G |
| SEQ ID NO: 2378 | SsSNP2006B_2378 | 121 | A | G |
| SEQ ID NO: 2379 | SsSNP2006B_2379 | 121 | A | G |
| SEQ ID NO: 2380 | SsSNP2006B_2380 | 121 | A | C |
| SEQ ID NO: 2381 | SsSNP2006B_2381 | 121 | A | G |
| SEQ ID NO: 2382 | SsSNP2006B_2382 | 121 | A | G |
| SEQ ID NO: 2383 | SsSNP2006B_2383 | 121 | A | G |
| SEQ ID NO: 2384 | SsSNP2006B_2384 | 121 | A | T |
| SEQ ID NO: 2385 | SsSNP2006B_2385 | 121 | C | G |
| SEQ ID NO: 2386 | SsSNP2006B_2386 | 121 | A | G |
| SEQ ID NO: 2387 | SsSNP2006B_2387 | 121 | A | G |
| SEQ ID NO: 2388 | SsSNP2006B_2388 | 121 | A | G |
| SEQ ID NO: 2389 | SsSNP2006B_2389 | 121 | A | G |
| SEQ ID NO: 2390 | SsSNP2006B_2390 | 121 | A | G |
| SEQ ID NO: 2391 | SsSNP2006B_2391 | 121 | A | G |
| SEQ ID NO: 2392 | SsSNP2006B_2392 | 121 | C | G |
| SEQ ID NO: 2393 | SsSNP2006B_2393 | 121 | A | G |
| SEQ ID NO: 2394 | SsSNP2006B_2394 | 121 | A | G |
| SEQ ID NO: 2395 | SsSNP2006B_2395 | 121 | A | G |
| SEQ ID NO: 2396 | SsSNP2006B_2396 | 121 | A | G |
| SEQ ID NO: 2397 | SsSNP2006B_2397 | 121 | A | G |
| SEQ ID NO: 2398 | SsSNP2006B_2398 | 121 | A | G |
| SEQ ID NO: 2399 | SsSNP2006B_2399 | 121 | A | G |
| SEQ ID NO: 2400 | SsSNP2006B_2400 | 121 | A | T |
| SEQ ID NO: 2401 | SsSNP2006B_2401 | 121 | A | G |
| SEQ ID NO: 2402 | SsSNP2006B_2402 | 121 | A | G |
| SEQ ID NO: 2403 | SsSNP2006B_2403 | 121 | A | G |
| SEQ ID NO: 2404 | SsSNP2006B_2404 | 121 | A | C |
| SEQ ID NO: 2405 | SsSNP2006B_2405 | 121 | A | G |
| SEQ ID NO: 2406 | SsSNP2006B_2406 | 121 | A | C |
| SEQ ID NO: 2407 | SsSNP2006B_2407 | 121 | A | G |
| SEQ ID NO: 2408 | SsSNP2006B_2408 | 121 | C | G |
| SEQ ID NO: 2409 | SsSNP2006B_2409 | 121 | A | C |
| SEQ ID NO: 2410 | SsSNP2006B_2410 | 121 | A | G |
| SEQ ID NO: 2411 | SsSNP2006B_2411 | 121 | A | G |
| SEQ ID NO: 2412 | SsSNP2006B_2412 | 121 | A | G |
| SEQ ID NO: 2413 | SsSNP2006B_2413 | 121 | A | G |
| SEQ ID NO: 2414 | SsSNP2006B_2414 | 121 | A | G |
| SEQ ID NO: 2415 | SsSNP2006B_2415 | 121 | A | C |
| SEQ ID NO: 2416 | SsSNP2006B_2416 | 121 | A | G |
| SEQ ID NO: 2417 | SsSNP2006B_2417 | 121 | A | G |
| SEQ ID NO: 2418 | SsSNP2006B_2418 | 121 | A | G |
| SEQ ID NO: 2419 | SsSNP2006B_2419 | 121 | A | G |
| SEQ ID NO: 2420 | SsSNP2006B_2420 | 121 | C | G |
| SEQ ID NO: 2421 | SsSNP2006B_2421 | 121 | A | T |
| SEQ ID NO: 2422 | SsSNP2006B_2422 | 121 | A | G |
| SEQ ID NO: 2423 | SsSNP2006B_2423 | 121 | C | G |
| SEQ ID NO: 2424 | SsSNP2006B_2424 | 121 | A | T |
| SEQ ID NO: 2425 | SsSNP2006B_2425 | 121 | A | G |
| SEQ ID NO: 2426 | SsSNP2006B_2426 | 121 | A | G |
| SEQ ID NO: 2427 | SsSNP2006B_2427 | 121 | A | G |
| SEQ ID NO: 2428 | SsSNP2006B_2428 | 121 | A | G |
| SEQ ID NO: 2429 | SsSNP2006B_2429 | 121 | A | G |
| SEQ ID NO: 2430 | SsSNP2006B_2430 | 121 | A | G |
| SEQ ID NO: 2431 | SsSNP2006B_2431 | 121 | A | G |
| SEQ ID NO: 2432 | SsSNP2006B_2432 | 121 | A | C |
| SEQ ID NO: 2433 | SsSNP2006B_2433 | 121 | A | G |
| SEQ ID NO: 2434 | SsSNP2006B_2434 | 121 | A | G |
| SEQ ID NO: 2435 | SsSNP2006B_2435 | 121 | A | G |
| SEQ ID NO: 2436 | SsSNP2006B_2436 | 121 | A | G |
| SEQ ID NO: 2437 | SsSNP2006B_2437 | 121 | A | C |
| SEQ ID NO: 2438 | SsSNP2006B_2438 | 121 | A | G |
| SEQ ID NO: 2439 | SsSNP2006B_2439 | 121 | A | G |
| SEQ ID NO: 2440 | SsSNP2006B_2440 | 121 | A | G |
| SEQ ID NO: 2441 | SsSNP2006B_2441 | 121 | A | G |
| SEQ ID NO: 2442 | SsSNP2006B_2442 | 121 | A | T |
| SEQ ID NO: 2443 | SsSNP2006B_2443 | 121 | A | C |
| SEQ ID NO: 2444 | SsSNP2006B_2444 | 121 | A | C |
| SEQ ID NO: 2445 | SsSNP2006B_2445 | 121 | A | G |
| SEQ ID NO: 2446 | SsSNP2006B_2446 | 121 | A | G |
| SEQ ID NO: 2447 | SsSNP2006B_2447 | 121 | A | C |
| SEQ ID NO: 2448 | SsSNP2006B_2448 | 121 | A | G |
| SEQ ID NO: 2449 | SsSNP2006B_2449 | 121 | A | G |
| SEQ ID NO: 2450 | SsSNP2006B_2450 | 121 | A | G |
| SEQ ID NO: 2451 | SsSNP2006B_2451 | 121 | A | G |
| SEQ ID NO: 2452 | SsSNP2006B_2452 | 121 | C | G |
| SEQ ID NO: 2453 | SsSNP2006B_2453 | 121 | A | G |
| SEQ ID NO: 2454 | SsSNP2006B_2454 | 121 | A | G |
| SEQ ID NO: 2455 | SsSNP2006B_2455 | 121 | A | C |
| SEQ ID NO: 2456 | SsSNP2006B_2456 | 121 | A | C |
| SEQ ID NO: 2457 | SsSNP2006B_2457 | 121 | A | G |
| SEQ ID NO: 2458 | SsSNP2006B_2458 | 121 | A | G |
| SEQ ID NO: 2459 | SsSNP2006B_2459 | 121 | A | G |
| SEQ ID NO: 2460 | SsSNP2006B_2460 | 121 | A | C |
| SEQ ID NO: 2461 | SsSNP2006B_2461 | 121 | A | G |
| SEQ ID NO: 2462 | SsSNP2006B_2462 | 121 | A | G |
| SEQ ID NO: 2463 | SsSNP2006B_2463 | 121 | A | T |
| SEQ ID NO: 2464 | SsSNP2006B_2464 | 121 | A | G |
| SEQ ID NO: 2465 | SsSNP2006B_2465 | 121 | A | C |
| SEQ ID NO: 2466 | SsSNP2006B_2466 | 121 | A | G |
| SEQ ID NO: 2467 | SsSNP2006B_2467 | 121 | A | G |
| SEQ ID NO: 2468 | SsSNP2006B_2468 | 121 | A | G |
| SEQ ID NO: 2469 | SsSNP2006B_2469 | 121 | A | G |
| SEQ ID NO: 2470 | SsSNP2006B_2470 | 121 | A | G |
| SEQ ID NO: 2471 | SsSNP2006B_2471 | 121 | C | G |
| SEQ ID NO: 2472 | SsSNP2006B_2472 | 121 | A | C |
| SEQ ID NO: 2473 | SsSNP2006B_2473 | 121 | A | G |
| SEQ ID NO: 2474 | SsSNP2006B_2474 | 121 | A | G |
| SEQ ID NO: 2475 | SsSNP2006B_2475 | 121 | A | G |
| SEQ ID NO: 2476 | SsSNP2006B_2476 | 121 | A | G |
| SEQ ID NO: 2477 | SsSNP2006B_2477 | 121 | A | G |
| SEQ ID NO: 2478 | SsSNP2006B_2478 | 121 | A | G |
| SEQ ID NO: 2479 | SsSNP2006B_2479 | 121 | A | T |
| SEQ ID NO: 2480 | SsSNP2006B_2480 | 121 | A | G |
| SEQ ID NO: 2481 | SsSNP2006B_2481 | 121 | A | G |
| SEQ ID NO: 2482 | SsSNP2006B_2482 | 121 | A | G |
| SEQ ID NO: 2483 | SsSNP2006B_2483 | 121 | A | G |
| SEQ ID NO: 2484 | SsSNP2006B_2484 | 121 | A | G |
| SEQ ID NO: 2485 | SsSNP2006B_2485 | 121 | A | G |
| SEQ ID NO: 2486 | SsSNP2006B_2486 | 121 | A | G |
| SEQ ID NO: 2487 | SsSNP2006B_2487 | 121 | A | G |
| SEQ ID NO: 2488 | SsSNP2006B_2488 | 121 | A | G |
| SEQ ID NO: 2489 | SsSNP2006B_2489 | 121 | A | T |
| SEQ ID NO: 2490 | SsSNP2006B_2490 | 121 | A | G |
| SEQ ID NO: 2491 | SsSNP2006B_2491 | 121 | A | G |
| SEQ ID NO: 2492 | SsSNP2006B_2492 | 121 | A | T |
| SEQ ID NO: 2493 | SsSNP2006B_2493 | 121 | A | G |
| SEQ ID NO: 2494 | SsSNP2006B_2494 | 121 | A | G |
| SEQ ID NO: 2495 | SsSNP2006B_2495 | 121 | A | G |
| SEQ ID NO: 2496 | SsSNP2006B_2496 | 121 | A | G |
| SEQ ID NO: 2497 | SsSNP2006B_2497 | 121 | A | G |
| SEQ ID NO: 2498 | SsSNP2006B_2498 | 121 | A | G |
| SEQ ID NO: 2499 | SsSNP2006B_2499 | 121 | A | G |
| SEQ ID NO: 2500 | SsSNP2006B_2500 | 121 | A | G |
| SEQ ID NO: 2501 | SsSNP2006B_2501 | 121 | A | G |
| SEQ ID NO: 2502 | SsSNP2006B_2502 | 121 | A | G |
| SEQ ID NO: 2503 | SsSNP2006B_2503 | 121 | A | G |
| SEQ ID NO: 2504 | SsSNP2006B_2504 | 121 | A | G |
| SEQ ID NO: 2505 | SsSNP2006B_2505 | 122 | A | G |
| SEQ ID NO: 2506 | SsSNP2006B_2506 | 121 | A | G |
| SEQ ID NO: 2507 | SsSNP2006B_2507 | 121 | A | G |
| SEQ ID NO: 2508 | SsSNP2006B_2508 | 121 | A | G |
| SEQ ID NO: 2509 | SsSNP2006B_2509 | 121 | A | G |
| SEQ ID NO: 2510 | SsSNP2006B_2510 | 121 | A | G |
| SEQ ID NO: 2511 | SsSNP2006B_2511 | 121 | A | G |
| SEQ ID NO: 2512 | SsSNP2006B_2512 | 121 | A | G |
| SEQ ID NO: 2513 | SsSNP2006B_2513 | 121 | A | C |
| SEQ ID NO: 2514 | SsSNP2006B_2514 | 121 | A | G |
| SEQ ID NO: 2515 | SsSNP2006B_2515 | 121 | A | C |
| SEQ ID NO: 2516 | SsSNP2006B_2516 | 121 | A | C |
| SEQ ID NO: 2517 | SsSNP2006B_2517 | 121 | A | G |
| SEQ ID NO: 2518 | SsSNP2006B_2518 | 121 | A | G |
| SEQ ID NO: 2519 | SsSNP2006B_2519 | 121 | A | G |
| SEQ ID NO: 2520 | SsSNP2006B_2520 | 121 | A | G |
| SEQ ID NO: 2521 | SsSNP2006B_2521 | 121 | A | G |
| SEQ ID NO: 2522 | SsSNP2006B_2522 | 121 | A | G |
| SEQ ID NO: 2523 | SsSNP2006B_2523 | 121 | A | G |
| SEQ ID NO: 2524 | SsSNP2006B_2524 | 121 | A | G |
| SEQ ID NO: 2525 | SsSNP2006B_2525 | 121 | A | C |
| SEQ ID NO: 2526 | SsSNP2006B_2526 | 121 | A | C |
| SEQ ID NO: 2527 | SsSNP2006B_2527 | 121 | A | C |
| SEQ ID NO: 2528 | SsSNP2006B_2528 | 121 | A | C |
| SEQ ID NO: 2529 | SsSNP2006B_2529 | 121 | A | G |
| SEQ ID NO: 2530 | SsSNP2006B_2530 | 121 | A | G |
| SEQ ID NO: 2531 | SsSNP2006B_2531 | 121 | A | G |
| SEQ ID NO: 2532 | SsSNP2006B_2532 | 121 | A | G |
| SEQ ID NO: 2533 | SsSNP2006B_2533 | 121 | A | G |
| SEQ ID NO: 2534 | SsSNP2006B_2534 | 121 | C | G |
| SEQ ID NO: 2535 | SsSNP2006B_2535 | 121 | A | G |
| SEQ ID NO: 2536 | SsSNP2006B_2536 | 121 | A | G |
| SEQ ID NO: 2537 | SsSNP2006B_2537 | 121 | A | C |
| SEQ ID NO: 2538 | SsSNP2006B_2538 | 121 | A | C |
| SEQ ID NO: 2539 | SsSNP2006B_2539 | 121 | A | T |
| SEQ ID NO: 2540 | SsSNP2006B_2540 | 121 | A | G |
| SEQ ID NO: 2541 | SsSNP2006B_2541 | 121 | A | G |
| SEQ ID NO: 2542 | SsSNP2006B_2542 | 121 | A | G |
| SEQ ID NO: 2543 | SsSNP2006B_2543 | 121 | A | C |
| SEQ ID NO: 2544 | SsSNP2006B_2544 | 121 | A | G |
| SEQ ID NO: 2545 | SsSNP2006B_2545 | 121 | A | G |
| SEQ ID NO: 2546 | SsSNP2006B_2546 | 121 | A | G |
| SEQ ID NO: 2547 | SsSNP2006B_2547 | 121 | A | G |
| SEQ ID NO: 2548 | SsSNP2006B_2548 | 121 | A | G |
| SEQ ID NO: 2549 | SsSNP2006B_2549 | 121 | A | G |
| SEQ ID NO: 2550 | SsSNP2006B_2550 | 121 | A | G |
| SEQ ID NO: 2551 | SsSNP2006B_2551 | 121 | A | C |
| SEQ ID NO: 2552 | SsSNP2006B_2552 | 121 | A | G |
| SEQ ID NO: 2553 | SsSNP2006B_2553 | 121 | A | G |
| SEQ ID NO: 2554 | SsSNP2006B_2554 | 121 | A | C |
| SEQ ID NO: 2555 | SsSNP2006B_2555 | 121 | A | G |
| SEQ ID NO: 2556 | SsSNP2006B_2556 | 121 | A | G |
| SEQ ID NO: 2557 | SsSNP2006B_2557 | 121 | A | T |
| SEQ ID NO: 2558 | SsSNP2006B_2558 | 121 | A | G |
| SEQ ID NO: 2559 | SsSNP2006B_2559 | 121 | A | G |
| SEQ ID NO: 2560 | SsSNP2006B_2560 | 121 | A | G |
| SEQ ID NO: 2561 | SsSNP2006B_2561 | 121 | A | G |
| SEQ ID NO: 2562 | SsSNP2006B_2562 | 121 | A | G |
| SEQ ID NO: 2563 | SsSNP2006B_2563 | 121 | A | G |
| SEQ ID NO: 2564 | SsSNP2006B_2564 | 121 | A | G |
| SEQ ID NO: 2565 | SsSNP2006B_2565 | 121 | A | C |
| SEQ ID NO: 2566 | SsSNP2006B_2566 | 121 | A | G |
| SEQ ID NO: 2567 | SsSNP2006B_2567 | 121 | C | G |
| SEQ ID NO: 2568 | SsSNP2006B_2568 | 121 | A | T |
| SEQ ID NO: 2569 | SsSNP2006B_2569 | 121 | A | G |
| SEQ ID NO: 2570 | SsSNP2006B_2570 | 115 | A | G |
| SEQ ID NO: 2571 | SsSNP2006B_2571 | 121 | A | G |
| SEQ ID NO: 2572 | SsSNP2006B_2572 | 121 | A | G |
| SEQ ID NO: 2573 | SsSNP2006B_2573 | 121 | A | T |
| SEQ ID NO: 2574 | SsSNP2006B_2574 | 121 | A | G |
| SEQ ID NO: 2575 | SsSNP2006B_2575 | 121 | A | G |
| SEQ ID NO: 2576 | SsSNP2006B_2576 | 121 | A | G |
| SEQ ID NO: 2577 | SsSNP2006B_2577 | 104 | A | C |
| SEQ ID NO: 2578 | SsSNP2006B_2578 | 121 | A | G |
| SEQ ID NO: 2579 | SsSNP2006B_2579 | 121 | A | G |
| SEQ ID NO: 2580 | SsSNP2006B_2580 | 121 | A | G |
| SEQ ID NO: 2581 | SsSNP2006B_2581 | 121 | A | G |
| SEQ ID NO: 2582 | SsSNP2006B_2582 | 121 | C | G |
| SEQ ID NO: 2583 | SsSNP2006B_2583 | 121 | A | G |
| SEQ ID NO: 2584 | SsSNP2006B_2584 | 121 | A | G |
| SEQ ID NO: 2585 | SsSNP2006B_2585 | 121 | A | G |
| SEQ ID NO: 2586 | SsSNP2006B_2586 | 121 | A | G |
| SEQ ID NO: 2587 | SsSNP2006B_2587 | 121 | A | G |
| SEQ ID NO: 2588 | SsSNP2006B_2588 | 121 | A | G |
| SEQ ID NO: 2589 | SsSNP2006B_2589 | 121 | A | G |
| SEQ ID NO: 2590 | SsSNP2006B_2590 | 121 | A | G |
| SEQ ID NO: 2591 | SsSNP2006B_2591 | 121 | A | G |
| SEQ ID NO: 2592 | SsSNP2006B_2592 | 111 | A | G |
| SEQ ID NO: 2593 | SsSNP2006B_2593 | 121 | A | G |
| SEQ ID NO: 2594 | SsSNP2006B_2594 | 121 | A | G |
| SEQ ID NO: 2595 | SsSNP2006B_2595 | 121 | A | G |
| SEQ ID NO: 2596 | SsSNP2006B_2596 | 121 | A | G |
| SEQ ID NO: 2597 | SsSNP2006B_2597 | 121 | A | G |
| SEQ ID NO: 2598 | SsSNP2006B_2598 | 72 | A | G |
| SEQ ID NO: 2599 | SsSNP2006B_2599 | 121 | A | C |
| SEQ ID NO: 2600 | SsSNP2006B_2600 | 121 | A | G |
| SEQ ID NO: 2601 | SsSNP2006B_2601 | 121 | A | G |

What is claimed is:

1. A method of evaluating an animal's genotype at one or more genomic locus/loci, the method comprising determining the animal's genotype for at least one locus; comprising a single nucleotide polymorphism (SNP) having at least two allelic variants; wherein at least one SNP is selected from the SNPs described in Tables 1 and 2.

2. The method of claim 1 wherein the animal's genotype is evaluated at 10 or more positions that contain SNPs selected from the SNPs described in Tables 1 and 2.

3. The method of claim 1 wherein the animal's genotype is evaluated at 50 or more positions that contain SNPs selected from the SNPs described in Tables 1 and 2.

4. The method of claim 1 wherein the animal's genotype is evaluated at 100 or more positions that contain SNPs selected from the SNPs described in Tables 1 and 2.

5. The method of claim 1 wherein the animal's genotype is evaluated at 200 or more positions that contain SNPs selected from the SNPs described in Tables 1 and 2.

6. A method for allocating animals for use comprising:
a. determining at least one animal's genotype at one or locus/loci; wherein the at least one locus contains a single nucleotide polymorphism (SNP), having at least two allelic variants; and wherein at least one SNP is selected from the SNPs described in Tables 1 and 2;
b. analyzing the determined genotype of at least one evaluated animal at one or more SNPs selected from the SNPs described in Tables 1 and 2 to determine which allelic variant is present;
c. allocating the animal for use according to its determined genotype.

7. The method of claim 6 wherein at least one animal's genotype is evaluated at 10 or more loci that contain SNPs selected from the SNPs described in Tables 1 and 2.

8. The method of claim 6 wherein at least one animal's genotype is evaluated at 50 or more loci that contain SNPs selected from the SNPs described in Tables 1 and 2.

9. The method of claim 6 wherein at least one animal's genotype is evaluated at 100 or more loci that contain SNPs selected from the SNPs described in Tables 1 and 2.

10. The method of claim 6 wherein at least one animal's genotype is evaluated at 200 or more loci that contain SNPs selected from the SNPs described in Tables 1 and 2.

11. A method for selecting a potential parent animal for breeding:
a. determining at least one potential parent animal's genotype at one or more genomic locus/loci; wherein at least one locus contains a single nucleotide polymorphism (SNP) that has at least two allelic variants, and wherein at least one SNP is selected from the SNPs described in Tables 1 and 2;
b. analyzing the determined genotype of at least one evaluated animal for one or more SNPs selected from the SNPs described in Tables 1 and 2 to determine which allele is present;
c. correlating the identified allele with a phenotype;
d. allocating at least one animal for breeding use based on its genotype.

12. The method of claim 11 wherein at least one potential parent animal's genotype is evaluated at 10 or more loci that contain SNPs selected from the SNPs described in Tables 1 and 2.

13. The method of claim 11 wherein at least one potential parent animal's genotype is evaluated at 50 or more loci that contain SNPs selected from the SNPs described in Tables 1 and 2.

14. The method of claim 11 wherein at least one potential parent animal's genotype is evaluated at 100 or more loci that contain SNPs selected from the SNPs described in Tables 1 and 2.

15. The method of claim 11 wherein at least one potential parent animal's genotype is evaluated at 200 or more loci that contain SNPs selected from the SNPs described in Tables 1 and 2.

16. A method of producing progeny animals comprising:
a) identifying at least one animal that has been allocating for breeding in accordance with the method of claim 11;
b) producing progeny from the allocated animal through a process comprising:
i) natural breeding;
ii) artificial insemination;
iii) in vitro fertilization; and/or
ii) collecting semen/spermatozoa or at least one ovum from the animal and contacting it, respectively, with ovum/ova or semen/spermatozoa from a second animal to produce a conceptus by any means.

17. The method of claim 16 comprising producing progeny through natural breeding.

18. The method of claim 16 comprising producing offspring through artificial insemination, embryo transfer, and/or in vitro fertilization.

19. The method of claim 16 wherein at least one potential parent animal's genotype is evaluated at 10 or more loci that contain SNPs selected from the SNPs described in Tables t and 2.

20. The method of claim 16 wherein at least one potential parent animal's genotype is evaluated at 50 or more loci that contain SNPs selected from the SNPs described in Tables 1 and 2.

21. The method of claim 16 wherein at least one potential parent animal's genotype is evaluated at 100 or more loci that contain SNPs selected from the SNPs described in Tables 1 and 2.

22. The method of claim 16 wherein at least one potential parent animal's genotype is evaluated at 200 or more loci that contain SNPs selected from the SNPs described in Tables 1 and 2.

23. An isolated nucleic acid comprising at least 17 contiguous nucleotides from an allele of a SNP selected from the group of SNPs described in Tables 1 and 2.

24. An isolated nucleic acid comprising 100 or more contiguous nucleotides with at least 90 percent identity to an allele of a SNP selected from the group of SNPs described in Tables 1 and 2.

25. A diagnostic kit for determining the presence of at least one allele of at least one single nucleotide polymorphism (SNP) having at least two variants; and wherein the SNP is selected from the group of SNPs described in Tables 1 and 2.

26. The kit of claim 25 comprising nucleic acid oligonucleotide primers useful for the polymerase chain reaction-based amplification of at least one allele of at least one SNP.

27. The kit of claim 25 comprising a purified nucleic acid fragment that specifically hybridizes with at least one allele of a SNP under stringent hybridization conditions.

28. A nucleic acid array for determining which allele of at least one of the SNPs described by Tables 1 and 2 are present in a sample; wherein the array comprises one or more nucleic acid sequences capable of hybridizing, under stringent conditions, with at least one variant of at least one SNP selected from the SNPs described in Tables 1 and 2.

29. The array of claim 28 wherein the array is capable of determining which allele is present for each of 10 or more SNPs selected from the group consisting of the SNPs described by Tables 1 and 2.

30. The array of claim 28 wherein the array is capable of determining which allele is present for each of 25 or more SNPs selected from the group consisting of the SNPs described by Tables 1 and 2.

31. The array of claim 28 wherein the array is capable of determining which allele is present for each of 50 or more SNPs selected from the group consisting of the SNPs described by Tables 1 and 2.

32. The array of claim 28 wherein the array is capable of determining which allele is present for each of 100 or more SNPs selected from the group consisting of the SNPs described by Tables 1 and 2.

33. A method of identifying at least one phenotypic trait associated with quantitative trait locus (QTL), the method comprising:
a. measuring one or more phenotypic traits in a plurality of animals;
b. determining the animal's genotype for at least one locus; wherein the locus comprises at least one a single nucleotide polymorphism (SNP) having at least two variants, and wherein the SNP is selected from the group of SNPs described in Tables 1 and 2.
c. statistically correlating the association of at least one phenotypic trait with the presence of an allele of at least one SNP selected from the group of SNPs described in Tables 1 and 2; wherein the presence of a different allele for that SNP has a different association for the phenotypic trait.

34. A method of identifying a genetic marker in allelic association with at least one SNP selected from the group of SNPs described in Tables 1 and 2, the method comprising:
a) identifying a genetic marker B₁ suspected of being in allelic association with a marker A₁ selected from the group of SNPs described in Tables 1 and 2;
b) determining whether A₁ and B₁ are in allelic association;
wherein allelic association exists if r²>0.2 for Equation 1 for a population sample of at least 100 animals and wherein Equation 1 is:
$${r}^{2} = \frac{{[ f ( {A}_{1} {B}_{1} ) - f ( {A}_{1} ) f ( {B}_{1} ) ]}^{2}}{f ( {A}_{1} ) ( 1 - f ( {A}_{1} ) ) ( f ( {B}_{1} ) ( 1 - f ( {B}_{1} ) )} \qquad [ Equation 1 ]$$
and wherein A₁ represents an allele of a SNP described in Tables 1 and 2; B₁ represents a genetic marker at another locus; f(A₁B₁) denotes frequency of having both A₁ and B₁; f(A₁) is the frequency of A₁ in the population; and f(B₁) is the frequency of B₁ in a population.

35. The method of claim 34 wherein the genetic marker B₁ is a SNP.

36. The method of claim 34 wherein the genetic marker identified is in linkage disequilibrium with at least one SNP selected from the group of SNPs described in Tables 1 and 2.

37. The method of claim 34 wherein B₁ is a causal mutation underlying a quantitative trait locus

38. The method of claim 37 wherein r²>0.5.

39. The method of claim 37 wherein r²>0.9.
